(19) United States
(12) Patent Application Publication
(10) Pub. No.: US 2026/0355343 A1
(43) Pub. Date: Nov. 12, 2026

(54) BIS-ALLENE LINKER, LINKER-DRUG CONJUGATE, ANTIBODY-DRUG CONJUGATE, AND USES THEREOF

(71) Applicant: TAICHENGSI (SHANGHAI) BIOMEDICAL CO., LTD., Shanghai (CN)

(72) Inventors: Bin ZOU, Shanghai (CN); Shaojun Liu, Shanghai (CN); Ping Du, Shanghai (CN)

(73) Assignee: TAICHENGSI (SHANGHAI) BIOMEDICAL CO., LTD., Shanghai (CN)

(21) Appl. No.: 19/529,623

(22) Filed: Feb. 4, 2026

Related U.S. Application Data
(63) Continuation of application No. PCT/CN2024/109676, filed on Aug. 6, 2024.

(30) Foreign Application Priority Data
Aug. 4, 2023 (CN) 202310981869.8

(51) Int. Cl. A61K 47/68; A61P 35/00

(52) U.S. Cl. CPC A61K 47/6889; A61K 47/68031; A61K 47/68033; A61P 35/00

(57) ABSTRACT

Provided are a novel bis-allene linker, a linker-drug conjugate, an antibody-drug conjugate, and uses thereof. The bis-allene linker is characterized by the structure represented by Formula (I) disclosed herein and can conveniently achieve site-specific conjugation to antibodies based on covalent thiol bridging, and is applicable to most antibodies. Compared with the cumbersome recombinant modification of antibodies, the method for site-specific conjugation using the bis-allene linker does not require recombinant modification of antibodies, thus saving a great deal of time, costs, and resources, and exhibiting broader application prospects.

### CROSS-REFERENCE TO RELATED APPLICATIONS

This application is a Continuation of PCT International Application No. PCT/CN2024/109676 filed on Aug. 3, 2024, which claims priority to Chinese Patent Application No. 202310981869.8 filed on Aug. 4, 2023. Each of the above applications is hereby expressly incorporated by reference, in its entirety, into the present application.

### TECHNICAL FIELD

The present invention relates to a bis-allene linker, a linker-drug conjugate, an antibody-drug conjugate, and uses thereof.

### BACKGROUND ART

Antibody-drug conjugates (ADCs) derive their design concept from the “Magic bullets” theory first proposed by German scientist Paul Ehrlich in 1913, the core idea of which is the targeted delivery of drugs to lesion sites via suitable carriers. Constrained by limitations in antibody and highly potent cytotoxic drug technologies, it was not until 2000 that the first antibody-drug conjugate (Mylotarg™) for the treatment of acute myeloid leukemia (AML) obtained marketing approval from the FDA. However, with the continuous development of antibody humanization and cleavable linker technologies, and the advent of novel toxin molecules, the FDA has approved 14 antibody-drug conjugates (ADCs) for the treatment of hematological malignancies and solid tumors to date. Despite the remarkable clinical successes achieved by currently marketed antibody-drug conjugates (ADCs), many widely used antibody conjugation methods still suffer from significant limitations. The preparation of homogeneous ADCs remains a challenge to be addressed. Common conjugation approaches rely on chemical reactions between lysine residues or cysteine moieties (via reduction of interchain disulfide bonds) and electrophilic reagents. Owing to the lack of selectivity in the above chemical reactions at present, the conjugation number and sites are uncertain, and thus the resulting conjugates are heterogeneous with multiple components. Moreover, ADCs with different DAR values exhibit different pharmacokinetics, toxicological profiles, and efficacy. Such heterogeneous ADC mixtures are not only difficult to develop but may also be accompanied by undesirable therapeutic windows.

Disulfide bridging is a strategy for the preparation of homogeneous ADCs. A variety of reagents have been employed for disulfide bridging, including bisthiones, next-generation maleimides (NGMs, a class of maleimide reagents modified with two halide or thiophenol leaving groups), pyridazinediones, e.g., dibromomethylquinoxaline, arylene dipropionitriles, divinylpyrimidines (DVP) (Chem. Soc. Rev., 2021, 50, 1305-1353), diacryloyl hexahydrotriazine from RC Biotechnologies, Inc. (CN107921030), bismaleimides from Shanghai New Concept Bio Medical Science and Technology Co., Ltd. (CN103933575), bis(sulfonyl) compounds from OBI Pharma Inc. (CN110290800), and the like. Despite these advances in disulfide bridging, there are still problems such as incomplete homogeneity of components, e.g., DAR4 ADCs are mixtures of “half-antibodies” and “full-antibodies”, failing to achieve either “half-antibodies” or “full-antibodies” as the major component, or the low content of DAR4 ADCs.

In view of the above, there remains an urgent need in the art for an efficient, simple, and practical chemical conjugation method based on disulfide bridging that can achieve site-specific conjugation while maintaining or improving the properties of antibody-drug conjugates, such as efficacy, stability, and safety.

### SUMMARY OF THE INVENTION

The objective of the present invention is to provide a novel bis-allene linker capable of covalent bridging to most antibodies. In addition, the present invention provides an antibody/active agent conjugation method and uses thereof, e.g., in antibody-drug conjugates.

In a first aspect of the present invention, provided is a bis-allene linker, comprising a compound represented by Formula (I) or an isotopically labeled form thereof:

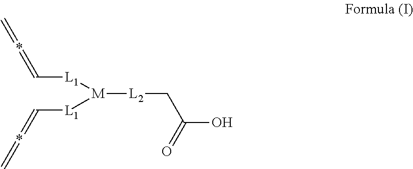

wherein L₁ is selected from

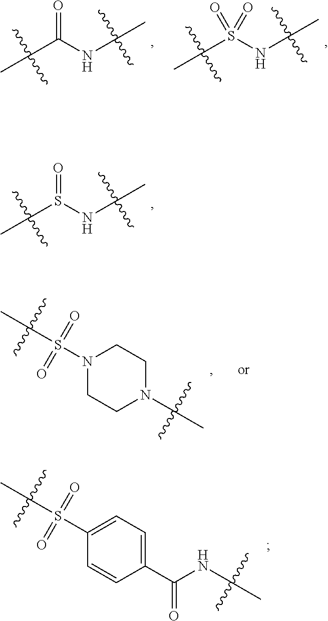 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

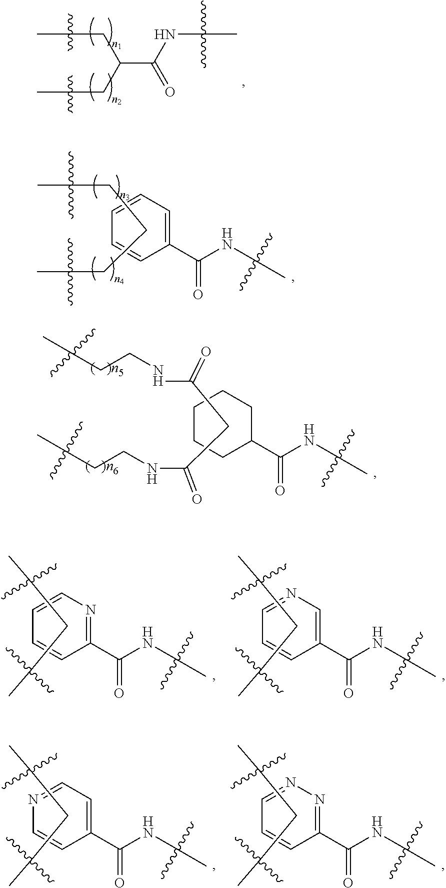

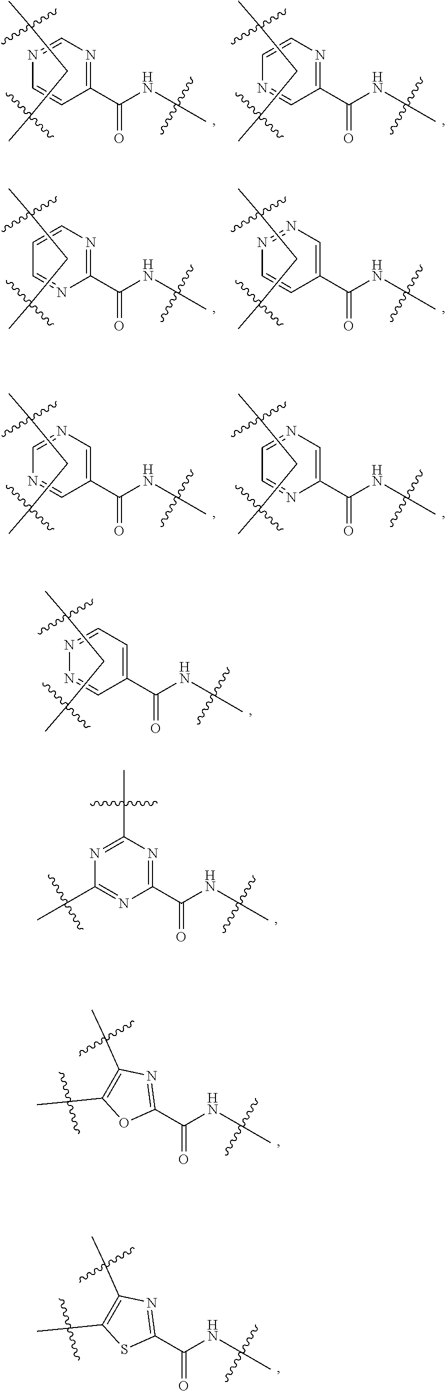

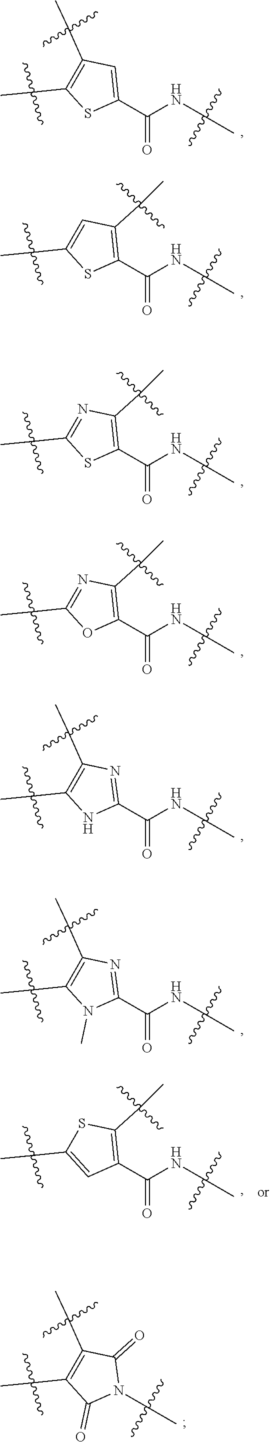

n₁ and n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

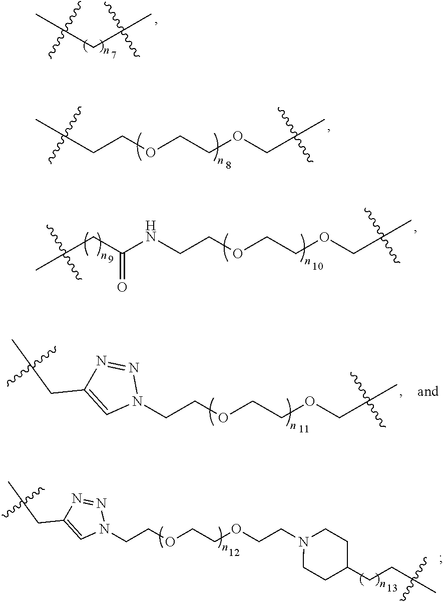

n₇=an integer from 1 to 20, n₈=an integer from 1 to 20, n₉=an integer from 1 to 20, N₁₀=an integer from 1 to 20, n₁₁=an integer from 1 to 20, n₁₂=an integer from 1 to 20, and n₁₃=an integer from 1 to 20.

According to an embodiment of the present invention, in Formula (I),
L₁ is selected from

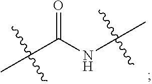 [SMILES: *NC(*)=O]

M is selected from

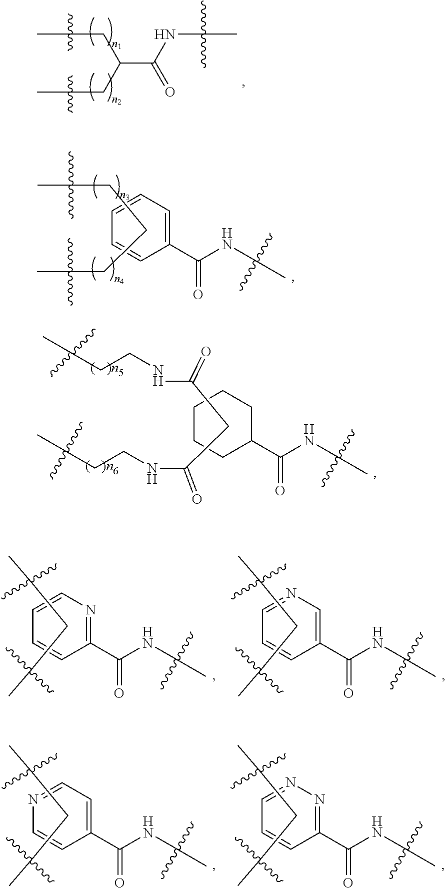

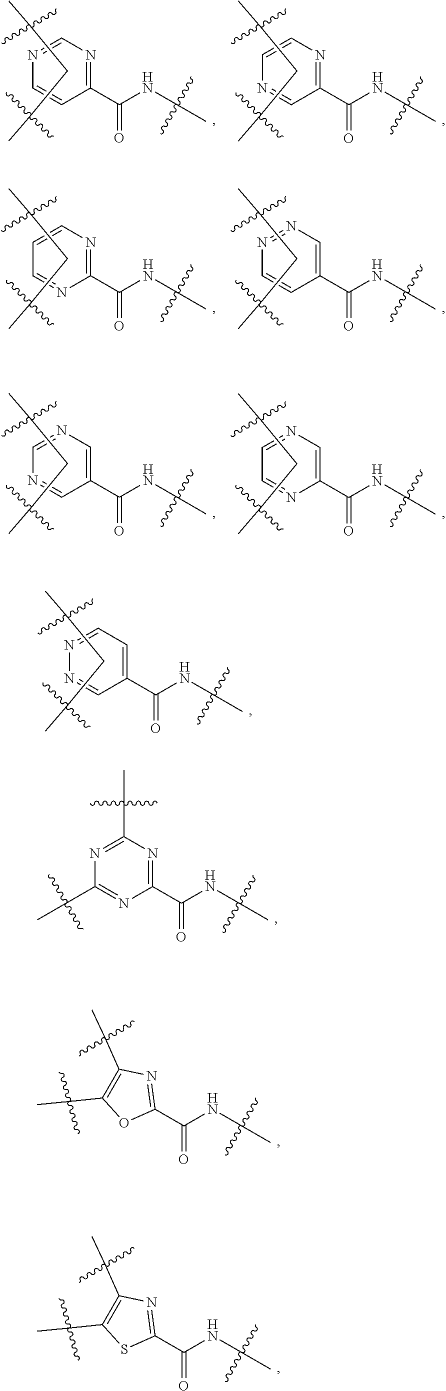

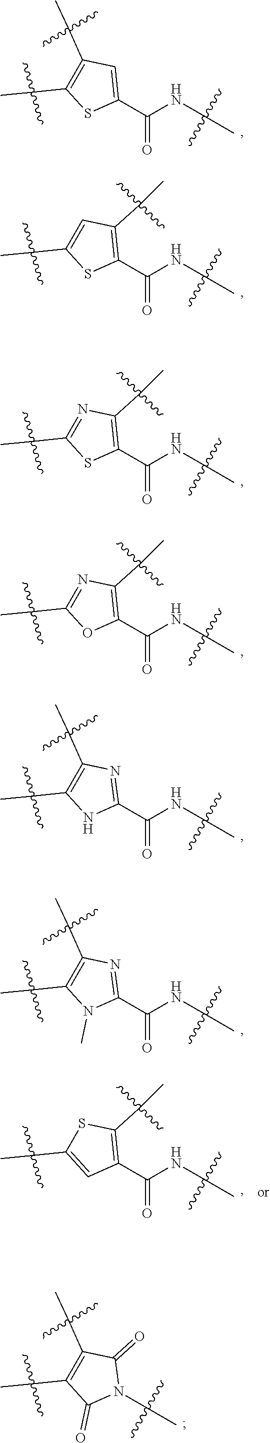

n₁ and n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

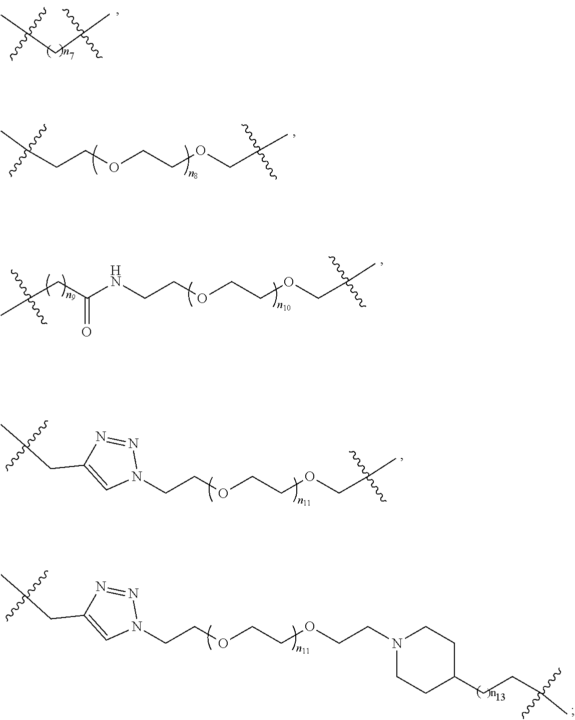

n₇=an integer from 1 to 20, n₈=an integer from 1 to 20, n₉=an integer from 1 to 20, n₁₀=an integer from 1 to 20, n₁₁=an integer from 1 to 20, n₁₂=an integer from 1 to 20, and n₁₃=an integer from 1 to 20.

According to an embodiment of the present invention, in Formula (I),
L₁ is selected from

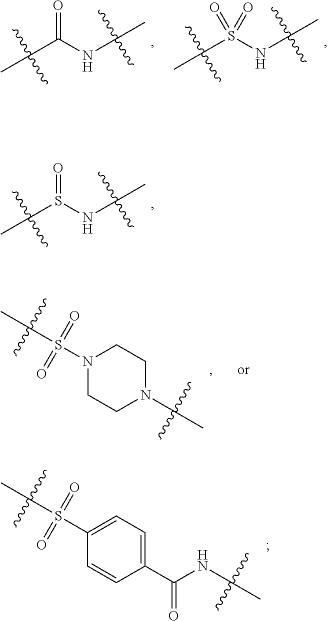 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

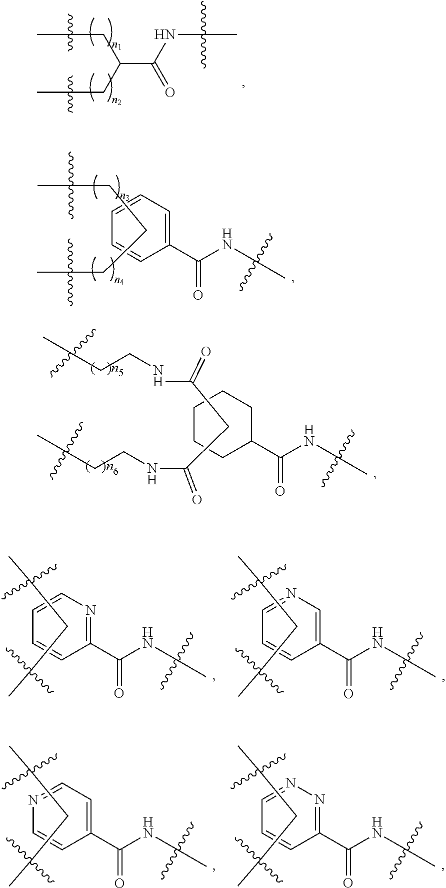

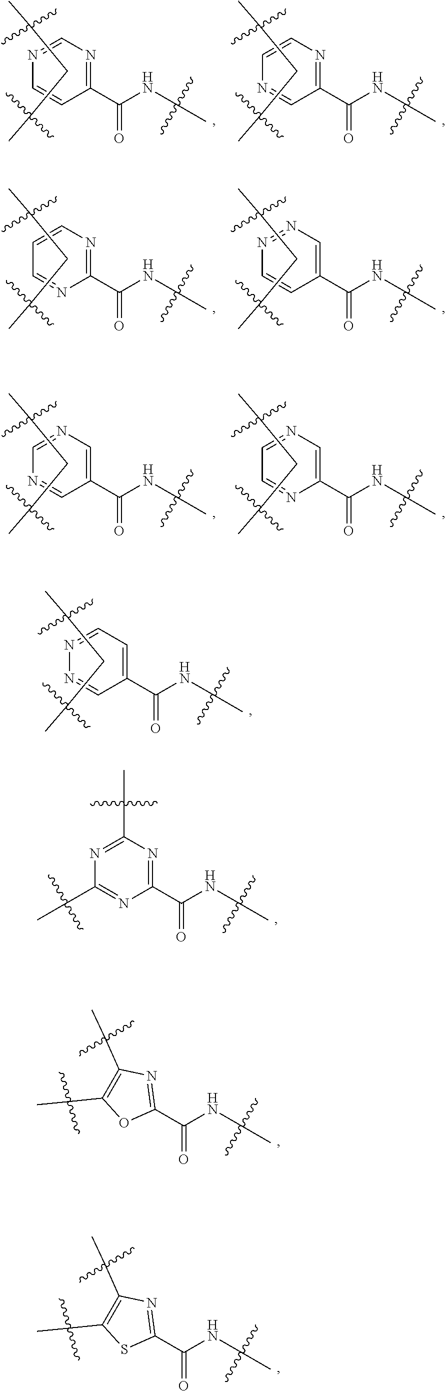

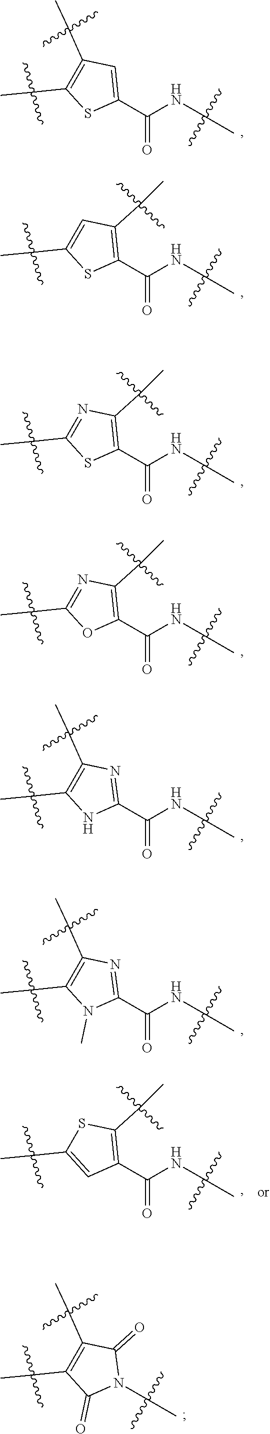

n₁ and n₂ are different, n₃ and n₄ are the same or different, and n₅ and n₆ are the same or different;

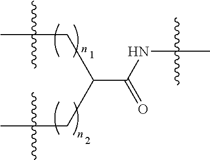

is a single chiral component or a racemate; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

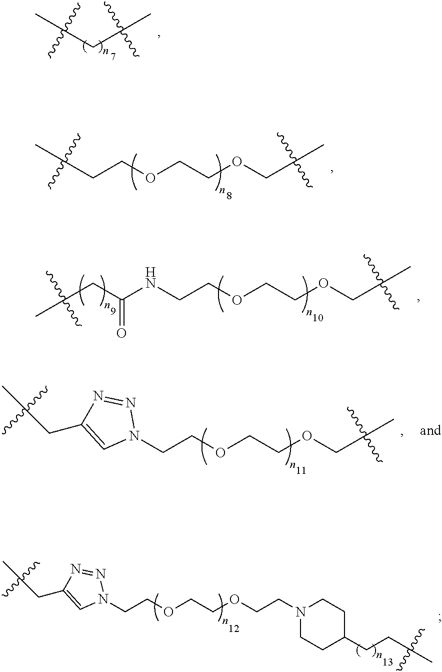

n₇=an integer from 1 to 20, n₈=an integer from 1 to 20, n₉=an integer from 1 to 20, n₁₀=an integer from 1 to 20, n₁=an integer from 1 to 20, n₁₂=an integer from 1 to 20, and n₁₃=an integer from 1 to 20.

According to an embodiment of the present invention, in Formula (I),
L₁ is selected from

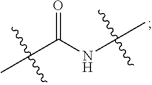 [SMILES: *NC(*)=O]

M is selected from

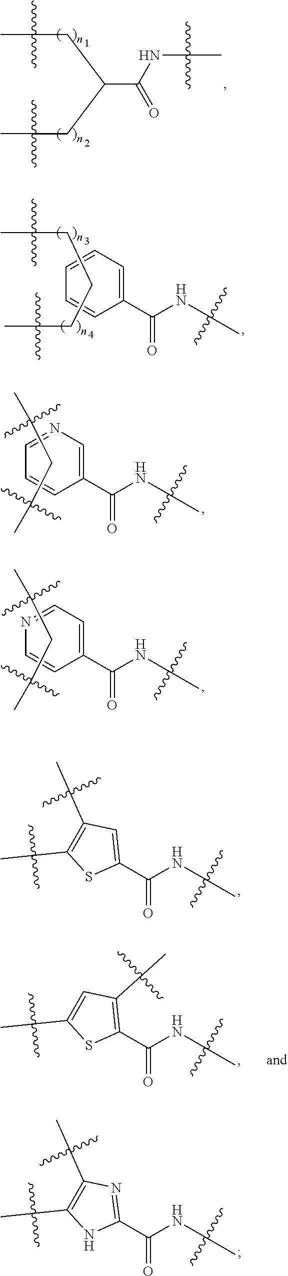

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

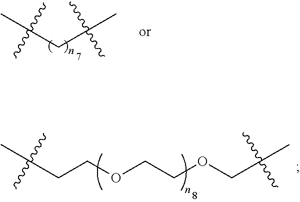

n₇=an integer from 1 to 10, and n₈=an integer from 1 to 10.

According to an embodiment of the present invention, the bis-allene linker of Formula (I) is selected from at least one of the compounds represented by the following structural formulas:

| No. | Structure |
|---|---|
| LK1 | 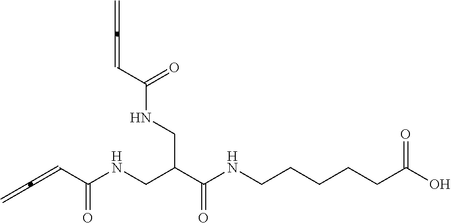 |
| LK2 | 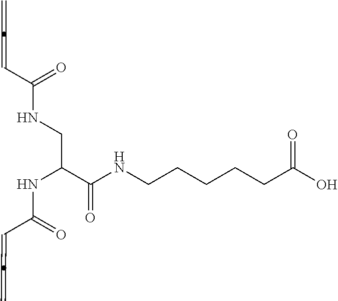 |
| LK3 | 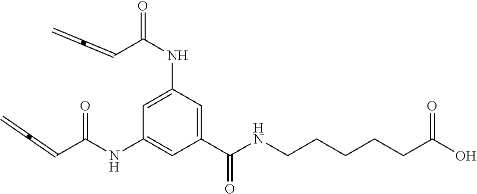 |
| LK4 | 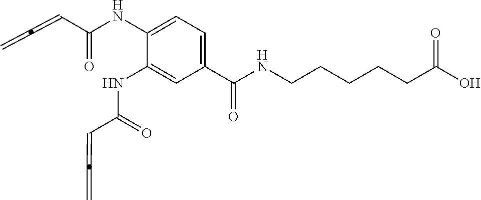 |
| LK5 | 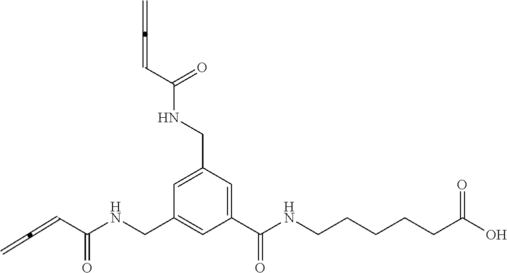 |
| LK6 | 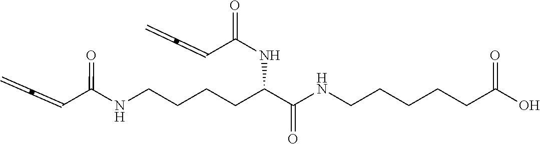 |
| LK7 | 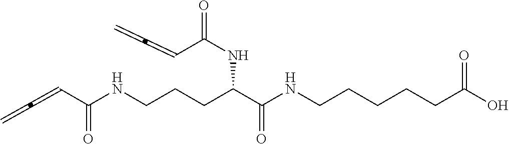 |
| LK8 | 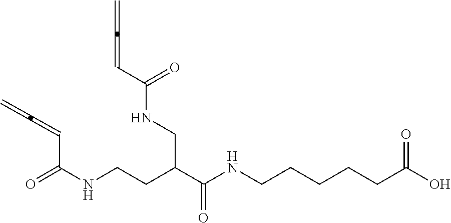 |
| LK9 | 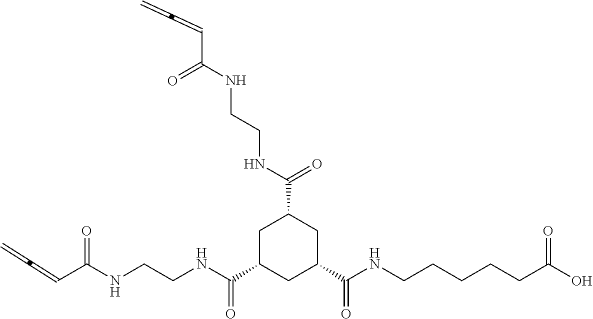 |
| LK10 | 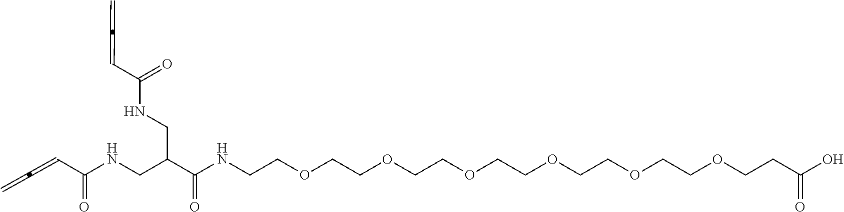 |
| LK11 | 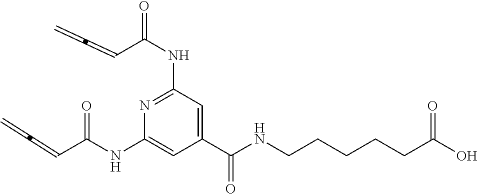 |
| LK12 | 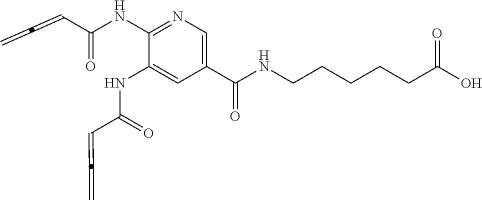 |
| LK13 | 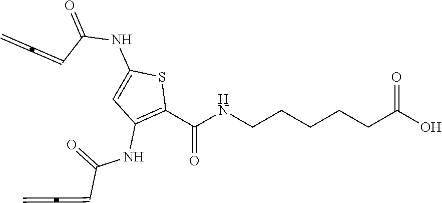 |
| LK14 | 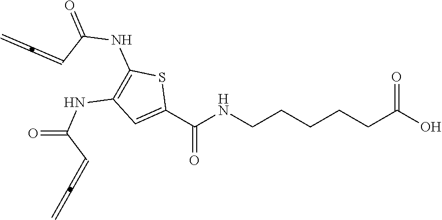 |

In another aspect of the present invention, provided is a linker-drug conjugate, wherein the conjugate is a compound formed by the bonding of the carboxyl group in the bis-allene linker as described above to the amino group in the amino acid residue of L₃ in

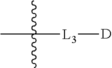

via an amide bond;
L₃ is

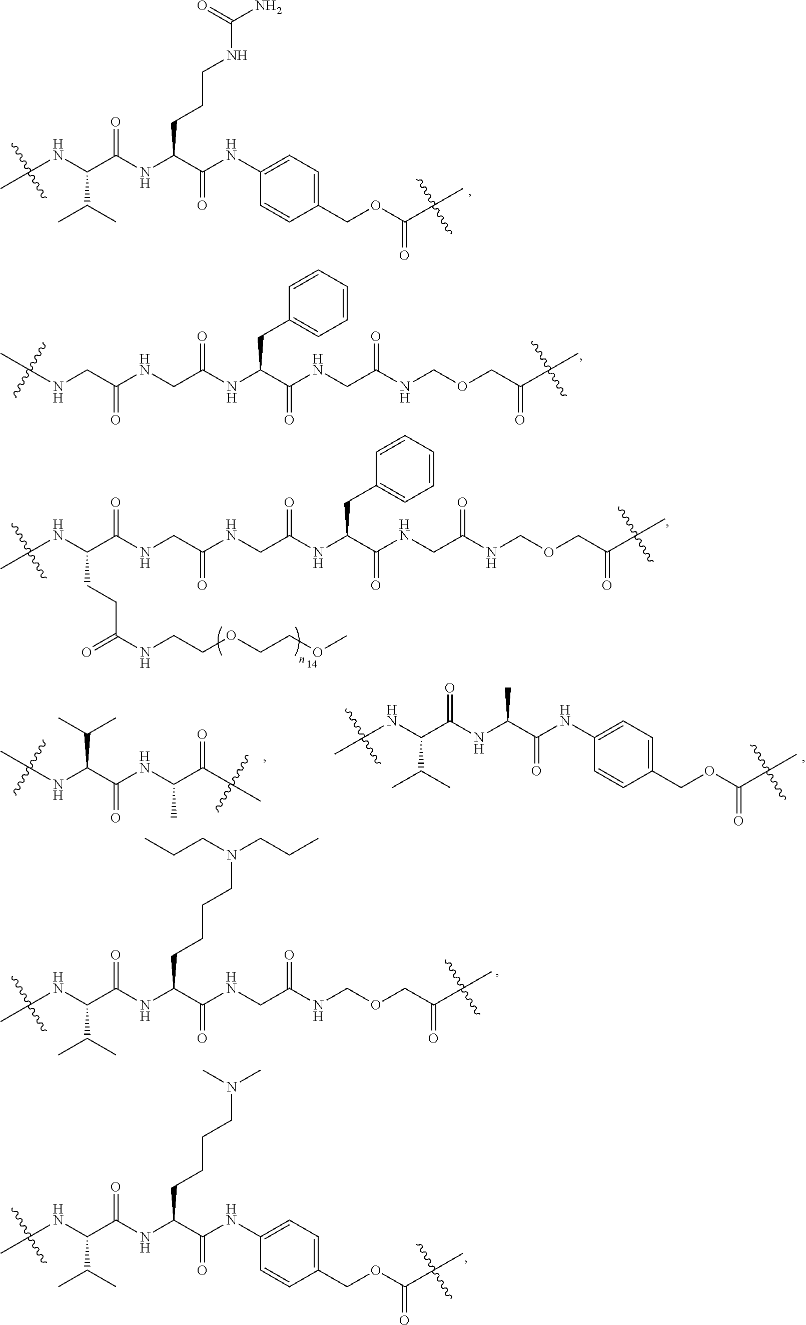

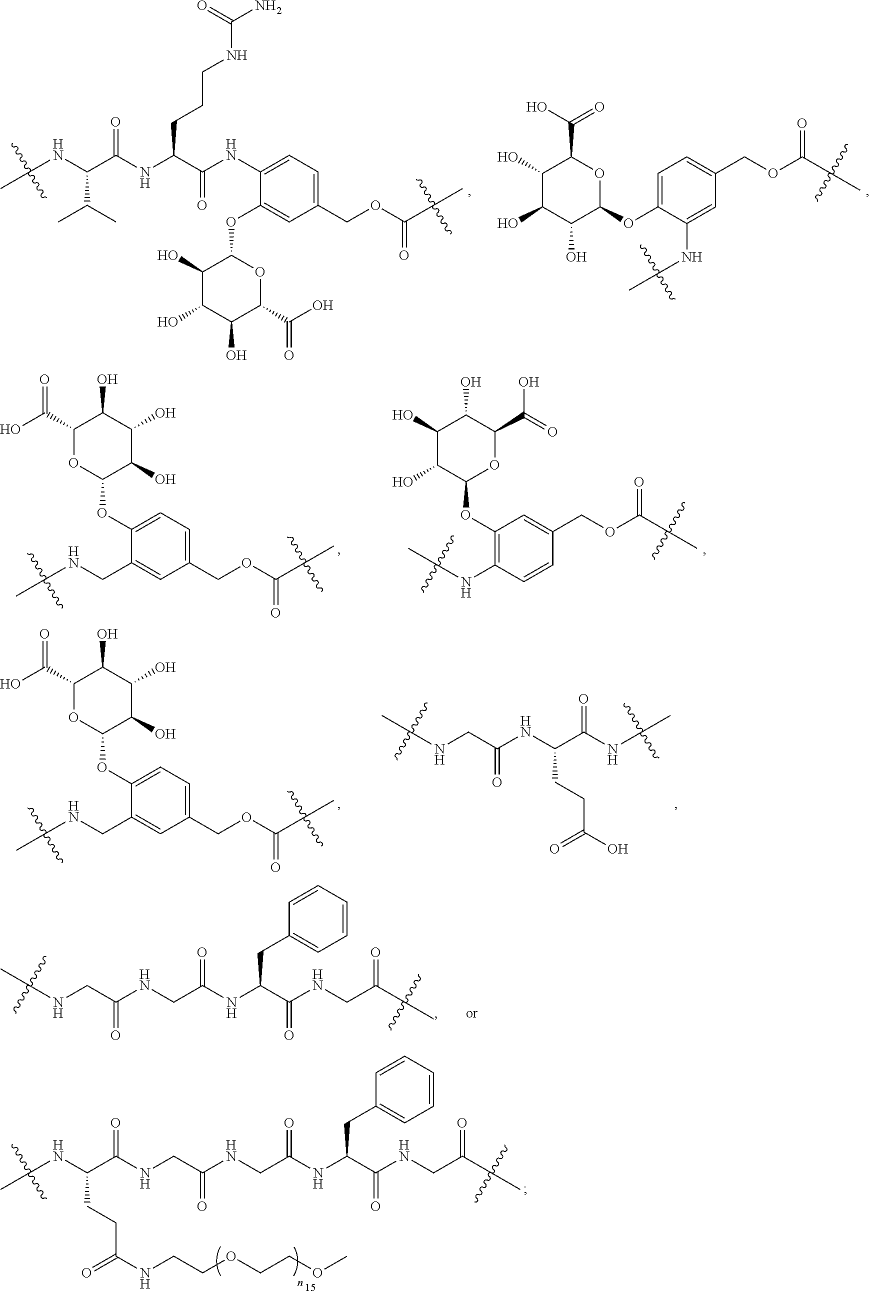

n₁₄=an integer from 1 to 20, and n₁₅=an integer from 1 to 20;
D is a fragment formed by the loss of one or more atoms or groups from a cytotoxic small-molecule drug or active agent, wherein D is covalently bonded to L₃ via sulfur, hydrogen, or oxygen in D.

According to an embodiment of the present invention, the cytotoxic small-molecule drug is a drug for the treatment of tumor diseases; the active agent is a tubulin inhibitor, an RNA polymerase inhibitor, a topoisomerase inhibitor, an intercalator, a DNA-reactive agent, a DNA alkylating agent, or an immunosuppressant.

The tubulin inhibitor includes, but is not limited to, monomethyl auristatin, maytansinoid, maytansinol, or DM1 maytansinoid. The monomethyl auristatin may be monomethyl auristatin E, monomethyl auristatin F, and the like; the RNA polymerase inhibitor includes, but is not limited to, α-amanitin and the like; the topoisomerase inhibitor includes, but is not limited to, etoposide, teniposide, amsacrine, SN-38, exatecan, and the like; the intercalator includes, but is not limited to, pyrrolobenzodiazepine (PBD) and the like; the DNA-reactive agent includes, but is not limited to, calicheamicin, tiancimycins or other enediynes, and the like; the DNA alkylating agent includes, but is not limited to, trabectedin, lurbinectedin, and other ecteinascidin derivatives.

In another aspect of the present invention, provided is a linker-drug conjugate, comprising a compound represented by Formula (II):

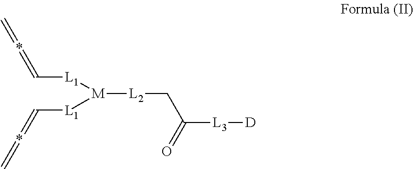

wherein L₁ is selected from

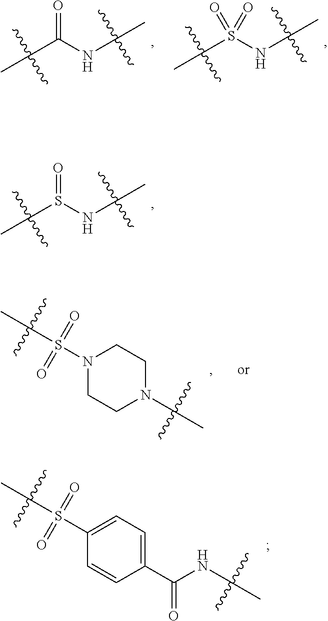 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

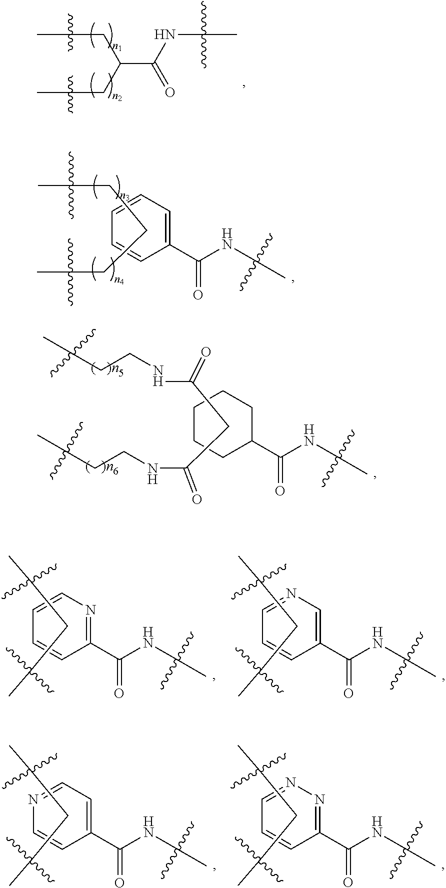

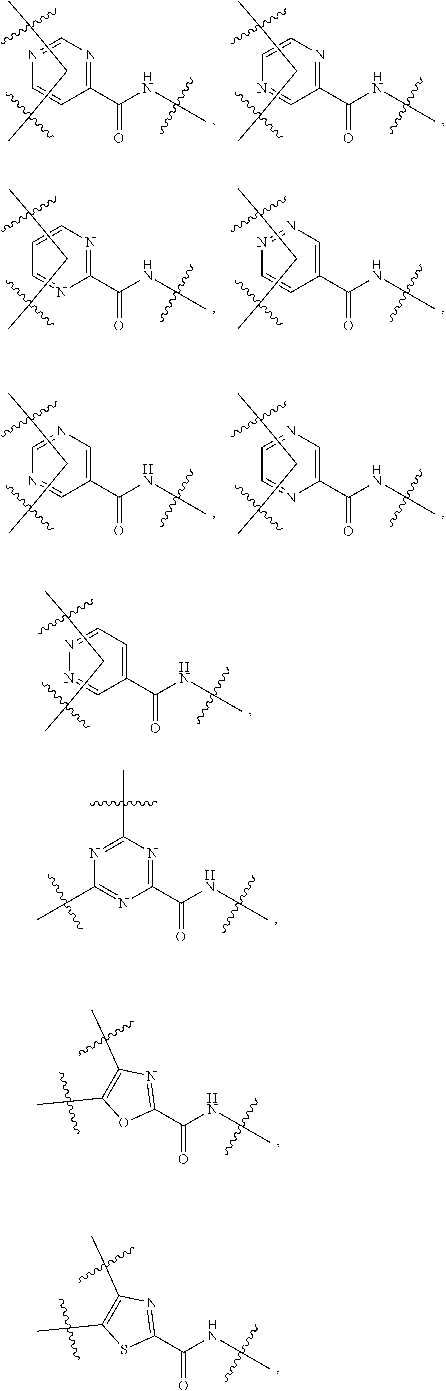

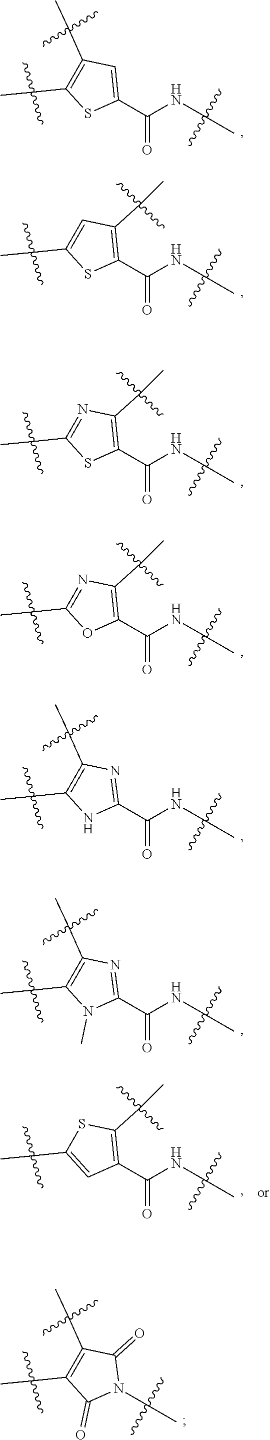

n₁ an n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

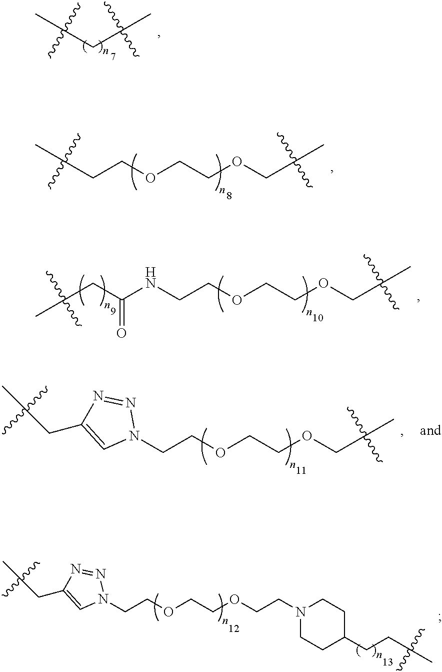

n₇=an integer from 1 to 20, n₈=an integer from 1 to 20, n₉=an integer from 1 to 20, n₁₀=an integer from 1 to 20, n₁₁=an integer from 1 to 20, n₁₂=an integer from 1 to 20, and n₁₃=an integer from 1 to 20;
L₃ is

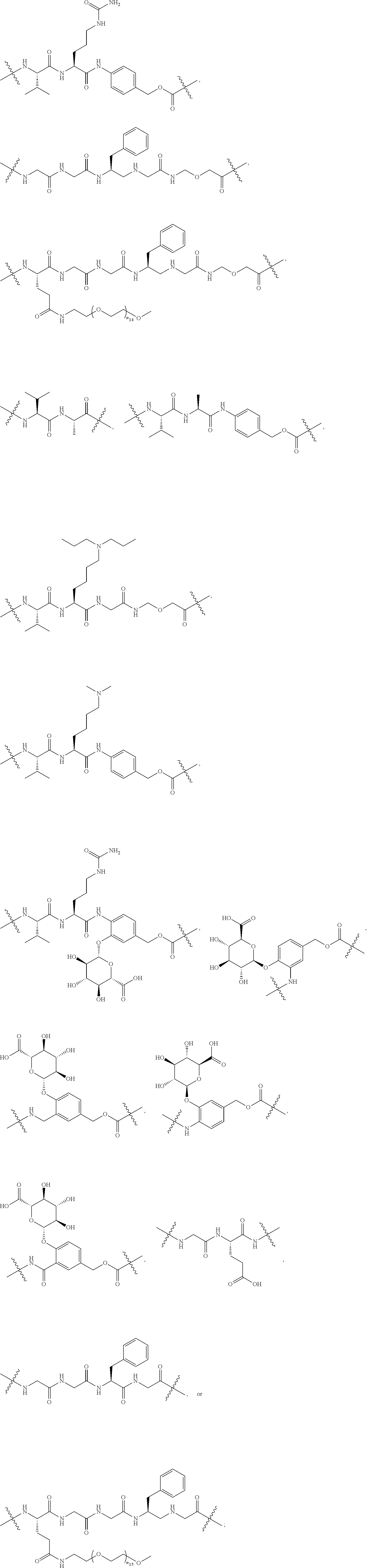

n₁₄=an integer from 1 to 20, and n₁₅=an integer from 1 to 20;
D is a fragment formed by the loss of one or more atoms or groups from a cytotoxic small-molecule drug or active agent,
wherein D is covalently bonded to L₃ via sulfur, nitrogen, or oxygen in D.

According to an embodiment of the present invention, in Formula (II),
L₁ is selected from

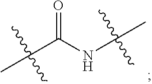 [SMILES: *NC(*)=O]

M is selected from

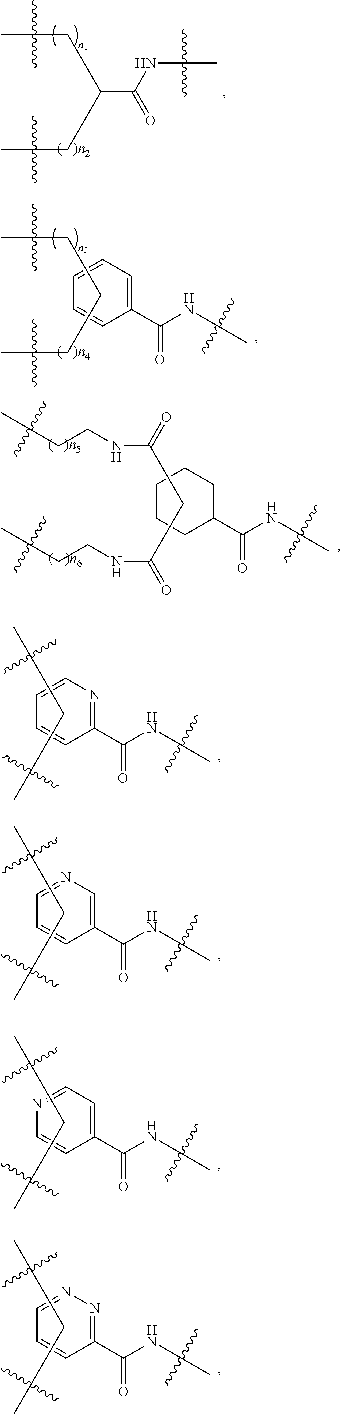

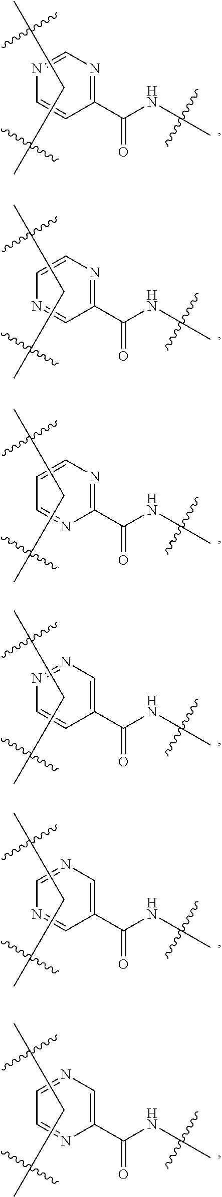

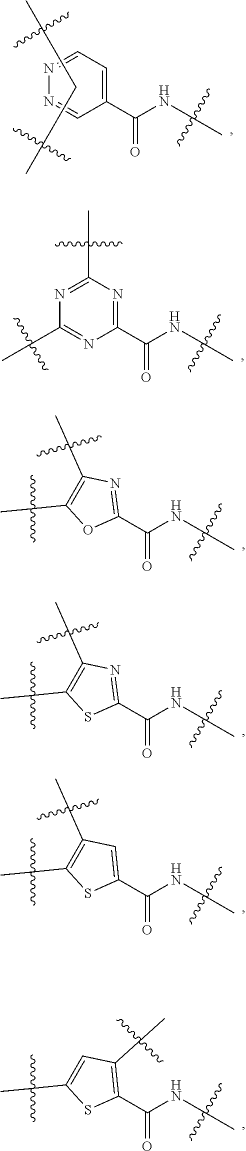

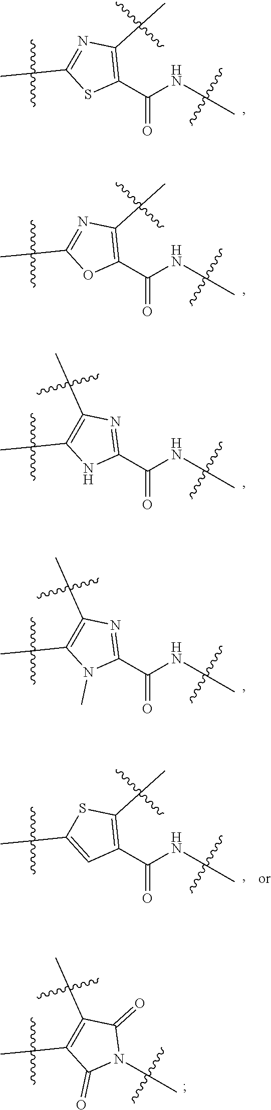 [SMILES: *NC(=O)c1sc(*)nc1* ; *NC(=O)c1oc(*)nc1* ; *NC(=O)c1nc(*)c(*)[nH]1 ; *NC(=O)c1nc(*)c(*)n1C ; *NC(=O)c1cc(*)sc1* ; *C1=C(*)C(=O)N(*)C1=O]

n₁ and n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

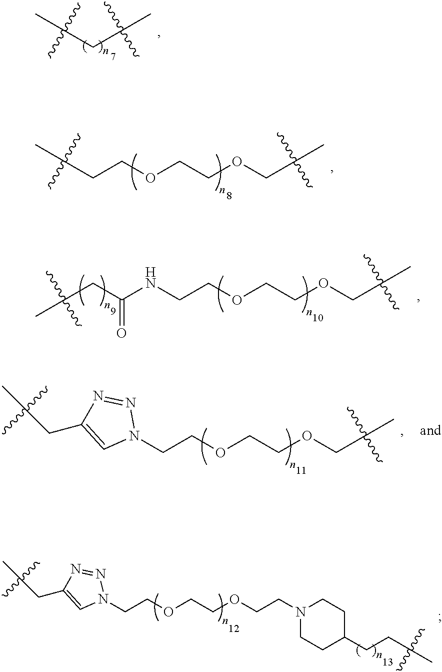

n₇=an integer from 1 to 20, n₈=an integer from 1 to 20, n₉=an integer from 1 to 20, n₁₀=an integer from 1 to 20, n₁=an integer from 1 to 20, n₁₂=an integer from 1 to 20, and n₁₃=an integer from 1 to 20;
L₃ is

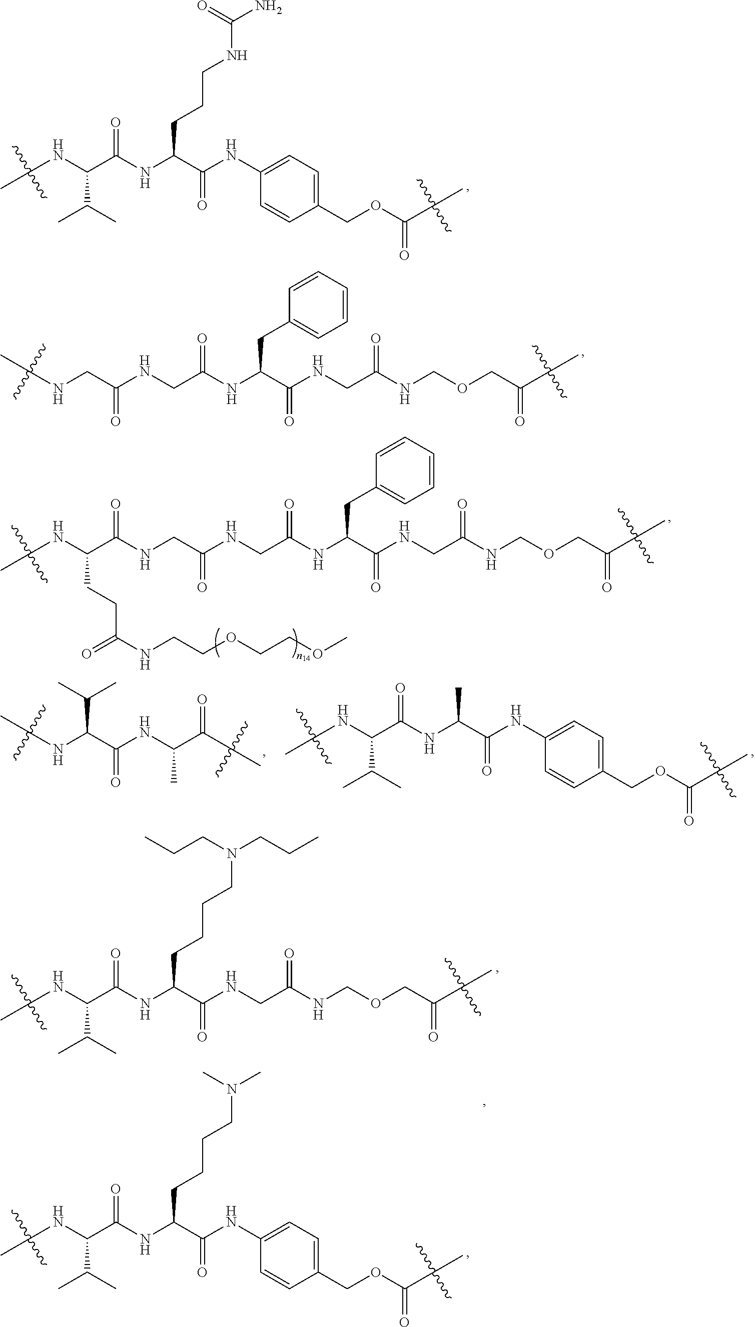

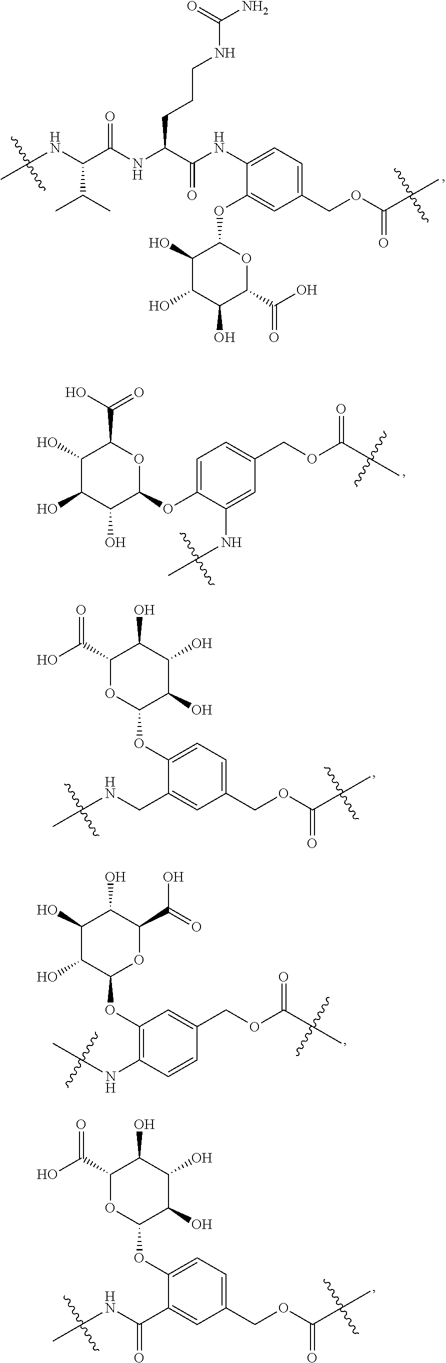 [SMILES: *N[C@H](C(=O)N[C@@H](CCCNC(N)=O)C(=O)Nc1ccc(COC(*)=O)cc1O[C@@H]1O[C@H](C(=O)O)[C@@H](O)[C@H](O)[C@H]1O)C(C)C ; *Nc1cc(COC(*)=O)ccc1O[C@@H]1O[C@H](C(=O)O)[C@@H](O)[C@H](O)[C@H]1O ; *NCc1cc(COC(*)=O)ccc1O[C@@H]1O[C@H](C(=O)O)[C@@H](O)[C@H](O)[C@H]1O ; *Nc1ccc(COC(*)=O)cc1O[C@@H]1O[C@H](C(=O)O)[C@@H](O)[C@H](O)[C@H]1O ; *NC(=O)c1cc(COC(*)=O)ccc1O[C@@H]1O[C@H](C(=O)O)[C@@H](O)[C@H](O)[C@H]1O]

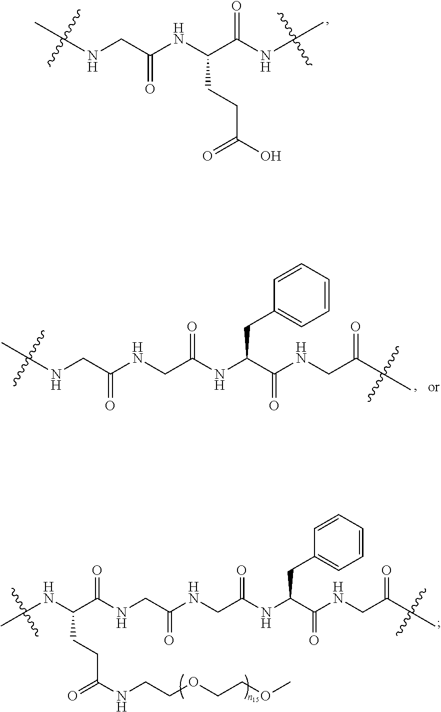

n₁₄=an integer from 1 to 20, and n₁₅=an integer from 1 to 20;
D is a fragment formed by the loss of one or more atoms or groups from a cytotoxic small-molecule drug or active agent,
wherein D is covalently bonded to L₃ via sulfur, nitrogen, or oxygen in D.

According to an embodiment of the present invention, in Formula (II),
L₁ is selected from

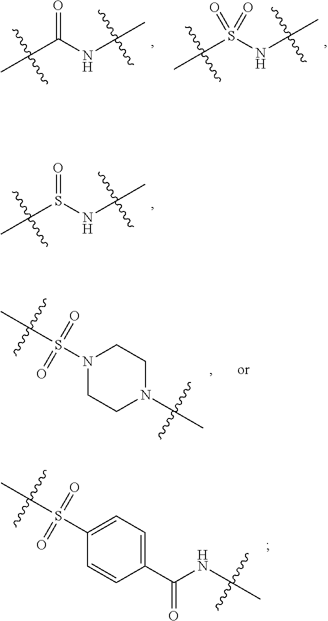 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

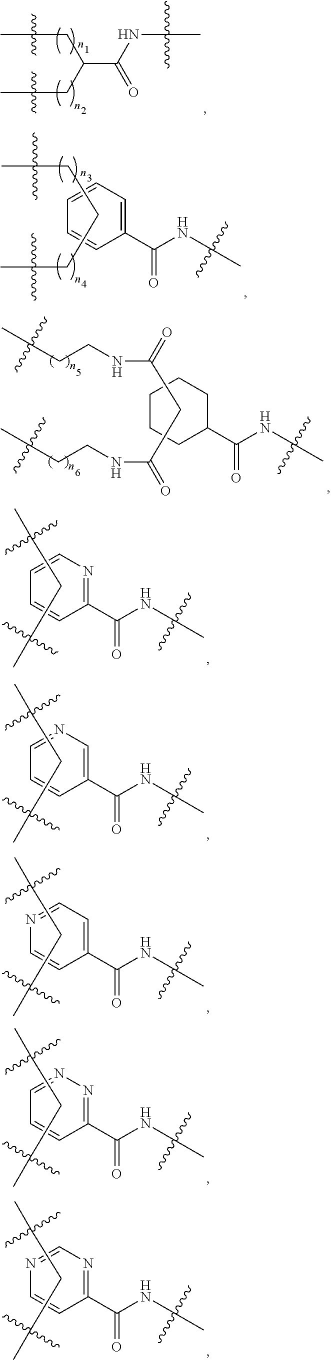

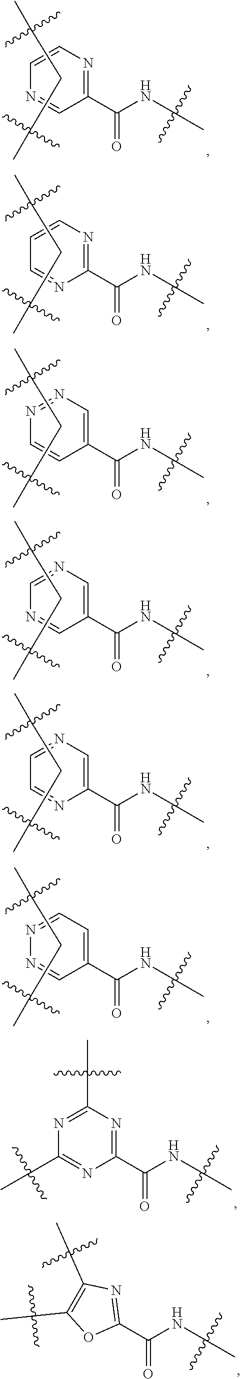

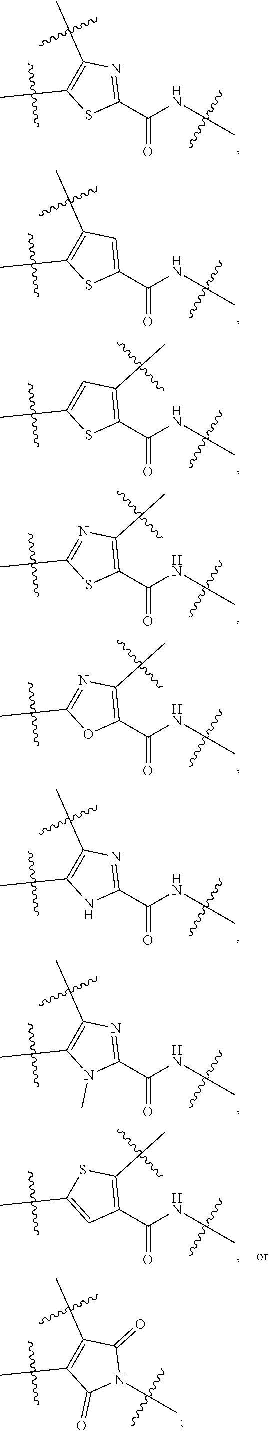

n₁ and n₂ are different, n₁ and n₄ are the same or different, and n₅ and n₆ are the same or different;

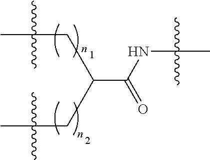

is a single chiral component or a racemate; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more of

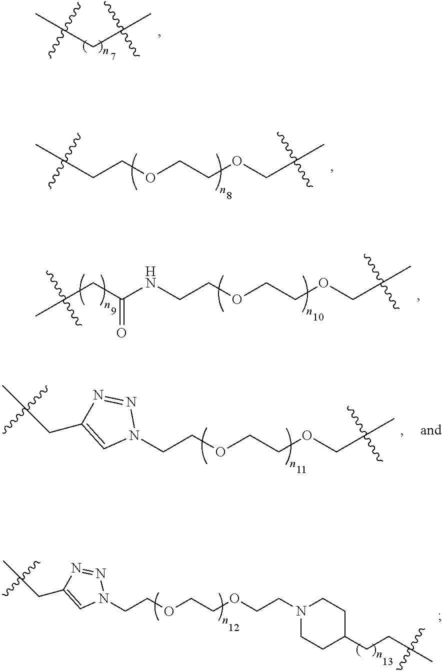

n₇=an integer from 1 to 20, n₈=an integer from 1 to 20, n₉=an integer from 1 to 20, n₁₀=an integer from 1 to 20, n₁=an integer from 1 to 20, n₁₂=an integer from 1 to 20, and n₁₃=an integer from 1 to 20;
L₃ is

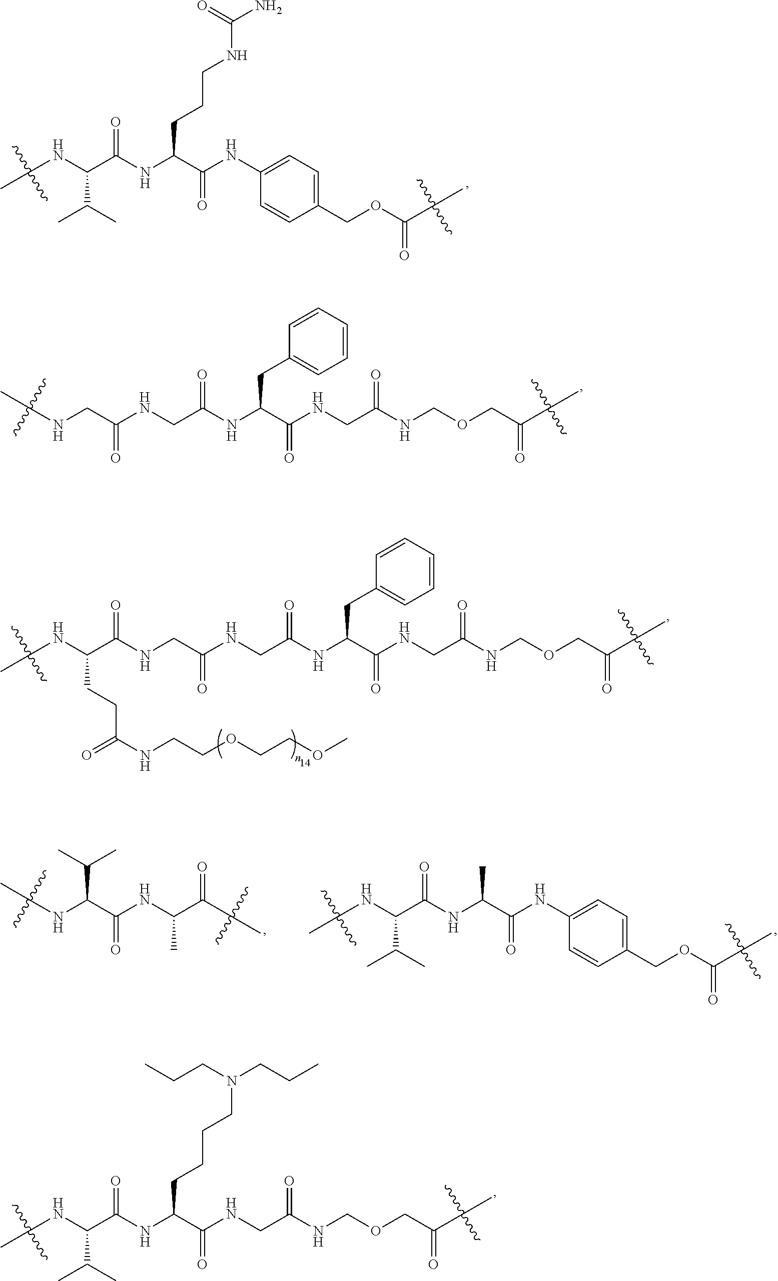

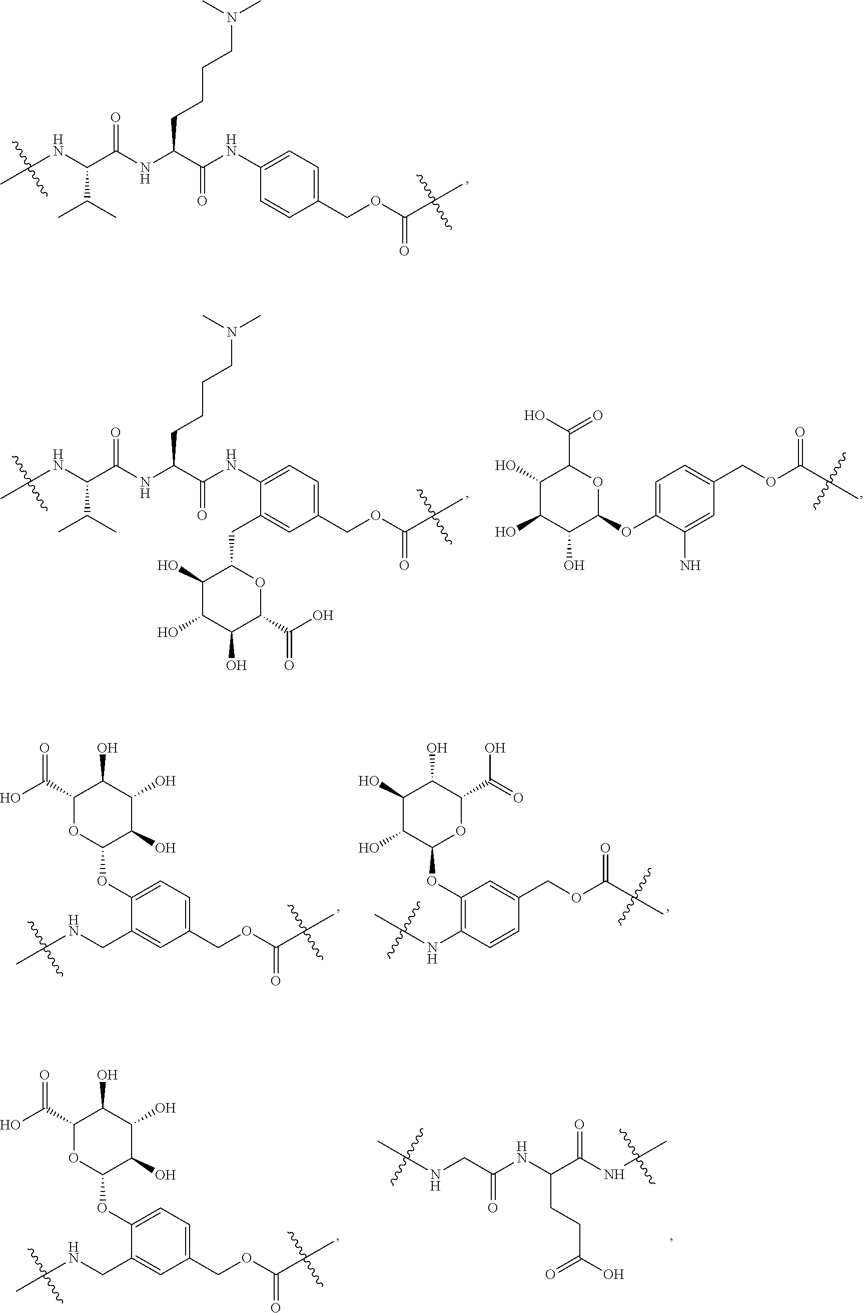

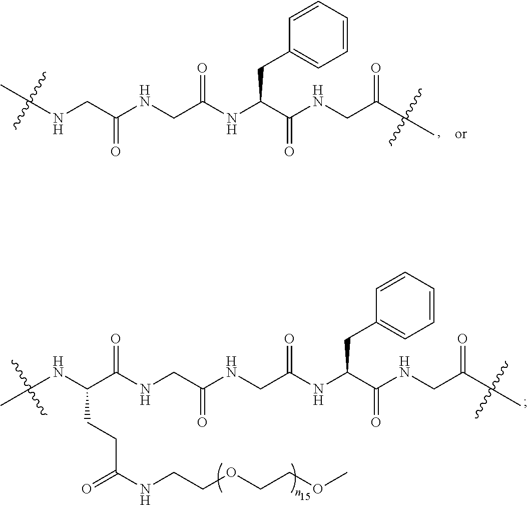

n₁₄=an integer from 1 to 20, and n₁₅=an integer from 1 to 20;
D is a fragment formed by the loss of one or more atoms or groups from a cytotoxic small-molecule drug or other active agent,
wherein D is covalently bonded to L₃ via sulfur, nitrogen, or oxygen in D.

According to an embodiment of the present invention, in Formula (II),
L₁ is selected from

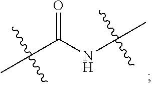 [SMILES: *NC(*)=O]

M is selected from

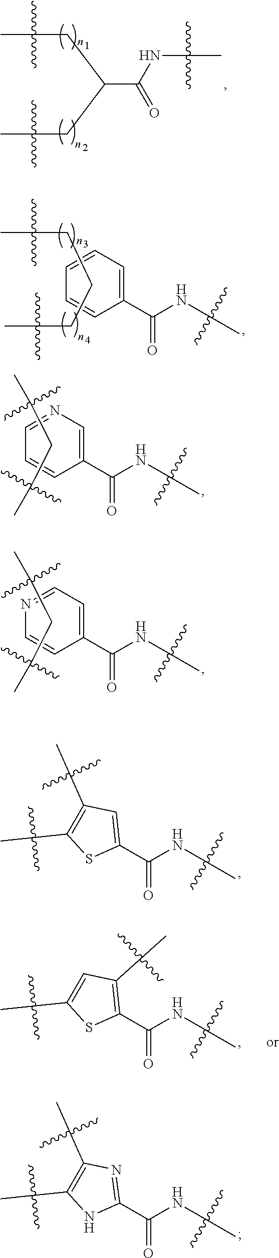

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

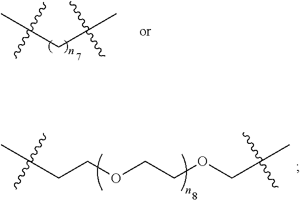

n₇=an integer from 1 to 10, and n₈=an integer from 1 to 10;
L₃ is

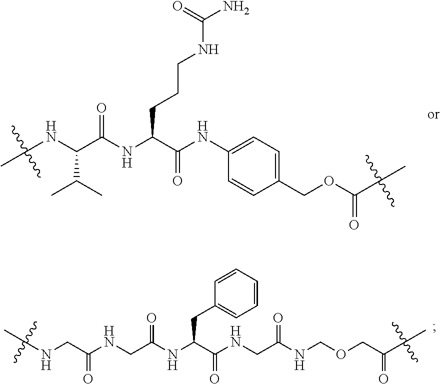 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]

D is a fragment formed by the loss of one or more atoms or groups from a cytotoxic small-molecule drug or other active agent,
wherein D is covalently bonded to L₃ via sulfur, nitrogen, or oxygen in D.

According to an embodiment of the present invention, in Formula (II),
L₁ is selected from

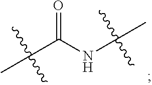 [SMILES: *NC(*)=O]

M is selected from

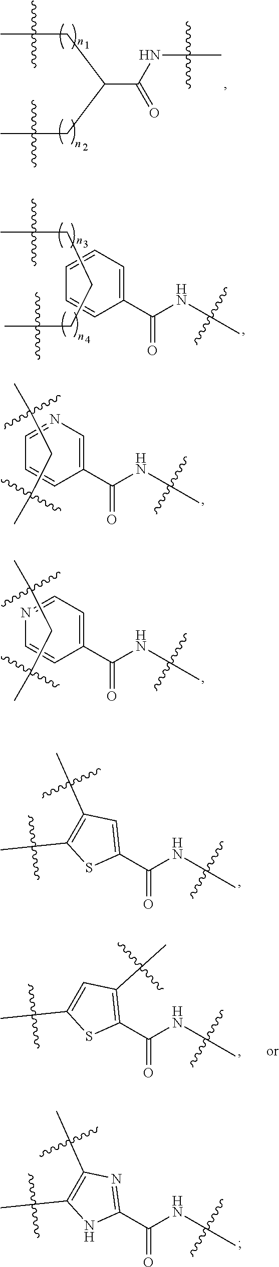

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

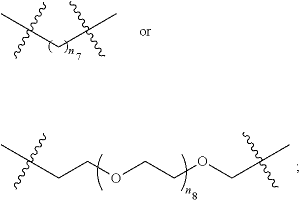

n₇=an integer from 1 to 10, and n₈=an integer from 1 to 10;
L₃ is

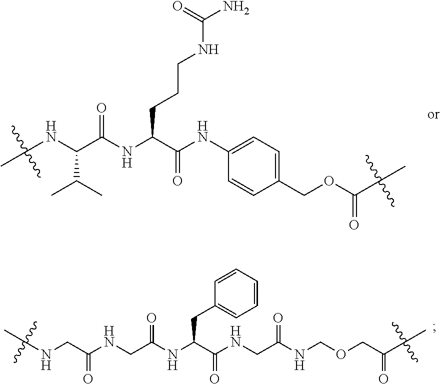 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]

the cytotoxic small-molecule drug is a drug for the treatment of tumor diseases; the active agent is a tubulin inhibitor, an RNA polymerase inhibitor, a topoisomerase inhibitor, an intercalator, a DNA-reactive agent, a DNA alkylating agent, or an immunosuppressant.

The tubulin inhibitor includes, but is not limited to, monomethyl auristatin, maytansinoid, maytansinol, or DM1 maytansinoid. The monomethyl auristatin includes, but is not limited to, monomethyl auristatin E, monomethyl auristatin F, and the like; such an RNA polymerase inhibitor includes, but is not limited to, α-amanitin and the like; the topoisomerase inhibitor includes, but is not limited to, etoposide, teniposide, amsacrine, SN-38, exatecan, and the like; the intercalator includes, but is not limited to, pyrrolobenzodiazepine (PBD) and the like; the DNA-reactive agent includes, but is not limited to, calicheamicin, tiancimycins or other enediynes, and the like; the DNA alkylating agent includes, but is not limited to, trabectedin, lurbinectedin, and other ecteinascidin derivatives.

According to an embodiment of the present invention, the compound of Formula (II) is selected from at least one of the compounds in the following table:

| No. | Structure |
|---|---|
| LP1 | 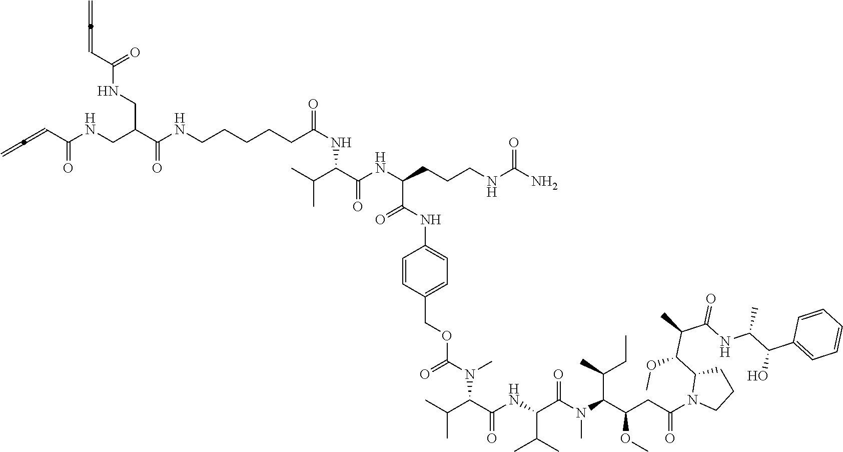 |
| LP2 | 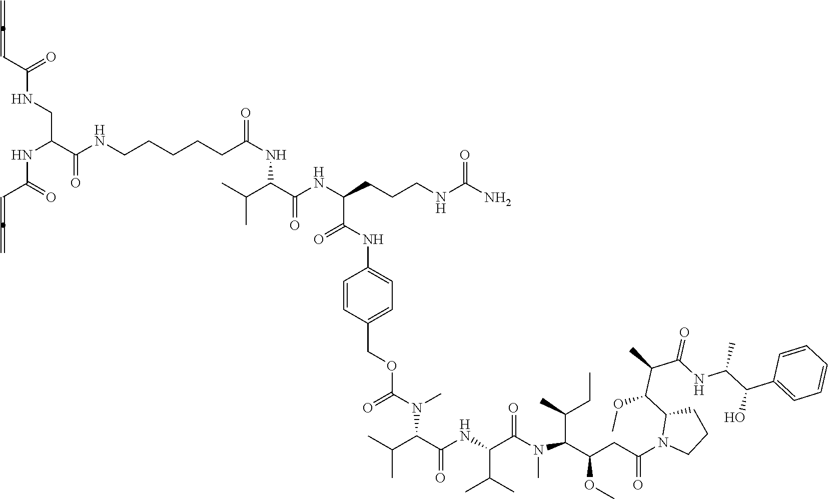 |
| LP3 | 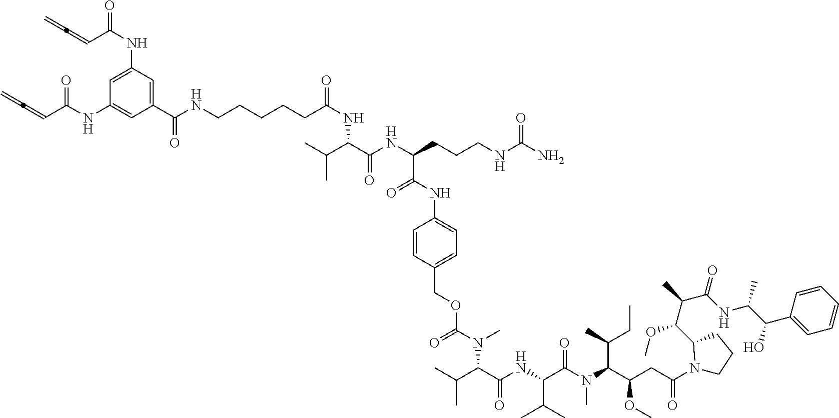 |
| LP4 | 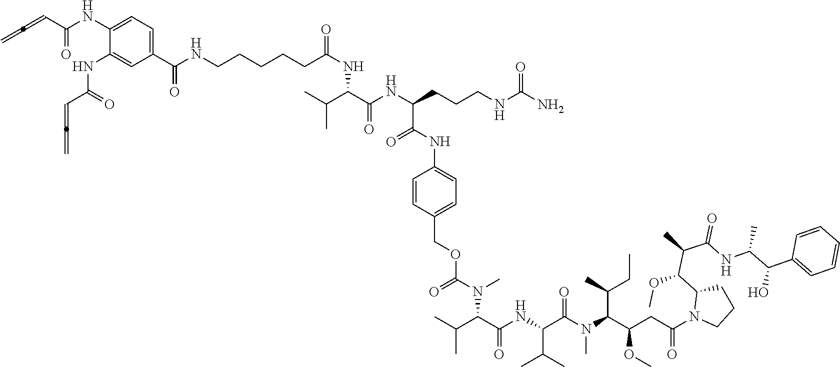 |
| LP5 | 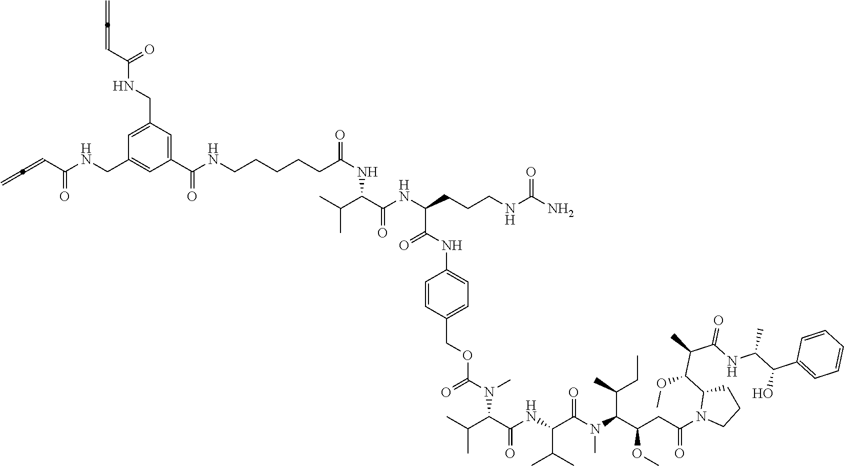 |
| LP6 | 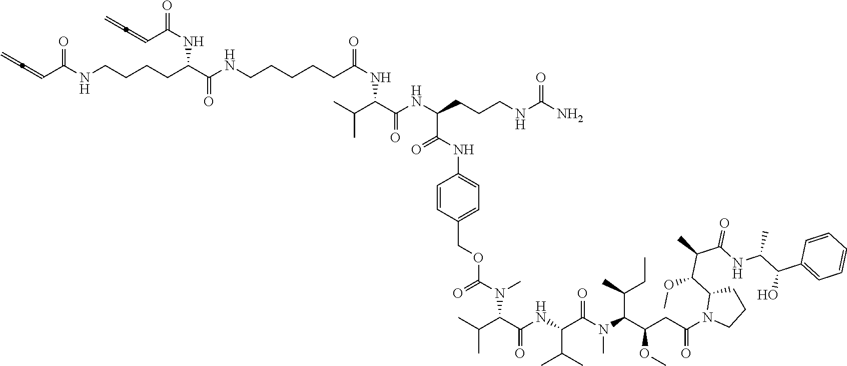 |
| LP7 | 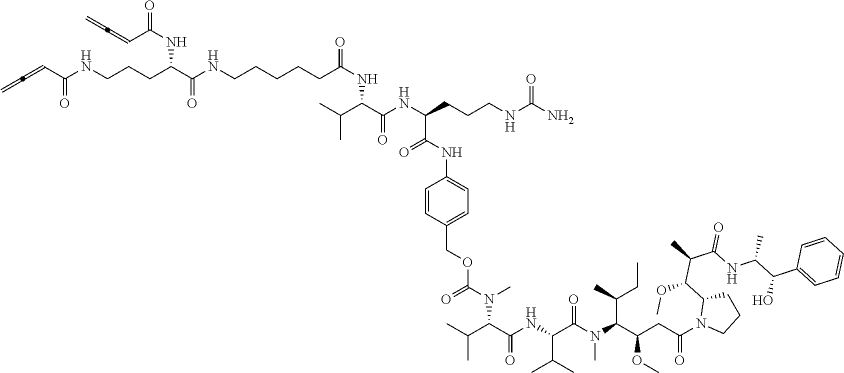 |
| LP8 | 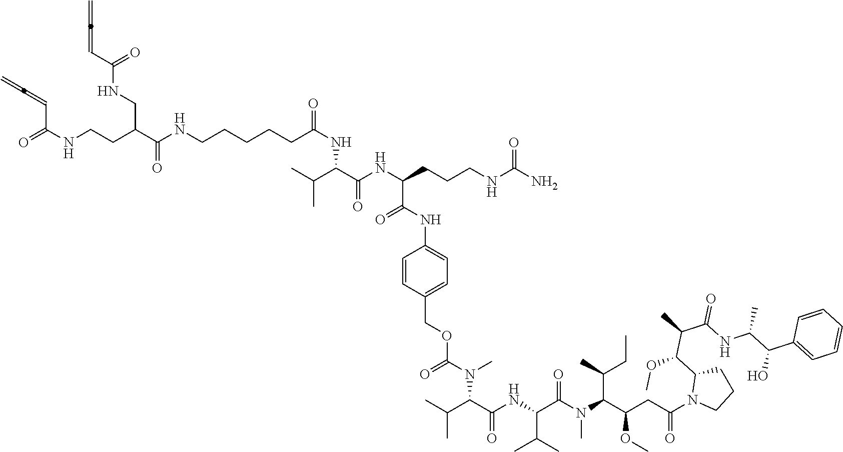 |
| LP9 | 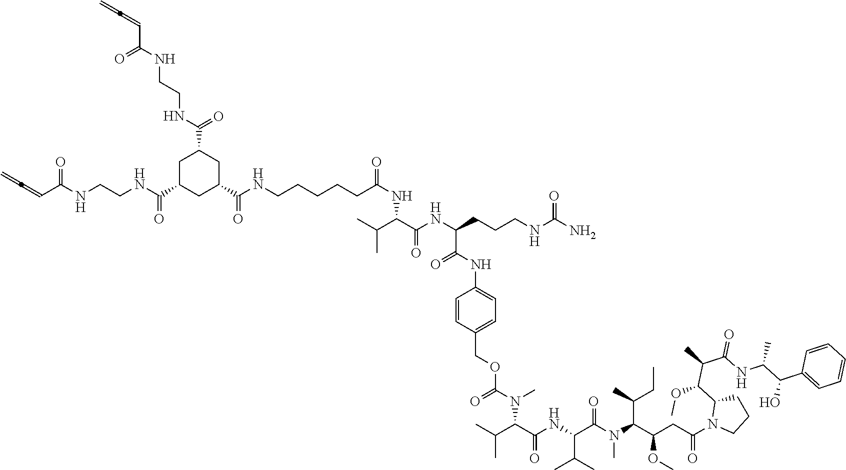 |
| LP10 | 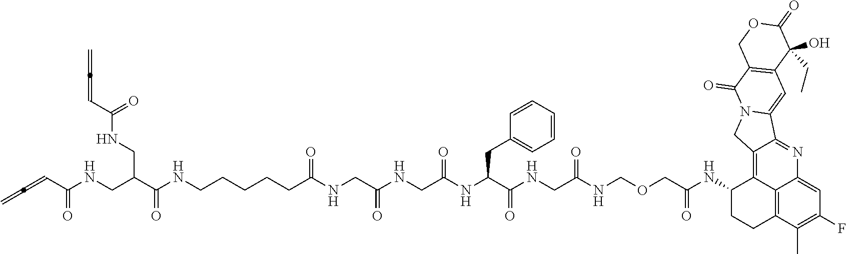 |
| LP11 | 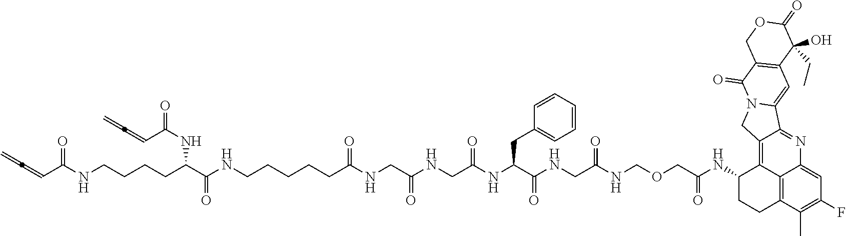 |
| LP12 | 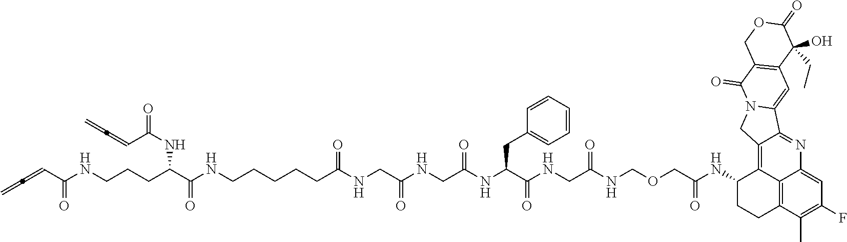 |
| LP13 | 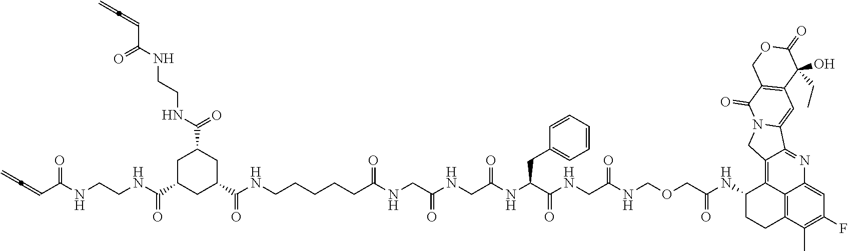 |
| LP14 | 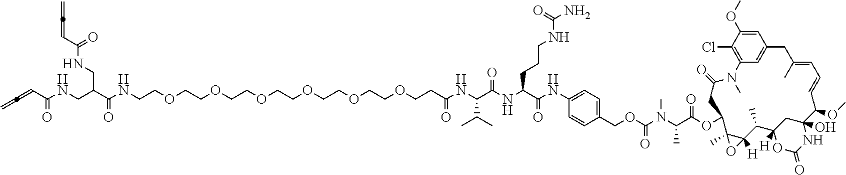 |
| LP15 | 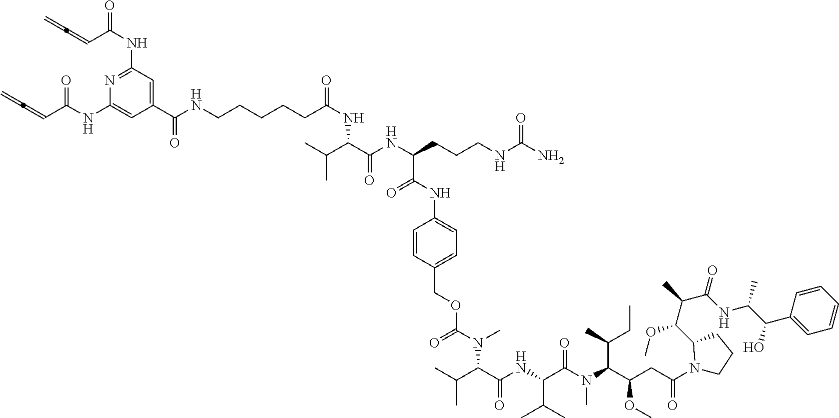 |
| LP16 | 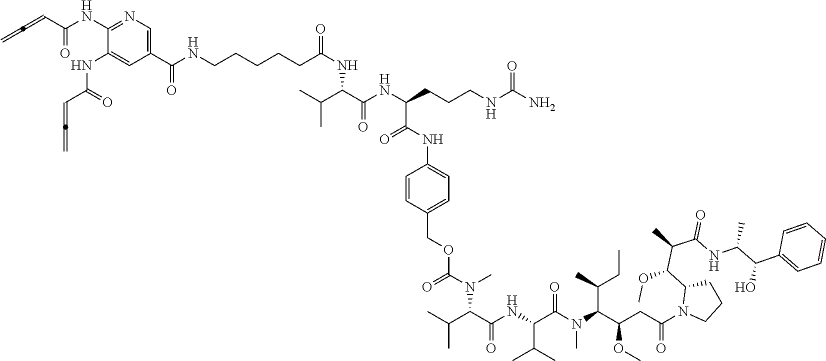 |
| LP17 | 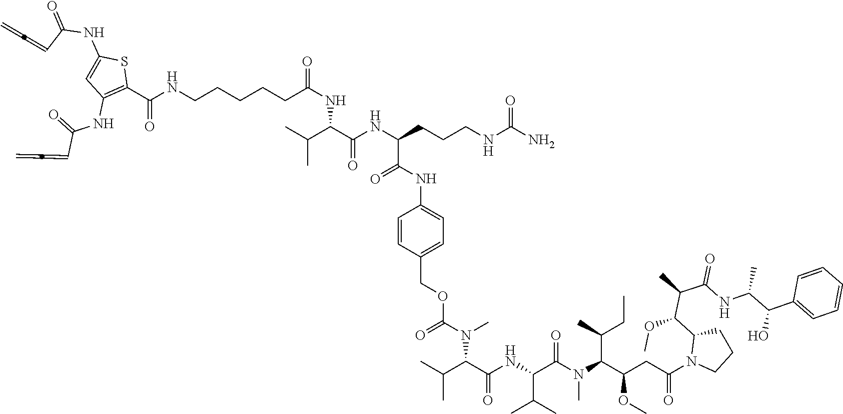 |
| LP18 | 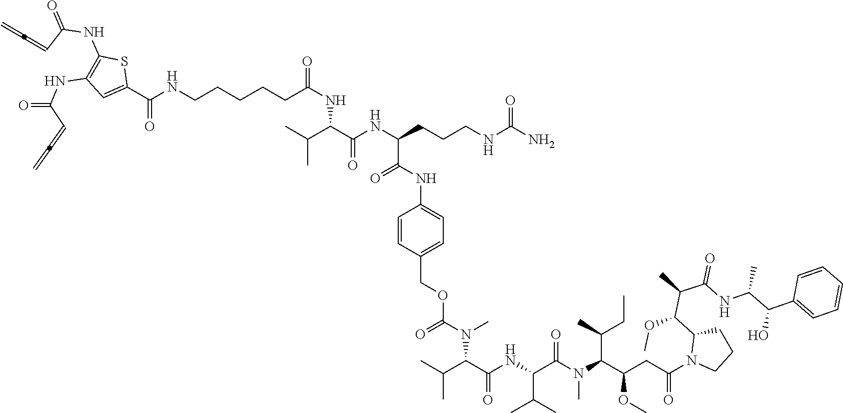 |

In another aspect of the present invention, provided is an antibody-drug conjugate, wherein the conjugate is formed by the conjugation of the bis-allene group in the linker-drug conjugate as described above with the thiol group of the reduced Ab;
the Ab is an antibody, antibody fragment, or protein.

According to an embodiment of the present invention, the antibody is selected from a murine antibody, a rabbit-derived antibody, a phage display-derived antibody, a yeast display-derived antibody, a chimeric antibody, a humanized antibody, a fully human antibody, an antibody fragment, a bispecific antibody, a multispecific antibody, an antigen-binding fragment thereof, an immunologically active portion thereof, or a mutant of the above antibodies.

In another aspect of the present invention, provided is an antibody-drug conjugate, comprising a structure represented by Formula (III):

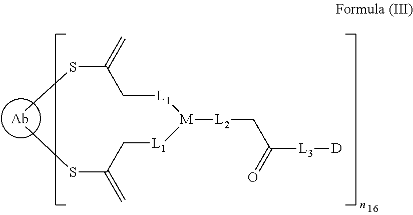

L₁ is selected from

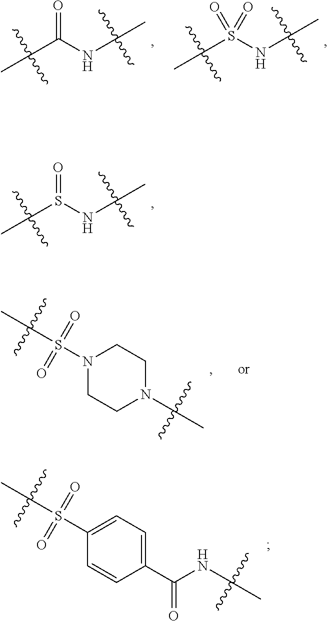 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

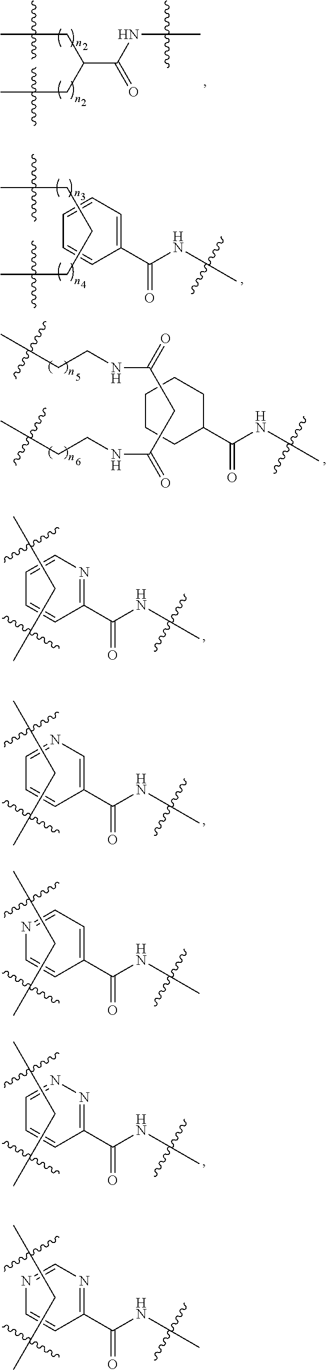

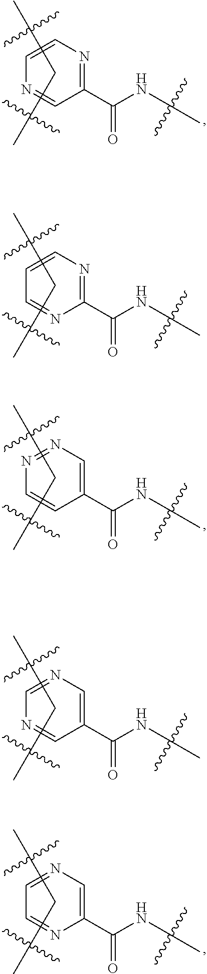

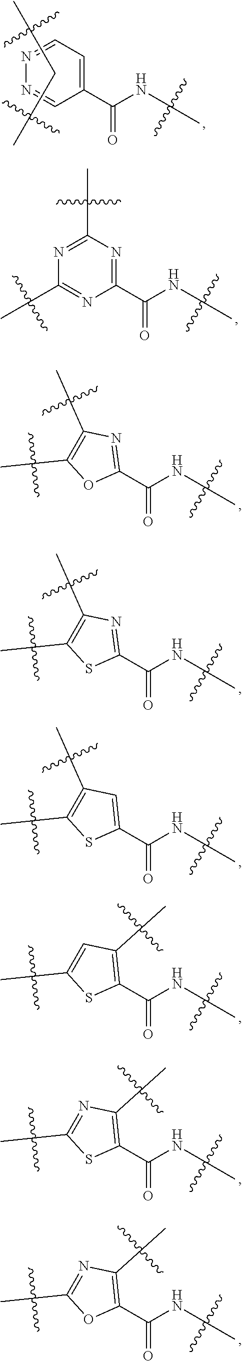

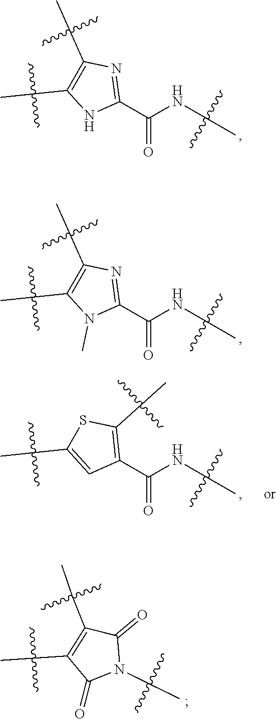 [SMILES: *NC(=O)c1nc(*)c(*)[nH]1 ; *NC(=O)c1nc(*)c(*)n1C ; *NC(=O)c1cc(*)sc1* ; *C1=C(*)C(=O)N(*)C1=O]

n₁ and n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more combinations of

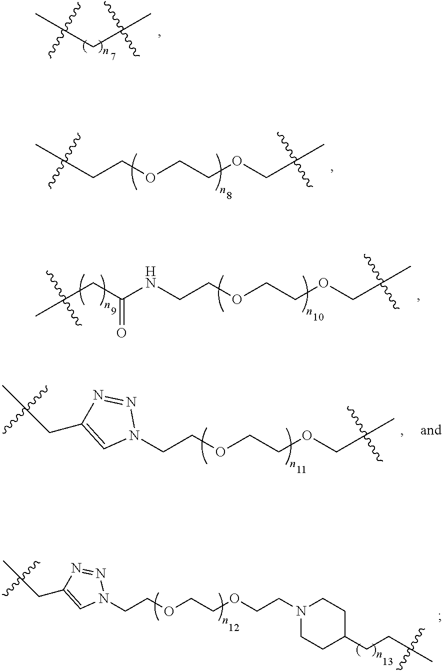

n₇=an integer from 1 to 20, n₈=an integer from 1 to 20, n₉=an integer from 1 to 20, n₁₀=an integer from 1 to 20, n₁₁=an integer from 1 to 20, n₁₂=an integer from 1 to 20, and n₁₃=an integer from 1 to 20;
L is

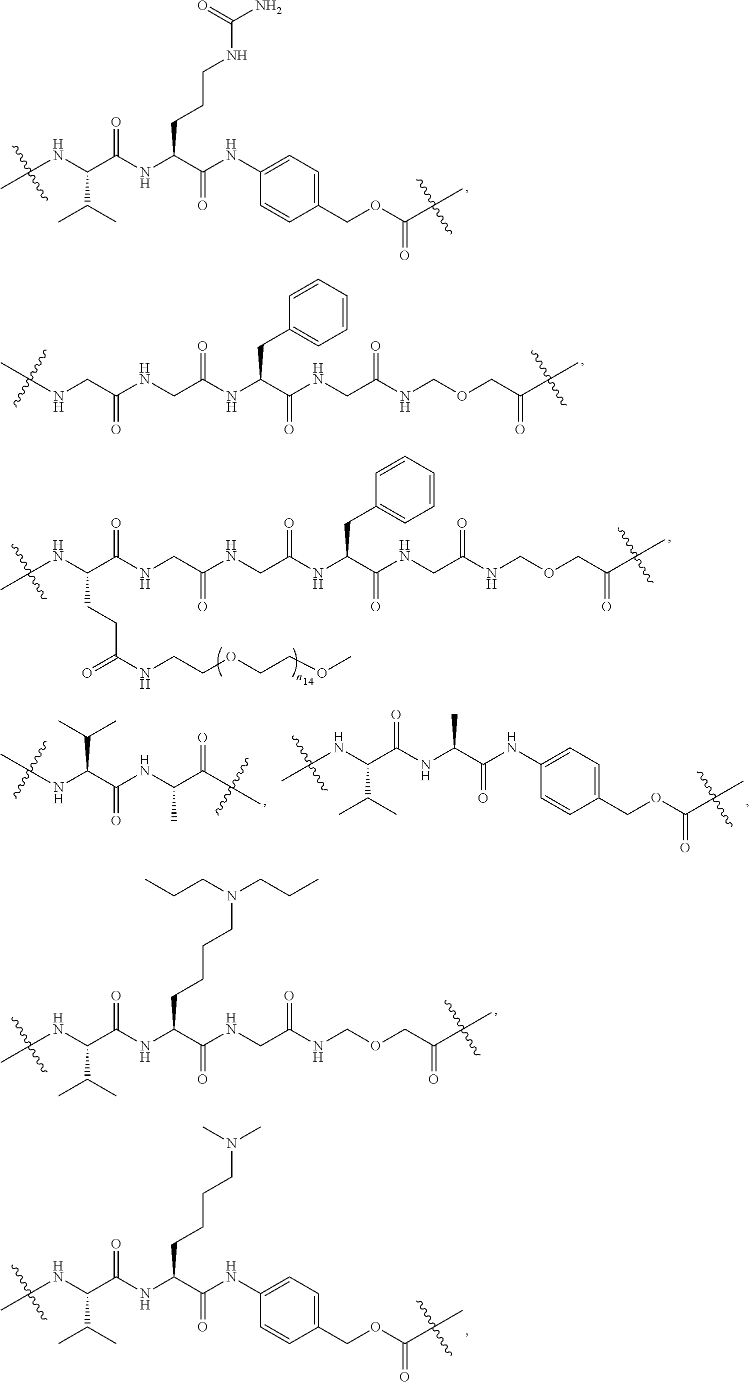

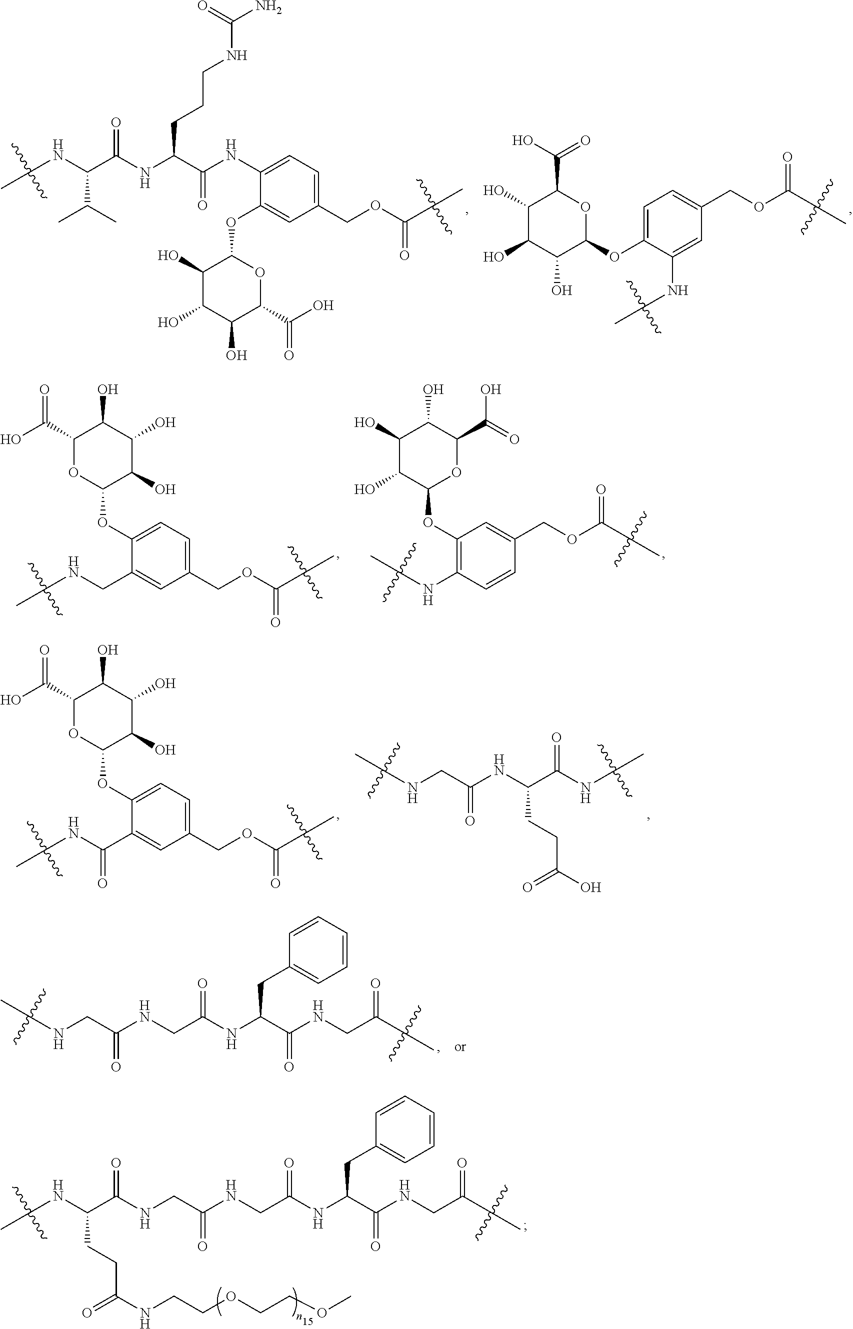

n₁₄=an integer from 1 to 20, and n₁₅=an integer from 1 to 20;
D is a fragment formed by the loss of one or more atoms or groups from a cytotoxic small-molecule drug or active agent,
wherein D is covalently bonded to L₃ via sulfur, nitrogen, or oxygen in D.
n₁₆ is selected from any number between 1 and 20;
the Ab is an antibody, antibody fragment, or protein.

The antibody is selected from a murine antibody, a rabbit-derived antibody, a phage display-derived antibody, a yeast display-derived antibody, a chimeric antibody, a humanized antibody, a fully human antibody, an antibody fragment, a bispecific antibody, a multispecific antibody, an antigen-binding fragment thereof, an immunologically active portion thereof, or a mutant of the above antibodies.

According to an embodiment of the present invention, in Formula (III),
L₁ is selected from

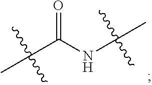 [SMILES: *NC(*)=O]

M is selected from

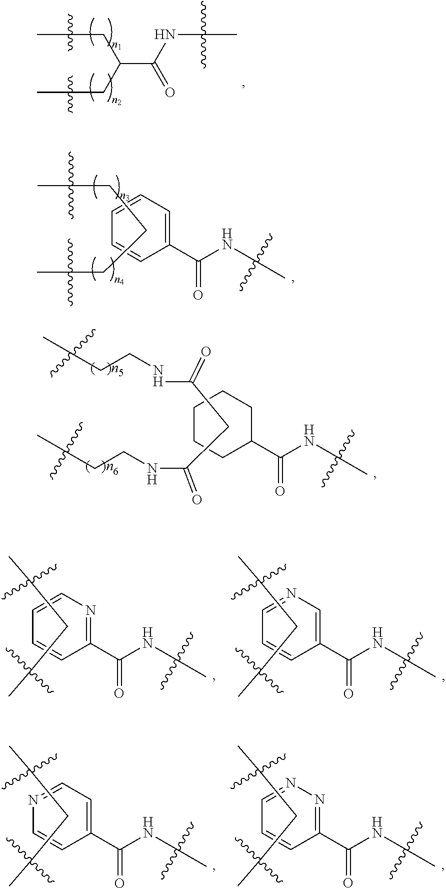

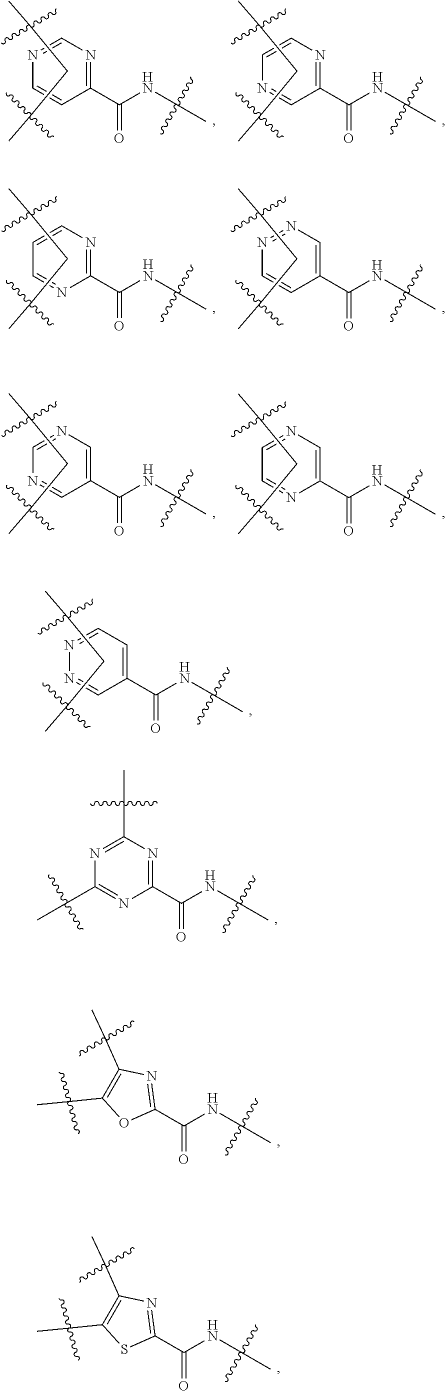

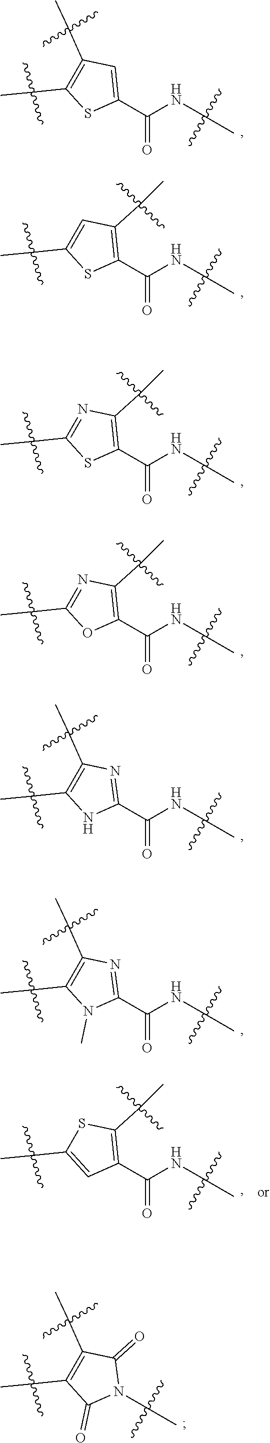

n₁ and n₂ are the same or different, n₃ and n₄ are the same or different, and n₅ and n₆ are the same or different; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more combinations of

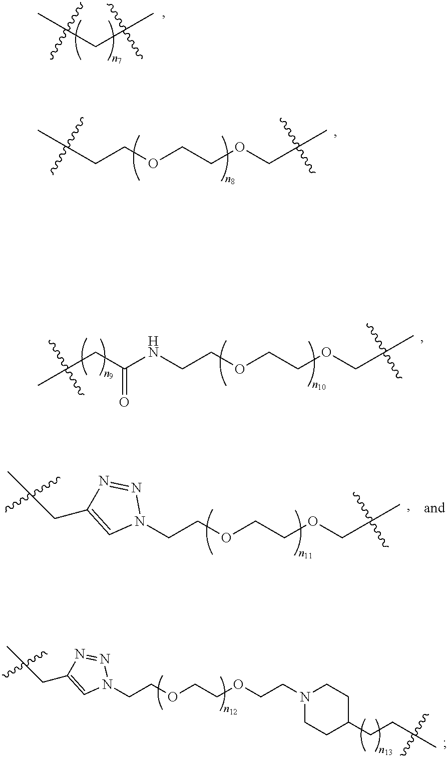

n₇=an integer from 1 to 20, n₈=an integer from 1 to 20, n₉=an integer from 1 to 20, n₁₀=an integer from 1 to 20, n₁₁=an integer from 1 to 20, n₁₂=an integer from 1 to 20, and n₁₃=an integer from 1 to 20;
L₃ is

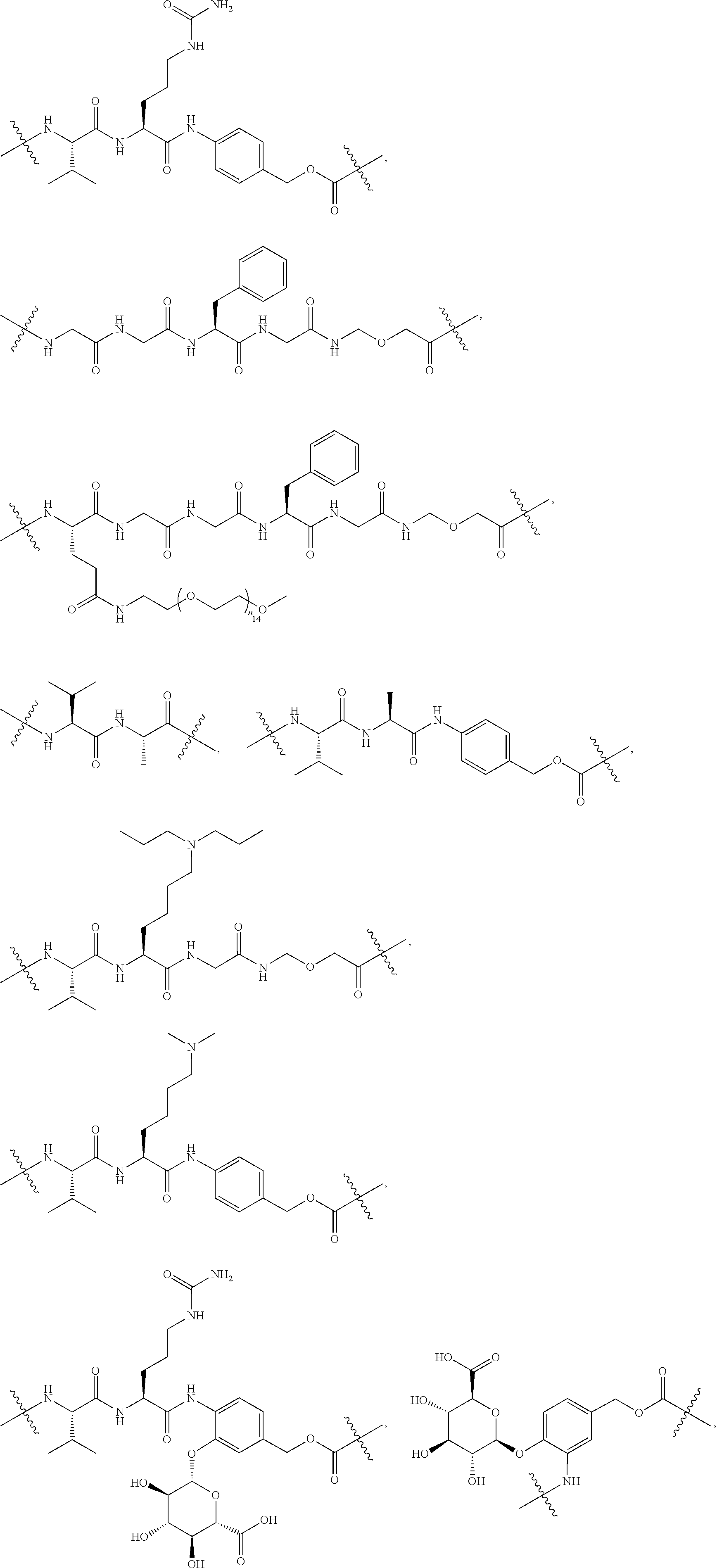

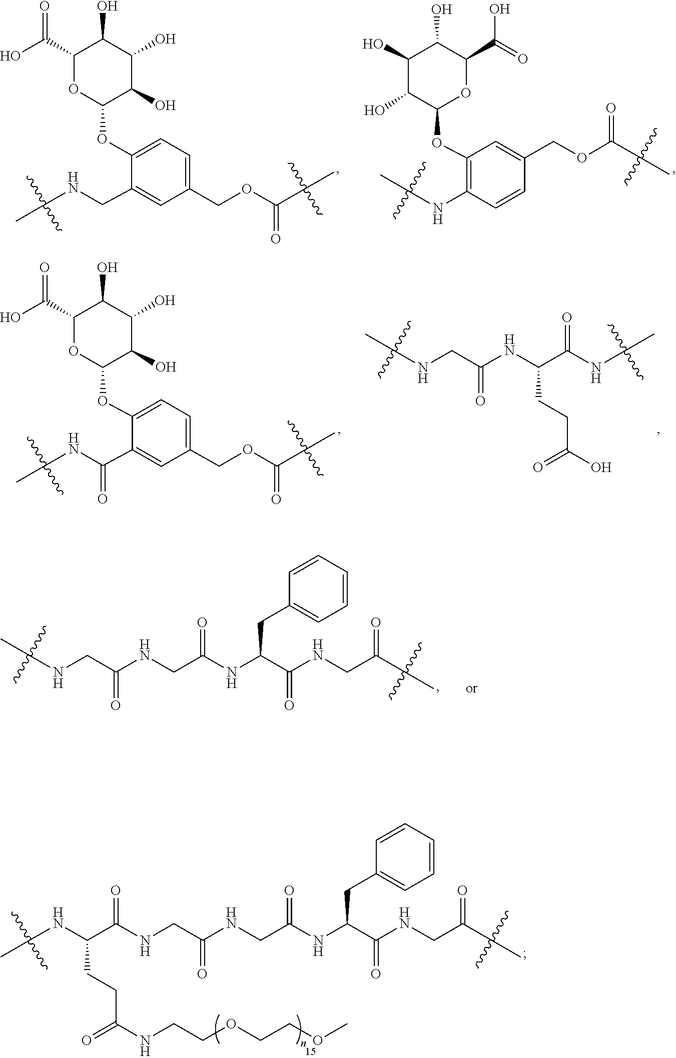

n₁₄=an integer from 1 to 20, and n₁₅=an integer from 1 to 20;
the cytotoxic small-molecule drug or active agent is a fragment formed by the loss of one or more atoms or groups from a cytotoxic small-molecule drug or other active agent;
wherein D is covalently bonded to L₃ via sulfur, nitrogen, or oxygen in D.
n₁₆ is selected from any number between 1 and 20;
the Ab is an antibody, antibody fragment, or protein.

The antibody is selected from a murine antibody, a rabbit-derived antibody, a phage display-derived antibody, a yeast display-derived antibody, a chimeric antibody, a humanized antibody, a fully human antibody, an antibody fragment, a bispecific antibody, a multispecific antibody, an antigen-binding fragment thereof, an immunologically active portion thereof, or a mutant of the above antibodies.

According to an embodiment of the present invention, in Formula (III),
L₁ is selected from

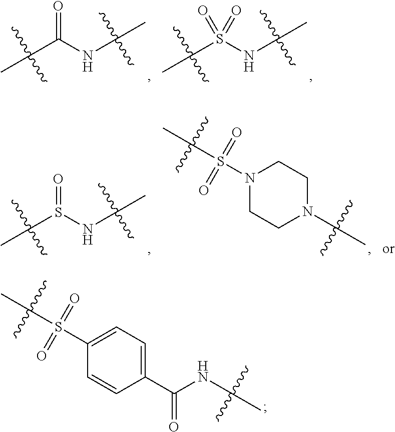 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]

M is selected from

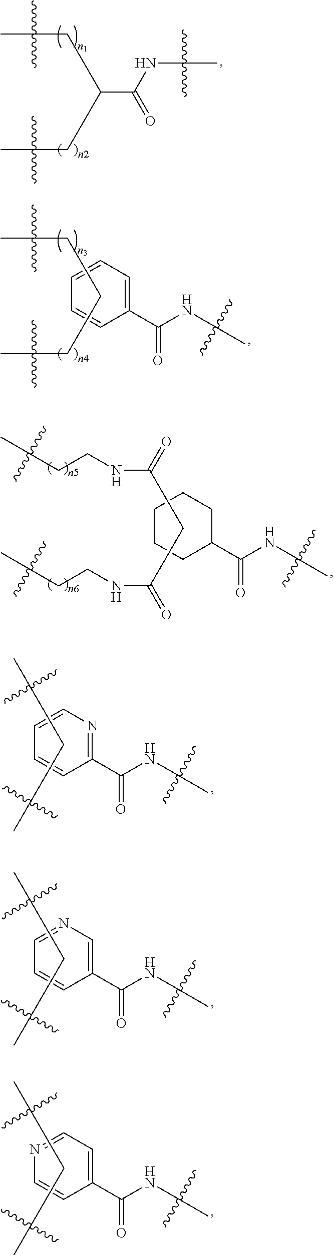

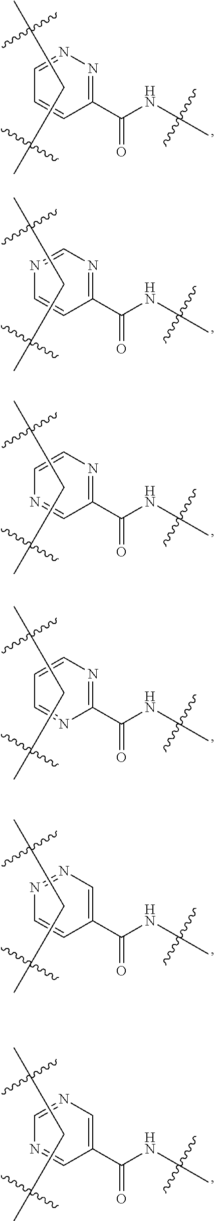

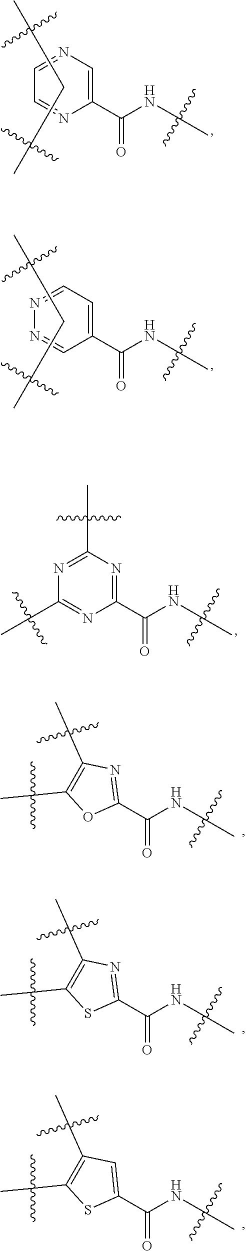

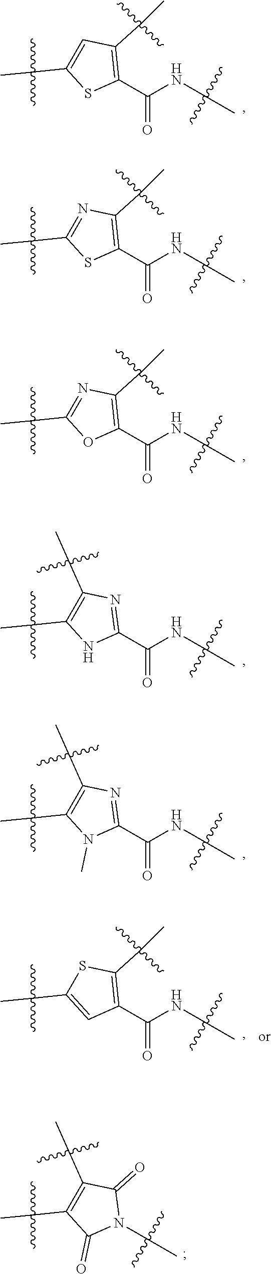

n₁ and n₂ are different, n₃ and n₄ are the same or different, and n₅ and n₆ are the same or different;

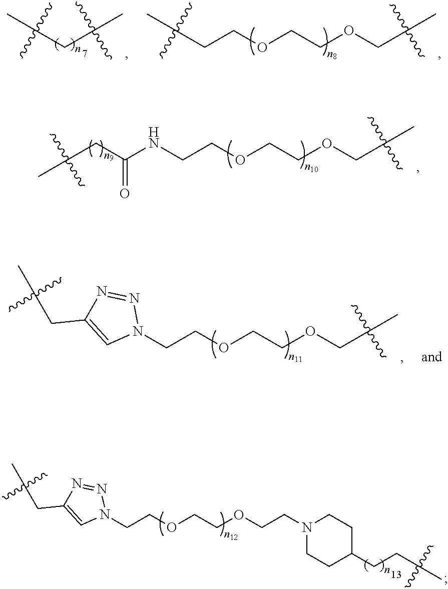

is a single chiral component or a racemate; n₁=an integer from 0 to 10, n₂=an integer from 0 to 10, n₃=an integer from 0 to 10, n₄=an integer from 0 to 10, n₅=an integer from 1 to 10, and n₆=an integer from 1 to 10;
L₂ is selected from one or more combinations of

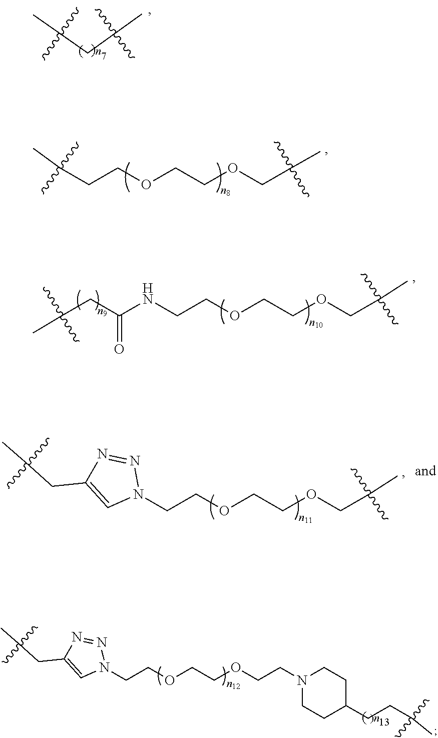

n₇=an integer from 1 to 20, n₈=an integer from 1 to 20, n₉=an integer from 1 to 20, n₁₀=an integer from 1 to 20, n₁₁=an integer from 1 to 20, n₁₂=an integer from 1 to 20, and n₁₃=an integer from 1 to 20;
L₃ is

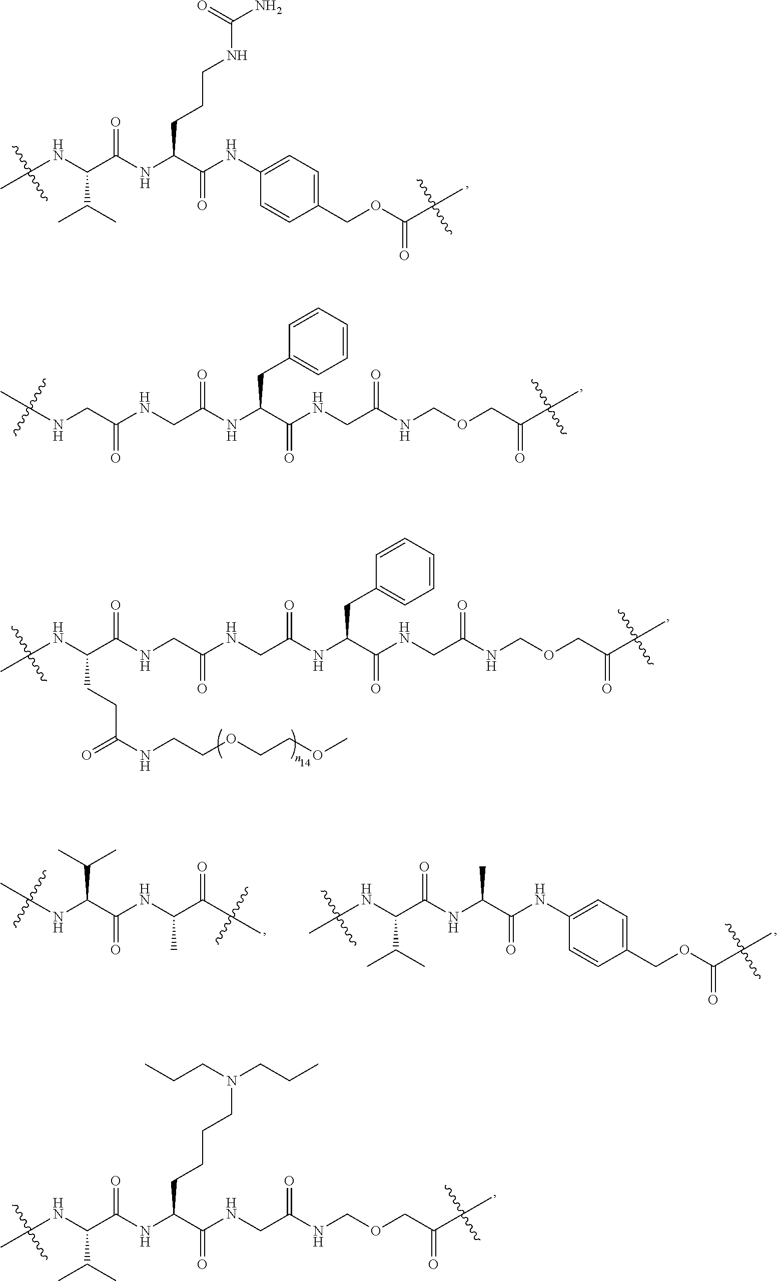

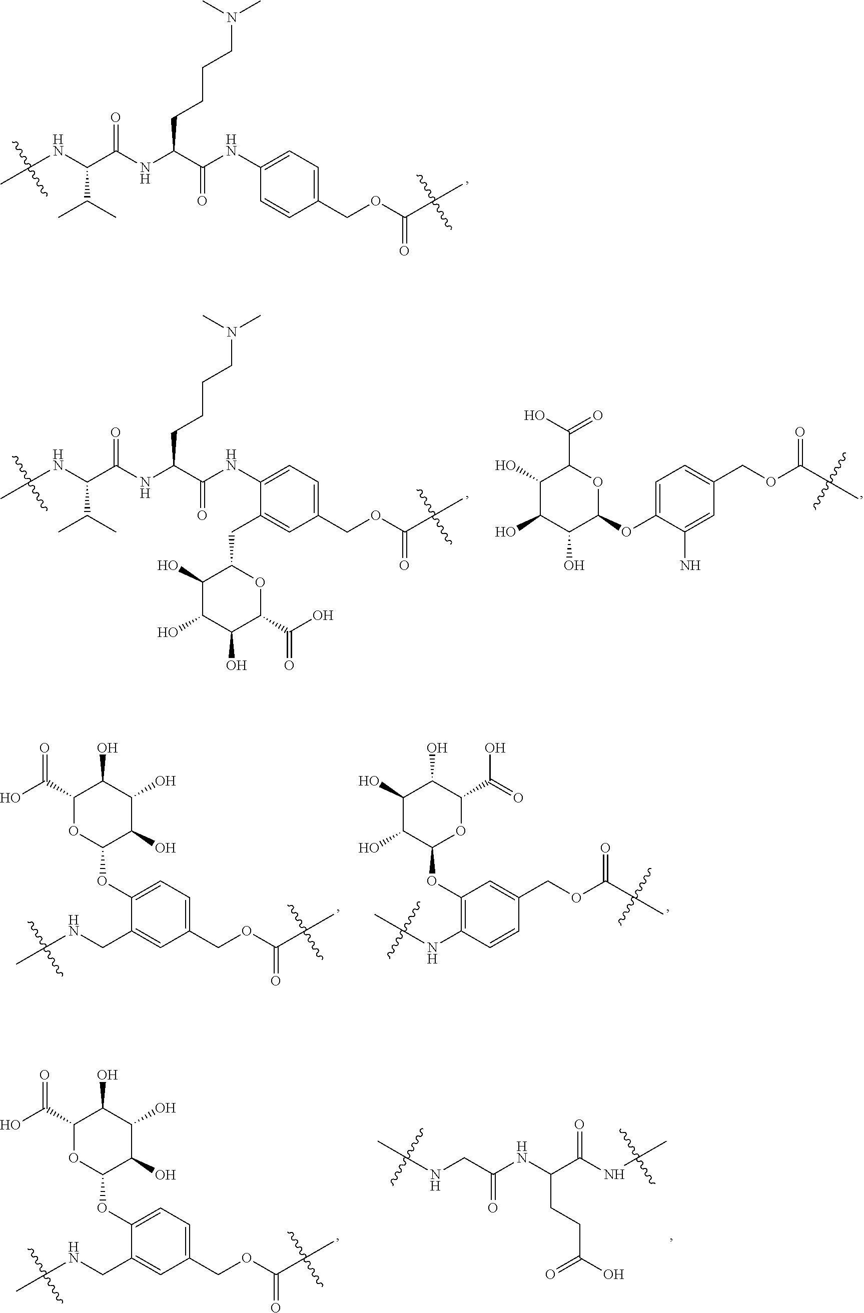

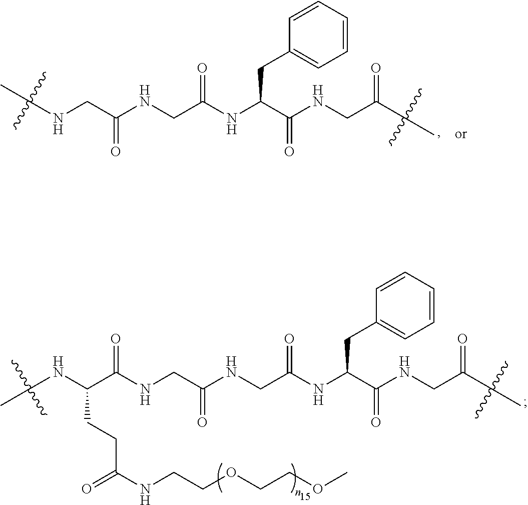

n₁₄=an integer from 1 to 20, and n₁₅=an integer from 1 to 20;
D is a fragment formed by the loss of one or more atoms or groups from a cytotoxic small-molecule drug or active agent;
wherein D is covalently bonded to L₃ via sulfur, nitrogen, or oxygen in D;
n₁₆ is selected from any number between 1 and 20;
the Ab is an antibody, antibody fragment, or protein.

The antibody is selected from a murine antibody, a rabbit-derived antibody, a phage display-derived antibody, a yeast display-derived antibody, a chimeric antibody, a humanized antibody, a fully human antibody, an antibody fragment, a bispecific antibody, a multispecific antibody, an antigen-binding fragment thereof, an immunologically active portion thereof, or a mutant of the above antibodies.

According to an embodiment of the present invention,
L₁ is selected from

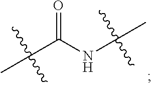 [SMILES: *NC(*)=O]

M is selected from

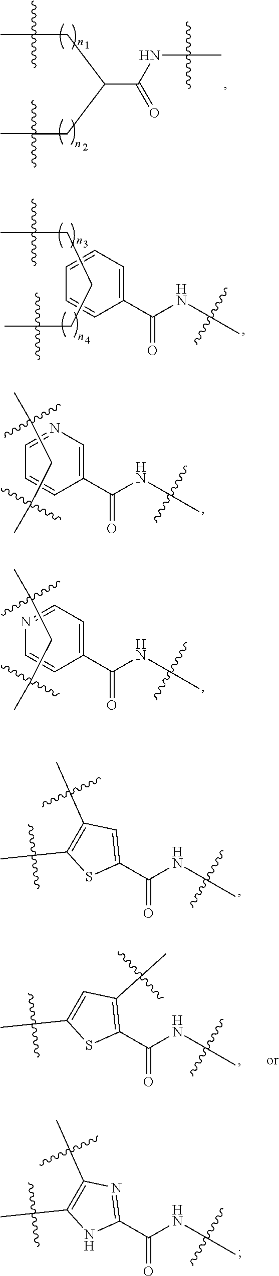

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

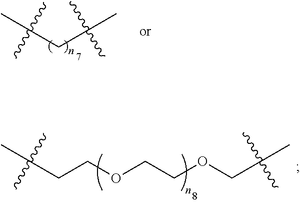

n₇=an integer from 1 to 10, and n₈=an integer from 1 to 10;
L₃ is

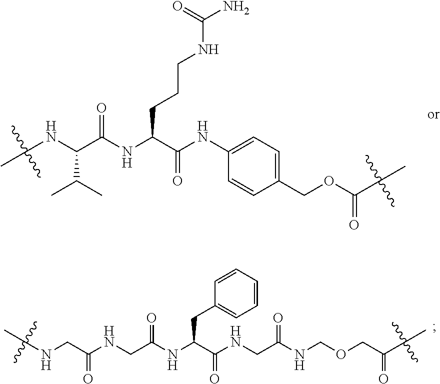 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]

the cytotoxic small-molecule drug is a drug for the treatment of tumor diseases; the active agent is a tubulin inhibitor, an RNA polymerase inhibitor, a topoisomerase inhibitor, an intercalator, a DNA-reactive agent, a DNA alkylating agent, or an immunosuppressant;
n₁₆ is selected from any number between 1 and 20;
the Ab is an antibody, antibody fragment, or protein.

The tubulin inhibitor includes, but is not limited to, monomethyl auristatin, maytansinoid, maytansinol, or DM1 maytansinoid. The monomethyl auristatin may be monomethyl auristatin E, monomethyl auristatin F, and the like; the RNA polymerase inhibitor includes, but is not limited to, α-amanitin and the like; the topoisomerase inhibitor includes, but is not limited to, etoposide, teniposide, amsacrine, SN-38, exatecan, and the like; the intercalator includes, but is not limited to, pyrrolobenzodiazepine (PBD) and the like; the DNA-reactive agent includes, but is not limited to, calicheamicin, tiancimycins or other enediynes, and the like; the DNA alkylating agent includes, but is not limited to, trabectedin, lurbinectedin, and other ecteinascidin derivatives.

The antibody is selected from a murine antibody, a rabbit-derived antibody, a phage display-derived antibody, a yeast display-derived antibody, a chimeric antibody, a humanized antibody, a fully human antibody, an antibody fragment, a bispecific antibody, a multispecific antibody, an antigen-binding fragment thereof, an immunologically active portion thereof, or a mutant of the above antibodies.

According to an embodiment of the present invention, in Formula (III),
L₁ is selected from

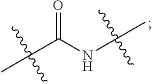 [SMILES: *NC(*)=O]

M is selected from

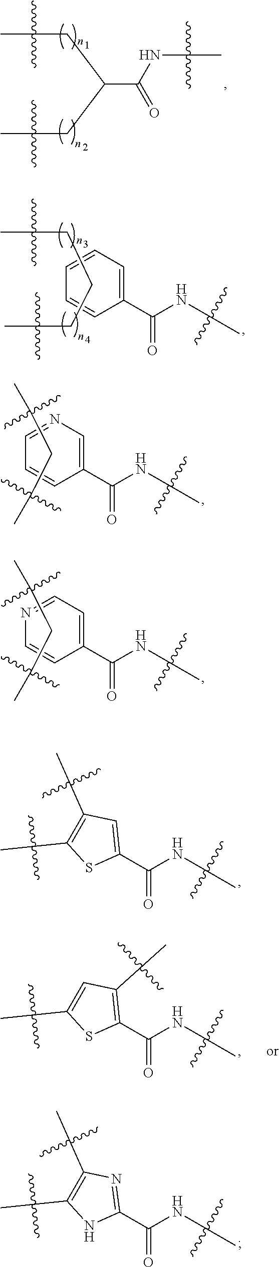

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

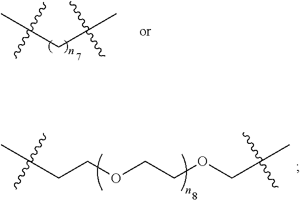

n₇=an integer from 1 to 10, and n₈=an integer from 1 to 10;
L₃ is

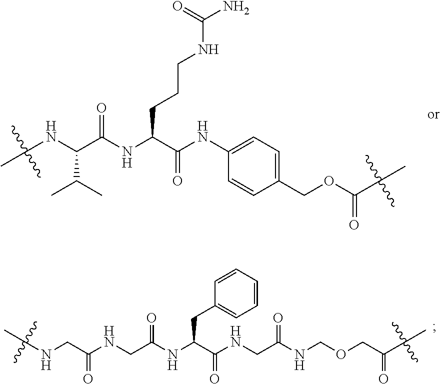 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]

n₁₆ is selected from any number between 1 and 20;
the cytotoxic small-molecule drug is a drug for inflammatory diseases, nervous system diseases, or cardiovascular diseases; the active agent is a tubulin inhibitor, an RNA polymerase inhibitor, a topoisomerase inhibitor, an intercalator, a DNA-reactive agent, a DNA alkylating agent, or an immunosuppressant;
the Ab is Abciximab, Alemtuzumab, Anetumab, Atezolizumab, Avelumab, Basiliximab, Bevacizumab, Blinatomumab, Brentuximab, Catumaxomab, Cetuximab, Cirmtuzumab, Coltuximab, Daclizumab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Omalizumab, Palivizumab, Panitumumab, Patritumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, Trastuzumab, an anti-B7-H3 monoclonal antibody, an anti-CD4 antibody, an anti-CD5 antibody, an anti-CD13 antibody, an anti-CD30 antibody, or a mutant of the above antibodies at positions 226 or 229 of the heavy chain, an antigen-binding fragment thereof, or an immunologically active portion thereof.

According to an embodiment of the present invention, in Formula (III),
L₁ is selected from

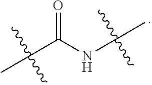 [SMILES: *NC(*)=O]

M is selected from

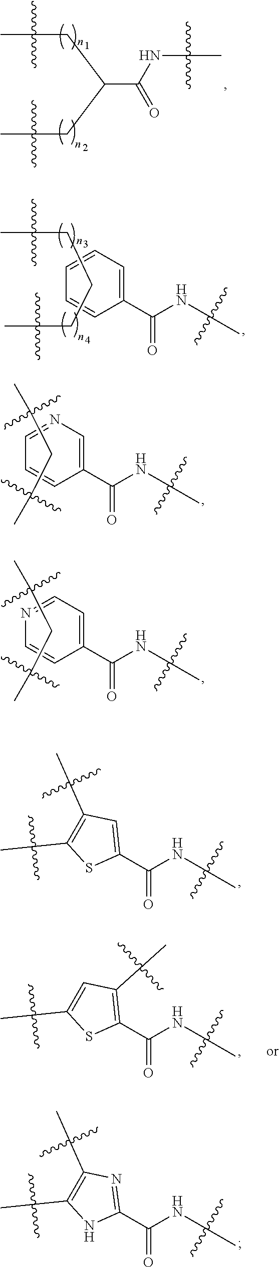

n₁ and n₂ are the same, and n₃ and n₄ are the same; n₁=an integer from 0 to 8, n₂=an integer from 0 to 8, n₃=an integer from 0 to 8, and n₄=an integer from 0 to 8;
L₂ is selected from

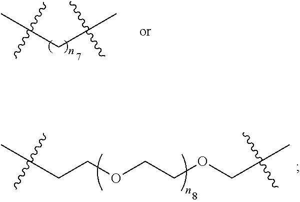

n₇=an integer from 1 to 10, and n₈=an integer from 1 to 10;
L₃ is

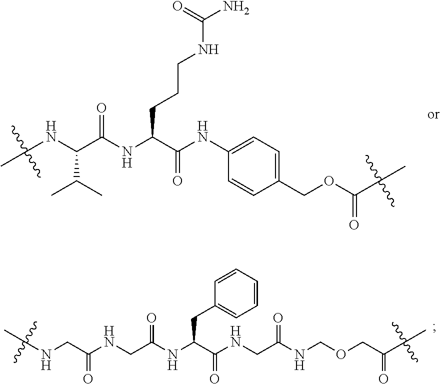 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]

the cytotoxic small-molecule drug is a drug for the treatment of tumor diseases; the active agent is a tubulin inhibitor, an RNA polymerase inhibitor, a topoisomerase inhibitor, an intercalator, a DNA-reactive agent, a DNA alkylating agent, or an immunosuppressant;
n₁₆ is selected from any number between 2 and 6;
the Ab is selected from Patritumab, Sacituzumab, Mirvetuximab, Cetuximab, Trastuzumab, an anti-B7-H3 monoclonal antibody, Cirmtuzumab, or a mutant of the above antibodies at EU numbering position 226 or EU numbering position 229 of the heavy chain.

The mutant is obtained by mutating the cysteine residue that forms a disulfide bond between heavy chains into a serine residue.

According to an embodiment of the present invention, the antibody-drug conjugate of Formula (III) is selected from at least one of the compounds represented by the following structural formulas:

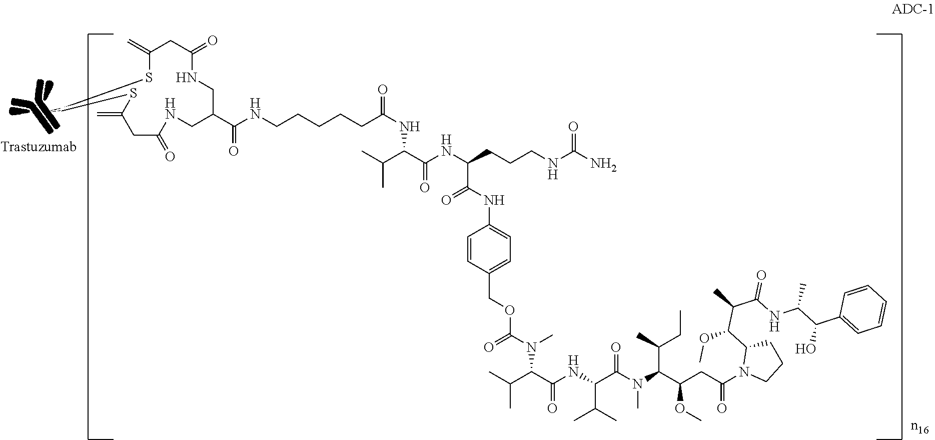

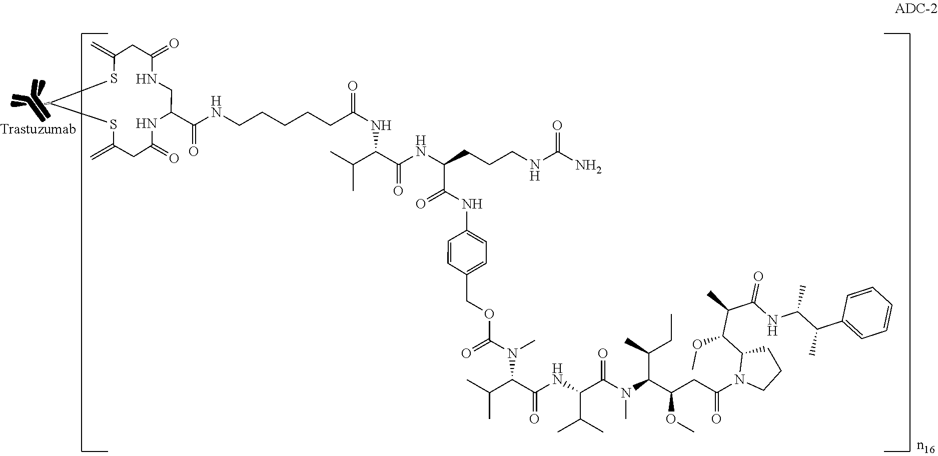

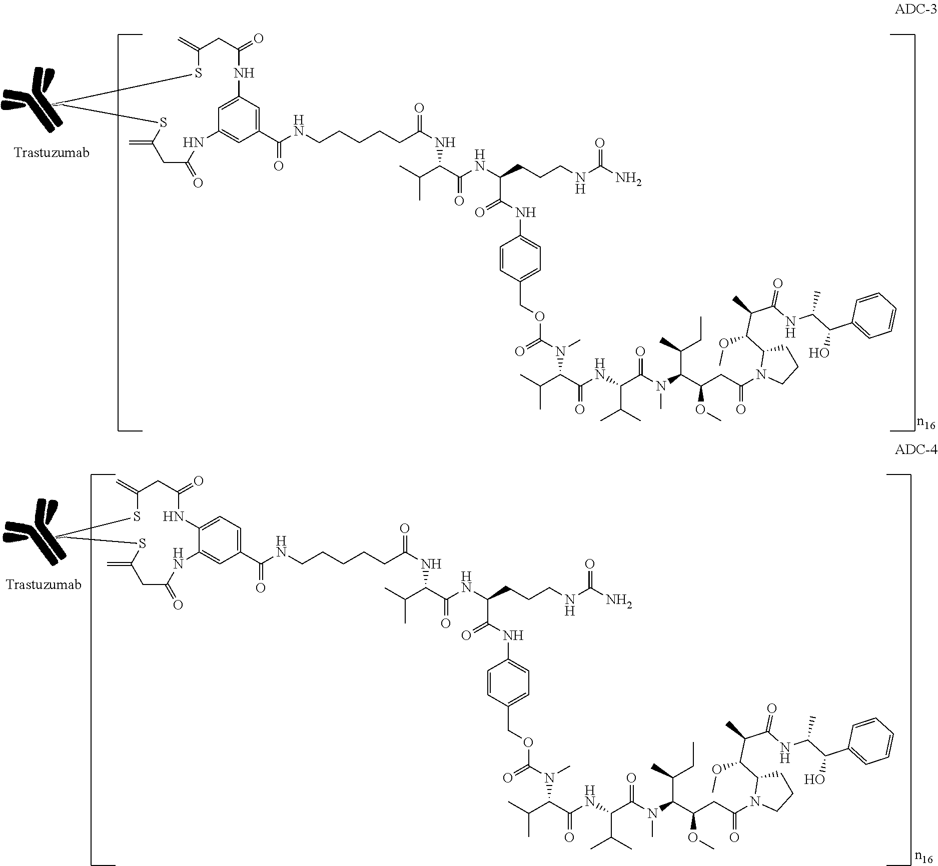

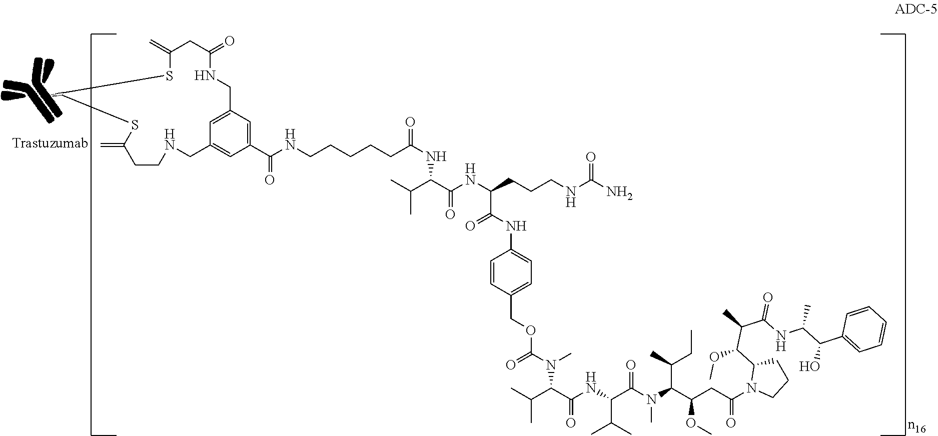

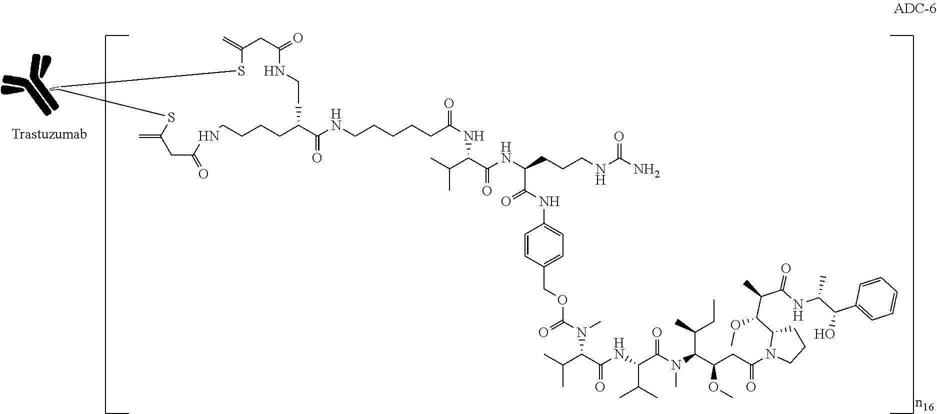

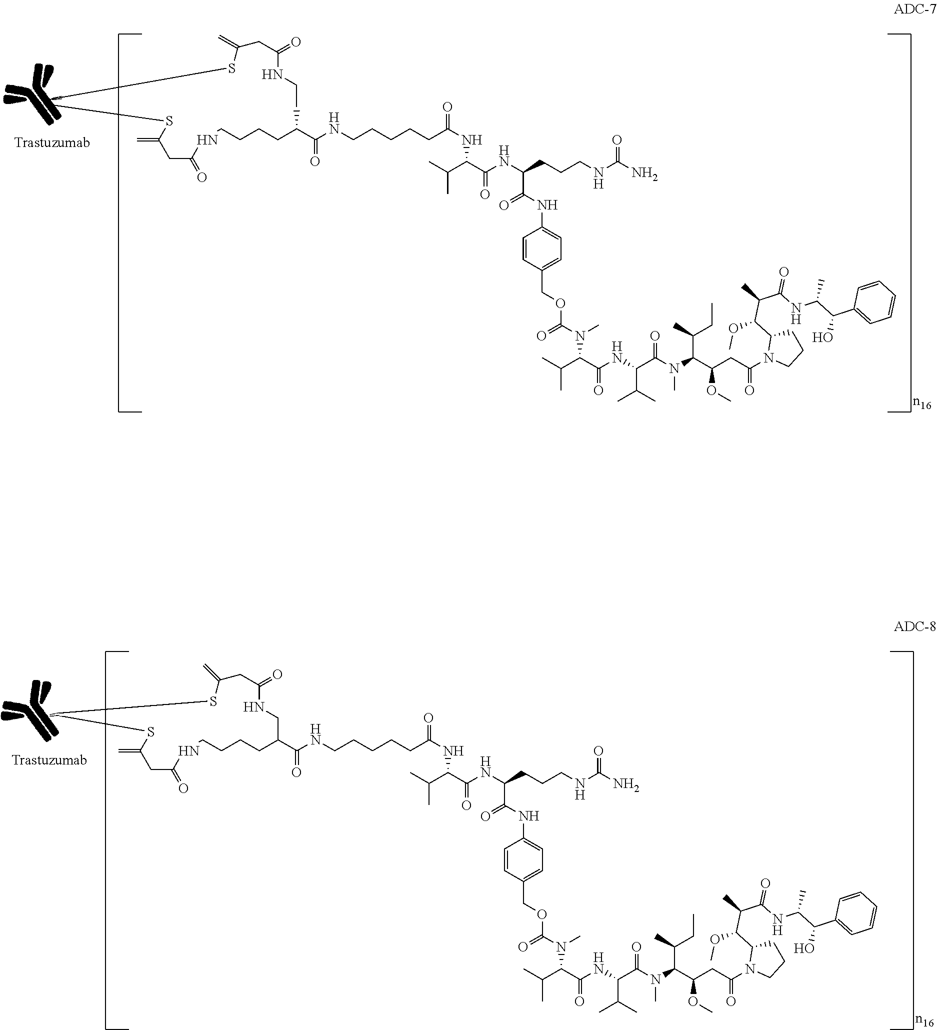

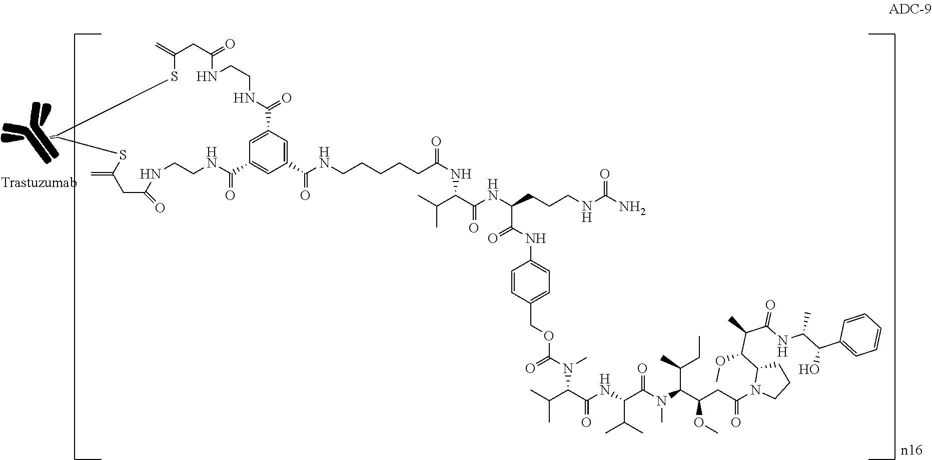

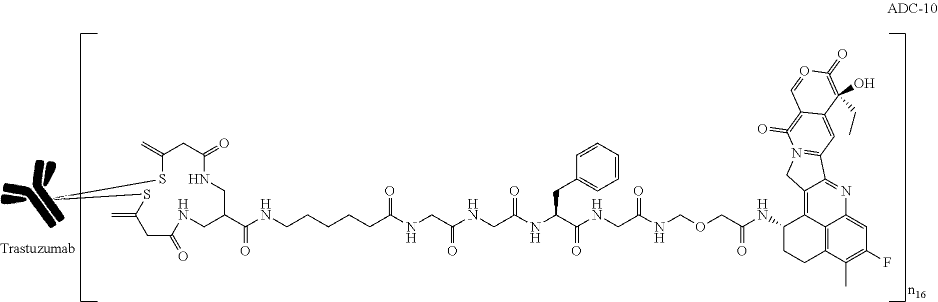

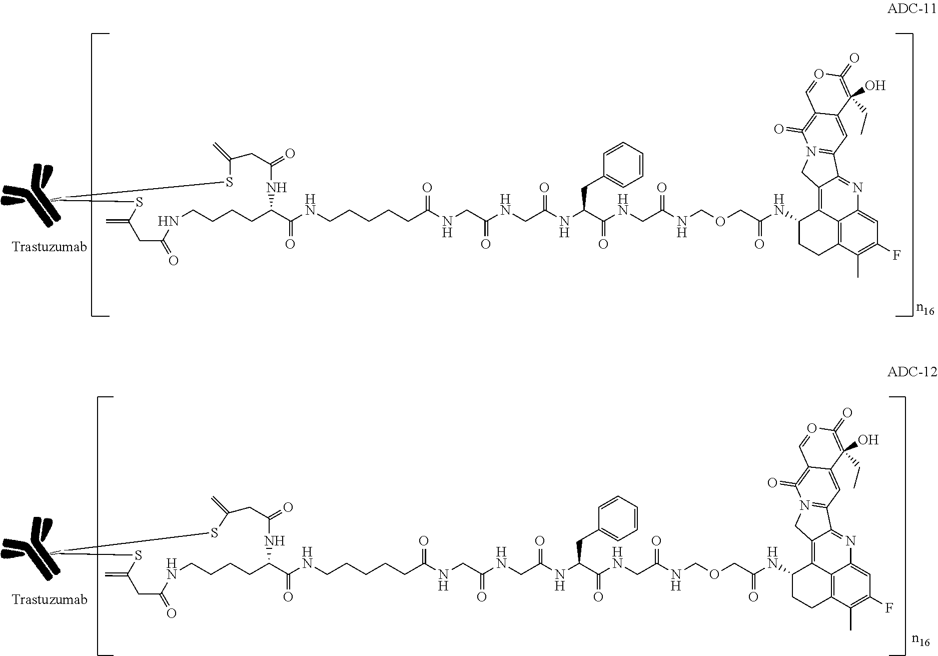

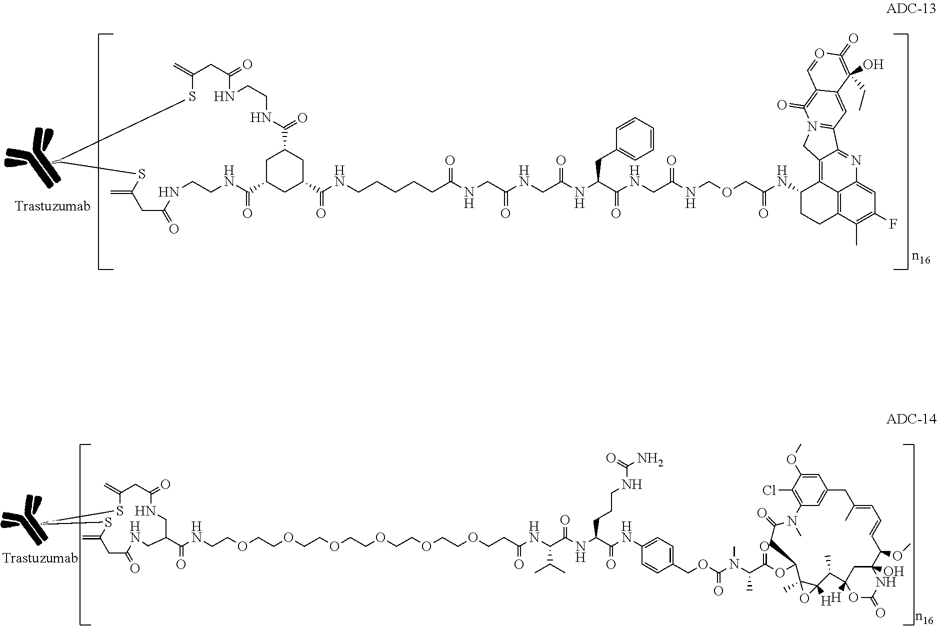

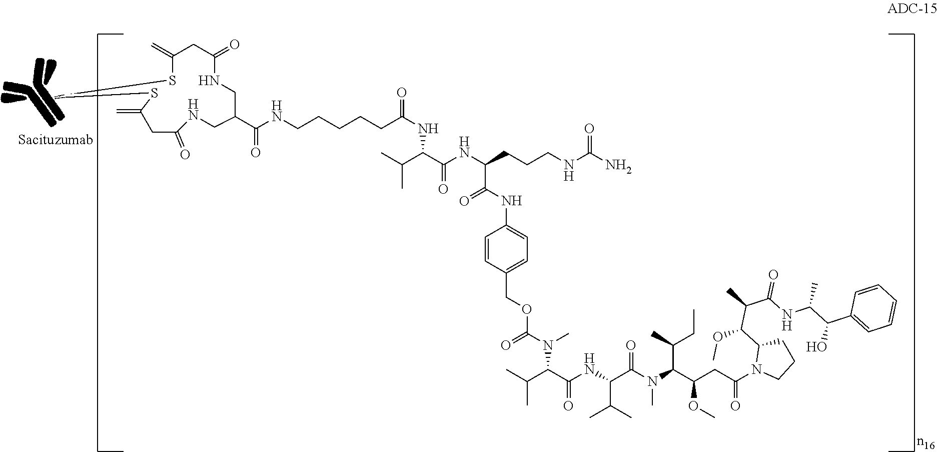

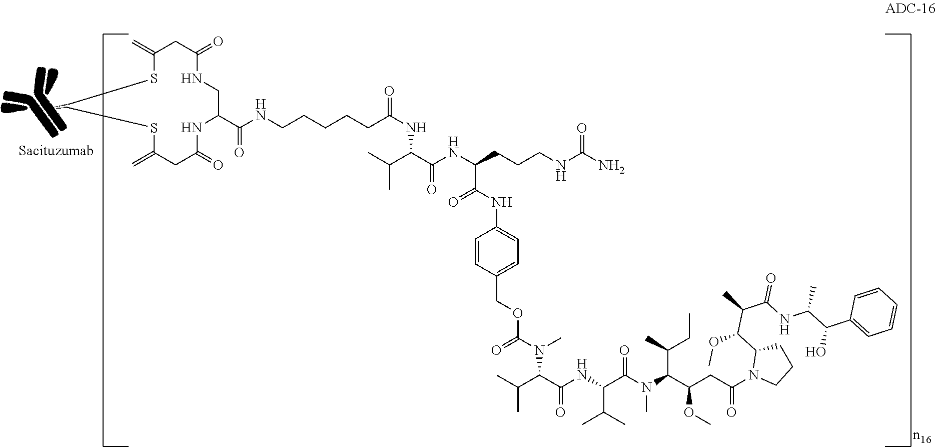

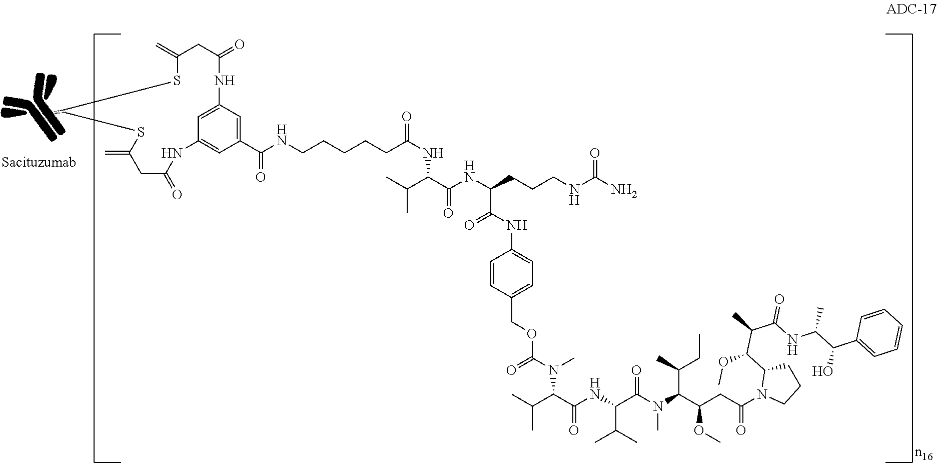

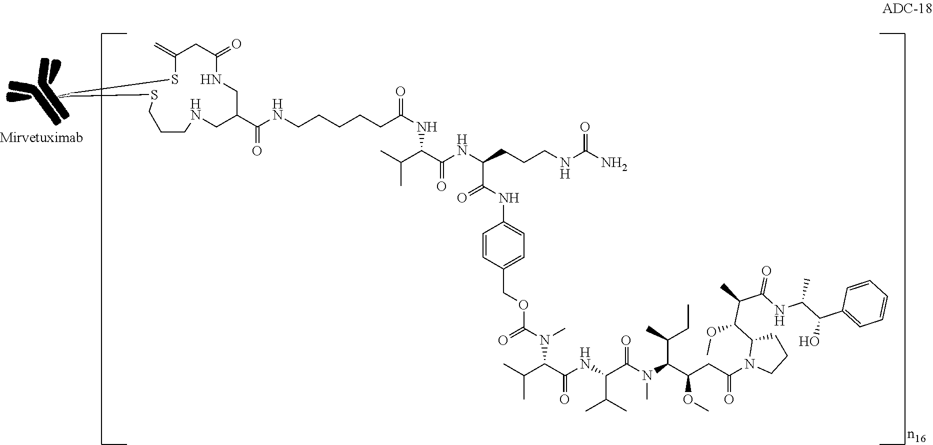

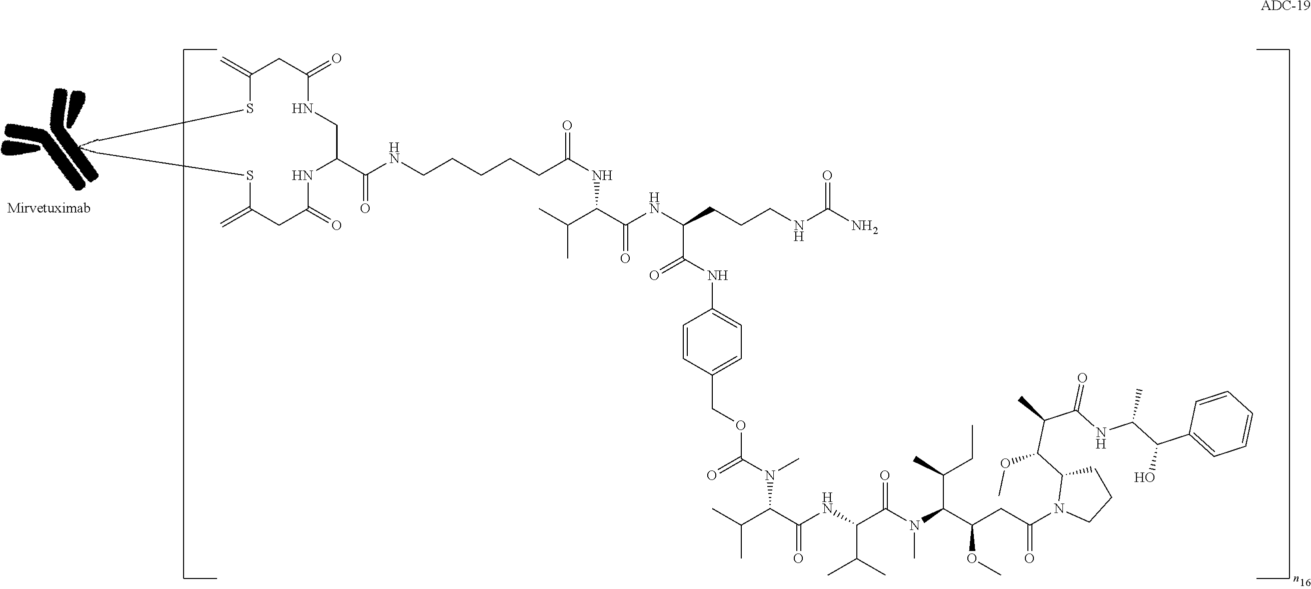

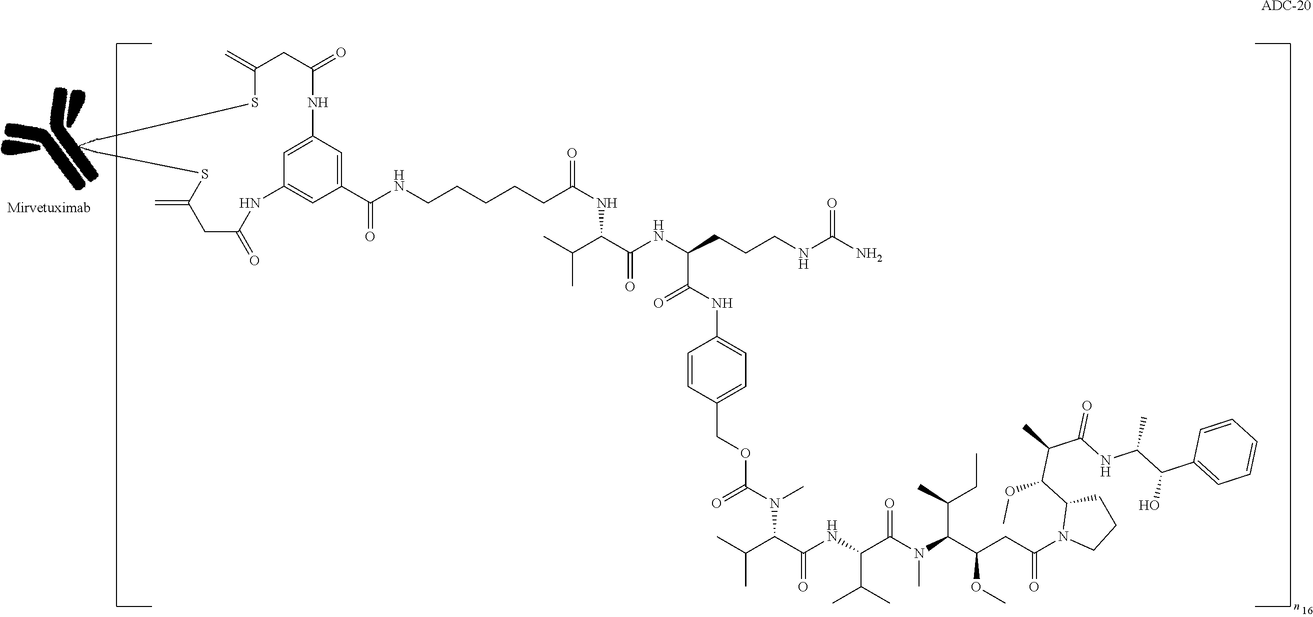

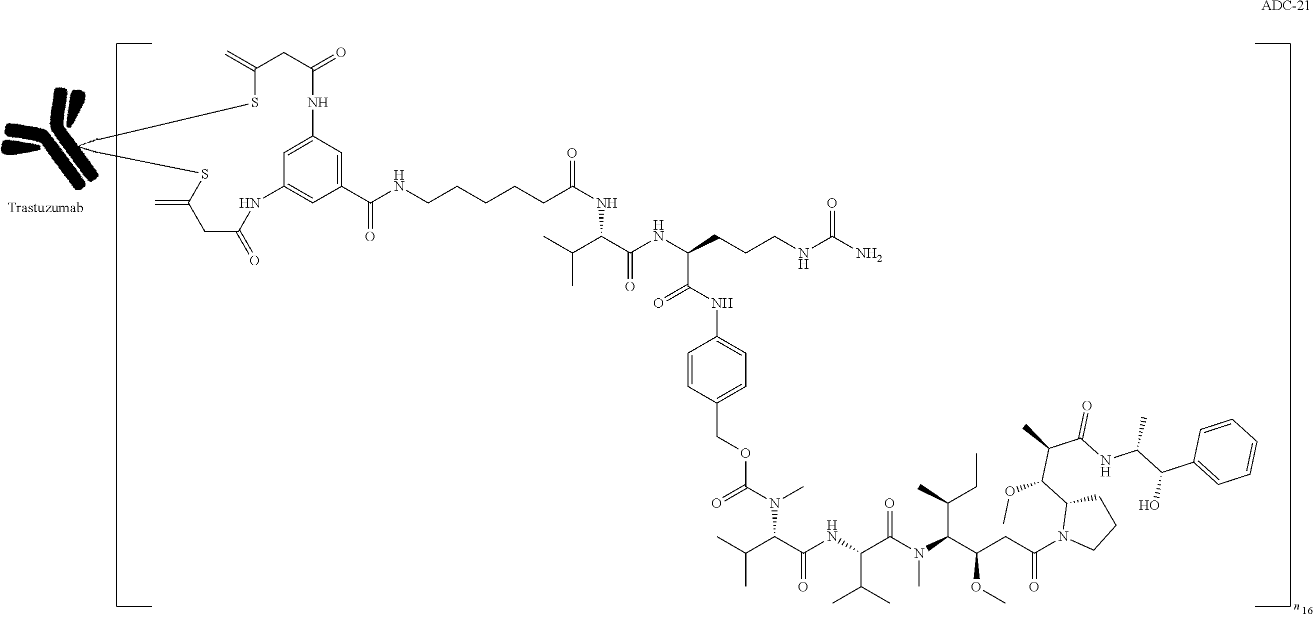

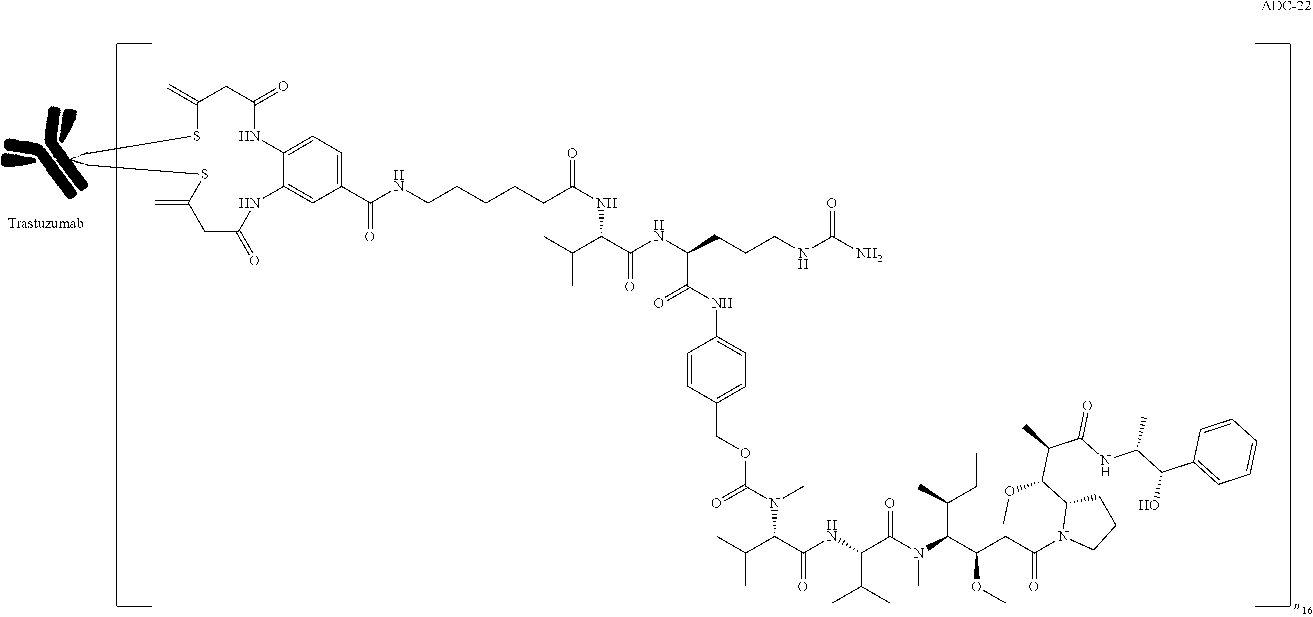

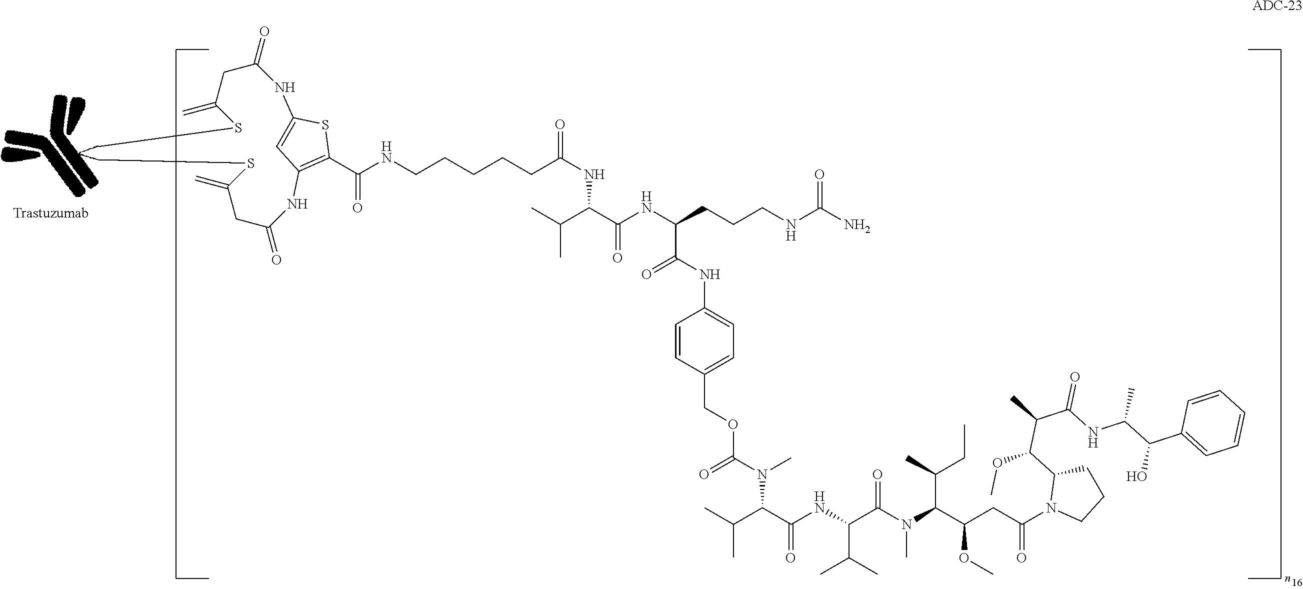

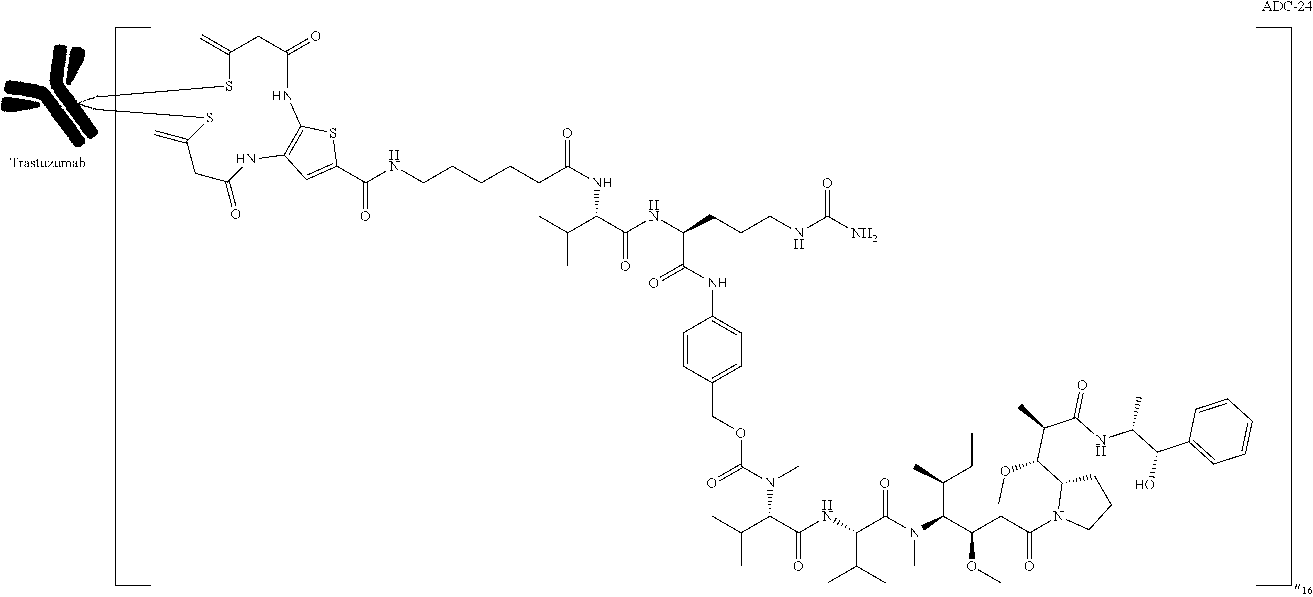

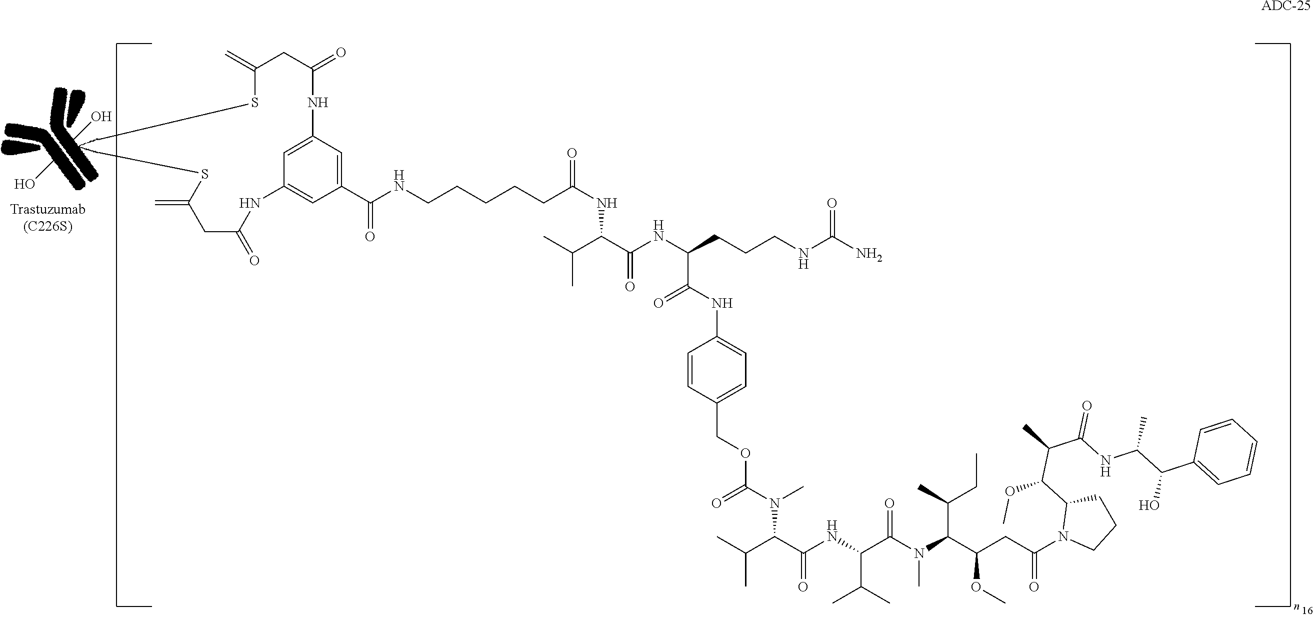

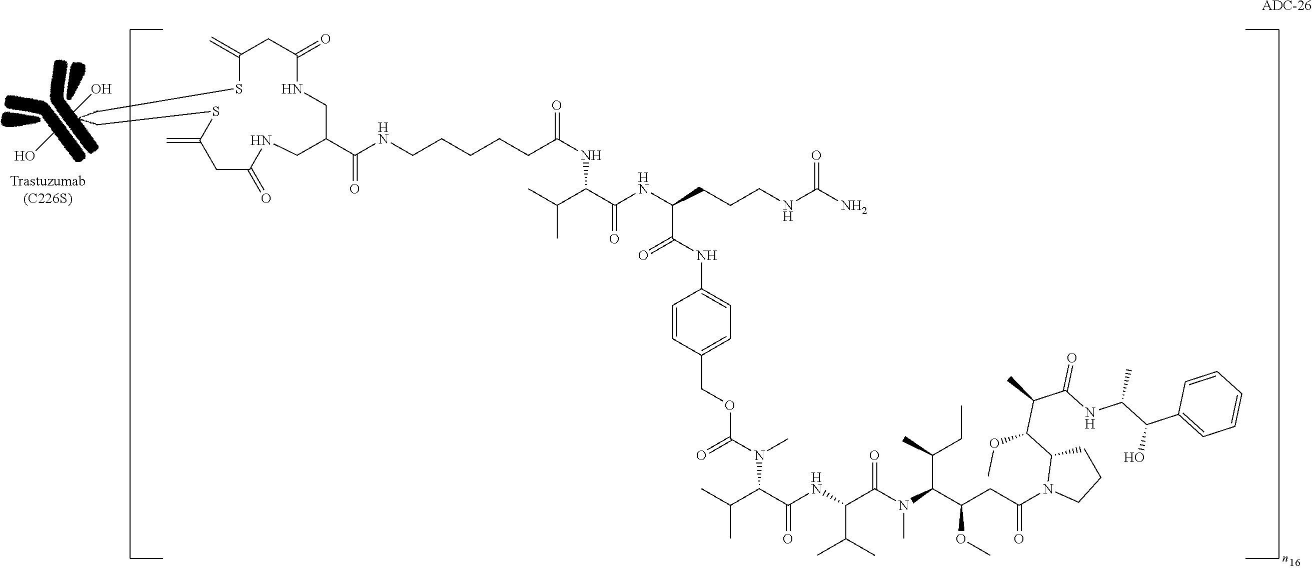

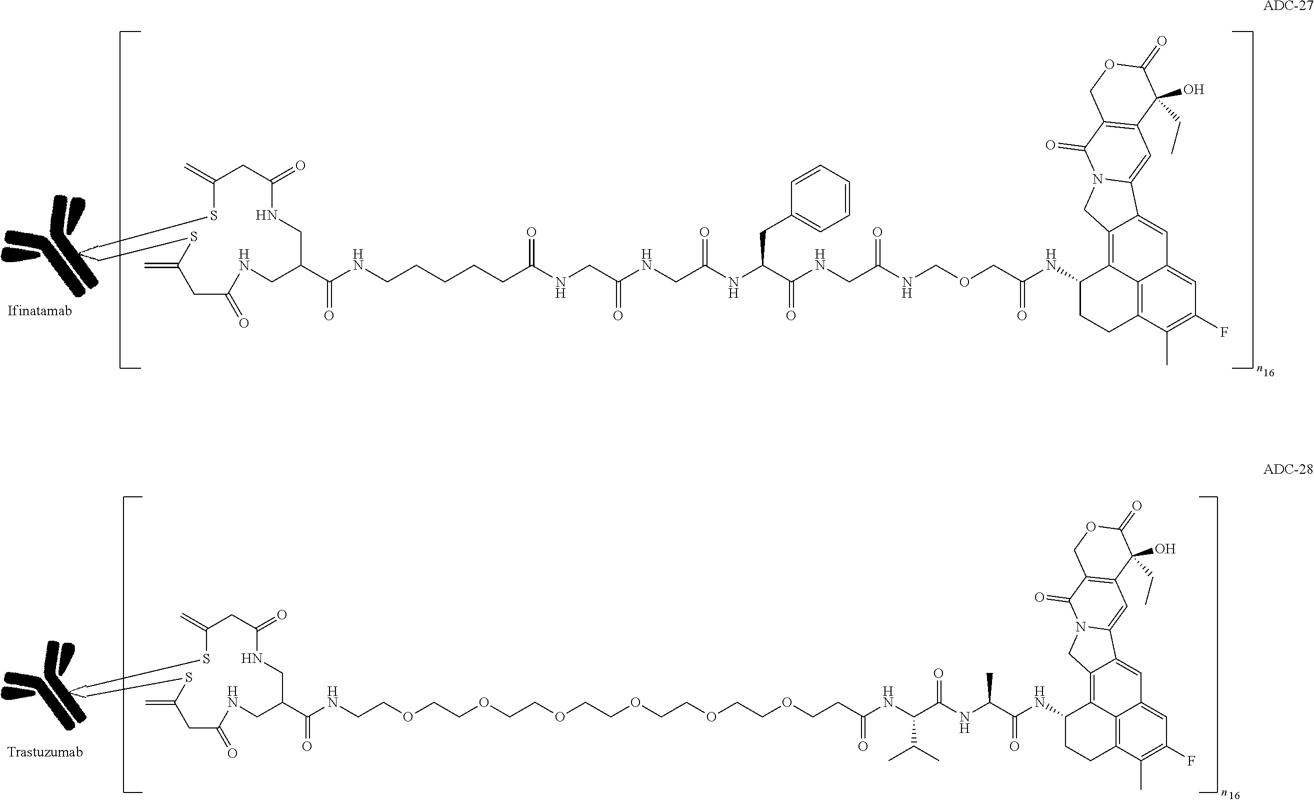

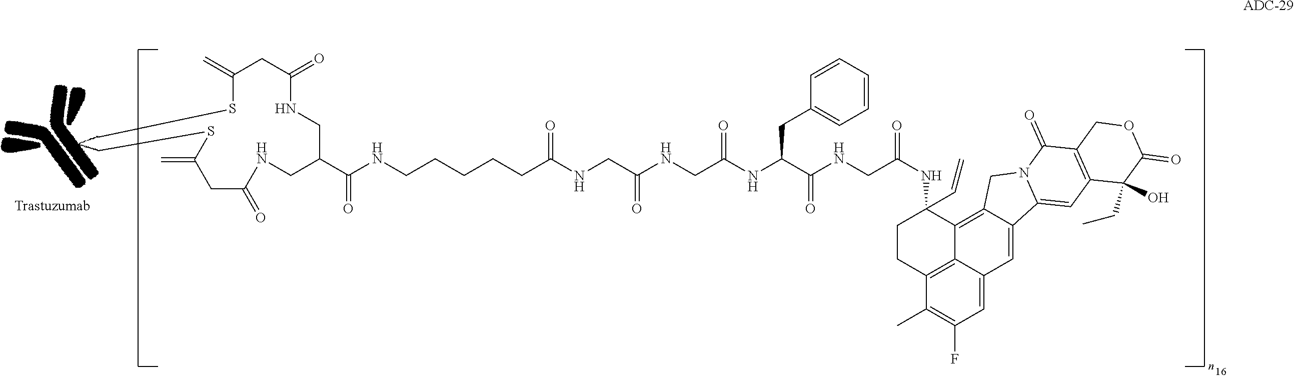

n₁₆ is selected from any number between 2 and 6.

The S in Formula (III) and the structural formulas of ADC-1 to ADC-29 described in the present invention represent the sulfur atom contained in the antibody Ab.

The tubulin inhibitor described in the present invention includes, but is not limited to, monomethyl auristatin, maytansinoid, maytansinol, or DM1 maytansinoid. The monomethyl auristatin may be monomethyl auristatin E, monomethyl auristatin F, and the like; the RNA polymerase inhibitor includes, but is not limited to, α-amanitin and the like; the topoisomerase inhibitor includes, but is not limited to, etoposide, teniposide, amsacrine, SN-38, exatecan, and the like; the intercalator includes, but is not limited to, pyrrolobenzodiazepine (PBD) and the like; the DNA-reactive agent includes, but is not limited to, calicheamicin, tiancimycins or other enediynes, and the like; the DNA alkylating agent includes, but is not limited to, trabectedin, lurbinectedin, and other ecteinascidin derivatives.

The English names of the antibodies described in the present invention are as follows: Abciximab, Alemtuzumab, Anetumab, Atezolizumab, Avelumab, Basiliximab, Bevacizumab, Blinatomumab, Brentuximab, Catumaxomab, Cetuximab, Cirmtuzumab, Coltuximab, Daclizumab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Omalizumab, Palivizumab, Panitumumab, Patritumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, Trastuzumab. The anti-B7-H3 monoclonal antibody is selected from Ifinatamab.

Provided is the use of the antibody-drug conjugate as described above in the treatment of cancer.

The cancer includes lymphoma, blastoma, sarcoma, leukemia or lymphoid malignancy, squamous cell carcinoma (e.g., epithelial squamous cell carcinoma), lung cancer (including small-cell lung cancer, non-small cell lung cancer, lung adenocarcinoma, and lung squamous cell carcinoma), peritoneal cancer, liver cancer, gastric cancer, stomach cancer (including gastrointestinal cancer), pancreatic cancer, glioma, cervical tumor, ovarian cancer, oral cancer, bladder cancer, urinary tract cancer, breast cancer, HER2-positive breast cancer, colon cancer, rectal cancer, colorectal cancer, endometrial cancer or uterine carcinoma, salivary gland cancer, kidney cancer, prostate cancer, thyroid cancer, melanoma, multiple myeloma, B-cell lymphoma, brain cancer, head and neck cancer, and related metastatic tumors.

The main advantages of the present invention are as follows:
1. The present invention provides a novel bis-allene linker, which can conveniently achieve site-specific conjugation to antibodies based on covalent thiol bridging, and is applicable to most antibodies. Compared with the cumbersome recombinant modification of antibodies, the method for site-specific conjugation using the bis-allene linker of the present invention does not require recombinant modification of antibodies, thus saving a great deal of time, costs, and resources, and exhibiting broader application prospects.
2. The novel bis-allene linker provided by the present invention can achieve covalent bridging conjugation with antibodies via a simple chemical method. Compared with the antibody-drug conjugates prepared by traditional random conjugation methods, the conjugates obtained by using the linker of the present invention exhibit a narrower distribution of DAR values and higher product homogeneity. The proportion of the homogeneous component (DAR4) in the obtained conjugates can exceed 80%, and even exceed 95% in some cases, with the “half-antibody” (DAR4) component being predominant.
3. The novel bis-allene linker provided by the present invention can also achieve covalent bridging conjugation with mutants of most antibodies, for example, mutants at positions 226 (Eu numbering) or 229 (Eu numbering) of the heavy chain. Since the cysteine residues that form disulfide bonds between heavy chains are mutated into other amino acids, homogeneous DAR3 antibody-drug conjugates can be generated.
4. With the same antibody employed, the antibody-drug conjugates based on the novel bis-allene linker provided by the present invention exhibit comparable in vitro cytotoxic activity and superior in vitro plasma stability, compared with the antibody-drug conjugates based on the maleimide linker.

### BRIEF DESCRIPTION OF THE DRAWINGS

FIG. 1 shows the HIC chromatogram of ADC-1 prepared in an example of the present invention. It can be seen from FIG. 1 that the proportion of the homogeneous component (DAR4) in the obtained conjugate can exceed 80%.

FIG. 2 shows the SEC chromatogram of ADC-1 prepared in an example of the present invention.

FIG. 3 shows the MS spectrum of ADC-1 prepared in an example of the present invention.

FIG. 4 shows the SDS-PAGE profile of ADC-1 prepared in an example of the present invention.

FIG. 5 shows the HIC chromatogram of ADC-2 prepared in an example of the present invention.

FIG. 6 shows the SEC chromatogram of ADC-2 prepared in an example of the present invention.

FIG. 7 shows the HIC chromatogram of ADC-3 prepared in an example of the present invention.

FIG. 8 shows the SEC chromatogram of ADC-3 prepared in an example of the present invention.

FIG. 9 shows the SDS-PAGE profile of ADC-3 prepared in an example of the present invention.

FIG. 10 shows the HIC chromatogram of ADC-5 prepared in an example of the present invention.

FIG. 11 shows the SEC chromatogram of ADC-5 prepared in an example of the present invention.

FIG. 12 shows the SDS-PAGE profile of ADC-5 prepared in an example of the present invention.

FIG. 13 shows the HIC chromatogram of ADC-10 prepared in an example of the present invention. It can be seen from FIG. 13 that the proportion of the homogeneous component (DAR4) in the obtained conjugate can exceed 95%.

FIG. 14 shows the SEC chromatogram of ADC-10 prepared in an example of the present invention.

FIG. 15 shows the SDS-PAGE profile of ADC-10 prepared in an example of the present invention.

FIG. 16 shows the HIC chromatogram of ADC-25 prepared in an example of the present invention.

FIG. 17 shows the SEC chromatogram of ADC-25 prepared in an example of the present invention.

FIG. 18 shows the SDS-PAGE profile of ADC-25 prepared in an example of the present invention.

FIG. 19 shows the HIC chromatogram of ADC-26 prepared in an example of the present invention.

FIG. 20 shows the SEC chromatogram of ADC-26 prepared in an example of the present invention.

FIG. 21 shows the SDS-PAGE profile of ADC-26 prepared in an example of the present invention.

FIG. 22 shows the HIC chromatogram of ADC-27 prepared in an example of the present invention. It can be seen from FIG. 22 that the proportion of the homogeneous component (DAR4) in the obtained conjugate can exceed 95%.

FIG. 23 shows the SEC chromatogram of ADC-27 prepared in an example of the present invention.

FIG. 24 shows the MS spectrum of ADC-27 prepared in an example of the present invention.

FIG. 25 shows the SDS-PAGE profile of ADC-27 prepared in an example of the present invention.

FIG. 26 shows the results of the in vitro plasma stability assay of ADCs based on different linkers.

### DETAILED DESCRIPTION OF THE INVENTION

The following are examples of the synthesis and analysis of the compounds (including linkers and linker-drug conjugates) and antibody-drug conjugates described in this patent. It should be noted that these examples are not intended to limit the scope of the present invention.

Unless otherwise stated, all anhydrous reagents were purchased directly from commercial suppliers and stored under a nitrogen atmosphere.

The definitions and schematic diagrams of the “half-antibody” and “full-antibody” described in this patent are as follows:

As shown on the right, the “full-antibody” is formed by conjugation via light chain-heavy chain bridging and heavy chain-heavy chain bridging:

[symbol]

As shown on the right, the “half-antibody” is formed by conjugation via light chain-heavy chain bridging and intra-heavy chain bridging:

[symbol]

### Synthesis and Characterization of Linkers

### Example 1: Synthesis of Compound LK1

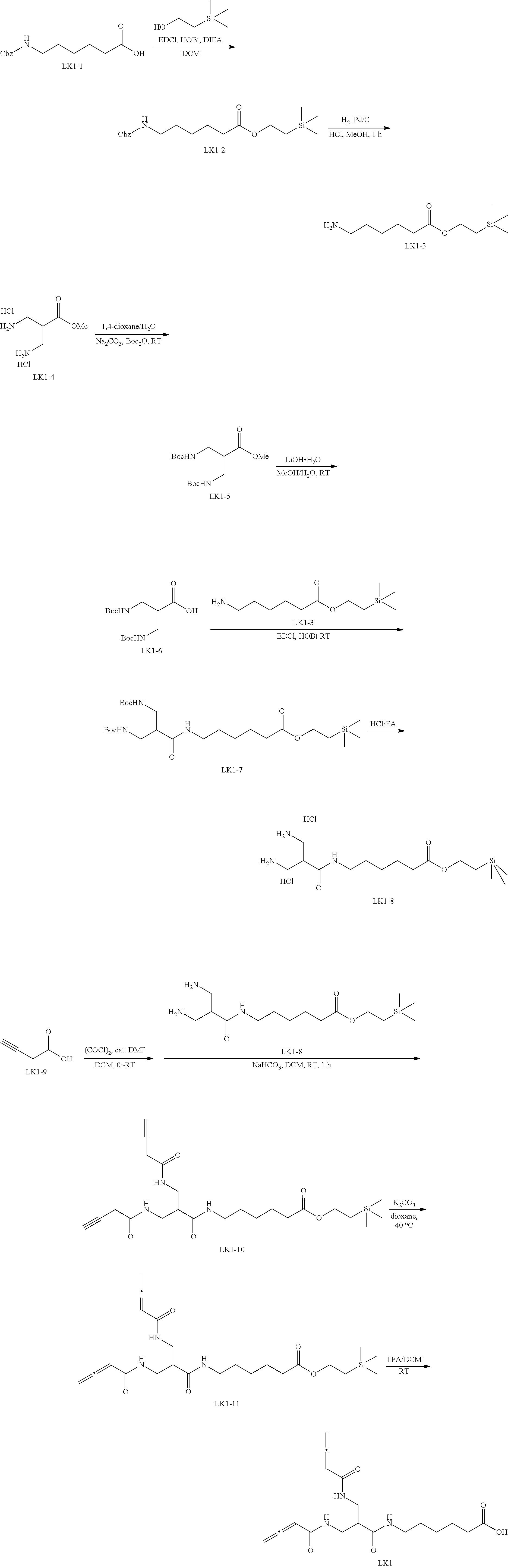

### Step 1: Synthesis of 2-(trimethylsilyl)ethyl 6-(((benzyloxy)carbonyl)amino)hexanoate

To a solution of 6-(((benzyloxy)carbonyl)amino)hexanoic acid (5.0 g, 0.0188 mmol), EDCI (3.98 g, 0.0207 mol), and HOBt (2.80 g, 0.0207 mol) in 30 mL of dichloromethane was added 2-(trimethylsilyl)ethan-1-ol (2.23 mg, 0.0188 mmol) and DIPEA (4.87 g, 0.0376 mol). After the addition was complete, the mixture was stirred at room temperature for 2 hours. The mixture was concentrated, redissolved in MTBE, washed with brine and saturated sodium bicarbonate solution, and then concentrated. The residue was purified by flash column chromatography on silica gel (eluted with ethyl acetate/heptane=1:3) to give the product (5.5 g, 79.8% yield) as a white solid.

LC-MS (ESI) m/z: 338.0 [M−2CH₃+3H]⁺.

¹H NMR (300 MHz, DMSO-d₆) δ 7.33 (q, J=5.6, 4.1 Hz, 5H), 7.21 (d, J=7.5 Hz, 1H), 4.99 (s, 2H), 4.19-4.01 (m, 2H), 2.96 (q, J=6.5 Hz, 2H), 2.23 (t, J=7.3 Hz, 2H), 1.57-1.44 (m, 2H), 1.38 (t, J=7.4 Hz, 2H), 1.24 (d, J=7.0 Hz, 3H), 1.02-0.85 (m, 2H), 0.01 (d, J=2.4 Hz, 9H).

### Step 2: Synthesis of 2-(trimethylsilyl)ethyl 6-aminohexanoate

2-(Trimethylsilyl)ethyl 6-(((benzyloxy)carbonyl)amino)hexanoate (2.5 g, 6.8 mmol) was diluted with 30 mL of MeOH and concentrated HCl (0.74 g, 7.4 mmol), followed by the addition of 0.3 g of 5% Pd/C under a N₂ atmosphere. The suspension was evacuated and purged with H₂ several times. The mixture was stirred under 1 MPa of H₂ for 1 hour. The suspension was filtered through a pad of Celite and washed with MeOH. The combined filtrate was concentrated to dryness to give an oily liquid, which was used directly in the next step without further purification.

¹H NMR (400 MHz, DMSO-d₆) δ 7.87 (s, 3H), 4.10 (t, J=8.4 Hz, 2H), 2.74 (s, 2H), 2.27 (t, J=7.4 Hz, 3H), 1.53 (h, J=7.2 Hz, 5H), 1.31 (q, J=7.9 Hz, 2H), 0.94 (t, J=8.4 Hz, 2H), 0.03 (d, J=1.8 Hz, 8H).

### Step 3: Synthesis of methyl 3-((tert-butoxycarbonyl)amino)-2-((tert-butoxycarbonyl)amino)methylpropanoate

To a solution of methyl 3-amino-2-(aminomethyl)propanoate (900 mg, 4.383 mmol) in 1,4-dioxane/H₂O (30 mL, 1:1) was added Na₂CO₃ (2.33 g, 21.9 mmol). The mixture was stirred for 0.5 hour and cooled to 0° C. Boc₂O (3.9 g, 17.53 mmol) was added to the reaction mixture, and the mixture was stirred at room temperature overnight. Upon completion of the reaction, the mixture was extracted with ethyl acetate (50 mL×3). The organic layer was dried over anhydrous sodium sulfate and concentrated. Purification was performed by column chromatography (heptane/ethyl acetate=10:1 to 5:1) to give the desired product (1.23 g, 83.9%) as a colourless oil.

¹H NMR (400 MHz, Chloroform-d) δ 5.23 (s, 1H), 3.70 (s, 3H), 3.56 (q, J=15.7, 12.1 Hz, 2H), 3.21 (dd, J=13.4, 6.8 Hz, 2H), 2.74 (p, J=5.6 Hz, 1H), 1.43 (s, 19H).

### Step 4: Synthesis of 3-((tert-butoxycarbonyl)amino)-2-((tert-butoxycarbonyl)amino)methylpropanoic Acid

To a solution of methyl 3-((tert-butoxycarbonyl)amino)-2-((tert-butoxycarbonyl)amino)methylpropanoate (1.23 g, 3.7 mmol) in MeOH/H₂O (2:1, 12 mL), maintained at 0° C., was added LiOH·H₂O (776.3 mg, 18.5 mmol). The mixture was stirred at room temperature for 0.5 hour and adjusted to pH 3-4 with 1N HCl. The mixture was extracted with ethyl acetate (50 mL×3), and the organic layer was washed with saturated brine. The organic layer was separated, dried over anhydrous sodium sulfate, concentrated, and then used in the next step without further purification.

¹H NMR (400 MHz, Chloroform-d) δ 5.38 (s, 1H), 3.65-3.15 (m, 4H), 2.76 (q, J=5.7 Hz, 1H), 1.43 (s, 19H).

### Step 5: Synthesis of 2-(trimethylsilyl)ethyl 6-(3-((tert-butoxycarbonyl)amino)-2-(((tert-butoxycarbonyl)amino)methyl)propanamido)hexanoate

To a solution of 3-((tert-butoxycarbonyl)amino)-2-((tert-butoxycarbonyl)amino)methylpropanoic acid (1.0 g, 3.14 mmol) in dichloromethane (20 mL) was added EDCI (658 mg, 3.45 mmol), HOBt (463.3 mg, 3.45 mmol), and 2-(trimethylsilyl)ethyl 6-aminohexanoate (1.46 g, 31.19 mmol). The mixture was stirred at room temperature overnight. The mixture was washed with 5% citric acid solution (50 mL×3) and then with water (50 mL×3). The organic layer was dried over anhydrous sodium sulfate, concentrated, and purified by column chromatography (ethyl acetate/n-heptane=1:1) to give the desired product (870 mg, 52.1%) as a white solid.

¹H NMR (400 MHz, Chloroform-d) δ 6.54 (s, 1H), 5.51 (s, 2H), 4.23-4.14 (m, 2H), 3.54-3.42 (m, 2H), 3.31-3.20 (m, 4H), 2.57 (p, J=5.4 Hz, 1H), 2.32 (t, J=7.4 Hz, 2H), 1.71-1.62 (m, 2H), 1.60-1.50 (m, 3H), 1.47 (s, 18H), 1.43-1.34 (m, 3H), 1.05-0.96 (m, 2H), 0.07 (s, 9H).

### Step 6: Synthesis of 2-(trimethylsilyl)ethyl 6-(3-amino-2-(aminomethyl)propanamido)hexanoate Dihydrochloride

To a solution of 2-(trimethylsilyl)ethyl 6-(3-((tert-butoxycarbonyl)amino)-2-(((tert-butoxycarbonyl)amino)methyl)propanamido)hexanoate (500 mg, 0.94 mmol) in ethyl acetate (7 mL) was added 2N HCl/EA (2.4 ml). The mixture was stirred at room temperature for 2 days. The solvent was removed by concentration, and acetonitrile was added for trituration. The solid was filtered and dried to give the desired product (300 mg, 79.1%) as a white solid salt.

¹H NMR (400 MHz, Chloroform-d) δ 8.16 (s, 4H), 4.14 (t, J=8.4 Hz, 2H), 3.58 (s, 4H), 3.21 (s, 2H), 2.64 (s, 2H), 2.29 (s, 2H), 1.48 (d, J=93.5 Hz, 6H), 1.04-0.92 (m, 2H), 0.04 (s, 10H).

### Step 7: Synthesis of 2-(trimethylsilyl)ethyl 6-(3-(but-3-ynamido)-2-(but-3-ynamidomethyl)propanamido)hexanoate

To a suspension of but-3-ynoic acid (623.6 mg, 7.4 mmol) in dichloromethane (10 mL) was added oxalyl chloride (939.1 mg, 7.4 mmol). After stirring at room temperature for 15 min, the reaction mixture was cooled to 0° C., and 0.15 mL of DMF was added. The reaction mixture was stirred at 0° C. and then at room temperature for another 90 min. The above solution was added dropwise to a solution of 2-(trimethylsilyl)ethyl 6-(3-amino-2-(aminomethyl)propanamido)hexanoate dihydrochloride (300 mg, 0.74 mmol) in saturated sodium bicarbonate solution (10 mL), and the reaction mixture was further stirred at room temperature for 2 h. The reaction mixture was extracted with dichloromethane (50 mL×3) and concentrated. Purification was performed by column chromatography (ethyl acetate/n-heptane=1:1) to give the desired product (292 mg, 85%) as a white solid.

LC-MS (ESI) m/z: 464 [M+H]⁺.

¹H NMR (400 MHz, Chloroform-d) δ 7.52 (s, 2H), 6.67 (s, 1H), 4.19-4.12 (m, 2H), 3.71 (ddd, J=14.1, 7.8, 4.7 Hz, 2H), 3.31-3.24 (m, 3H), 3.23 (d, J=2.7 Hz, 5H), 2.63 (p, J=5.5 Hz, 1H), 2.41 (t, J=2.7 Hz, 2H), 2.28 (t, J=7.4 Hz, 2H), 1.67 (s, 8H), 1.62 (q, J=7.6 Hz, 2H), 1.53 (p, J=7.3 Hz, 2H), 1.39-1.32 (m, 3H), 1.04-0.93 (m, 2H), 0.04 (s, 9H).

### Step 8: Synthesis of 2-(trimethylsilyl)ethyl 6-(3-(buta-2,3-dienamido)-2-(buta-2,3-dienamidomethyl)propanamido)hexanoate

2-(trimethylsilyl)ethyl 6-(3-(but-3-ynamido)-2-(but-3-ynamidomethyl)propanamido)hexanoate (292 mg, 0.64 mmol) was dissolved in a solution of 18% aqueous K₂CO₃/dioxane (8 mL/8 mL). The reaction mixture was heated to 40° C. for 2 hours and extracted with citric acid (60 mL, 10% in water) and ethyl acetate (500 mL×3). The organic layer was dried over anhydrous sodium sulfate and concentrated to give an oily compound (210 mg, 71.9%).

¹H NMR (400 MHz, Chloroform-d) δ 7.08 (t, J=6.6 Hz, 2H), 6.96 (s, 1H), 5.63 (t, J=6.6 Hz, 2H), 5.29 (dd, J=6.6, 1.6 Hz, 4H), 4.19-4.10 (m, 2H), 3.80-3.71 (m, 2H), 3.21 (dt, J=14.1, 5.6 Hz, 4H), 2.63 (q, J=5.2, 4.3 Hz, 1H), 2.34-2.23 (m, 3H), 1.63 (p, J=7.5 Hz, 2H), 1.52 (p, J=7.3 Hz, 2H), 1.42-1.30 (m, 3H), 1.01-0.94 (m, 2H), 0.04 (s, 9H).

### Step 9: Synthesis of LK1

2-(trimethylsilyl)ethyl 6-(3-(buta-2,3-dienamido)-2-(buta-2,3-dienamidomethyl)propanamido)hexanoate (60 mg, 0.129 mmol) was dissolved in TFA/DCM (1:1, 1 mL) and stirred at room temperature for 1 hour. The solvent was removed by concentration, and purification was performed on a C₁₈ column (ACN/H₂O) to give 20 mg of a white solid.

LC-MS (ESI) m/z: 364 [M+H]⁺.

¹H NMR (400 MHz, DMSO-d₆) δ 11.95 (s, 1H), 7.84 (q, J=5.6 Hz, 3H), 5.75 (t, J=6.6 Hz, 2H), 5.28 (d, J=6.6 Hz, 4H), 3.18 (dh, J=26.9, 6.3 Hz, 5H), 3.02 (q, J=6.6 Hz, 2H), 2.57 (t, J=6.9 Hz, 1H), 2.18 (t, J=7.4 Hz, 2H), 1.47 (p, J=7.4 Hz, 2H), 1.38 (p, J=7.2 Hz, 2H), 1.24 (q, J=8.8, 8.2 Hz, 2H).

### Example 2: Synthesis of Compound LK2

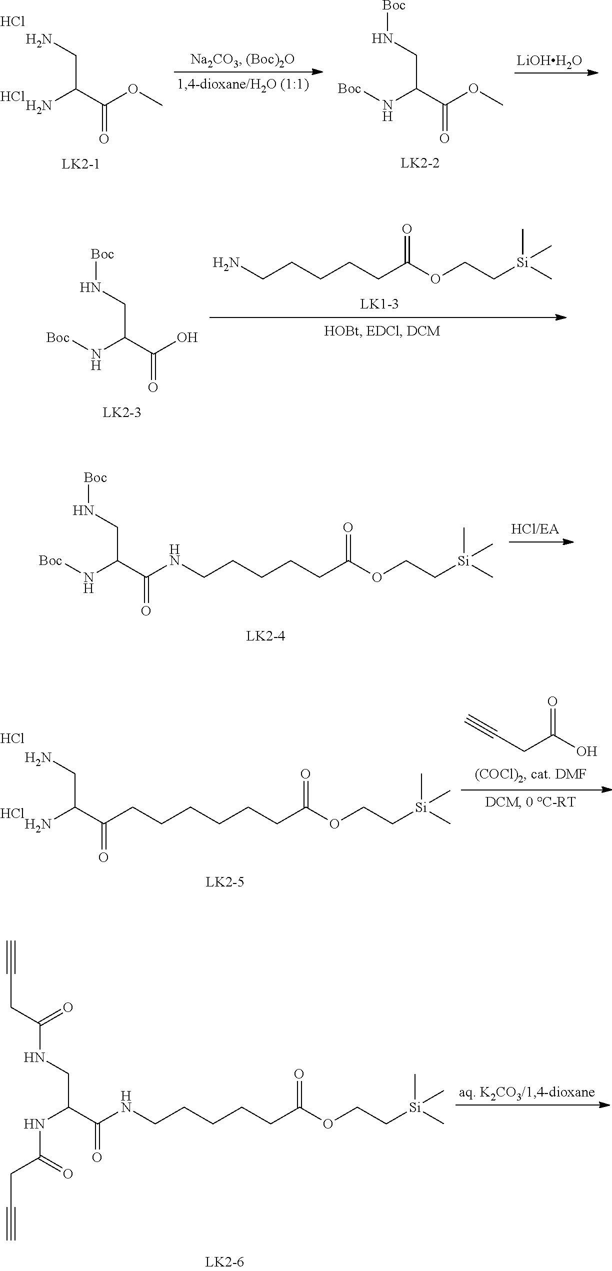

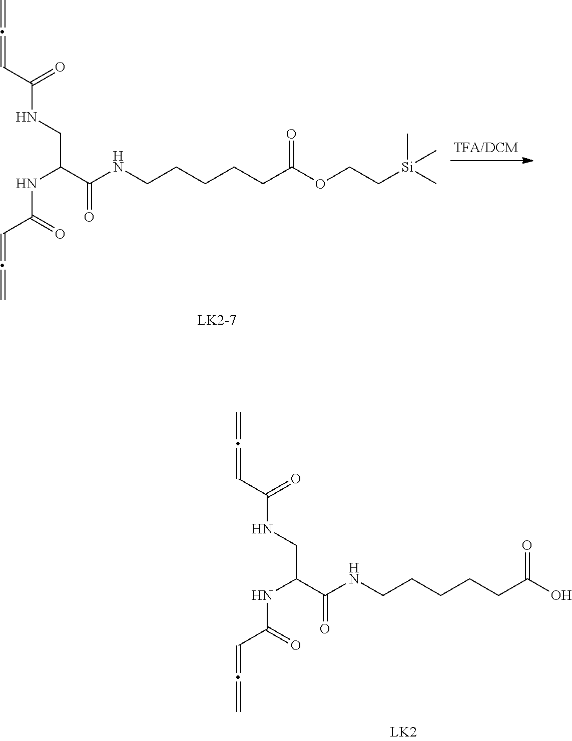 [SMILES: C=C=CC(=O)NCC(NC(=O)C=C=C)C(=O)NCCCCCC(=O)OCC[Si](C)(C)C ; C=C=CC(=O)NCC(NC(=O)C=C=C)C(=O)NCCCCCC(=O)O]

### Step 1: Synthesis of methyl 2,3-bis((tert-butoxycarbonyl)amino)propanoate

To a solution of methyl 2,3-diaminopropanoate dihydrochloride (2.5 g, 13.1 mmol) in 1,4-dioxane/H₂O (30 mL, 1:1) was added Na₂CO₃ (6.9 g, 65.7 mmol). The reaction mixture was stirred at 0° C. for 0.5 hour. Boc₂O (11.4 g, 52.4 mmol) was added to the reaction mixture, and the mixture was stirred at room temperature overnight. Upon completion of the reaction, the mixture was extracted with ethyl acetate (50 mL×3). The organic layers were combined, dried over anhydrous sodium sulfate, and concentrated. The residue was purified by flash column chromatography on silica gel (heptane/ethyl acetate=10:1 to 5:1) to give the desired product (2.0 g, 47.7% yield) as a colorless oil.

¹H NMR (400 MHz, DMSO-d₆) δ 7.06 (d, J=7.8 Hz, 1H), 6.84 (t, J=6.3 Hz, 1H), 4.03 (qd, J=7.1, 6.6, 3.8 Hz, 1H), 3.59 (s, 3H), 3.25 (ddt, J=19.4, 13.7, 6.6 Hz, 2H), 1.37 (d, J=5.2 Hz, 18H).

### Step 2: Synthesis of 2,3-bis((tert-butoxycarbonyl)amino)propanoic Acid

To a solution of methyl 2,3-bis((tert-butoxycarbonyl)amino)propanoate (1.23 g, 3.7 mmol) in MeOH/H₂O (2:1, 12 mL) was added LiOH·H₂O (776.3 mg, 18.5 mmol) at 0° C. The mixture was stirred at room temperature for 0.5 hour and adjusted to pH 3-4 with 1 HCl. The mixture was extracted with ethyl acetate (50 mL×3), and the organic phase was washed with saturated brine. The organic phase was separated, dried over anhydrous sodium sulfate, concentrated, and then used in the next step without further purification.

¹H NMR (400 MHz, DMSO-d₆) δ 6.80 (dd, J=16.6, 7.0 Hz, 2H), 3.96 (q, J=6.6 Hz, 1H), 3.27-3.15 (m, 2H), 1.37 (d, J=3.9 Hz, 20H).

### Step 3: Synthesis of 2-(trimethylsilyl)ethyl 6-(2,3-bis((tert-butoxycarbonyl)amino)propanamido)hexanoate

To a solution of 2,3-bis((tert-butoxycarbonyl)amino)propanoic acid (1.0 g, 3.14 mmol) and LK1-3 (1.46 g, 31.19 mmol) in dichloromethane (20 mL) was added EDCI (658 mg, 3.45 mmol) and HOBt (463.3 mg, 3.45 mmol). The mixture was stirred at room temperature overnight. The reaction mixture was washed with aq. 5% citric acid (50 mL×3) and then with water (50 mL×3). The organic phase was dried over anhydrous sodium sulfate, concentrated, and purified by flash column chromatography on silica gel (ethyl acetate/n-heptane=1:1) to give the desired product (1.0 g, 58.8% yield) as a white solid.

¹H NMR (400 MHz, Chloroform-d) δ 6.66 (s, 1H), 5.77 (s, 1H), 5.14 (s, 1H), 4.19-4.10 (m, 4H), 3.56-3.40 (m, 3H), 3.25 (dp, J=13.7, 6.5 Hz, 2H), 2.27 (t, J=7.5 Hz, 2H), 1.63 (p, J=7.5 Hz, 3H), 1.52 (p, J=7.3 Hz, 2H), 1.02-0.94 (m, 2H), 0.04 (s, 11H).

### Step 4: Synthesis of 2-(trimethylsilyl)ethyl 9,10-diamino-8-oxodecanoate Dihydrochloride

To a solution of 2-(trimethylsilyl)ethyl 6-(2,3-bis((tert-butoxycarbonyl)amino)propanamido)hexanoate (500 mg, 0.94 mmol) in ethyl acetate (7 mL) was added 2N HCl/ethyl acetate (2.4 mL, 5 eq). The mixture was stirred at room temperature for 2 days. The solvent was removed by concentration, and the solid was precipitated with acetonitrile. The solid was filtered and dried to give a white solid (230 mg, 75.2% yield).

¹H NMR (400 MHz, Deuterium Oxide) δ 4.29 (dd, J=6.2, 5.4 Hz, 1H), 4.25-4.18 (m, 2H), 3.58-3.46 (m, 2H), 3.37-3.26 (m, 1H), 3.22-3.12 (m, 1H), 2.35 (t, J=7.3 Hz, 2H), 1.57 (dp, J=22.6, 7.3 Hz, 4H), 1.33 (qd, J=7.3, 6.3, 4.1 Hz, 2H), 1.04-0.96 (m, 2H), 0.01 (s, 9H).

### Step 5: Synthesis of 2-(trimethylsilyl)ethyl 6-(2,3-bis(but-3-ynamido)propanamido)hexanoate

To a suspension of but-3-ynoic acid (623.6 mg, 7.4 mmol) in dichloromethane (10 mL) was added oxalyl chloride (939.1 mg, 7.4 mmol). After stirring at room temperature for 15 minutes, the reaction mixture was cooled to 0° C., and DMF (0.15 mL) was added. Subsequently, the mixture was stirred at room temperature for 90 minutes. This solution was added dropwise to a solution of 2-(trimethylsilyl)ethyl 9,10-diamino-8-oxodecanoate dihydrochloride (300 mg, 0.74 mmol) in 10 mL of saturated sodium bicarbonate solution, and the mixture was stirred at room temperature for 2 hours. The mixture was extracted with dichloromethane (50 mL×3). The organic phases were combined, dried, and concentrated. Purification was performed by flash column chromatography on silica gel (ethyl acetate/n-heptane=1:1) to give the desired product (292 mg, 85% yield) as a white solid.

¹H NMR (400 MHz, Chloroform-d) δ 7.75 (d, J=6.2 Hz, 1H), 7.30 (s, 1H), 6.96 (s, 1H), 4.45-4.36 (m, 1H), 4.22-4.09 (m, 2H), 3.81 (ddd, J=14.3, 7.1, 3.7 Hz, 1H), 3.52 (dt, J=14.3, 5.5 Hz, 1H), 3.28 (d, J=7.0 Hz, 1H), 3.26 (d, J=2.7 Hz, 3H), 3.24 (d, J=2.6 Hz, 2H), 2.40 (dt, J=6.3, 2.7 Hz, 2H), 2.28 (t, J=7.4 Hz, 2H), 1.63 (p, J=7.5 Hz, 7H), 1.41-1.29 (m, 4H), 1.02-0.94 (m, 2H), 0.04 (s, 9H).

### Step 6: Synthesis of 2-(trimethylsilyl)ethyl 6-(2,3-bis(buta-2,3-dienamido)propanamido)hexanoate

A solution of 2-(trimethylsilyl)ethyl 6-(2,3-bis(but-3-ynamido)propanamido)hexanoate (292 mg, 0.64 mmol) and K₂CO₃ (18% aq, 8 mL) in 1,4-dioxane (8 mL) was heated to 40° C. and stirred for 2 hours, followed by the addition of citric acid (60 mL, 10% in water) and ethyl acetate (500 mL×3). The organic layers were combined, dried, and concentrated to give the desired product (210 mg, 72% yield) as an oil.

¹H NMR (400 MHz, Chloroform-d) δ 7.45 (d, J=5.7 Hz, 1H), 7.12 (s, 1H), 6.72 (s, 1H), 5.64 (q, J=6.4 Hz, 2H), 5.35-5.23 (m, 4H), 4.42-4.34 (m, 1H), 4.20-4.10 (m, 2H), 3.80 (ddd, J=14.4, 7.2, 3.2 Hz, 1H), 3.53 (dt, J=14.3, 5.5 Hz, 1H), 3.24 (dt, J=7.9, 6.3 Hz, 2H), 2.27 (t, J=7.5 Hz, 2H), 1.52 (q, J=7.4 Hz, 2H), 1.36 (s, 3H), 1.01-0.94 (m, 2H), 0.04 (s, 9H).

### Step 7: 6-(2,3-Bis(buta-2,3-dienamido)propanamido)hexanoic Acid

A solution of 2-(trimethylsilyl)ethyl 6-(2,3-bis(buta-2,3-dienamido)propanamido)hexanoate (70 mg, 0.129 mmol) in TFA/DCM (1:1, 1 mL) was stirred at room temperature for 1 hour. The solvent was removed by concentration, and purification was performed on a C18 column (ACN/H₂O) to give the desired product (30 mg, 55%) as a white solid.

### Example 3: Synthesis of Compound LK3

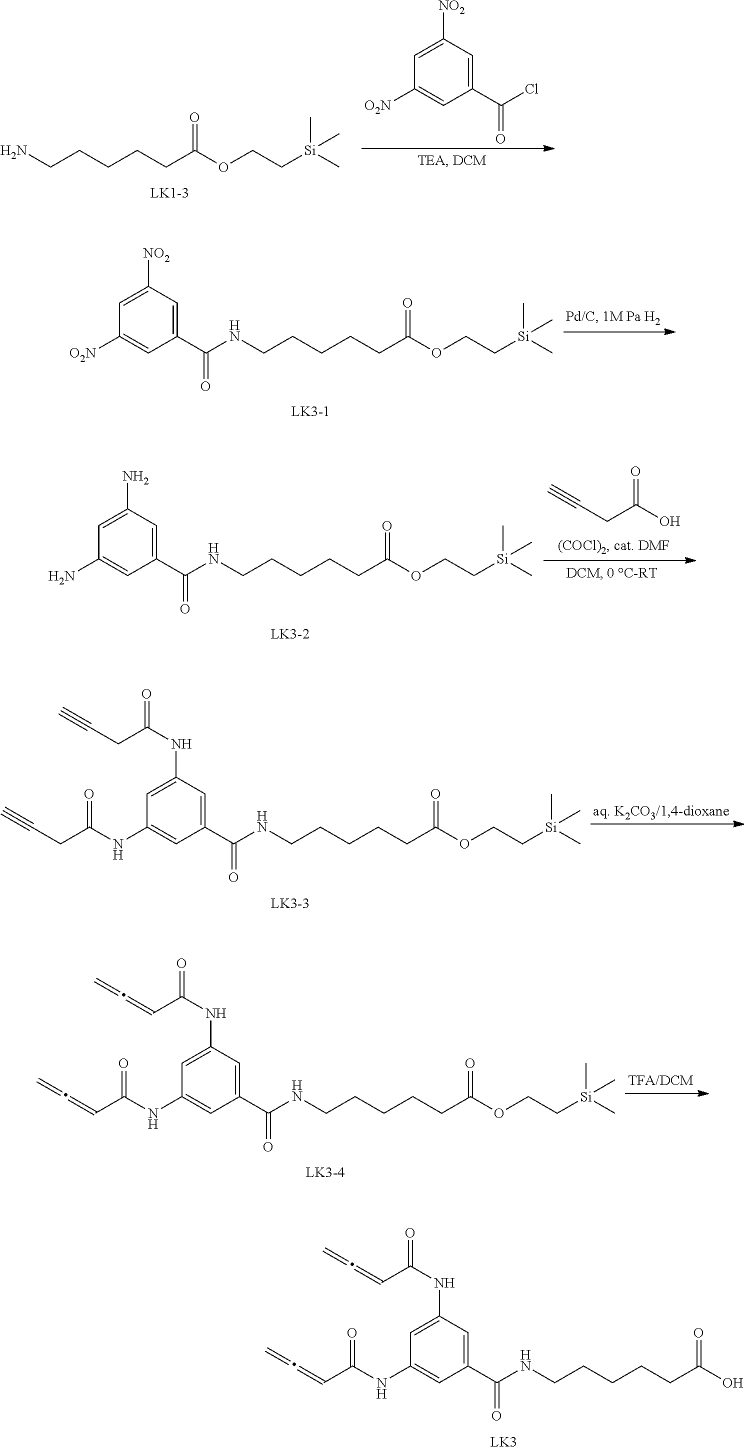

### Step 1: Synthesis of 2-(trimethylsilyl)ethyl 6-(3,5-dinitrobenzamido)hexanoate

To a solution of LK1-3 (455 mg, 1.97 mmol) and triethylamine (300 mg, 2.96 mmol) in dichloromethane (20 mL) was slowly added 3,5-dinitrobenzoyl chloride (500 mg, 2.16 mmol) dropwise at 0° C. After the addition was complete, the mixture was stirred at room temperature for 2 hours, and the completion of the reaction was monitored by LCMS. The reaction mixture was washed sequentially with 1N HCl, saturated NaHCO₃, and brine. The organic phases were combined. The organic layer was dried over sodium sulfate, filtered, concentrated, and purified by flash column chromatography on silica gel to give the desired compound (429 mg, 51% yield) as a yellow solid.

¹H NMR (400 MHz, Chloroform-d) δ 9.15 (t, J=2.1 Hz, 1H), 9.04 (s, 1H), 9.05 (s, 1H), 7.05 (m, 1H), 4.22-4.16 (m, 2H), 3.59 (q, J=6.5 Hz, 2H), 2.36 (t, J=6.8 Hz, 2H), 1.66-1.74 (m, 4H), 1.46-1.52 (m, 2H), 1.03-0.94 (m, 2H), 0.04 (s, 9H).

### Step 2: Synthesis of 2-(trimethylsilyl)ethyl 6-(3,5-diaminobenzamido)hexanoate

To a solution of 2-(trimethylsilyl)ethyl 6-(3,5-dinitrobenzamido)hexanoate (420 mg, 1.15 mmol) in methanol (10 mL) was added 10% wet Pd/C (42 mg, 10% w/w). The mixture was stirred at room temperature overnight under H₂, filtered, and concentrated to give the desired product (377 mg, crude) as a yellow solid.

LC-MS (ESI) m/z: 366 [M+H]⁺.

¹H NMR (400 MHz, Methanol-d₄) δ 6.51 (s, 1H), 6.50 (s, 1H), 6.29 (t, J=2.0 Hz, 1H), 4.59 (brs, 4H), 4.25-4.15 (m, 2H), 3.36-3.38 (m, 2H), 2.35 (t, J=7.4 Hz, 2H), 1.60-1.73 (m, 4H), 1.41-1.47 (m, 2H), 1.04-0.98 (m, 2H), 0.08 (s, 9H).

### Step 3: Synthesis of 2-(trimethylsilyl)ethyl 6-(3,5-di(but-3-ynamido)benzamido)hexanoate

To a suspension of but-3-ynoic acid (850.8 mg, 10.12 mmol) in dichloromethane (50 mL) was added oxalyl chloride (1.28 g, 10.12 mmol). After stirring at room temperature for 15 minutes, the reaction mixture was cooled to 0° C., and a catalytic amount of DMF (0.15 mL) was added. The mixture was slowly warmed to room temperature and stirred for an additional 90 minutes. This solution was added dropwise to a solution of 2-(trimethylsilyl)ethyl 6-(3,5-diaminobenzamido)hexanoate (370 mg, 1.012 mmol) in saturated sodium bicarbonate solution (10 mL). The mixture was stirred at room temperature for 2 hours. The mixture was extracted with dichloromethane (50 mL×3). The organic layers were combined, dried, filtered, and concentrated to give a yellow oily compound.

¹H NMR (400 MHz, Chloroform-d) δ 8.45 (s, 2H), 8.06 (t, J=2.1 Hz, 1H), 7.78 (s, 1H), 7.77 (s, 1H), 6.53-6.56 (m, 1H), 4.18-4.10 (m, 2H), 3.40-3.46 (m, 2H), 3.38 (d, J=2.8 Hz, 4H), 2.49 (t, J=2.7 Hz, 2H), 2.29 (t, J=7.4 Hz, 2H), 1.59-1.69 (m, 4H), 1.36-1.44 (m, 2H), 1.00-0.93 (m, 2H), 0.02 (s, 9H).

### Step 4: Synthesis of 2-(trimethylsilyl)ethyl 6-(3,5-bis(buta-2,3-dienamido)benzamido)hexanoate

A solution of 2-(trimethylsilyl)ethyl 6-(3,5-di(but-3-ynamido)benzamido)hexanoate (1.15 g, 2.32 mol) in K₂CO₃ (18% in water, 10 mL) was mixed with 1,4-dioxane (10 mL). The reaction mixture was heated to 40° C. and stirred for 2 hours, followed by the addition of citric acid (60 mL, 10% in water) and ethyl acetate (500 mL×3). The organic phases were combined, dried, filtered, and concentrated to give an oil (1.15 g, crude).

¹H NMR (400 MHz, Chloroform-d) δ 8.06 (t, J=2.0 Hz, 1H), 8.00 (brs, 2H), 7.77 (s, 1H), 7.76 (s, 1H), 6.64 (t, J=5.8 Hz, 1H), 5.76 (t, J=6.6 Hz, 2H), 5.37 (d, J=6.6 Hz, 4H), 4.18-4.13 (m, 2H), 3.39-3.44 (m, 2H), 2.28 (t, J=7.4 Hz, 2H), 1.58-1.69 (m, 4H), 1.43-1.35 (m, 2H), 1.01-0.92 (m, 2H), 0.02 (s, 9H).

### Step 5: Synthesis of 6-(3,5-bis(buta-2,3-dienamido)benzamido)hexanoic Acid

2-(Trimethylsilyl)ethyl 6-(3,5-bis(buta-2,3-dienamido)benzamido)hexanoate (1.15 g, 2.32 mmol) was dissolved in a solution of TFA/DCM (1:1, 1 mL) and stirred at room temperature for 1 hour. The solvent was removed by concentration, and purification was performed on a C18 column (ACN/H₂O) to give the desired product (330 mg, 35.9% yield over 3 steps) as a white solid.

¹H NMR (400 MHz, DMSO-d₆) δ 9.70 (s, 1H), 8.12 (t, J=1.9 Hz, 1H), 7.72 (d, J=2.0 Hz, 2H), 7.69 (s, 1H), 7.66 (t, J=5.8 Hz, 1H), 5.89 (t, J=6.5 Hz, 2H), 5.19 (d, J=6.5 Hz, 4H), 3.23-3.28 (m, 2H), 2.19 (d, J=7.6 Hz, 2H), 1.59-1.48 (m, 4H), 1.36-1.29 (m, 2H).

### Example 4: Synthesis of Compound LK4

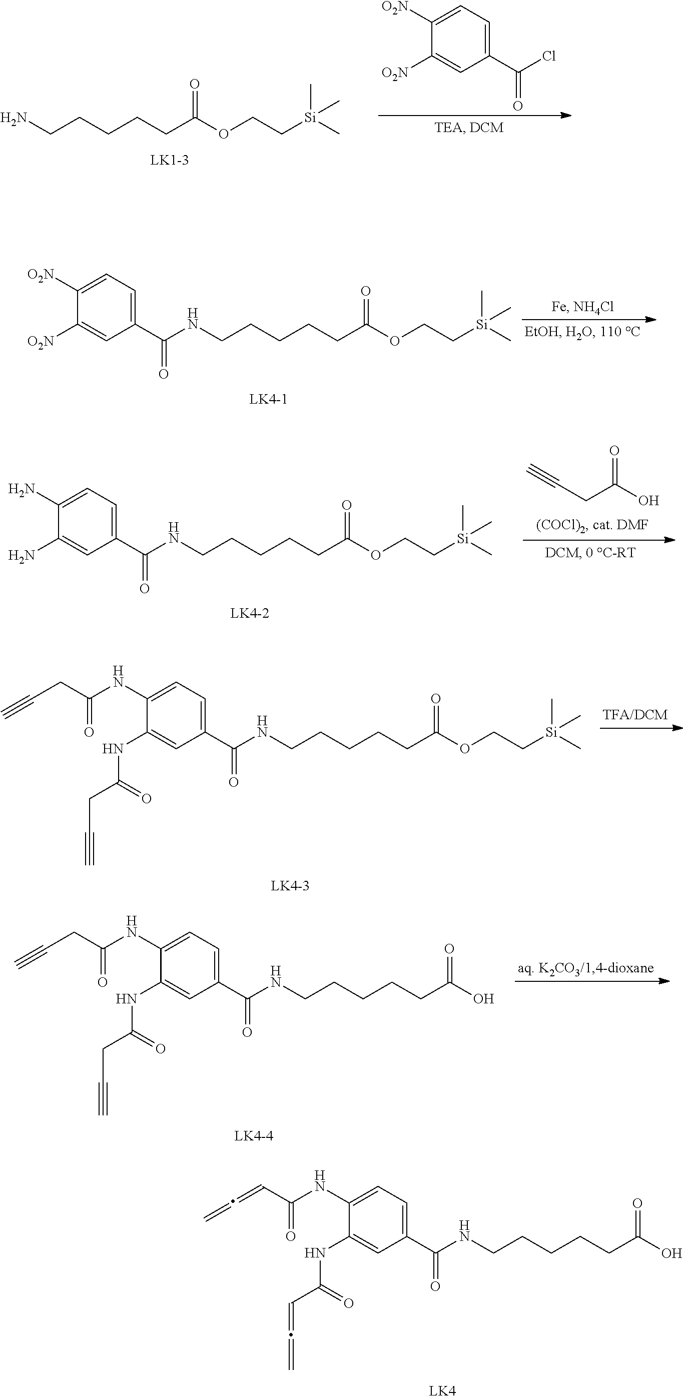

### Step 1: Synthesis of 2-(trimethylsilyl)ethyl 6-(3,4-dinitrobenzamido)hexanoate

To a solution of LK1-3 (884 mg, 3.82 mmol) and triethylamine (527 mg, 5.2 mmol) in dichloromethane (15 mL) was slowly added 3,4-dinitrobenzoyl chloride (800 mg, 3.47 mmol) dropwise at 0° C. The reaction mixture was stirred at room temperature for 2 hours, and the completion of the reaction was monitored by LCMS. The reaction mixture was washed sequentially with 1N HCl, saturated NaHCO₃, and brine. The organic phases were combined, dried over sodium sulfate, filtered, concentrated, and purified by flash column chromatography on silica gel to give the desired product (1.38 g, 93% yield) as a yellow solid. LC-MS (ESI) m/z: 448 [M+Na]⁺.

¹H NMR (400 MHz, Chloroform-d) δ 8.46 (d, J=1.8 Hz, 1H), 8.26 (dd, J=8.3, 1.8 Hz, 1H), 7.96 (d, J=8.3 Hz, 1H), 7.30 (s, 1H), 4.20-4.12 (m, 2H), 3.50 (q, J=6.6 Hz, 2H), 2.32 (t, J=7.0 Hz, 2H), 1.70-1.62 (m, 4H), 1.47-1.40 (m, 2H), 1.01-0.93 (m, 2H), 0.03 (s, 9H).

### Step 2: Synthesis of 2-(trimethylsilyl)ethyl 6-(3,4-diaminobenzamido)hexanoate

A solution of 2-(trimethylsilyl)ethyl 6-(3,4-dinitrobenzamido)hexanoate (1.23 g, 2.9 mmol), iron powder (812 mg, 14.5 mmol), and NH₄Cl (1.55 g, 29 mmol) in 12 mL of EtOH/H₂O (5/1, v/v) was added to a sealed tube and heated to 110° C. for 1 hour. Upon completion of the reaction, the mixture was filtered and washed with ethyl acetate and water. The organic layers were combined, dried over anhydrous sodium sulfate, filtered, concentrated, and purified by flash column chromatography on silica gel to give the desired compound (485 mg, 45.9% yield) as a yellow solid.

LC-MS (ESI) m/z: 366 [M+H]⁺.

### Step 3: Synthesis of 2-(trimethylsilyl)ethyl 6-(3,4-bis(but-3-ynamido)benzamido)hexanoate

To a suspension of but-3-ynoic acid (644 mg, 7.66 mmol) in dichloromethane (30 mL) was added oxalyl chloride (972 mg, 7.66 mmol). After stirring at room temperature for 15 minutes, the reaction mixture was cooled to 0° C., and DMF (0.15 mL) was added. The reaction mixture was gradually warmed to room temperature and stirred for an additional 90 minutes. This solution was added dropwise to a solution of 2-(trimethylsilyl)ethyl 6-(3,4-diaminobenzamido)hexanoate (280 mg, 0.766 mmol) in saturated sodium bicarbonate solution (10 mL), and the mixture was stirred at room temperature for 2 hours. The mixture was extracted with dichloromethane (50 mL×3), dried, filtered, and concentrated. Purification was performed by flash column chromatography on silica gel to give the desired compound (270 mg, 71% yield) as a yellow solid.

¹H NMR (400 MHz, Chloroform-d) δ 8.82 (s, 1H), 8.74 (s, 1H), 7.82 (d, J=2.0 Hz, 1H), 7.68-7.57 (m, 2H), 6.52-6.49 (m, 1H), 4.20-4.12 (m, 2H), 3.47-3.38 (m, 4H), 2.49 (m, 2H), 2.30 (t, J=7.3 Hz, 2H), 1.47-1.27 (m, 6H), 1.01-0.94 (m, 2H), 0.04 (s, 9H).

### Step 4 and Step 5: Synthesis of 6-(3,4-bis(buta-2,3-dienamido)benzamido)hexanoic Acid

2-(Trimethylsilyl)ethyl 6-(3,4-bis(but-3-ynamido)benzamido)hexanoate (50 mg, 0.1 mmol) was dissolved in TFA/DCM (1:1, 2 mL) and stirred at room temperature for 1 hour. The solvent was removed by concentration, and the residue was dissolved in 1,4-dioxane (10 mL), followed by the addition of K₂CO₃ (18% in water, 10 mL). The reaction mixture was heated to 40° C. and stirred for 2 hours, followed by the addition of citric acid (60 mL, 10% in water) and ethyl acetate (500 mL×3). The organic phases were combined, dried, filtered, concentrated, and purified on a C18 column to give the desired product (38 mg, 95.2% yield over two steps) as a yellow solid.

LC-MS (ESI) m/z: 398 [M+H]⁺.

¹H NMR (400 MHz, Chloroform-d) δ 9.35-9.31 (m, 1H), 9.20-9.16 (m, 1H), 7.78 (m, 1H), 7.64 (m, 1H), 7.57 (m, 1H), 5.69 (s, 2H), 5.20 (d, J=6.6 Hz, 4H), 3.99 (d, J=7.4 Hz, 2H), 3.28 (m, 2H), 2.19-3.32-3.25 (m, 2H), 1.92-1.88 (m, 4H).

### Example 5: Synthesis of Compound LK5

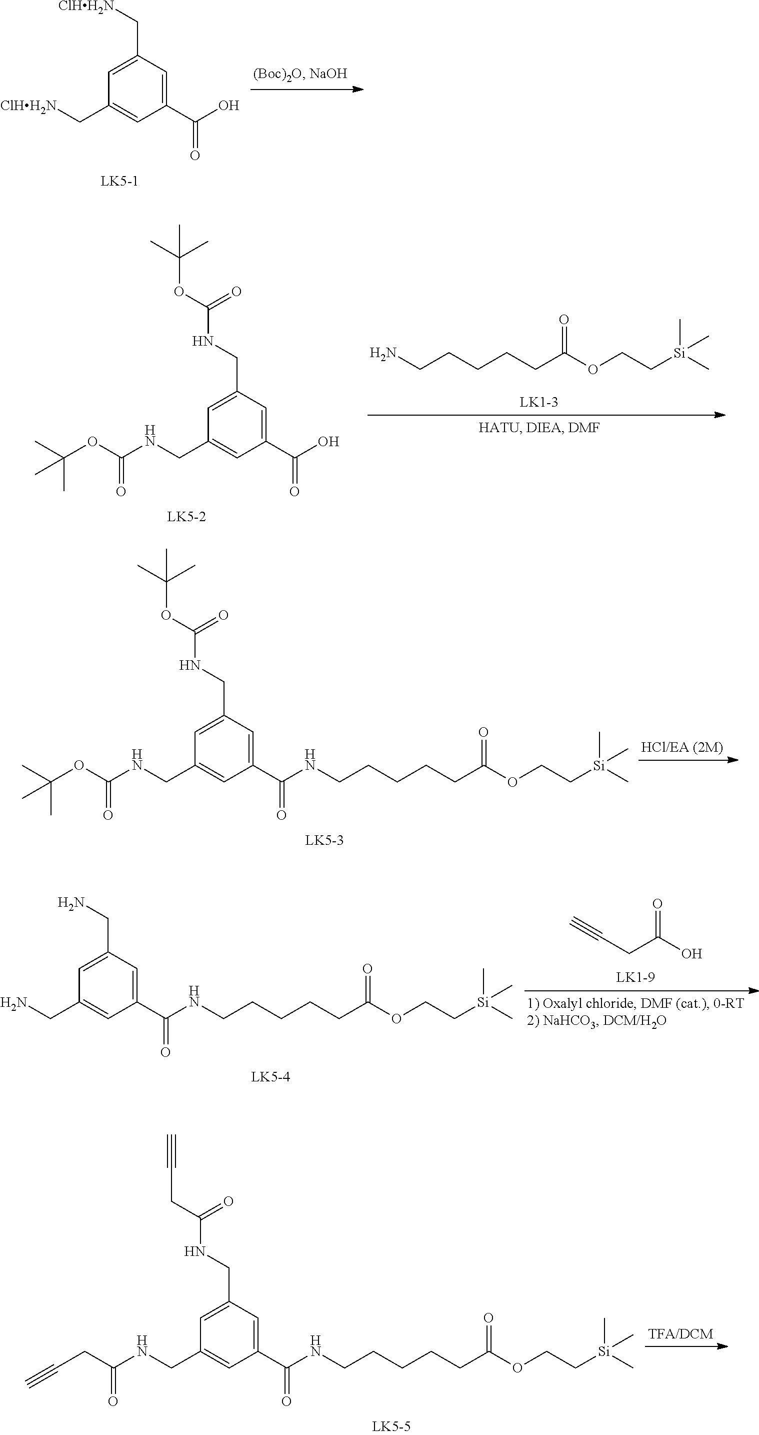

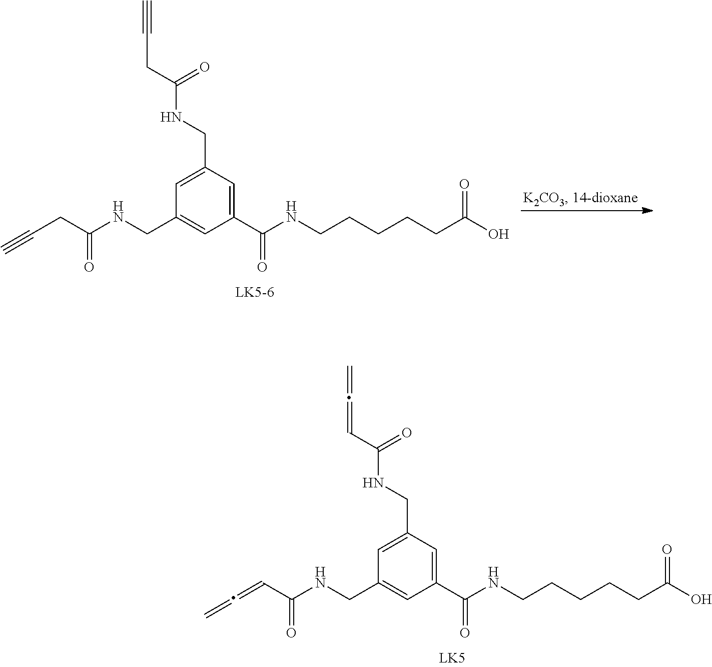 [SMILES: C#CCC(=O)NCc1cc(CNC(=O)CC#C)cc(C(=O)NCCCCCC(=O)O)c1 ; C=C=CC(=O)NCc1cc(CNC(=O)C=C=C)cc(C(=O)NCCCCCC(=O)O)c1]

### Step 1: Synthesis of 3,5-bis(((tert-butoxycarbonyl)amino)methyl)benzoic Acid

3,5-Bis(aminomethyl)benzoic acid dihydrochloride (450 mg, 1.78 mmol) and sodium hydroxide (216 mg, 5.4 mmol) were dissolved in water (1 mL). (Boc)₂O (1164 mg, 5.3 mmol) dissolved in 1,4-dioxane (16 mL) was added to the reaction mixture. The mixture was stirred overnight and then extracted with ethyl acetate (20 mL×2). The aqueous layer was then adjusted to pH 4-5 with HCl, extracted with ethyl acetate (30 mL×2), dried over anhydrous sodium sulfate, and concentrated to give 3,5-bis(((tert-butoxycarbonyl)amino)methyl)benzoic acid (410 mg, 60.5% yield) as a white solid.

### Characterization Data:

LC-MS (ESI) m/z: 403.2 [M+Na]⁺.

¹H NMR (400 MHz, DMSO-d₆) δ 12.85 (s, 1H), 7.70 (s, 2H), 7.45-7.42 (m, 2H), 7.33 (s, 1H), 4.16 (s, 4H), 1.39 (s, 18H).

### Step 2: Synthesis of 2-(trimethylsilyl)ethyl 6-(3,5-bis(((tert-butoxycarbonyl)amino)methyl)benzamido)hexanoate

A mixture of 3,5-bis(((tert-butoxycarbonyl)amino)methyl)benzoic acid (200 mg, 0.53 mmol), 2-(trimethylsilyl)ethyl 6-aminohexanoate (120 mg, 0.52 mmol), and HATU (300 mg, 0.79 mmol) was stirred at room temperature for 10 min, and DIEA (280 μL, 1.62 mmol) was added. The resulting mixture was stirred for 4 hours. The mixture was extracted twice with ethyl acetate (30 mL×2), and the ethyl acetate layer was washed 4 times with water. After drying over anhydrous sodium sulfate, the mixture was concentrated to give 290 mg of crude 2-(trimethylsilyl)ethyl 6-(3,5-bis(((tert-butoxycarbonyl)amino)methyl)benzamido)hexanoate as a white solid.

### Characterization Data:

LC-MS (ESI) m/z: 394.2 [M+H−200]⁺.

¹H NMR (400 MHz, DMSO-d₆) δ 8.38 (m, 1H), 7.53 (s, 2H), 7.41-7.38 (m, 2H), 7.22 (s, 1H), 4.113-4.05 (m, 6H), 3.24-3.19 (m, 2H), 2.25 (t, J=8, 2H), 1.53-1.48 (m, 4H), 1.37 (s, 18H), 1.28-1.22 (m, 2H), 0.93-0.89 (m, 2H), 0.0 (s, 9H).

### Step 3: 2-(trimethylsilyl)ethyl 6-(3,5-bis(aminomethyl)benzamido)hexanoate

To the crude 2-(trimethylsilyl)ethyl 6-(3,5-bis(((tert-butoxycarbonyl)amino)methyl)benzamido)hexanoate (290 mg) was added a solution of hydrogen chloride in ethyl acetate (3 mL, 2 M), and the mixture was reacted at room temperature for 30 min. The reaction mixture was directly purified by preparative liquid chromatography to give 80 mg of 2-(trimethylsilyl)ethyl 6-(3,5-bis(aminomethyl)benzamido)hexanoate as an oil.

### Characterization Data:

LC-MS (ESI) m/z: 394.2 [M+H]⁺.

¹H NMR (400 MHz, DMSO-d₆) δ 8.38 (s, 1H), 7.53 (s, 2H), 7.41-7.38 (m, 2H), 7.22 (s, 1H), 4.13-4.05 (m, 6H), 2.25 (t, J=8 Hz, 2H), 1.53-1.48 (m, 4H), 1.37 (s, 18H), 1.28-1.22 (m, 2H), 0.93-0.89 (m, 2H), 0.0 (s, 9H).

Step 4: 2-(trimethylsilyl)ethyl 6-(3,5-bis(but-3-ynamidomethyl)benzamido)hexanoate but-3-ynoic acid (42 mg, 0.51 mmol) was dissolved in dichloromethane (2 mL), and oxalyl chloride (85 μL, 1.02 mmol) was added at 0° C. The reaction mixture was stirred at room temperature for 1 hour. The reaction mixture was added to a solution of 2-(trimethylsilyl)ethyl 6-(3,5-bis(aminomethyl)benzamido)hexanoate (20 mg, 0.051 mmol) in saturated aqueous sodium bicarbonate solution (5 mL), and the mixture was stirred for 2 hours. The solution was extracted with dichloromethane (20 mL×2). The dichloromethane layer was dried over anhydrous sodium sulfate and concentrated to give 28 mg of crude 2-(trimethylsilyl)ethyl 6-(3,5-bis(but-3-ynamidomethyl)benzamido)hexanoate as a gray solid.

### Characterization Data:

LC-MS (ESI) m/z: 498.2 [M+3H−2CH₃]⁺.

¹H NMR (400 MHz, DMSO-d₆) δ 8.47-8.41 (m, 3H), 7.58 (s, 2H), 7.26 (s, 1H), 4.28 (d, J=4 Hz, 4H), 4.10-4.05 (m, 2H), 3.40-3.29 (m, 6H), 3.00 (d, J=4 Hz, 2H), 2.25 (t, J=8 Hz, 18H), 1.53-1.36 (m, 4H), 1.32-1.28 (m, 4H), 0.98-0.84 (m, 2H).

### Step 5: 6-(3,5-bis(but-3-ynamidomethyl)benzamido)hexanoic Acid

The crude 2-(trimethylsilyl)ethyl 6-(3,5-bis(but-3-ynamidomethyl)benzamido)hexanoate (28 mg) was dissolved in DCM/TFA (1 mL/1 mL), and the mixture was reacted for 1 hour. The reaction mixture was concentrated to give 20 mg of the desired compound 6-(3,5-bis(but-3-ynamidomethyl)benzamido)hexanoic acid as an oil.

### Characterization Data:

LC-MS (ESI) m/z: 426.2 [M+H]⁺.

¹H NMR (400 MHz, DMSO-d₆) δ 8.51-8.43 (m, 3H), 7.60 (s, 2H), 7.28 (s, 1H), 4.30 (d, J=4 Hz, 2H), 3.24-3.18 (m, 6H), 3.02 (d, J=4 Hz, 2H), 2.23 (t, J=8 Hz, 1H), 1.54-1.49 (m, 4H), 1.32-1.26 (m, 6H).

### Step 6: 6-(3,5-bis(buta-2,3-dienamidomethyl)benzamido)hexanoic Acid

6-(3,5-bis(but-3-ynamidomethyl)benzamido)hexanoic acid was dissolved in 1,4-dioxane (0.5 mL) and potassium carbonate solution (1 mL) and stirred at 40° C. for 1 hour. The mixture was extracted with ethyl acetate (10 mL×2). The aqueous layers were adjusted to pH 4-5, combined, extracted with ethyl acetate (10 mL×2), and then concentrated to give 10 mg of the desired product 6-(3,5-bis(buta-2,3-dienamidomethyl)benzamido)hexanoic acid as a white solid.

### Characterization Data:

LC-MS (ESI) m/z: 426.2 [M+H]⁺.

¹H NMR (400 MHz, DMSO-d₆) δ 8.46 (m, 3H), 7.61 (s, 2H), 7.27 (s, 1H), 5.81-5.78 (m, 2H), 5.33 (d, J=4 Hz, 4H), 4.33 (d, J=4 Hz, 2H), 2.22-2.19 (m, 2H), 1.52-1.50 (m, 4H), 1.30-1.24 (m, 6H).

### Synthesis and Characterization of Linker-Drug Conjugates

### Example 6: Synthesis of Compound LP1

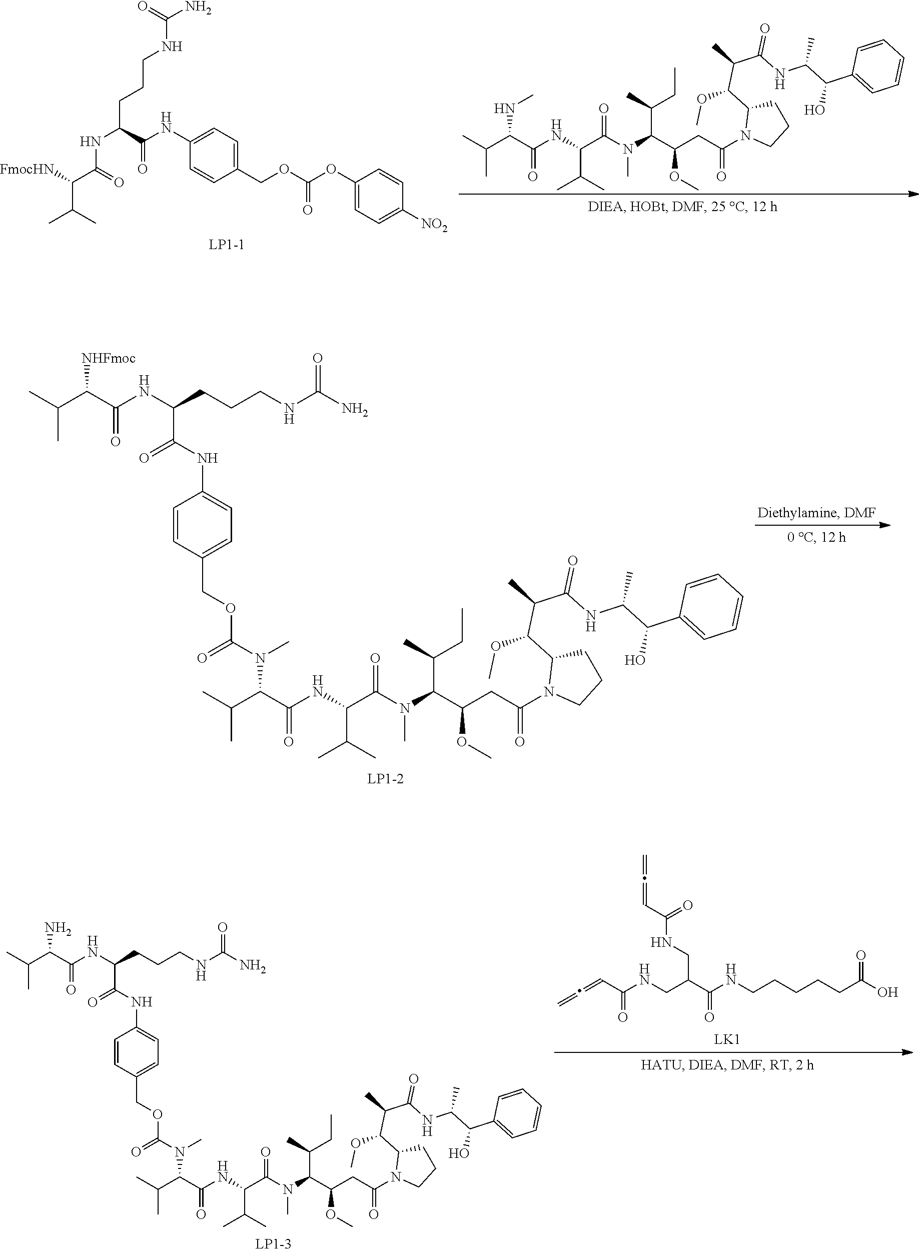

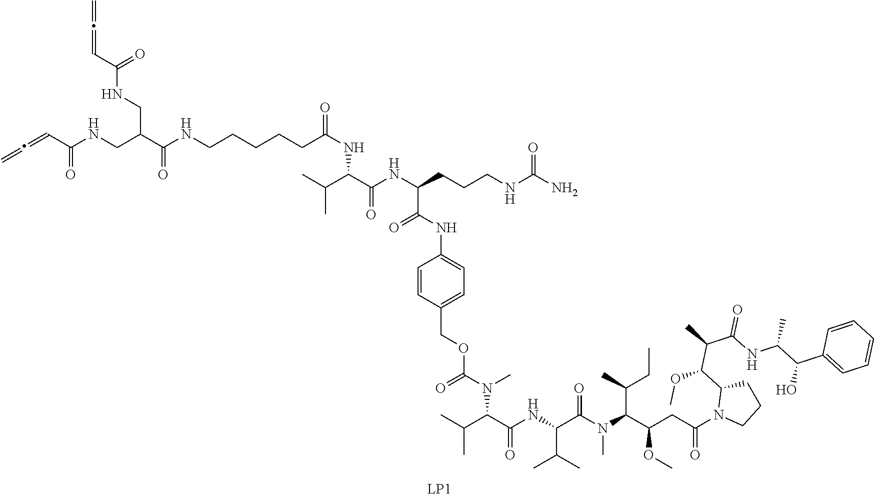 [SMILES: C=C=CC(=O)NCC(CNC(=O)C=C=C)C(=O)NCCCCCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

### Step 1: Synthesis of LP1-2:

At 0° C., to a solution of (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (587 mg, 0.770 mmol) in DMF (10 mL) was added (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (500 mg, 0.690 mmol), HOBt (211 mg, 1.53 mmol), and DIEA (198 mg, 1.53 mmol). After the addition, the mixture was stirred at 25° C. for 12 hours. LCMS indicated the completion of the reaction. The mixture was concentrated to afford a residue, which was purified by C₁₈ (0-60% ACN in water) to give LP1-2 (600 mg, 64% yield) as a white solid.

### Characterization Data:

LC-MS (ESI) m/z: 1346.8 [M+H]⁺.

### Step 2: Synthesis of LP1-3

At 0° C., to a solution of 4-((S)-2-((S)-2-((((9H-fluoren-9-yl)methoxy)carbonyl)amino)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (600 mg, 0.45 mmol) in DMF (5 mL) was added diethylamine (66.0 mg, 0.90 mmol). After the addition was complete, the mixture was stirred at 0° C. for 2 hours. LCMS indicated the completion of the reaction. The mixture was concentrated to afford a residue, which was purified by C₁₈ (0-45% acetonitrile in H₂O) to give LP1-3 (300 mg, 59% yield) as a white solid.

### Characterization Data:

LC-MS (ESI) m/z: 1123.8 [M+H]⁺.

### Step 3: Synthesis of LP1

To a solution of LP1-3 (20 mg, 0.0178 mmol) and LK1 (10 mg, 0.035 mmol) in 1 mL of DMF was added HATU (20.3 mg, 0.0534 mmol) and DIPEA (12 mg, 0.009 mmol). After the addition was complete, the reaction mixture was stirred at room temperature overnight. Purification was performed by preparative high-performance liquid chromatography to give compound LP1 (5 mg, 19.2% yield) as a white solid.

LC-MS (ESI) m/z: 734 [M/2+H]⁺.

¹H NMR (400 MHz, DMSO-d₆) δ 9.99 (s, 1H), 8.11 (d, J=7.3 Hz, 1H), 7.94-7.78 (m, 4H), 7.57 (d, J=8.1 Hz, 2H), 7.28 (dd, J=15.7, 6.7 Hz, 6H), 7.17 (d, J=7.1 Hz, 1H), 6.02 (s, 1H), 5.74 (t, J=6.6 Hz, 2H), 5.42 (s, 2H), 5.27 (d, J=6.5 Hz, 4H), 5.01 (d, J=22.4 Hz, 2H), 4.48 (s, 1H), 4.45-4.32 (m, 1H), 4.29-4.14 (m, 1H), 3.99 (s, 2H), 3.76 (d, J=8.4 Hz, 1H), 3.20 (dd, J=18.4, 8.8 Hz, 8H), 3.10 (s, 1H), 3.06-2.92 (m, 4H), 2.85 (d, J=15.0 Hz, 1H), 2.14 (dd, J=17.8, 7.8 Hz, 2H), 1.55-1.32 (m, 6H), 1.00 (ddd, J=11.8, 9.8, 6.6 Hz, 5H), 0.91-0.70 (m, 23H).

### Example 7: Synthesis of Compound LP2

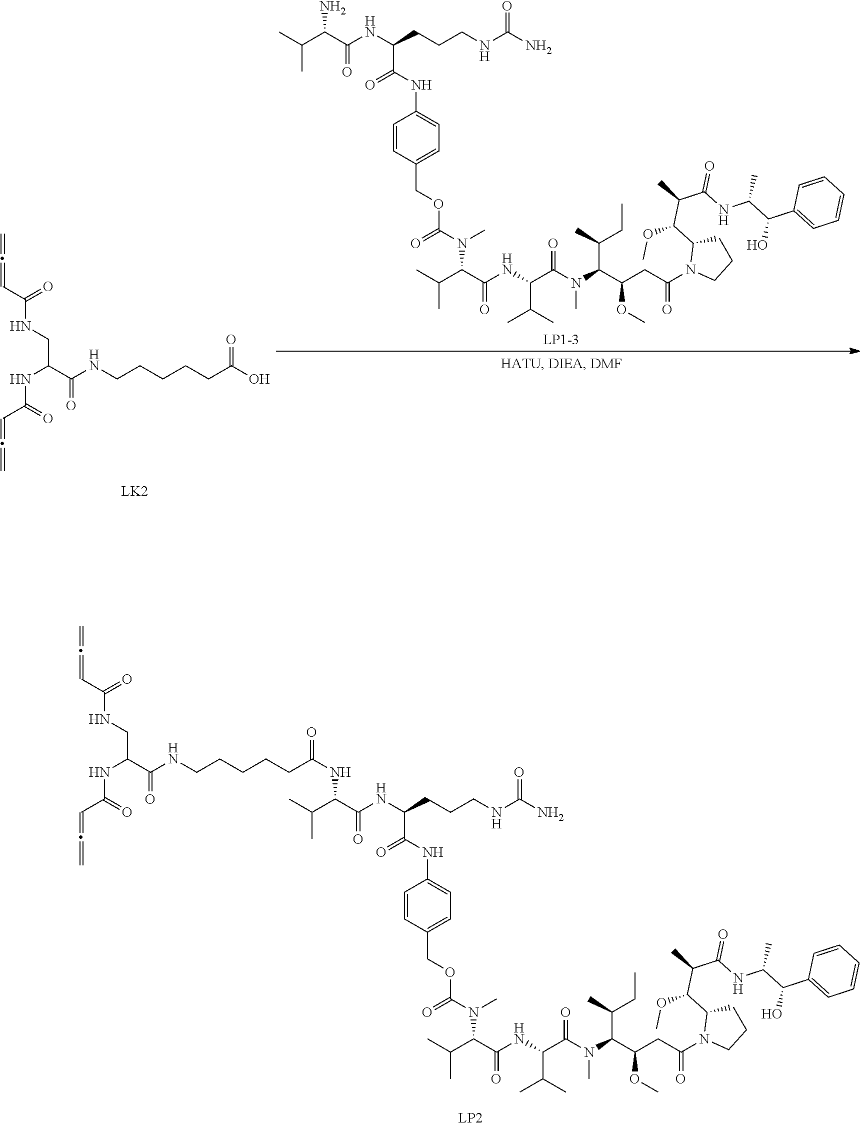 [SMILES: C=C=CC(=O)NCC(NC(=O)C=C=C)C(=O)NCCCCCC(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; C=C=CC(=O)NCC(NC(=O)C=C=C)C(=O)NCCCCCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

### Synthesis of LP2:

LP1-3 (20 mg, 0.0178 mmol) and LK2 (10 mg, 0.035 mmol) were dissolved in DMF (1 mL), and HATU (20.3 mg, 0.0534 mmol) and DIPEA (12 mg, 0.009 mmol) were added. After the addition was complete, the reaction mixture was stirred at room temperature overnight. Purification was performed by preparative high-performance liquid chromatography to give LP2 (3.8 mg, 14.9% yield) as a white solid.

LC-MS (ESI) m/z: 727 [M/2+H]⁺.

### Example 8: Synthesis of Compound LP3

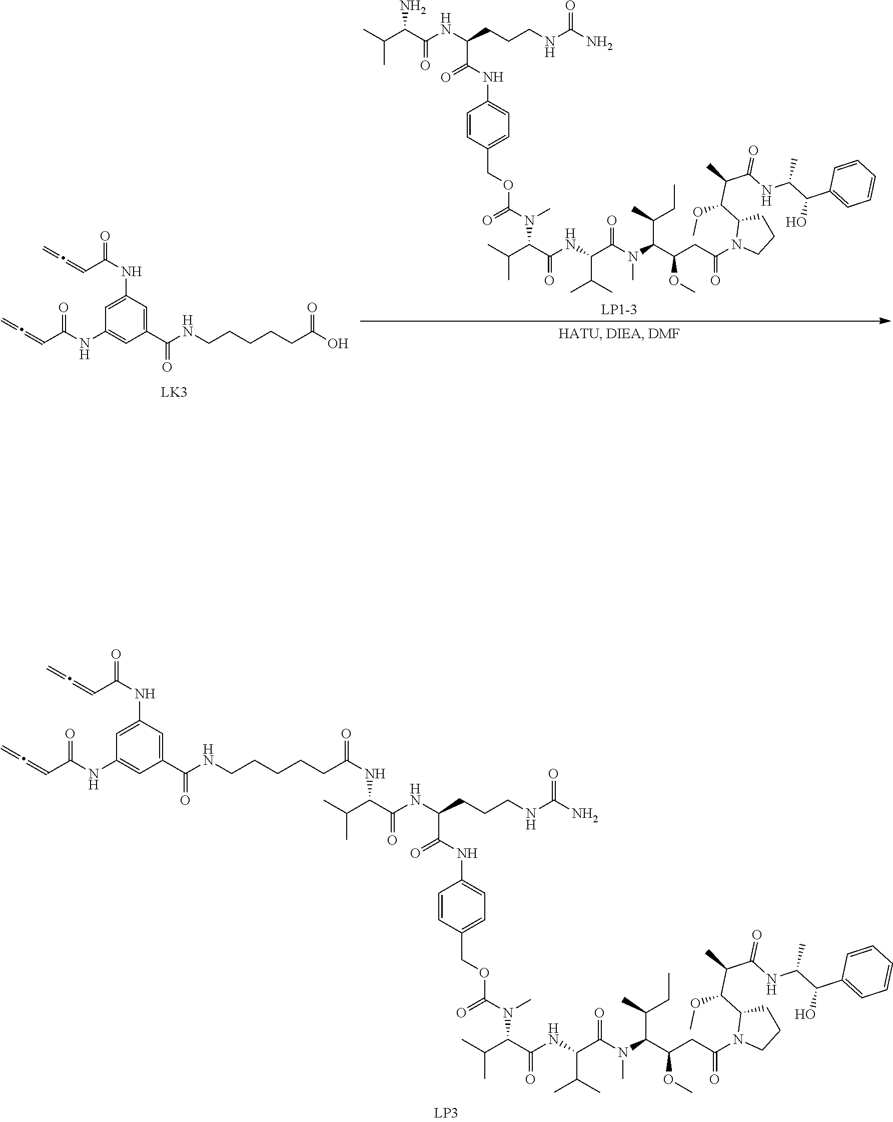

### Synthesis of LP3:

LK3 (14.2 mg, 0.036 mmol), HOBt (8.4 mg, 0.063 mmol), and EDCI (7.4 mg, 0.072 mmol) were dissolved in a DMF (2 mL) solution and stirred at 0° C. for 15 minutes. LP1-3 (30 mg, 0.018 mmol) was added to the solution, and the mixture was stirred at 0° C. for 5 hours. Upon completion of the reaction, the mixture was purified by preparative high-performance liquid chromatography to give LP3 (4.98 mg, 12.4% yield) as a white solid.

LC-MS (ESI) m/z: 752 [M/2+H]⁺.

¹H NMR (400 MHz, DMSO-d₆) δ 10.13 (s, 2H), 9.96 (s, 1H), 8.36 (s, 1H), 8.10 (d, J=2.0 Hz, 1H), 8.07 (d, J=7.5 Hz, 1H), 7.79 (d, J=8.6 Hz, 1H), 7.69 (d, J=2.0 Hz, 2H), 7.58 (d, J=8.7 Hz, 2H), 7.36-7.22 (m, 7H), 7.17 (t, J=7.4 Hz, 1H), 5.98 (t, J=6.5 Hz, 3H), 5.43 (d, J=6.5 Hz, 4H), 5.39 (s, 3H), 5.33 (d, J=4.9 Hz, 1H), 5.04 (t, J=18.0 Hz, 2H), 4.49 (s, 1H), 4.40 (dd, J=18.0, 6.7 Hz, 2H), 4.27 (d, J=11.0 Hz, 1H), 4.20 (dd, J=14.7, 7.0 Hz, 1H), 4.06-3.94 (m, 2H), 3.21 (dd, J=19.2, 7.8 Hz, 9H), 3.11 (s, 1H), 2.98 (td, J=15.1, 14.3, 7.7 Hz, 5H), 2.86 (d, J=14.2 Hz, 3H), 2.28 (t, J=7.5 Hz, 1H), 2.16 (ddd, J=21.8, 14.1, 7.3 Hz, 2H), 2.02-1.91 (m, 1H), 1.51 (q, J=7.0 Hz, 5H), 1.32 (d, J=16.2 Hz, 2H), 1.07-0.97 (m, 6H), 0.91-0.72 (m, 27H).

### Example 9: Synthesis of Compound LP4

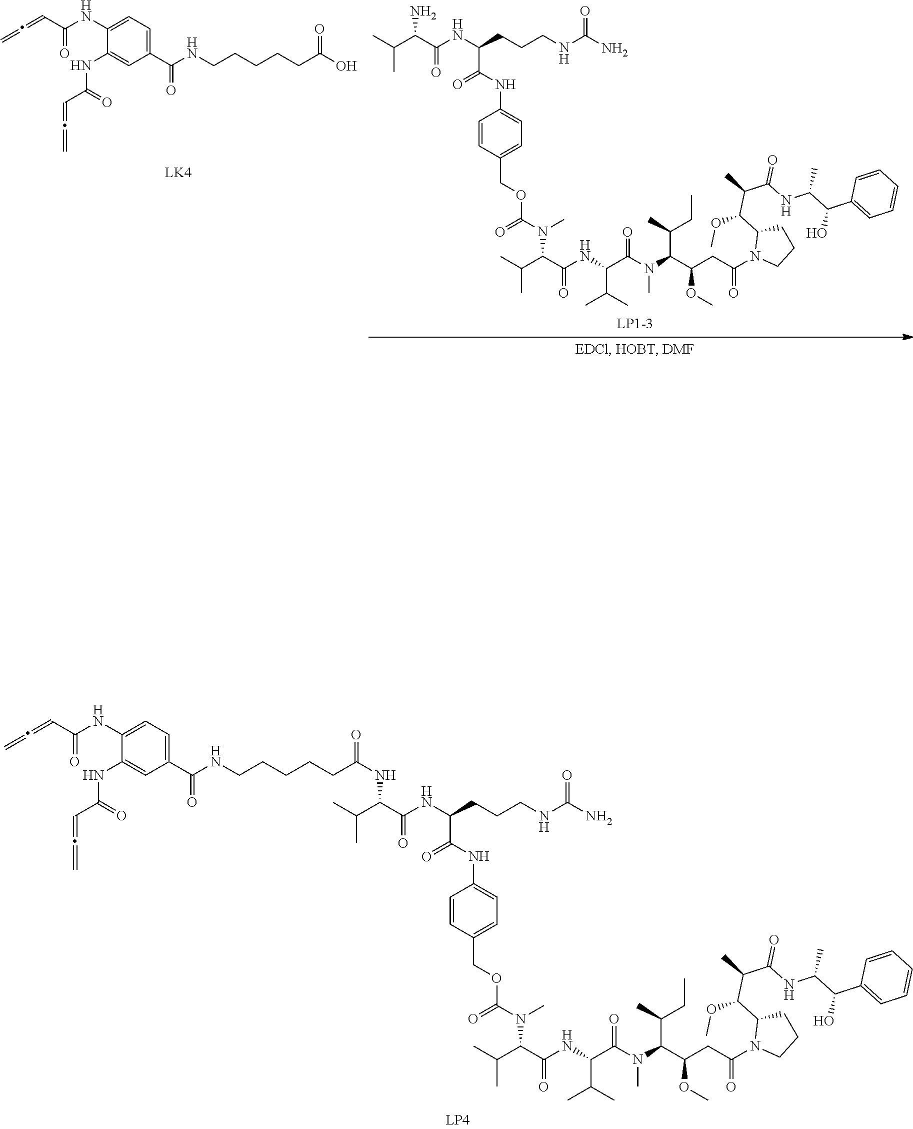

### Synthesis of LP4:

LK4 (3.54 mg, 0.009 mmol), HOBt (2.1 mg, 0.016 mmol), and EDCI (1.85 mg, 0.002 mmol) were dissolved in DMF (0.5 mL) and stirred at 0° C. for 15 minutes. LP1-3 (5 mg, 0.0045 mmol) was added to the solution, and the mixture was reacted at 0° C. for 5 hours. Upon completion of the reaction, the mixture was purified by preparative high-performance liquid chromatography to give compound LP4 (0.71 mg, 10.6% yield) as a white solid.

LC-MS (ESI) m/z: 752 [M/2+H]⁺.

### Example 10: Synthesis of Compound LP5

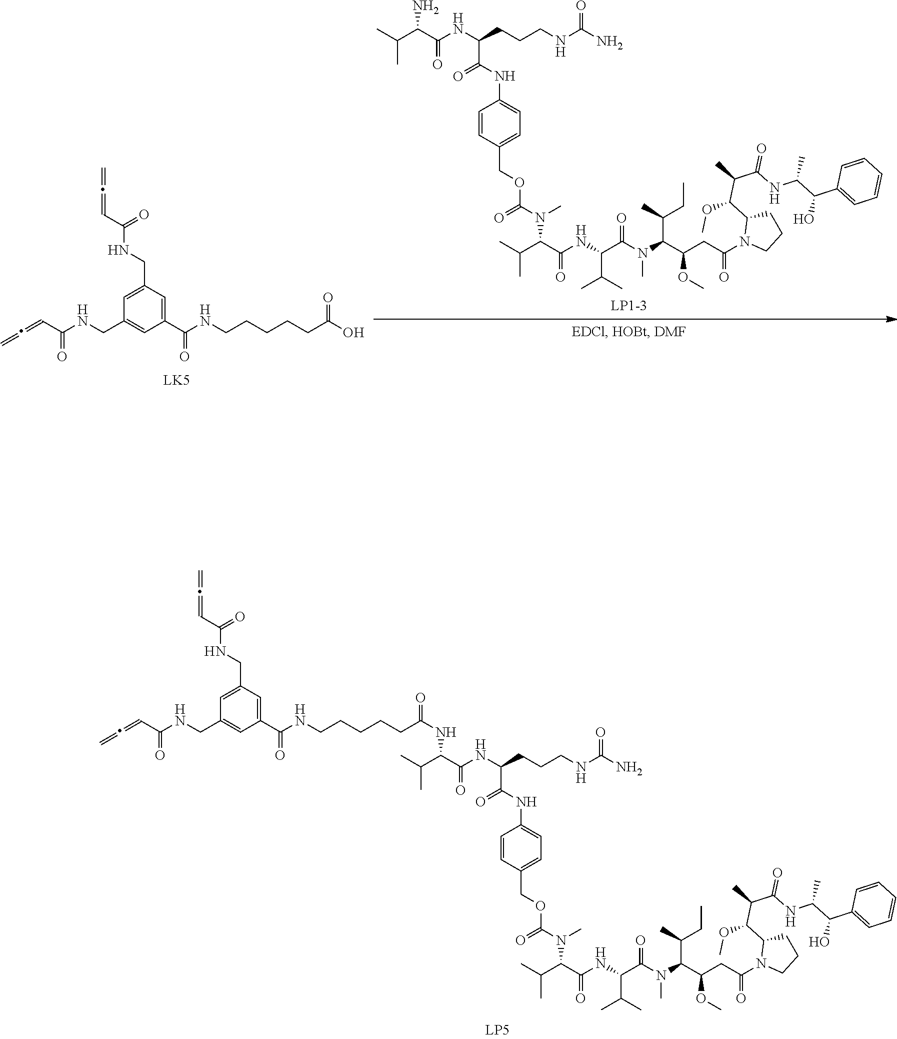

### Synthesis of LP5:

6-(3,5-bis(buta-2,3-dienamidomethyl)benzamido)hexanoic acid (8 mg, 0.019 mmol), HOBt (6 mg, 0.044 mmol), and EDCI (10 mg, 0.052 mmol) were dissolved in DMF (0.5 mL), and the reaction mixture was stirred at room temperature for 10 min. LP1-3 (15 mg, 0.013 mmol) was added to the reaction mixture, and the mixture was further stirred for 4 hours. LCMS indicated the formation of the desired product, which was purified by preparative high-performance liquid chromatography to give the desired product LP5 (2.0 mg, 10.0% yield) as a white solid.

### Characterization Data:

LC-MS (ESI) m/z: 1530.98 [M+H]⁺.

¹H NMR (400 MHz, DMSO-d₆) δ 9.90 (s, 1H), 8.45-8.42 (m, 3H), 7.62-7.58 (m, 5H), 7.32-7.22 (m, 10H), 7.10 (s, 1H), 6.97 (s, 1H), 5.99-5.95 (m, 1H), 5.83-5.79 (m, 2H), 5.41 (s, 2H), 5.33-5.32 (m, 4H), 4.34 (d, J=8 Hz, 5H), 3.38-2.85 (m, 42H), 2.34 (s, 4H), 2.08 (s, 2H), 2.06-2.00 (m, 4H), 1.55-1.52 (m, 7H), 1.30-1.24 (m, 12H), 1.06-0.98 (m, 13H), 0.85-0.75 (m, 32H).

### Example 11: Synthesis of Compound LP10

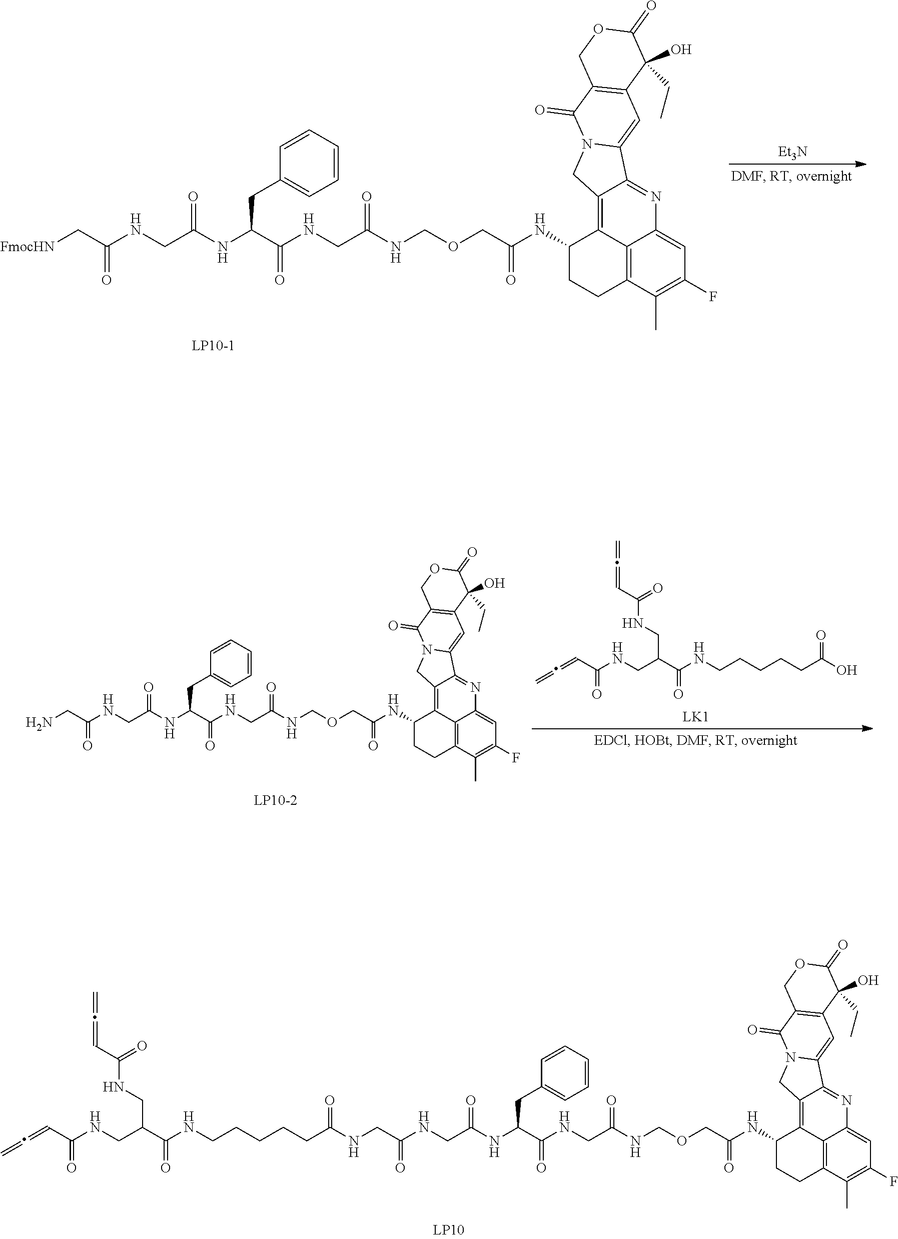

### Step 1: Synthesis of LP10-2:

To a solution of LP10-1 (88 mg, 0.0828 mmol) in DMF (1.5 mL) was added triethylamine (0.5 mL). After the addition was complete, the reaction mixture was stirred at room temperature overnight. LCMS indicated the completion of the reaction. The mixture was concentrated to afford a residue, which was purified by C₁₈ (0-60% ACN in water) to give LP10-2 (45 mg, 65% yield) as a white solid.

### Characterization Data:

LC-MS (APCI) m/z: 841.20 [M+H]⁺.

¹H NMR (400 MHz, MeOD-d₄) δ 7.99 (s, 3H), 7.56□7.46 (m, 2H), 7.33□7.09 (m, 4H), 5.73 (dd, J=8.4, 4.6 Hz, 1H), 5.55 (d, J=16.3 Hz, 1H), 5.36□5.19 (m, 1H), 4.65 (d, J=10.6 Hz, 1H), 4.44 (dd, J=8.5, 6.2 Hz, 1H), 4.30□4.11 (m, 1H), 4.08□3.99 (m, 1H), 3.96□3.81 (m, 3H), 3.67 (d, J=1.0 Hz, 1H), 3.54 (s, 1H), 3.28□3.16 (m, 9H), 2.37 (d, J=1.8 Hz, 2H), 0.97 (t, J=7.4 Hz, 2H) ppm.

### Step 2: Synthesis of LP10:

To a solution of LP10-2 (45 mg, 53.6 mmol) and LK1 (9.9 mg, 27.2 mmol) in 2 mL DMF was added HOBt (6.4 mg, 47.7 mmol) and EDCI (10.4 mg, 54.5 mmol). After the addition was complete, the reaction mixture was stirred at room temperature overnight. LCMS indicated the completion of the reaction. The mixture was concentrated to afford a residue, which was purified by C₁₈ (0-60%6 ACN in water) to give LP10 (20 mg, 32% yield) as a white solid.

### Characterization Data:

LC-MS (ESI) m/z: 1186.17 [M+H]⁺.

¹H NMR (400 MHz, DMSO-d₆) δ 8.63 (t, J=6.6 Hz, 1H), 8.51 (d, J=8.8 Hz, 1H), 8.37-08.26 (m, 1H), 8.12 (d, J=8.0 Hz, 1H), 8.06□08.02 (m, 1H), 8.00 (t, J=5.7 Hz, 1H), 7.87□7.79 (m, 2H), 7.29□7.13 (m, 5H), 5.75 (t, J=6.6 Hz, 2H), 5.33□5.14 (m, 4H), 4.64 (d, J=6.6 Hz, 4H), 4.54□4.39 (m, 2H), 3.83□3.57 (m, 6H), 3.28□3.09 (m, 5H), 3.07□2.94 (m, 4H), 2.39 (d, J=1.9 Hz, 3H), 2.22□2.14 (m, 2H), 2.14□2.05 (m, 2H), 1.92□1.79 (m, 2H), 1.53□1.43 (m, 2H), 1.41□1.31 (m, 2H), 1.28□1.19 (m, 2H), 0.87 (t, J=7.3 Hz, 3H).

### Preparation and Characterization of Antibody-Drug Conjugates

The following are examples of preparation and analytical characterization methods for antibody-drug conjugates. It should be noted that the following examples are merely partial embodiments and are not limited to the embodiments described herein. For example, the described antibody is not limited to the antibody shown herein, but may also be any other antibody.

### General Conjugation Process for the Preparation of Bis-Allene Covalently Bridged Antibody-Drug Conjugates

The concentration of the antibody solution was adjusted to 5-10 mg/mL, and the pH was adjusted to about 7.5. A clean centrifuge tube was used as the reaction vessel, and the monoclonal antibody (1.0 eq.) was placed in the tube. TCEP (5 eq. to 8 eq.) was added and mixed well, followed by incubation in a thermomixer at 20-25° C. for 2-16 hours to achieve complete reduction of the interchain disulfide bonds of the antibody. The pH was raised to 8.0 either by adding an equal volume of 500 mM Tris buffer (pH 8.8) or by buffer-exchanging the antibody into 25 mM Na₂B₄O₇ buffer (pH 8.0) using a desalting column. Subsequently, a toxin-linker solution dissolved in DMSO at a concentration of 10 mg/mL was added (5 eq. to 8 eq.), and DMSO was supplemented at 10-20% of the final total volume of the reaction mixture. After vortex mixing, the mixture was placed on a heating and cooling thermomixer for conjugation at 25° C. for 16-24 hours.

Unconjugated residual toxin-linker in the reaction mixture was removed using activated carbon coated with 300 mg/mL dextran (manufacturer: Sigma). A 10% volume of activated carbon solution was added to the reaction mixture, mixed well, and then incubated at room temperature for 1 hour. The mixture was centrifuged, and the supernatant was collected. The content of residual toxin-linker in the supernatant was determined. If the content of free toxin-linker exceeded 1%, the above activated carbon treatment should be repeated multiple times until the test result meets the specification (typically, three treatments are sufficient to meet the requirement). After completion of the treatment, the desired antibody-drug conjugate was buffer-exchanged into an appropriate storage buffer via 3-6 rounds of ultrafiltration, and stored at −80° C.

### General Characterization Method for Antibody-Drug Conjugates

### (a) Analysis of DAR Value for ADC by HIC-HPLC

High-Performance Liquid Chromatograph: e2695 High-Performance Liquid Chromatography System or Agilent 1260 Liquid Chromatograph.

Chromatographic Column: MabPac™ HIC-Butyl, 5 μm, 4.6×100 mm (Manufacturer: Thermo).

Mobile Phase: Mobile Phase A (MPA): 1.5M (NH₄)₂SO₄+50 mM potassium phosphate (pH 7.0); Mobile Phase B (MPB): 50 mM sodium phosphate (pH 7.0)/isopropanol (75:25, VN); elution was performed according to the following elution procedure (5%-95%): 100%-95% Mobile Phase A and 0%-5% Mobile Phase B for 0-2 min; 95%-5% Mobile Phase A and 5%-95% Mobile Phase B for 2-22 min; 5%-0% Mobile Phase A and 95%-100% Mobile Phase B for 22-24 min; 0%-100% Mobile Phase A and 100%-1% Mobile Phase B for 24-26 min; 100%-100% Mobile Phase A and 0%-0% Mobile Phase B for 26-30 min.

Detection Conditions: The flow rate of the mobile phase was set at 1 ml/min, the detection wavelength was 280 nm, and the column temperature was 30° C.

Experimental Procedure: 50 μg of the conjugated sample (volume dependent on sample concentration) was injected into the high-performance liquid chromatograph, eluted using the above elution procedure, and the chromatogram was recorded.

DAR=Σ(relative peak area×number of loaded drugs)/100 Calculation Formula for DAR Value:

(b) Analysis of Free Linker Payload (mol/mol %) Value by C18-HPLC

High-Performance Liquid Chromatograph: e2695 High-Performance Liquid Chromatography System.

Chromatographic Column: XBridge® C18, 3.5 μm, 4.6×150 mm (Manufacturer: Waters).

Mobile Phase: Mobile Phase A (MPA): 0.1% TFA-H₂O; Mobile Phase B (MPB): 0.1% TFA-ACN; elution was performed according to the following elution procedure (5%-95%): 90%-20% Mobile Phase A and 10%-80% Mobile Phase B for 0-30 min; 20%-90% Mobile Phase A and 80%-10% Mobile Phase B for 30-31 min; 90%-90% Mobile Phase A and 10%-10% Mobile Phase B for 31-35 min.

Detection Conditions: The flow rate of the mobile phase was set at 0.5 ml/min, the detection wavelength was 254 nm, and the column temperature was 30° C.

Example of Reagent I Preparation: 30 mL of anhydrous methanol and 50 mL of acetonitrile were measured, and 10 g of sodium chloride was weighed. All were mixed in a vessel, stirred at room temperature for more than 1 hour, and then allowed to stand for 1 hour. The supernatant was collected, filtered through a 0.22 μm organic membrane, and stored at room temperature, with a shelf life of 3 months.

Example of Reagent II Preparation: 100 mL of Reagent I, 15 mL of DMSO, and 85 mL of ADC sample storage buffer were measured separately, mixed uniformly in a vessel, and stored at room temperature, with a shelf life of 2 months.

### Experimental Procedure:

### Preparation of Sample Solutions:

1) Free Toxin Reference Standard: The toxin corresponding to the test sample was used as the reference standard and prepared to a final concentration of 1.0 mg/ml using the above Reagent II.
2) 10 μl of the above toxin reference standard with a concentration of 1.0 mg/ml was added to 90 μl of Reagent II to achieve a concentration of 100 μg/ml; the sample was then serially diluted with Reagent II according to the table below to prepare samples of the required concentrations.

| | Concentration | Volume of | Volume of | Concentration |
|---|---|---|---|---|
| Sample | of Sample | Sample to | Reagent II | for Sample |
| No. | to be Taken | be Taken | to be Taken | Preparation |
| 1 | 100 μg/ml | 40 μl | 360 μl | 10 μg/ml |
| 2 | 10 μg/ml | 180 μl | 180 μl | 5.0 μg/ml |
| 3 | 5.0 μg/ml | 120 μl | 180 μl | 2.0 μg/ml |
| 4 | 2.0 μg/ml | 180 μl | 180 μl | 1.0 μg/ml |
| 5 | 1.0 μg/ml | 180 μl | 180 μl | 0.5 μg/ml |
| 6 | 0.5 μg/ml | 120 μl | 180 μl | 0.2 μg/ml |
| 7 | 0.2 μg/ml | 180 μl | 180 μl | 0.1 μg/ml |

Note:
The above samples with prepared required concentrations were injected into the HPLC in order from the lowest to the highest concentration.

Sample Testing: 85 μg of the conjugated sample was taken and mixed with 3 μl of DMSO for 5 min. Then, 60 μl of Reagent I was added to the sample system and mixed thoroughly for 5-10 min, followed by centrifugation at 2000 rpm for 2 min. 20 μL of the supernatant was taken and injected into the high-performance liquid chromatograph. Elution was performed using the above elution procedure, and the chromatogram was recorded.

The content of free toxin in the ADC was calculated according to the calculated standard linear regression equation:

Free Drug (mol/mol %)=(Molar Concentration of Residual Small Molecules/Molar Concentration of Antibody)×100.

### (c) Determination of Free Linker-Payload Content by LC-MS/MS Method

Internal Standard Compounds: Tolbutamide, labetalol, etofexamide, and buspirone at a concentration of 50 ng/ml

### Extractant: Ethyl Acetate

Serial Dilution of Reference Standards: The reference standards were diluted with 50% aqueous methanol solution to 20000, 16000, 10000, 4000, 1000, 200, 40, and 20 ng/ml, respectively.

Sample Pretreatment Prior to Instrument Analysis: 20 μL of the sample was taken, followed by the addition of 20 μL of blank buffer and 240 μL of the extractant. After vortex mixing, the mixture was allowed to stand until layer separation was achieved. Then, 160 μL of the supernatant was transferred into a 96-well plate and dried by blowing. 200 μL of 50% aqueous acetonitrile solution was added and mixed thoroughly prior to LC-MS/MS analysis.

Standard Pretreatment Prior to Instrument Analysis: 2 μL of the working solution was added to 38 μL of blank buffer, followed by the addition of 240 μL of the extractant. After vortex mixing, the mixture was allowed to stand until layer separation was achieved. 160 μL of the supernatant was transferred into a 96-well plate and dried by blowing. 200 μL of 50% aqueous acetonitrile solution was added and mixed thoroughly.

Mass Spectrometry Conditions:

| | |
|---|---|
| LC-MS/MS System | LC-MS/MS (API6500 + ABSciex ExionAC: SH-LCMS-005) |

Liquid Chromatography Method

| Mobile Phase A | 0.1% FA & 5 mM NH4FA in Water | | | |
|---|---|---|---|---|
| Mobile Phase B | 0.5% FA in ACN | | | |
| Chromatographic | ZORBAX Eclipse XDB-C18 2.1 × 50 mm, 3.5 μm | | | |
| column | | | | |
| Liquid | AB Sciex | | | |
| Chromatograph | | | | |
| Autosampler | AB Sciex | | | |
| Injection Volume (μL) | 20 | | | |
| | Time | Flow Rate | Mobile Phase | Mobile Phase |
| | (min) | (mL/min) | A (%) | B (%) |
| Elution Method | 0 | 0.600 | 70 | 30 |
| | 0.30 | 0.600 | 70 | 30 |
| | 2.00 | 0.600 | 5 | 95 |
| | 2.50 | 0.600 | 5 | 95 |
| | 2.51 | 0.600 | 70 | 30 |
| | 3.00 | 0.600 | 70 | 30 |

Mass Spectrometric Method

| Ion Source | ESI | |
|---|---|---|
| Scans | MRM | |
| Polarity | Positive | |
| Mass Spectrometry | CUR (psi) | 40 |
| Parameters | CAD | 8 |
| | IS (V) | 5500 |
| | TEM (°C) | 500 |
| | GS1 (psi) | 50 |
| | GS2 (psi) | 50 |
| | EP (V) | 10 |
| | CXP (V) | 6 |

Data Processing: A standard curve was plotted based on the concentrations of reference standards and their corresponding mass spectrometric response values. The residual content of free linker-payload was calculated via the linear fitting curve (formula) and the corresponding mass spectrometric response values of test samples.

### (d) Analysis of ADC Aggregates by SEC-HPLC

High-Performance Liquid Chromatograph: e2695 High-Performance Liquid Chromatography System or Agilent 1260 Liquid Chromatograph.
Chromatographic column: Waters Xbridge BEH200 SEC (7.8×300 mm, 3.5 μm)
Mobile Phase: 50 mM PB+200 mM Arg (pH 6.80)+10% IPA; elution was performed according to the following elution procedure: 100%-100% Mobile Phase A for 0-30 min;
Detection Conditions: The flow rate of the mobile phase was set at 0.5 ml/min, the detection wavelength was 280 nm, and the column temperature was 26° C.

Experimental Procedure: 20 μg of the conjugated sample (volume dependent on sample concentration) was injected into the high-performance liquid chromatograph, eluted using the above elution procedure, and the chromatogram was recorded.

Monomer Purity (%)=A_{Monomer}/A_{Total}×100%; Aggregate Purity (%)=A_{Aggregate}/A_{Total}×100% Calculation formulas:

### (e) Analysis of ADC Linker Conjugation Mode and ADC Components by Reductive SDS-PAGE

Electrophoresis Apparatus: Tanon EPS-600.
Polyacrylamide Gel: GenScript M00656
Imaging System Analyzer: Tanon 4600SF

Experimental Procedure: 10 μg of the sample was mixed with the corresponding loading buffer, followed by heating in a boiling water bath for 5 minutes. The samples and Protein Marker (10 μL/well) were sequentially loaded into the wells of a 4-20% gradient gel. Electrophoresis was performed at a constant voltage of 120 V for 60 minutes. The gel was removed and rinsed once with deionized water. An appropriate volume of Coomassie Brilliant Blue G250 staining solution was added to submerge the gel, followed by staining on a shaker for 0.5 hour. The stained gel was rinsed three times with deionized water, with shaking on a horizontal shaker for 30 minutes per rinse. After destaining, the gel was removed and then transferred to the imaging analyzer to record the gel image.

Result Analysis: The presence of bands ≥50 kDa indicates that thiol-bridged conjugation is efficient (H, L). The band at 75 kDa represents the fraction composed of a heavy chain and a light chain linked by a pair of bridged thiols (LH). The band at 100 kDa corresponds to two heavy chains linked by one or two pairs of bridged thiols (HH). The band at 125 kDa represents only one heavy chain and one light chain with no bridging (LHH). Finally, the band at 150 kDa represents the antibody with all chains bridged (LHHL).

### (f) Analysis of Bridged Products by LC/TOF (Agilent, 6230 LC/TOF) LC-MS System Liquid Chromatographic Column: PLRP-S, 1000A Column (Agilent, 8 μm, 2.1×150 mm)

Mobile Phase A: 0.1% formic acid
Mobile Phase B: 0.1% formic acid in acetonitrile. Flow rate: 0.4 mL/min.
Mass spectrometric analysis was performed in positive ion mode with the following parameters: Scan range: 500-5000 m/z,
Gas temperature: 320° C.
VCap: 4500 V
Fragmentor: 200 V
Skimmer: 65 V.

### Example 12: Preparation of Antibody-Drug Conjugate ADC-1

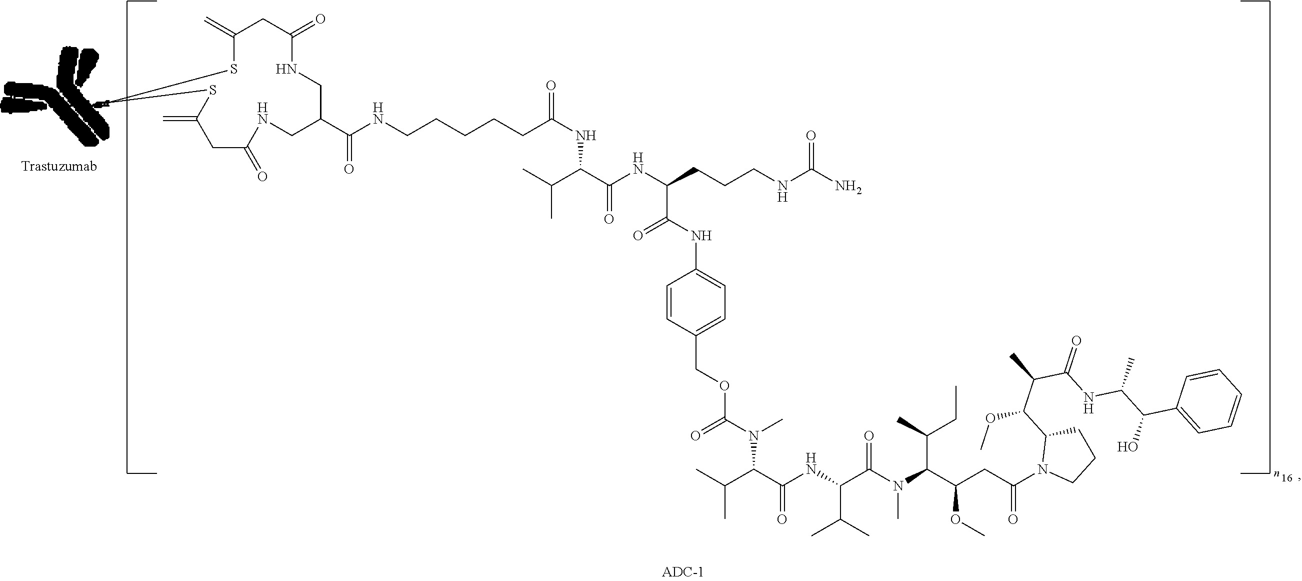

Following the general conjugation procedure for the preparation of bis-allene covalently bridged antibody-drug conjugates, an aqueous solution of TCEP (10 mmol/L, 6 eq.) was added to a buffer solution of trastuzumab (1.0 mg, 38.9 mg/mL, 1.0 eq.) in 20 mM PBS containing 1 mM EDTA (pH 7.4), and the reduction was performed at 22° C. for 2 hours. TCEP was then removed via a desalting column, and the buffer was exchanged to 25 mM Na₂B₄O₇ (pH 8.0). A solution of LP1 (10 mg/mL, 6 eq.) in DMSO was then added, and the conjugation reaction was performed at 25° C. for 16 hours. After 2 rounds of purification via treatment of activated carbon coated with dextran, the buffer of the desired antibody-drug conjugate was ultrafiltered 3 times to give ADC-1 (C_{ADC} (mg/mL): 6.06, V (mL): 0.08, 48.5% yield).

HIC-DAR: 4.05, SEC Purity: 99.2%, Free Linker Payload (mol/mol %): not detected.

DAR 3: Rt=14.85 min (proportion: 3.28%), DAR 4: Rt=16.65 min (proportion: 88.58%), DAR 5: Rt=18.20 min (proportion: 8.15%).

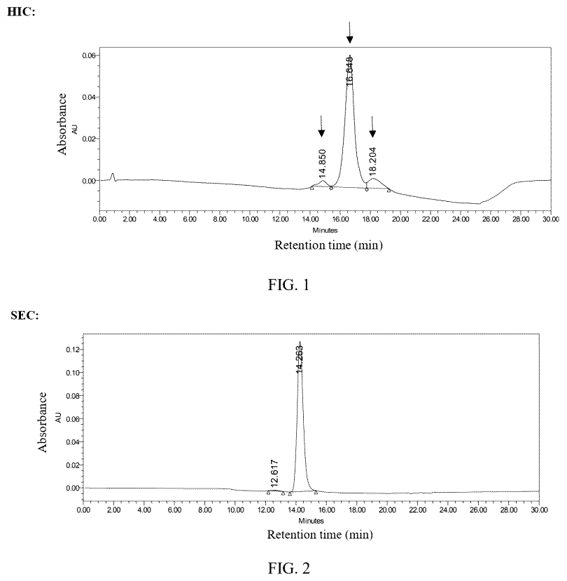

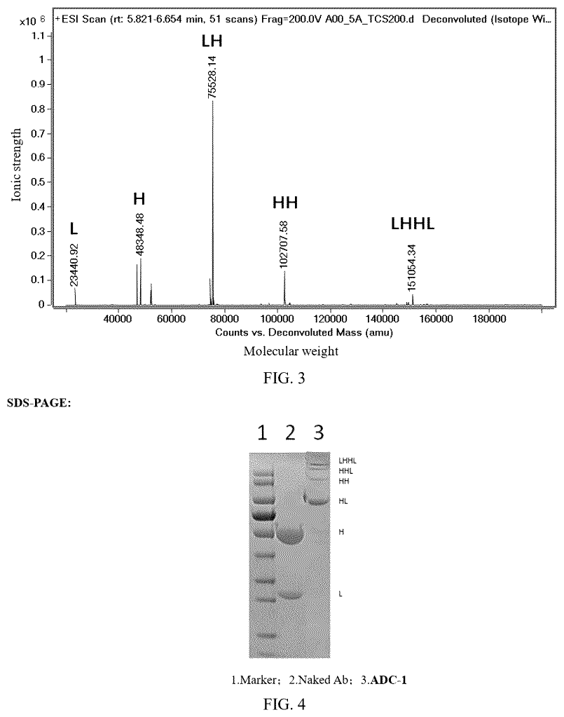

It can be seen from FIGS. 1 and 2 that the proportion of the homogeneous component (DAR4) in the obtained ADC-1 was 88.58%, with aggregates accounting for less than 1%. In addition, FIGS. 3 and 4 showed that the “half-antibody” (DAR4) component formed by conjugation via light chain-heavy chain bridging and intra-heavy chain bridging was predominant.

### Example 13: Preparation of Antibody-Drug Conjugate ADC-2

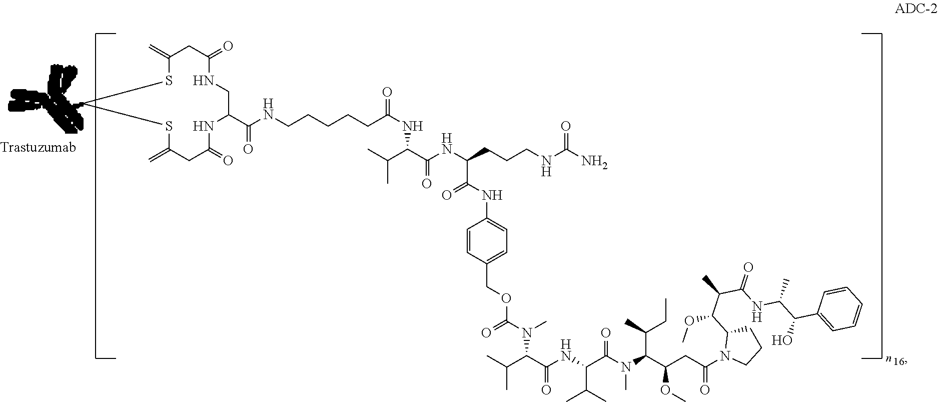

n₁₆=3.86

Following the general conjugation procedure for the preparation of bis-allene covalently bridged antibody-drug conjugates, an aqueous solution of TCEP (2 mg/mL, 6 eq.) was added to a buffer solution of trastuzumab (1.0 mg, 38.9 mg/mL, 1.0 eq.) in 25 mM Na₂B₄O₇ containing 25 mM NaCl and 1 mM DTPA (pH 7.4), and the reduction was performed at 22° C. for 2 hours. An equal volume of 500 mM Tris buffer (pH 8.8) was then added, followed by the addition of a solution of LP2 (10 mg/mL, 7 eq.) in DMSO, and the conjugation reaction was performed at 25° C. for 16 hours. After 2 rounds of purification via treatment of activated carbon coated with dextran, the buffer of the desired antibody-drug conjugate was ultrafiltered 3 times to give ADC-2 (C_{ADC} (mg/mL): 5.2, V (mL): 0.8, 41.6% yield).

HIC-DAR: 3.86, SEC Purity: 93.68%, Free Linker Payload (mol/mol %): not detected.

DAR 3: Rt=15.84 min (proportion: 29.79%), DAR 4: Rt=16.99 min (proportion: 54.92%), DAR 5: Rt=18.90 min (proportion: 15.29%).

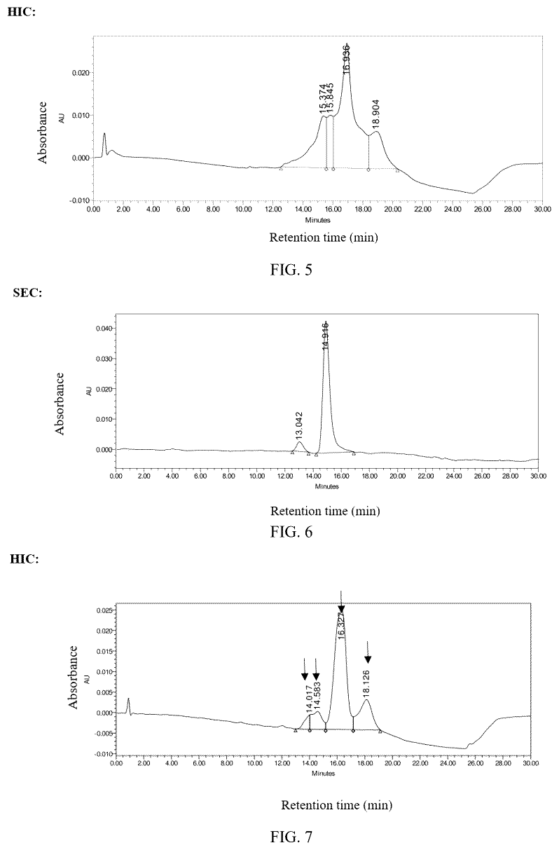

It can be seen from FIGS. 5 and 6 that the proportion of the homogeneous component (DAR4) in the obtained ADC-2 was 54.92%, which was decreased compared with ADC-1, with aggregates accounting for more than 6%.

### Example 14: Preparation of Antibody-Drug Conjugate ADC-3

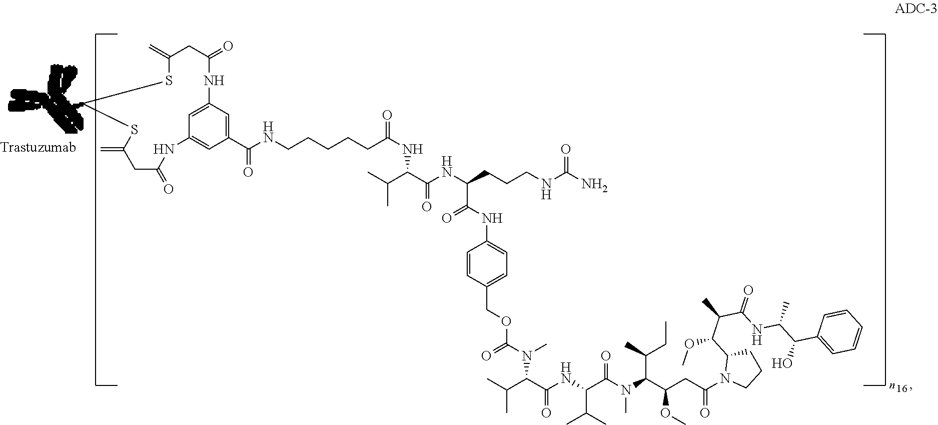

n₁₆=4.1

Following the general conjugation procedure for the preparation of bis-allene covalently bridged antibody-drug conjugates, an aqueous solution of TCEP (10 mmol/L, 6 eq.) was added to a buffer solution of trastuzumab (1.0 mg, 20 mg/mL, 1.0 eq.) in 20 mM PBS containing 1 mM EDTA (pH 7.4), and the reduction was performed at 22° C. for 2 hours. TCEP was then removed via a desalting column, and the buffer was exchanged to 25 mM Na₂B₄O₇ (pH 8.0). A solution of LP3 (10 mg/mL, 5 eq.) in DMSO was then added, and the conjugation reaction was performed at 25° C. for 16 hours. After 2 rounds of purification via treatment of activated carbon coated with dextran, the buffer of the desired antibody-drug conjugate was ultrafiltered 3 times to give ADC-3 (C_{ADC} (mg/mL): 9.32, V (mL): 0.07, 65.24% yield).

HIC-DAR: 4.1, SEC Purity: 98.3%, Free Linker Payload (mol/mol %): not detected.

DAR 2: Rt=14.02 min (proportion: 3.52%), DAR 3: Rt=14.58 min (proportion: 9.03%), DAR 4: Rt=16.33 min (proportion: 68.5%), DAR 5: Rt=18.13 min (proportion: 18.93%).

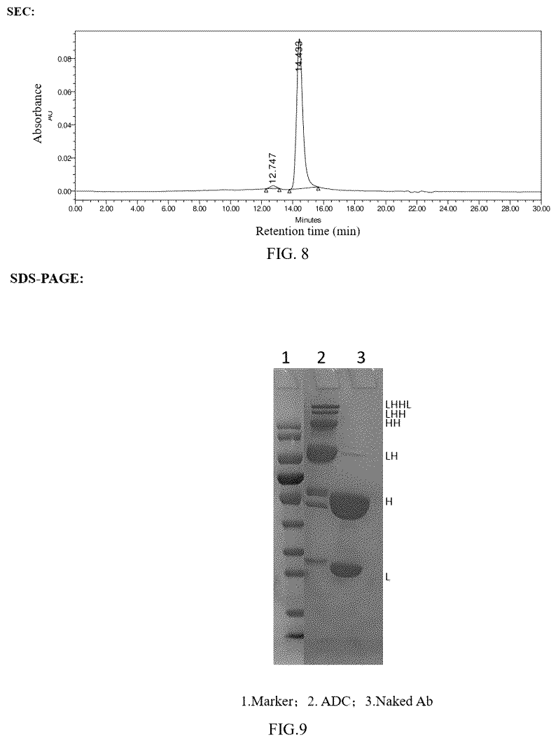

It can be seen from FIGS. 7 and 8 that the proportion of the homogeneous component (DAR4) in the obtained ADC-3 was 68.5%, which was decreased compared with ADC-1, with aggregates accounting for 1.7%. In addition, FIG. 9 showed that the “half-antibody” (DAR4) component formed by conjugation via light chain-heavy chain bridging and intra-heavy chain bridging was predominant.

### Example 15: Preparation of Antibody-Drug Conjugate ADC-5

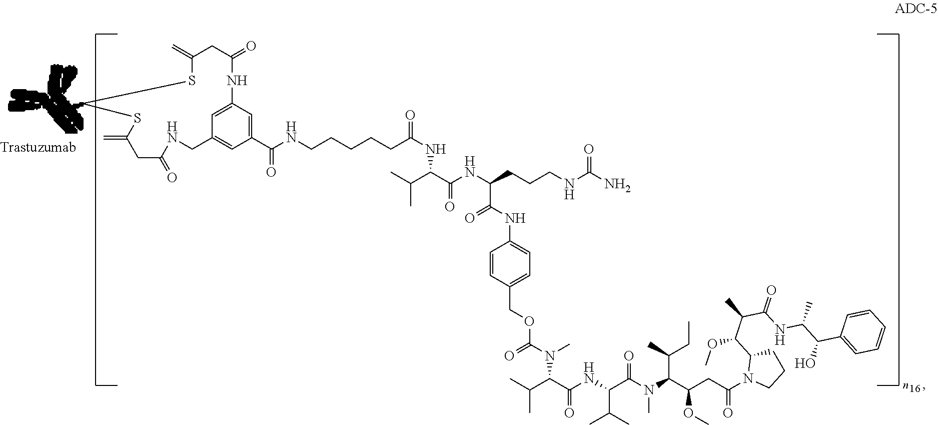

n₁₆=3.89

Following the general conjugation procedure for the preparation of bis-allene covalently bridged antibody-drug conjugates, an aqueous solution of TCEP (2 mg/mL, 6 eq.) was added to a buffer solution of trastuzumab (1.0 mg, 38.9 mg/mL, 1.0 eq.) in 25 mM Na₂B₄O₇ containing 25 mM NaCl and 1 mM DTPA (pH 7.4), and the reduction was performed at 22° C. for 2 hours. An equal volume of 500 mM Tris buffer (pH 8.8) was then added, followed by the addition of a solution of LP5 (10 mg/mL, 6 eq.) in DMSO, and the conjugation reaction was performed at 25° C. for 16 hours. After 2 rounds of purification via treatment of activated carbon coated with dextran, the buffer of the desired antibody-drug conjugate was ultrafiltered 3 times to give ADC-5 (C_{ADC} (mg/mL): 8.2, V (mL): 0.5, 41.0% yield).

HIC-DAR: 3.89

DAR 1: Rt=11.05 min (proportion: 0.55%); DAR 2: Rt=12.80 min (proportion: 2.53%); DAR 3: Rt=15.36 min (proportion: 11.87%); DAR 4: Rt=16.978 min (proportion: 50.24%); DAR 5: Rt=16.978 min (proportion: 29.28%).

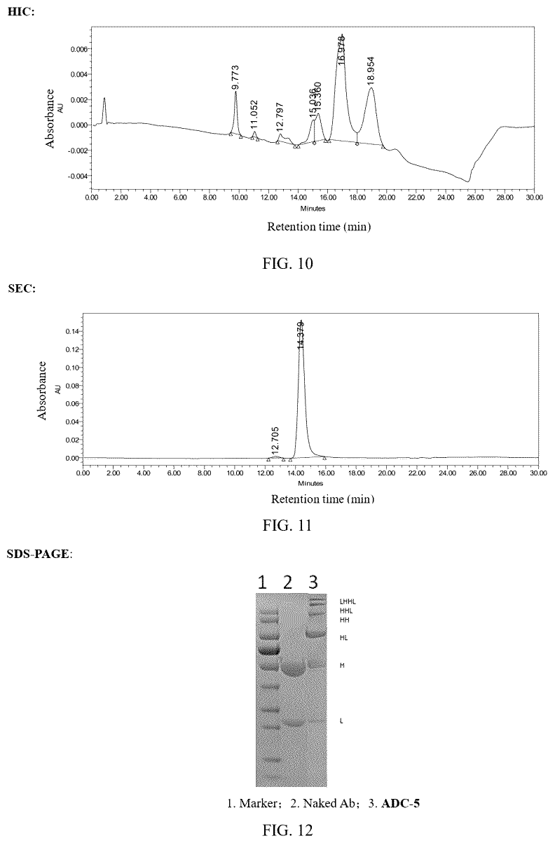

It can be seen from FIGS. 10 to 12 that the proportion of the homogeneous component (DAR4) in the obtained ADC-5 was 50.24%, which was decreased compared with ADC-1, with aggregates accounting for 1.1%. In addition, the proportion of the “half-antibody” (DAR4) component formed by conjugation via light chain-heavy chain bridging and intra-heavy chain bridging was also reduced compared with ADC-1.

### Example 16: Preparation of Antibody-Drug Conjugate ADC-10

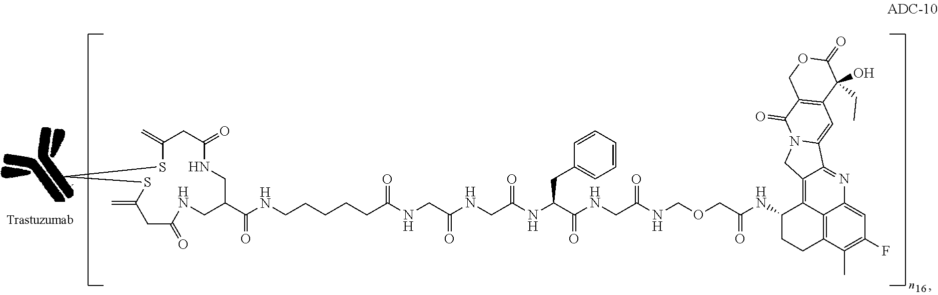

n₁₆=3.98

Following the general conjugation procedure for the preparation of antibody-drug conjugates, an aqueous solution of TCEP (2 mg/mL, 8 eq.) was added to a buffer solution of trastuzumab (0.9 mg, 10 mg/mL, 1.0 eq.) in 50 mM PBS (pH 7.4), and the reduction was performed at 22° C. for 18 hours. The antibody was then buffer-exchanged to sodium borate buffer (pH 8), followed by the addition of a solution of LP10 (10 mg/mL, 7.0 eq.) in DMSO, and the conjugation reaction was performed at 22° C. for 18 hours. After 3 rounds of purification via activated carbon treatment, the buffer of the desired antibody-drug conjugate was ultrafiltered 5 times to give ADC-10 (C_{A}DC (mg/mL): 5.528, V (mL): 0.05, 30.7% yield).

HIC-DAR: 3.98, SEC Purity: 100%, Free Linker Payload (mol/mol %): not detected.

DAR 3: Rt=12.01 min (proportion: 1.64%), DAR 4: Rt=13.48 min (proportion: 98.36%), DAR 5: none.

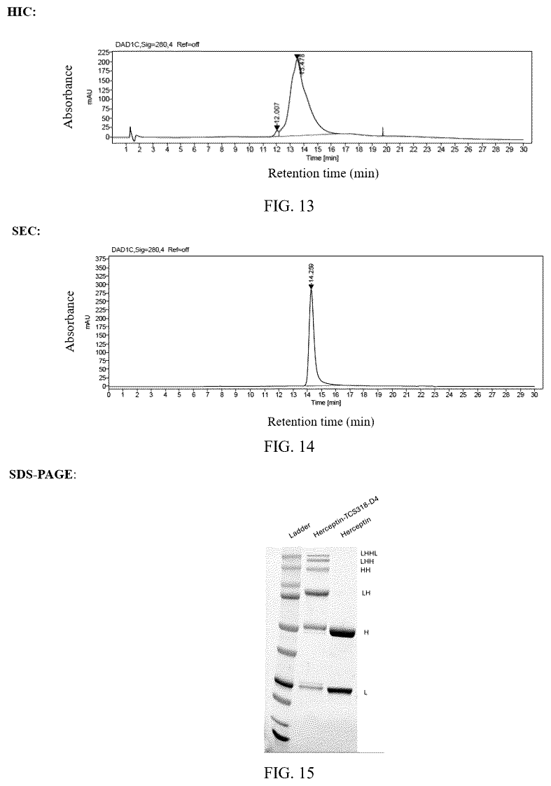

It can be seen from FIGS. 13 and 14 that the proportion of the homogeneous component (DAR4) in the obtained ADC-10 was 98.36%, which was increased compared with ADC-1, with no aggregates detected. In addition, FIG. 15 showed that the “half-antibody” (DAR4) component formed by conjugation via light chain-heavy chain bridging and intra-heavy chain bridging was predominant.

### Example 17: Preparation of Antibody-Drug Conjugate ADC-25

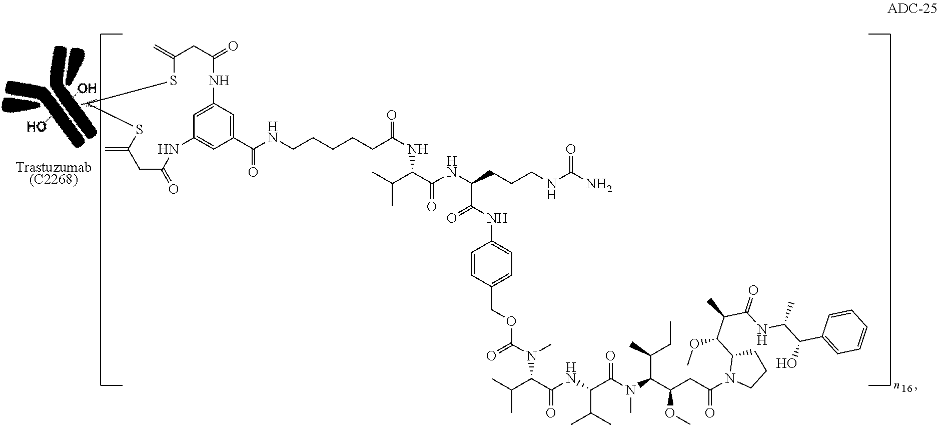

n₁₆=3.00

Following the general conjugation procedure for the preparation of bis-allene covalently bridged antibody-drug conjugates, an aqueous solution of TCEP (10 mmol/L, 6 eq.) was added to a buffer solution of C226S-mutated trastuzumab (1.0 mg, 20 mg/mL, 1.0 eq.) in 20 mM PBS containing 1 mM EDTA (pH 7.4), and the reduction was performed at 22° C. for 2 hours. TCEP was then removed via a desalting column, and the buffer was exchanged to 25 mM Na₂B₄O₇ (pH 8.0). A solution of LP3 (10 mg/mL, 3 eq.) in DMSO was then added, and the conjugation reaction was performed at 25° C. for 16 hours. After 2 rounds of purification via treatment of activated carbon coated with dextran, the buffer of the desired antibody-drug conjugate was ultrafiltered 3 times to give ADC-25 (C_{ADC} (mg/mL): 8.5, V (mL): 0.07, 59.5% yield). The C226S-mutated trastuzumab described in the present invention refers to the trastuzumab in which the cysteine at position 226 of the heavy chain (Eu numbering) is mutated to serine.

HIC-DAR: 3.00, SEC Purity: 98.1%, Free Linker Payload (mol/mol %): not detected.

DAR 2: Rt=12.59 min (proportion: 12.29%), DAR 3: Rt=14.25 min (proportion: 74.33%), DAR 4: Rt=16.45 min (proportion: 13.38%).

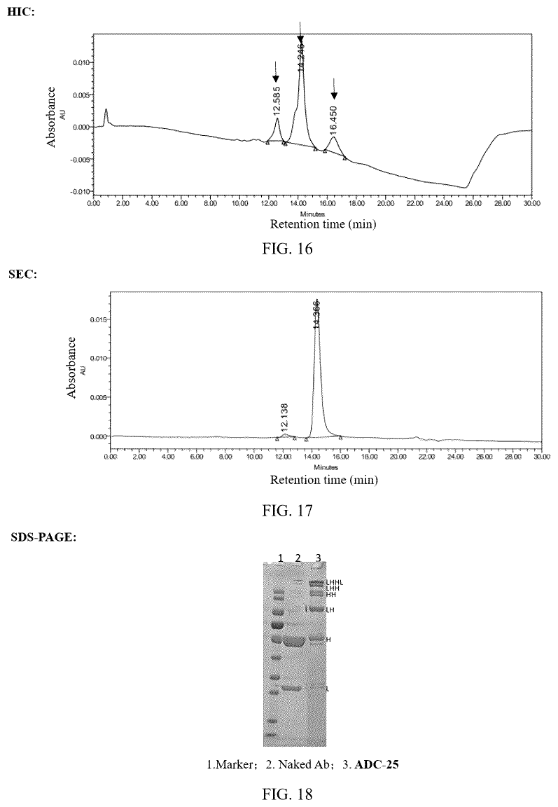

It can be seen from FIGS. 16 and 17 that the proportion of the homogeneous component (DAR3) in the obtained ADC-25 was 74.33%, with aggregates accounting for 1.9%.

### Example 18: Preparation of Antibody-Drug Conjugate ADC-26

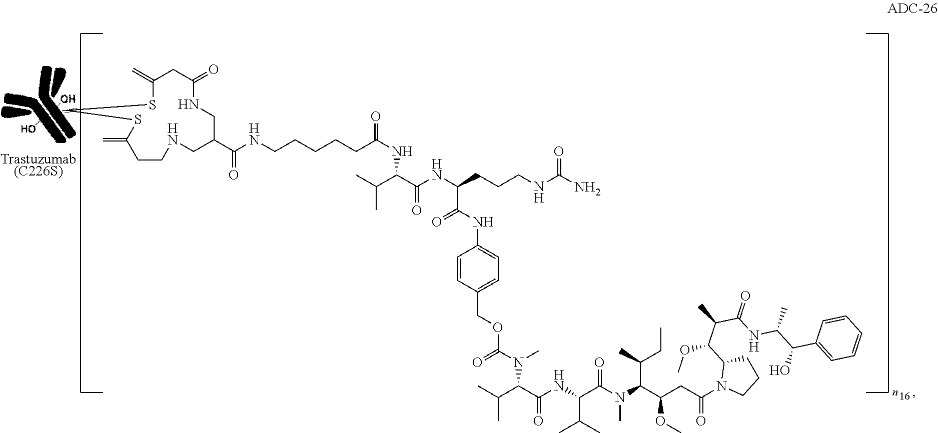

n₁₆=2.94

Following the general conjugation procedure for the preparation of bis-allene covalently bridged antibody-drug conjugates, an aqueous solution of TCEP (10 mmol/L, 6 eq.) was added to a buffer solution of C226S-mutated trastuzumab (1.0 mg, 20 mg/mL, 1.0 eq.) in 20 mM PBS containing 1 mM EDTA (pH 7.4), and the reduction was performed at 22° C. for 2 hours. TCEP was then removed via a desalting column, and the buffer was exchanged to 25 mM Na₂B₄O₇ (pH 8.0). A solution of LP1 (10 mg/mL, 6 eq.) in DMSO was then added, and the conjugation reaction was performed at 25° C. for 16 hours. After 2 rounds of purification via treatment of activated carbon coated with dextran, the buffer of the desired antibody-drug conjugate was ultrafiltered 3 times to give ADC-26 (C_{ADC} (mg/mL): 8.50, V (mL): 0.07, 59.5% yield).

HIC-DAR: 2.94, SEC Purity: 96.3%, Free Linker Payload (mol/mol %): not detected.

DAR 1: Rt=10.88 min (proportion: 1.95%), DAR 2: Rt=12.53 mi (proportion: 12.32%), DAR 3: Rt=14.18 min (proportion: 75.13%), DAR 4: Rt=16.58 mmi (proportion: 10.61%).

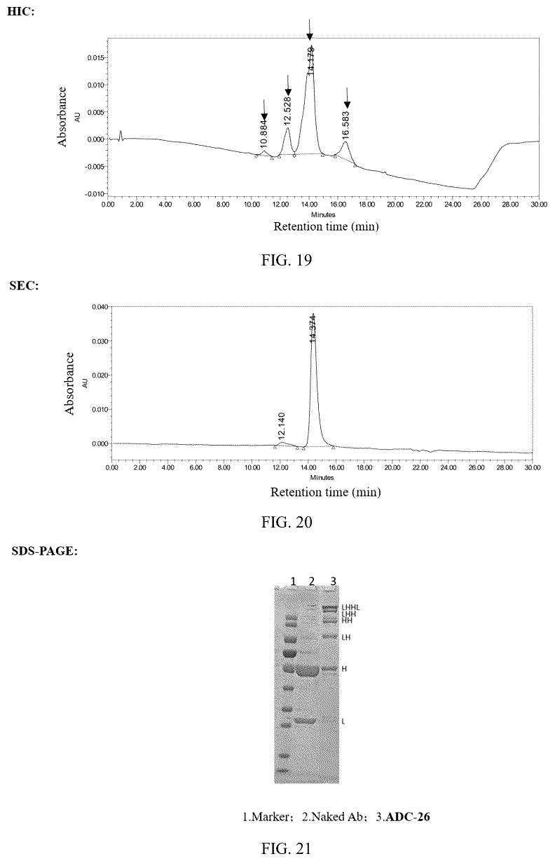

It can be seen from FIGS. 19 and 20 that the proportion of the homogeneous component (DAR3) in the obtained ADC-26 was 75.13%, with aggregates accounting for 3.7%.

### Example 19: Preparation of Antibody-Drug Conjugate ADC-27

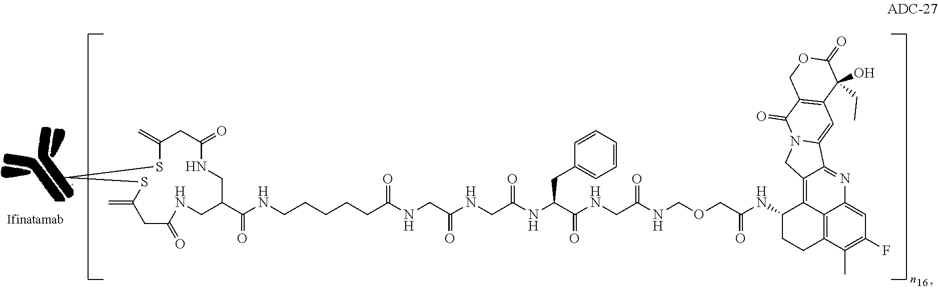

n₁₆=4.00

Following the general conjugation procedure for the preparation of bis-allene covalently bridged antibody-drug conjugates, an aqueous solution of TCEP (10 mmol/L, 6 eq.) was added to a buffer solution of ifinatamab (1.0 mg, 15 mg/mL, 1.0 eq.) in 20 mM PBS containing 1 mM EDTA (pH 7.4), and the reduction was performed at 22° C. for 2 hours. TCEP was then removed via a desalting column, and the buffer was exchanged to 25 mM Na₂B₄O₇ (pH 8.0). A solution of LP10 (10 mg/mL, 6 eq.) in DMSO was then added, and the conjugation reaction was performed at 25° C. for 16 hours. After 2 rounds of purification via treatment of activated carbon coated with dextran, the buffer of the desired antibody-drug conjugate was ultrafiltered 3 times to give ADC-27 (C_{ADC} (mg/mL): 10.4, V (mL): 0.07, 72.8% yield).

HIC-DAR: 4, SEC Purity: 98.8%, Free Linker Payload (mol/mol %): not detected.

DAR 4: Rt=12.90 min (proportion: 100%).

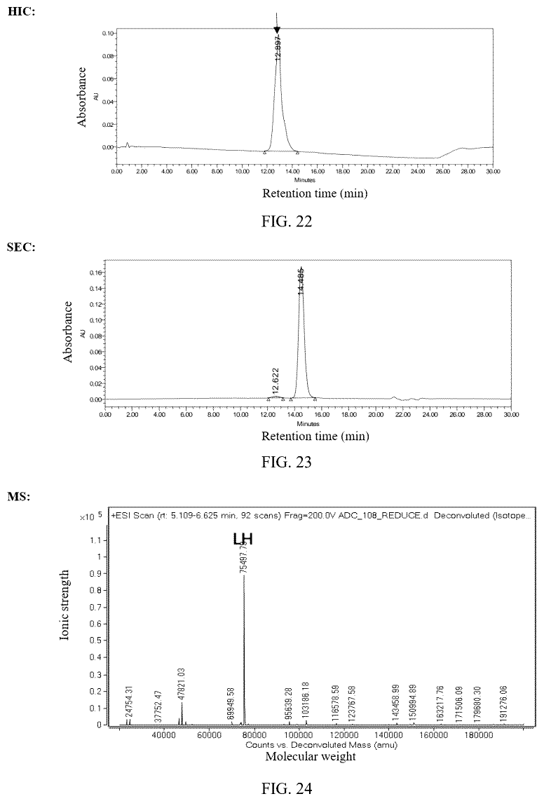

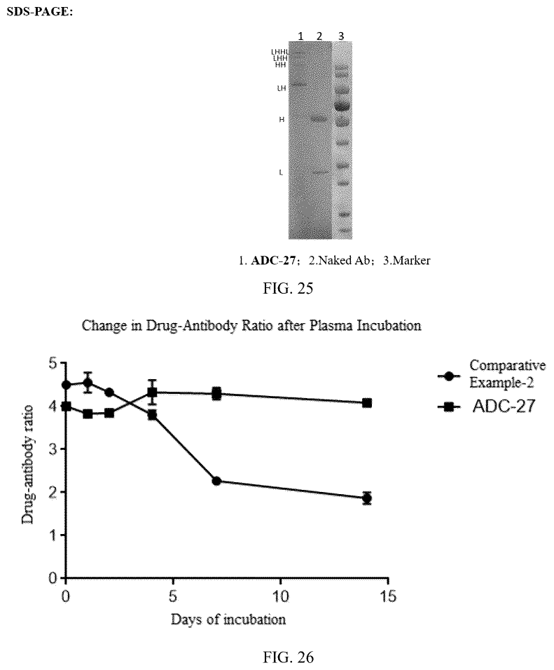

It can be seen from FIGS. 22 and 23 that the proportion of the homogeneous component (DAR4) in the obtained ADC-27 was 100%, with aggregates accounting for 1.2%. In addition, FIGS. 24 and 25 showed that the “half-antibody” (DAR4) component formed by conjugation via light chain-heavy chain bridging and intra-heavy chain bridging was predominant.

### Example 20: Preparation of Anti-Drug Conjugate Comparative Example-1

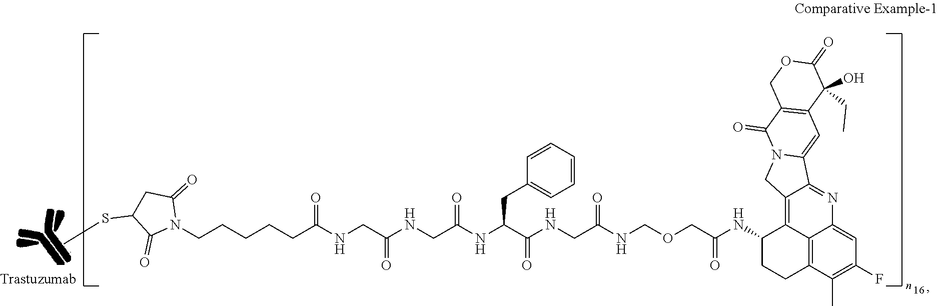

n₁₆=7.96

Following the general conjugation procedure for the preparation of antibody-drug conjugates, an aqueous solution of TCEP (10 mg/mL, 12 eq.) was added to a buffer solution of trastuzumab (5 mg, 21.07 mg/mL, 1.0 eq.) in 50 mM PBS (pH 7.0), and the reduction was performed at 22° C. for 2 hours. A solution of Deruxtecan (10 mg/mL, 10 eq.) in DMSO was then added, and the conjugation reaction was performed at 22° C. for 1 hour. After 2 rounds of purification via treatment of activated carbon coated with dextran, the buffer of the desired antibody-drug conjugate was ultrafiltered 3 times to give Comparative Example-1 (C_{A}Dc (mg/mL): 6.74, V (mL): 0.6, 80.9% yield).

Through the general characterization method for antibody-drug conjugates, the following characterization results were obtained: HIC-DAR: 7.96, SEC Purity: 97.7%, Free Linker Payload (mol/mol %): 0.04221.

### Example 21: Preparation of Antibody-Drug Conjugate Comparative Example-2

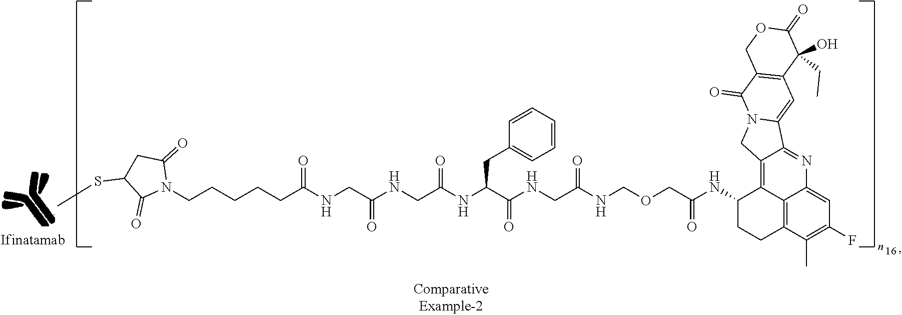

n₁₆=4.5

Following the general conjugation procedure for the preparation of antibody-drug conjugates, an aqueous solution of TCEP (10 mM, 12 eq.) was added to a buffer solution of ifinatamab (20 mg, 21.07 mg/mL, 1.0 eq.) in 50 mM PBS (pH 7.0), and the reduction was performed at 22° C. for 2 hours. A solution of Deruxtecan (10 mg/mL, 10 eq.) in DMSO was then added, and the conjugation reaction was performed at 22° C. for 1 hour. After 2 rounds of purification via treatment of activated carbon coated with dextran, the buffer of the desired antibody-drug conjugate was ultrafiltered 3 times to give Comparative Example-2 (C_{ADC} (mg/mL): 7.47, V (mL): 2.2, 82.2% yield).

Through the general characterization method for antibody-drug conjugates, the following characterization results were obtained:
HIC-DAR: 4.5, SEC Purity: 99.92%, Free Linker Payload (mol/mol %): 0.21077.

### Example 22: In Vitro Plasma Stability Assay of ADCs Based on Different Linkers

This example evaluated the plasma stability of antibody-drug conjugates based on the bis-allene linker and the maleimide linker, with the monitoring index being the change in DAR values over time.

The test antibody-drug conjugates were mixed with human plasma to a final concentration of 0.3 mg/ml and incubated in a constant temperature incubator at 37° C. 50 μL samples were collected at each preset time point (0, 1, 2, 4, 7, 14 days). After sampling, the samples were frozen at −80° C. Upon completion of samplings, the concentrations of antibody-drug conjugates and total antibody were determined uniformly by ELISA. The method was as follows: First, antigen B7-H3 (Sino Biological, 11188-H08H) and anti-Dxd antibody (GenScript, A02217) were coated overnight in the wells of an ELISA plate for the determination of the concentrations of total antibody and ADCs, respectively. The next day, the plate was washed 3 times with 250 μL of PBS/T, followed by the addition of 150 μL of 4% nonfat milk powder and incubation at 37° C. for 2 hours. Thereafter, the sample dilutions to be tested or reference standards were added to the ELISA wells and incubated for an additional 2 hours. The plate was then washed 3 times with 250 μL of PBS/T, and 100 μL of a dilution (1:3000) of Anti-human IgG secondary antibody (GenScript, A01855) was added, followed by incubation at 37° C. for 1 hour. The plate was washed 3 times again with 250 μL of PBS/T, and 50 μL of TMB chromogenic solution was added to each well for color development at 37° C. for 5 minutes. The chromogenic reaction was terminated with 100 μL of stop solution. The absorbance signal at OD450 nm was collected, and a standard curve was plotted. The concentrations of antibody-drug conjugates and total antibody in the samples were calculated based on the standard curve. The change curve of DAR values was further derived, as shown in FIG. 26.

It can be seen from FIG. 26 that ADC-27, which is based on the bis-allene linker, exhibited excellent stability in plasma with DAR values remaining essentially unchanged. In contrast, the antibody-drug conjugate based on the maleimide linker (Comparative Example-2) exhibited a continuous decrease in DAR values during incubation with plasma, with a 50% reduction in DAR values after 2 weeks. Thus, the bis-allene linker exhibits superior stability and has the application potential for further enhancing the safety and efficacy.

### Biological Evaluation

### Example 23: Tumor Cell Proliferation Inhibition Assay of Antibody-Drug Conjugates Based on Bis-Allene Covalent Bridging

1500 NCI-N87 cells were seeded into a 96-well plate (edge wells filled with PBS were excluded). The cells were cultured in 80 μL of appropriate medium and incubated in a constant temperature incubator at 37° C. with 5% CO₂. After overnight incubation of the cells in the constant temperature incubator (37° C., 5% CO₂), 20 μL of the diluted bis-allene-based antibody-drug conjugate solution was added to each well, resulting in a final conjugate concentration range of 0.2 pM-12.5 nM or 3.0 pM-200 nM. After co-incubation of the cells with the conjugate for 5 days, the 96-well plate was equilibrated at room temperature for 30 min. 40 μL of CellTiter-Glo® (Promega, G7572) reagent was added to each well, and after a 10 min reaction in the dark, the fluorescence value was determined using a SpectraMax i3X microplate reader. The curve of luminescence values versus conjugate concentration (nM) was fitted using GraphPad Prism software.

In vitro cytotoxicity of antibody-drug conjugates (++++: <1 nM; +++: 1-10 nM; ++: 10-200 nM; +: >200 nM)

| | | | | NCI-N87 |
|---|---|---|---|---|
| Entry | ADC code | antibody | LP | IC50 (nM) |
| 1 | Comparative | Trastuzumab | Deruxtecan | ++++ |
| | Example-1 | | | |
| 2 | Comparative | Ifinatamab | Deruxtecan | N.A. |
| | Example-2 | | | |
| 3 | ADC-1 | Trastuzumab | LP1 | ++++ |
| 4 | ADC-2 | Trastuzumab | LP2 | ++++ |
| 5 | ADC-3 | Trastuzumab | LP3 | ++++ |
| 6 | ADC-5 | Trastuzumab | LP5 | ++++ |
| 7 | ADC-10 | Trastuzumab | LP10 | ++++ |
| 8 | ADC-25 | Trastuzumab(C226S) | LP3 | ++++ |
| 9 | ADC-26 | Trastuzumab(C226S) | LP1 | ++++ |
| 10 | ADC-27 | Ifinatamab | LP10 | N.A. |

Most ADCs exhibited a certain level of anti-tumor activity; for example, in NCI-N87 cells, the in vitro cytotoxic activity of ADC-1, ADC-2, ADC-3, ADC-5, ADC-10, ADC-25, ADC-26, and ADC-27 was comparable to that of Comparative Example-1.

The main advantages of the present invention are as follows:
1. The present invention provides a novel bis-allene linker, which can conveniently achieve site-specific conjugation to antibodies based on covalent thiol bridging, and is applicable to most antibodies. Compared with the cumbersome recombinant modification of antibodies, the method for site-specific conjugation using the bis-allene linker of the present invention does not require recombinant modification of antibodies, thus saving a great deal of time, costs, and resources, and exhibiting broader application prospects.
2. The novel bis-allene linker provided by the present invention can achieve covalent bridging conjugation with antibodies via a simple chemical method. Compared with the antibody-drug conjugates prepared by traditional random conjugation methods, the conjugates obtained by using the linker of the present invention exhibit a narrower distribution of DAR values and higher product homogeneity. The proportion of the homogeneous component (DAR4) in the obtained conjugates can exceed 80%, and even exceed 95% in some cases, with the “half-antibody” (DAR4) component being predominant.
3. The novel bis-allene linker provided by the present invention can also achieve covalent bridging conjugation with mutants of most antibodies, for example, mutants at positions 226 (Eu numbering) or 229 (Eu numbering) of the heavy chain. Since the cysteine residues that form disulfide bonds between heavy chains are mutated into other amino acids, homogeneous DAR3 antibody-drug conjugates can be generated.
4. With the same antibody employed, the antibody-drug conjugates based on the novel bis-allene linker provided by the present invention exhibit comparable in vitro cytotoxic activity and superior in vitro plasma stability, compared with the antibody-drug conjugates based on the maleimide linker.

The above are merely preferred embodiments of the present invention and are not intended to limit the scope of the present invention. Any modification, equivalent replacement, improvement, and the like made within the spirit of the present invention shall fall within the scope of the claims of the present invention.

1. A bis-allene linker, comprising a compound represented by Formula (I) or an isotopically labeled form thereof: 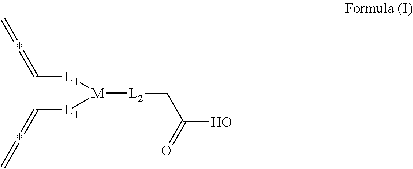
wherein L₁ is selected from the group consisting of 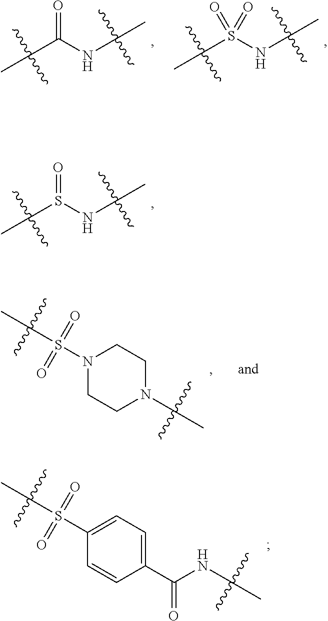 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]
M is selected from the group consisting of 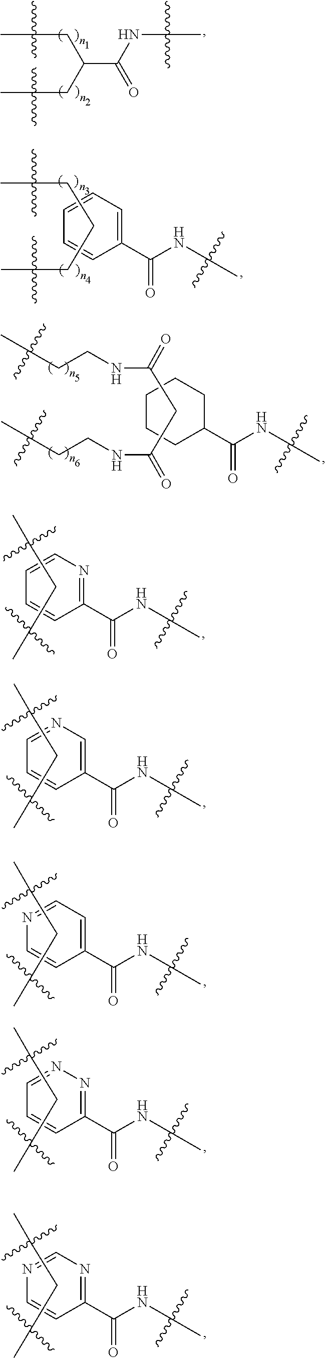 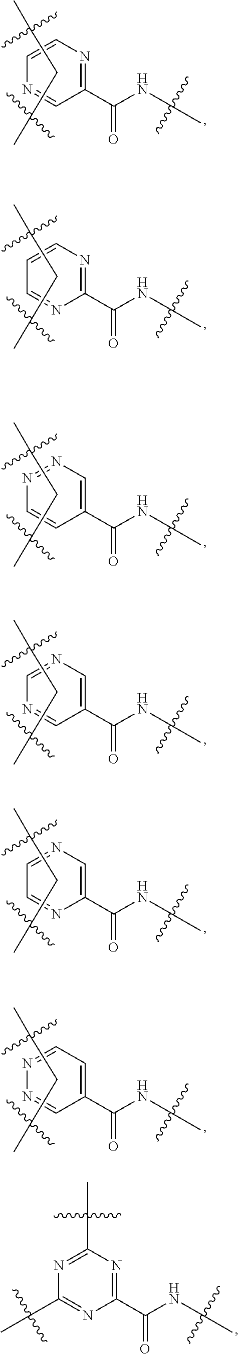 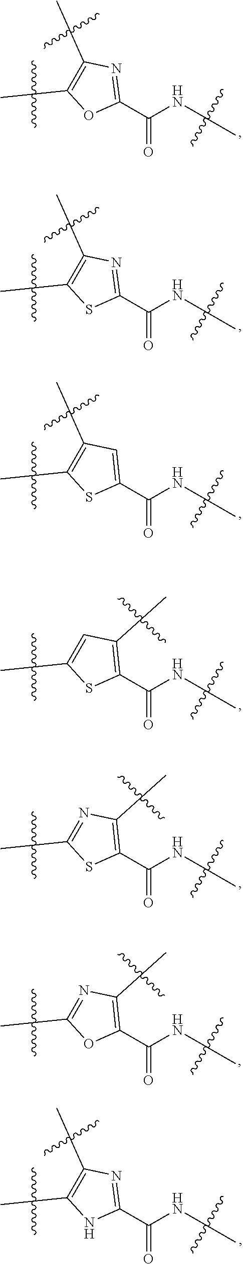 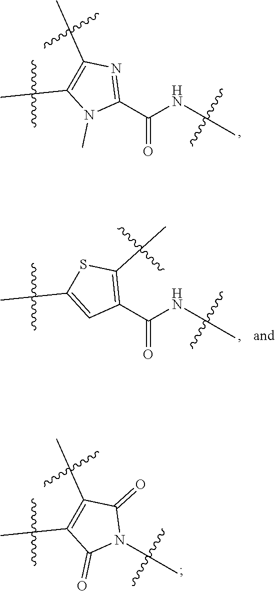 [SMILES: *NC(=O)c1nc(*)c(*)n1C ; *NC(=O)c1cc(*)sc1* ; *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 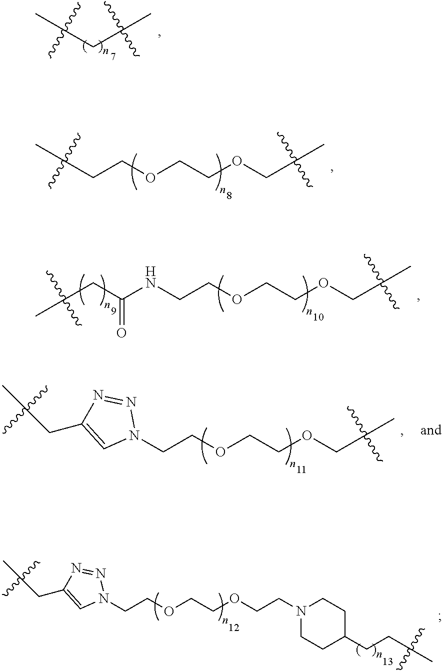
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20.

2. The bis-allene linker according to claim 1, wherein in Formula (I),
L₁ is selected from the group consisting of 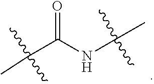 [SMILES: *NC(*)=O]
M is selected from 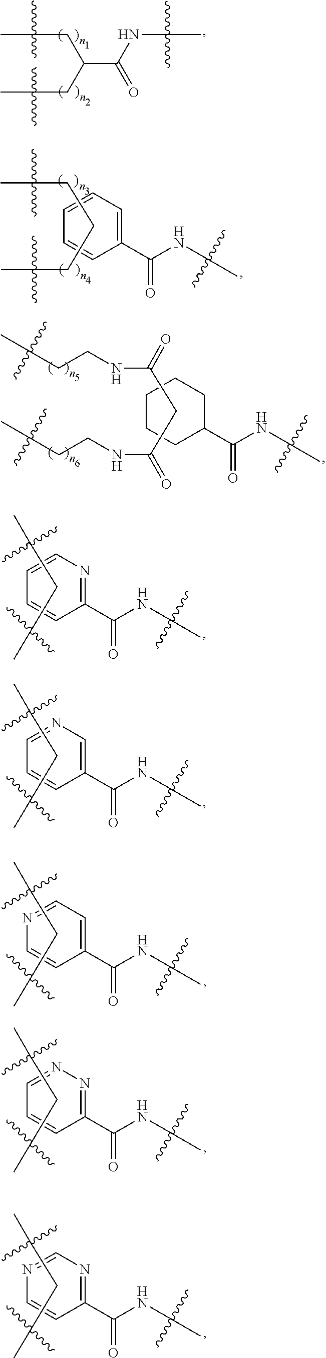 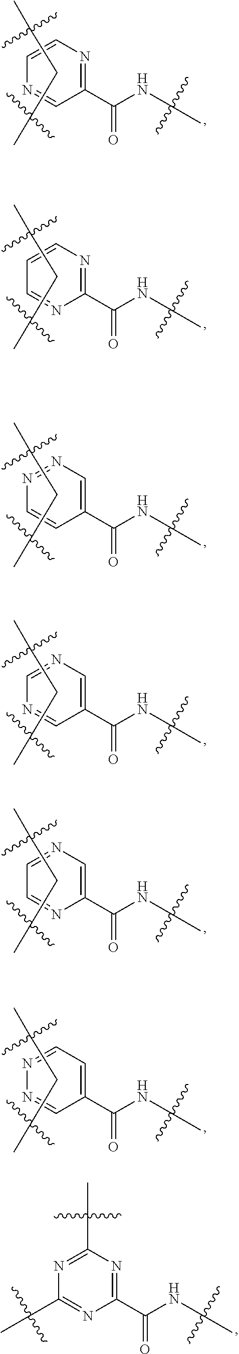 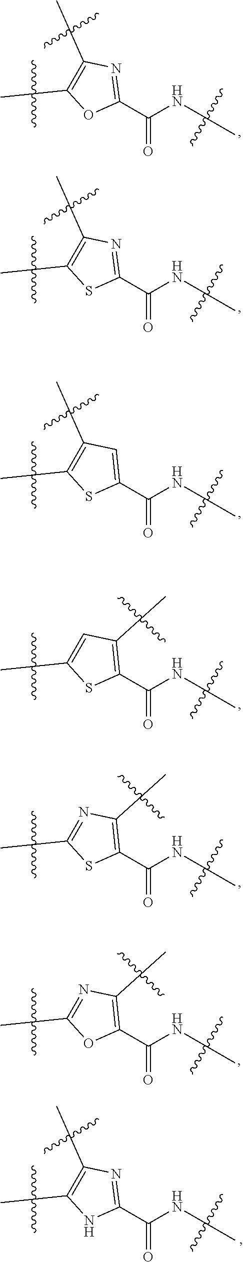 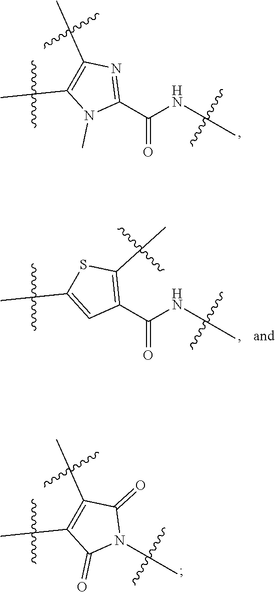 [SMILES: *NC(=O)c1nc(*)c(*)n1C ; *NC(=O)c1cc(*)sc1* ; *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 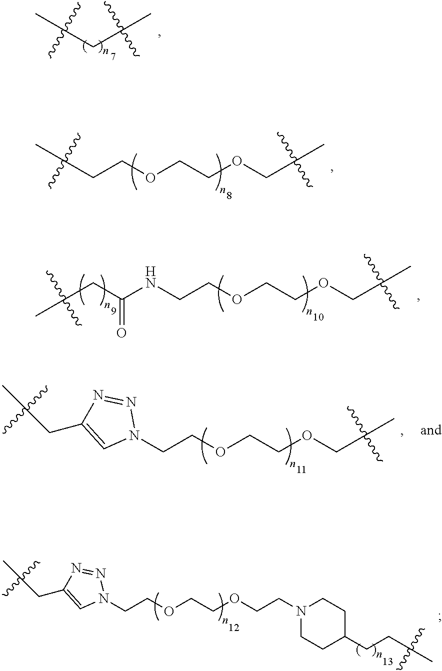
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20.

3. The bis-allene linker according to claim 1, wherein in Formula (I),
L₁ is selected from the group consisting of 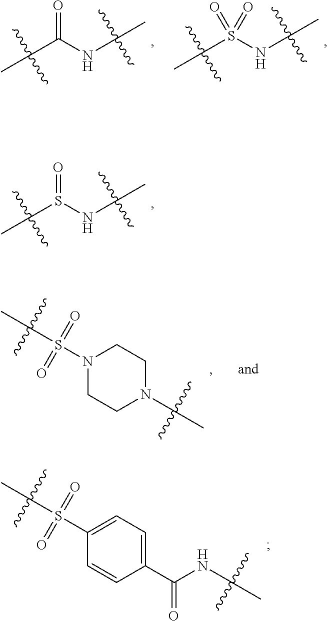 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]
M is selected from the group consisting of 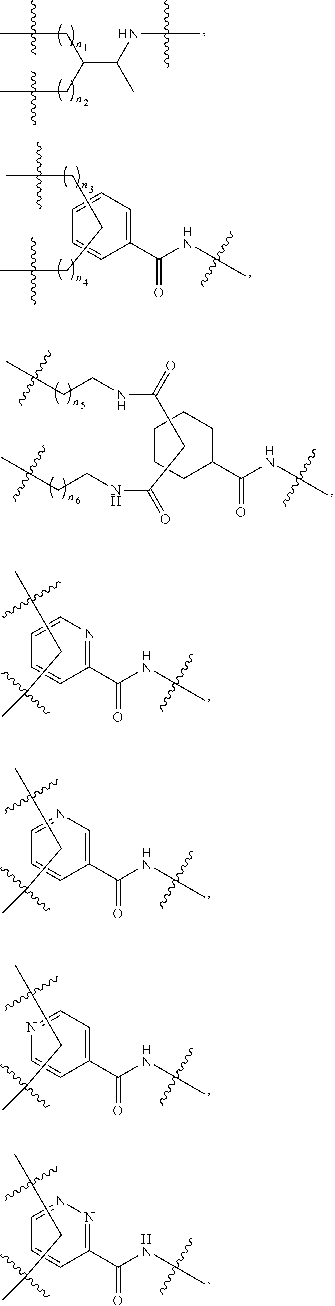 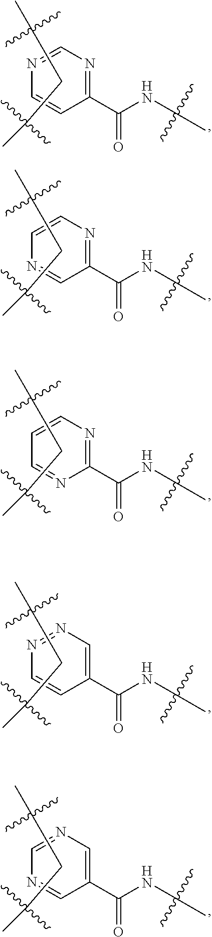 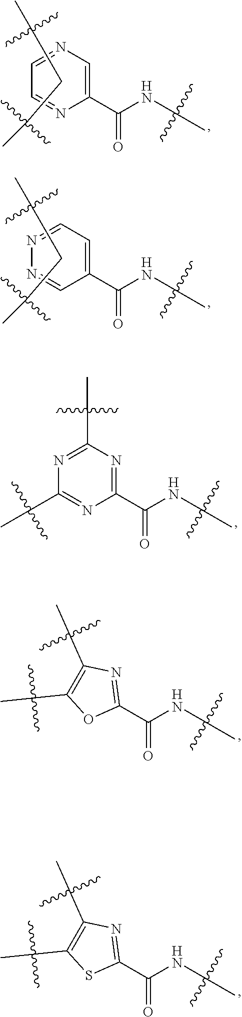 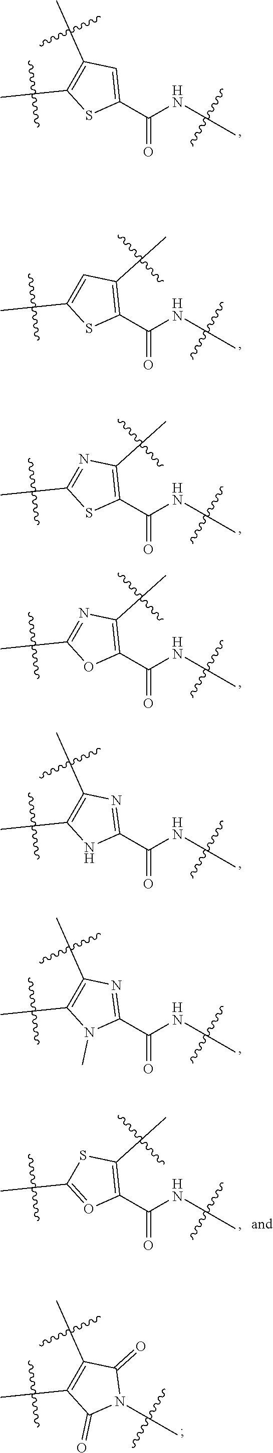
n₁ and n₂ are different, n₃ and n₄ are the same or different, and n₅ and n₆ are the same or different; 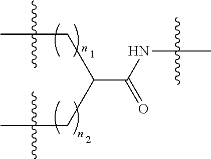
is a single chiral component or a racemate; n₁ is an integer from 0 to 10, n₂ is an integer from 0 to 10, n₃ is an integer from 0 to 10, n₄ is an integer from 0 to 10, n₅ is an integer from 1 to 10, and n₆ is an integer from 1 to 10;
L₂ is selected from the group consisting of one or more combinations of 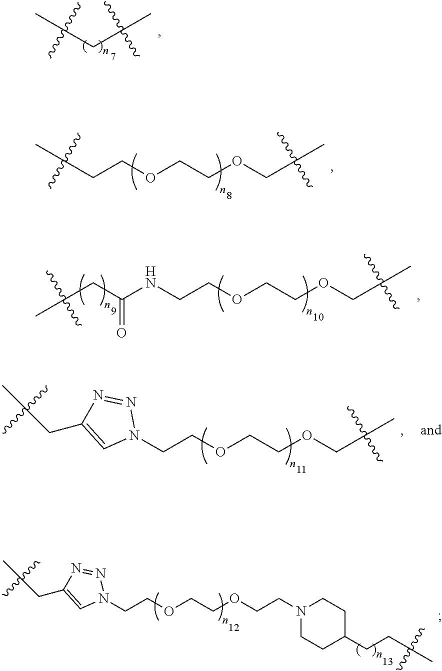
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20.

4. The bis-allene linker according to claim 1, wherein in Formula (I),
L₁ is 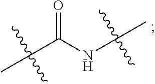 [SMILES: *NC(*)=O]
M is selected from the group consisting of 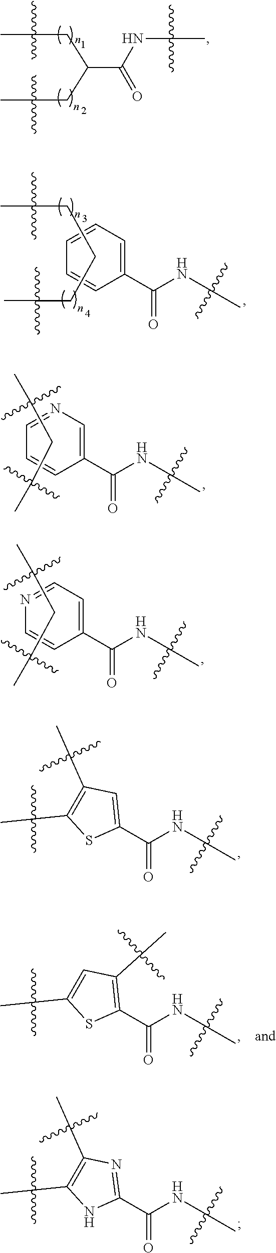
n₁ and n₂ are the same and each independently represent an integer from 0 to 8, and
n₃ and n₄ are the same and each independently represent an integer from 0 to 8;
L₂ is 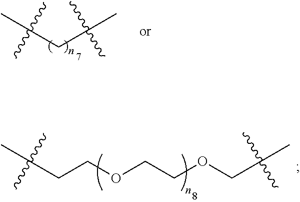
n₇ is an integer from 1 to 10, and n₈ is an integer from 1 to 10.

5. The bis-allene linker according to claim 1, wherein the bis-allene linker of Formula (I) is one or more selected from the group consisting of the compounds represented by the following structural formulas:

| No. | Structure |
|---|---|
| LK1 | 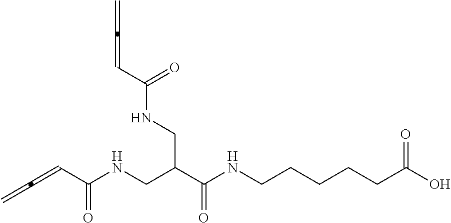 |
| LK2 | 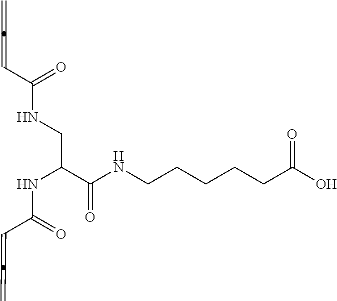 |
| LK3 | 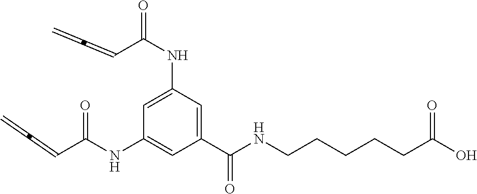 |
| LK4 | 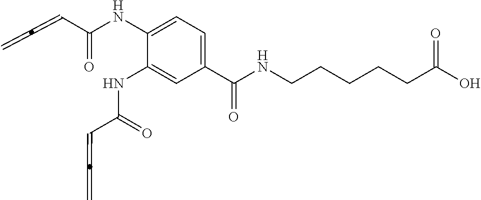 |
| LK5 | 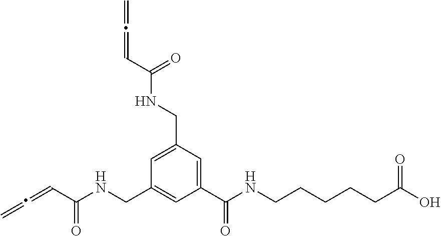 [SMILES: C=C=CC(=O)NCc1cc(CNC(=O)C=C=C)cc(C(=O)NCCCCCC(=O)O)c1] |
| LK6 | 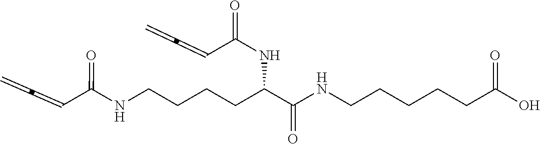 |
| LK7 | 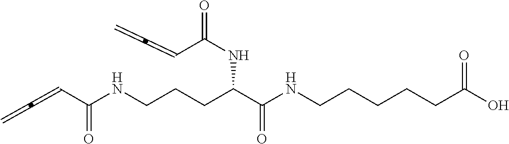 |
| LK8 | 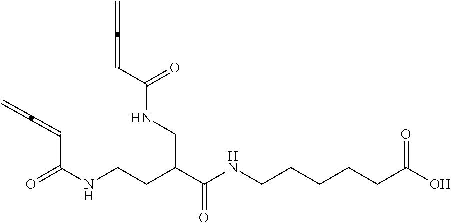 |
| LK9 | 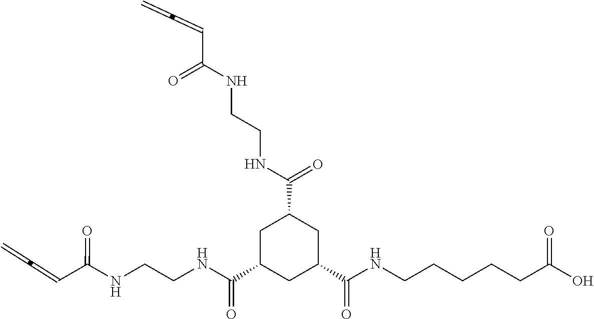 |
| LK10 | 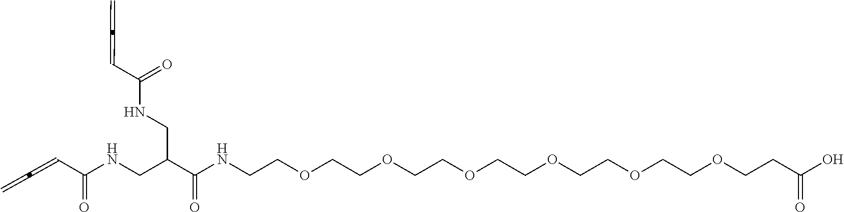 |
| LK11 | 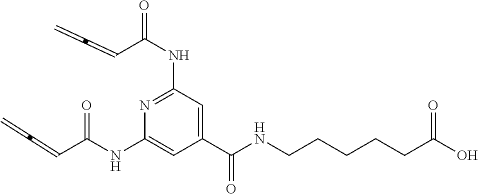 |
| LK12 | 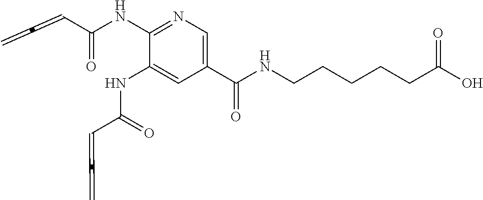 |
| LK13 | 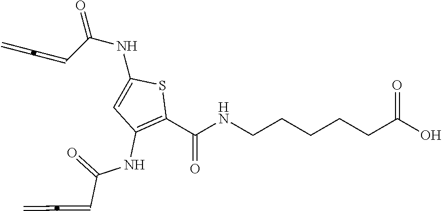 |
| LK14 | 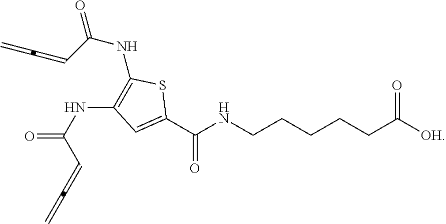 |

6. A linker-drug conjugate, which is a compound formed by the bonding of the carboxyl group in the bis-allene linker according to claim 1 to the amino group in the amino acid residue of L₃ in 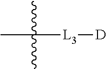
via an amide bond,
wherein L₃ is 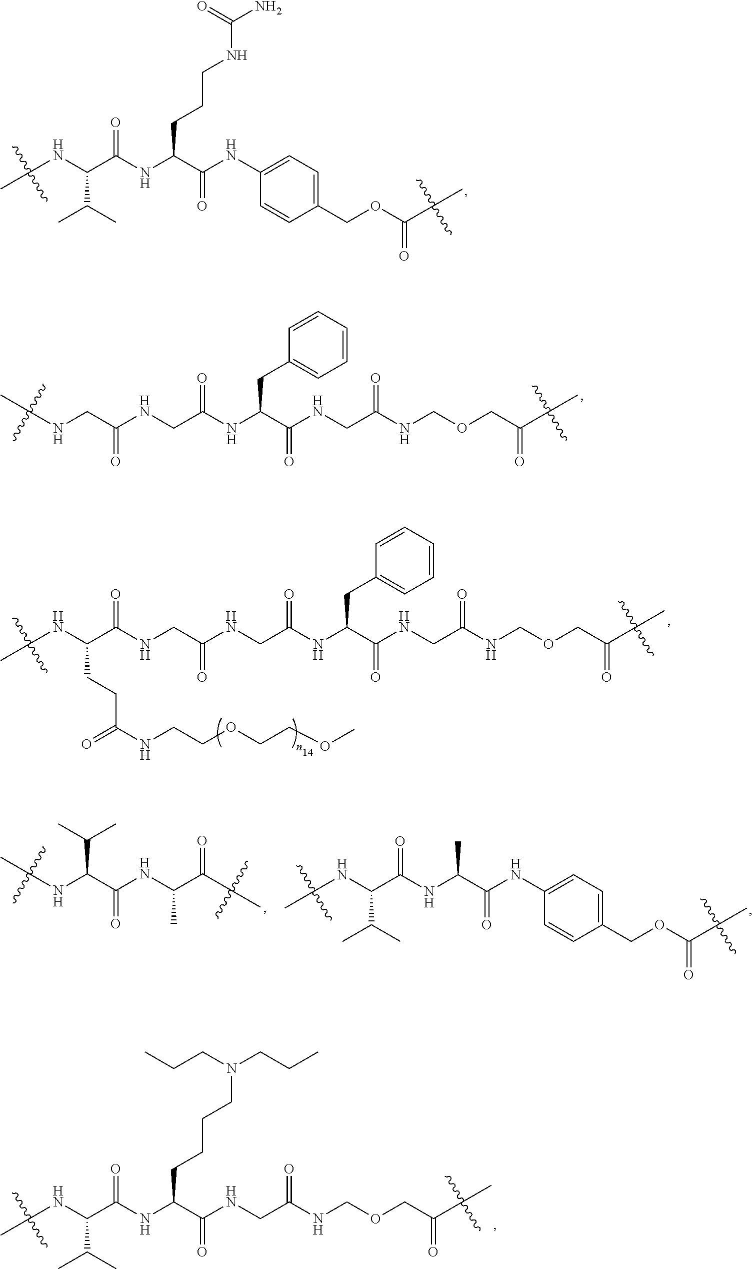 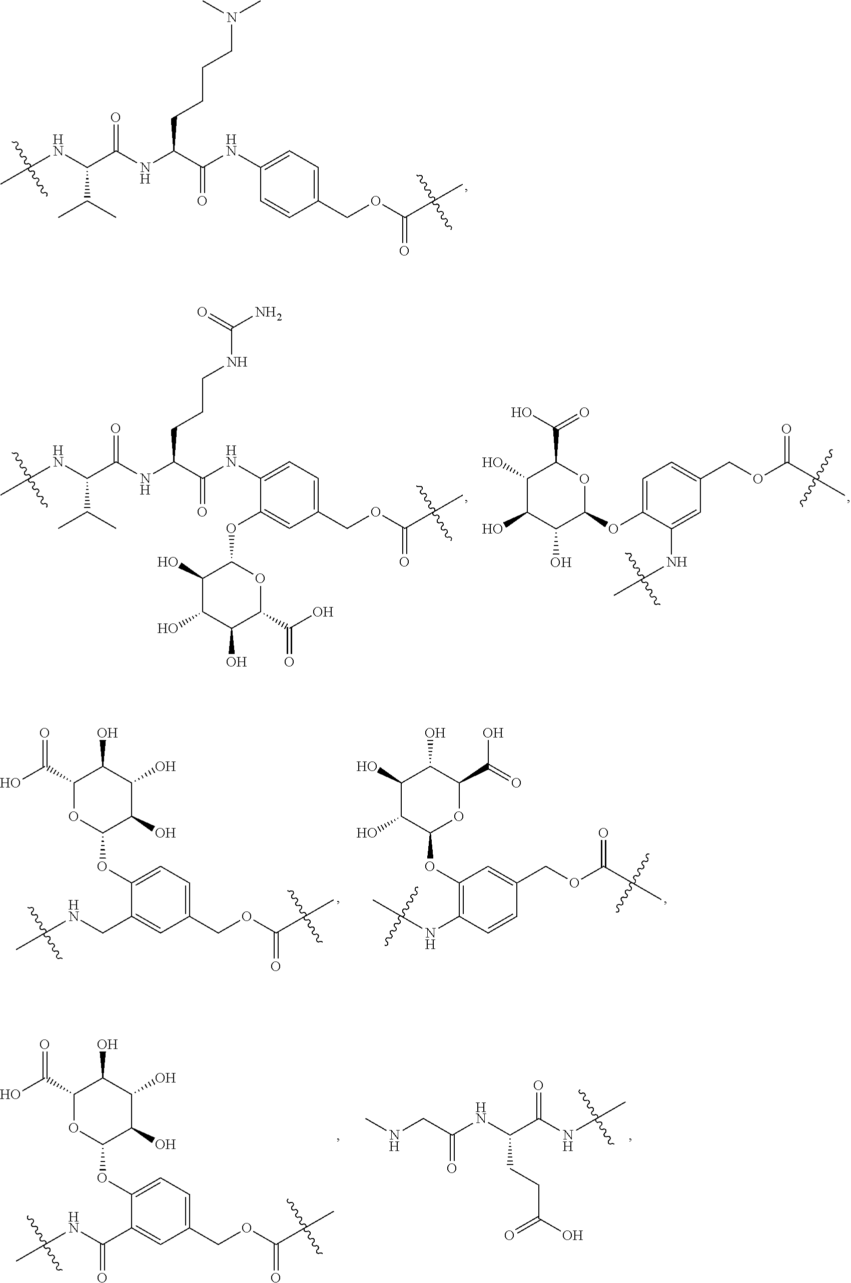 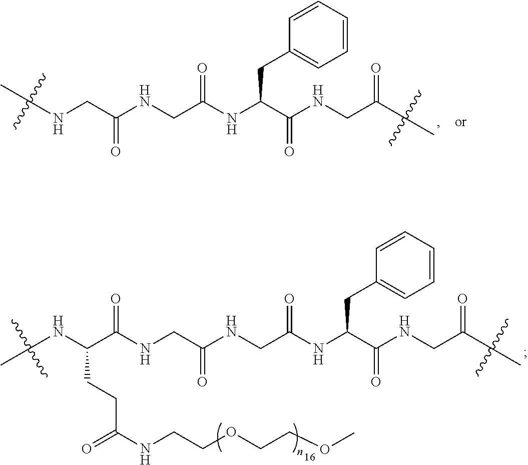
n₁₄ is an integer from 1 to 20, and n₁₅ is an integer from 1 to 20;
D is a fragment formed by the loss of one or more atoms or groups from a cytotoxic small-molecule drug or active agent; wherein D is covalently bonded to L₃ via sulfur, nitrogen, or oxygen in D.

7. The linker-drug conjugate according to claim 6, wherein the active agent is a tubulin inhibitor, an RNA polymerase inhibitor, a topoisomerase inhibitor, an intercalator, a DNA-reactive agent, a DNA alkylating agent, or an immunosuppressant; and the cytotoxic small-molecule drug is a drug for the treatment of tumor diseases.

8. A linker-drug conjugate, comprising a compound represented by Formula (II): 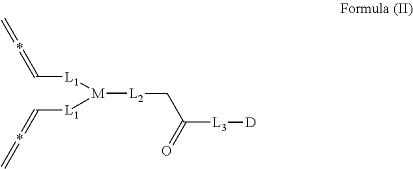
wherein L₁ is selected from the group consisting of 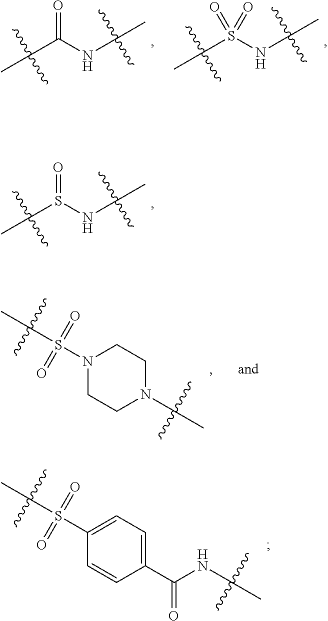 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]
M is selected from the group consisting of 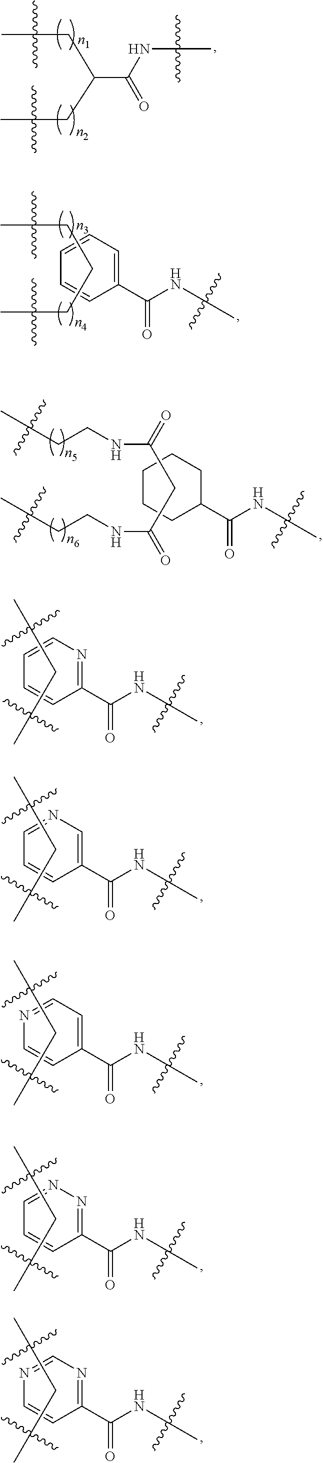 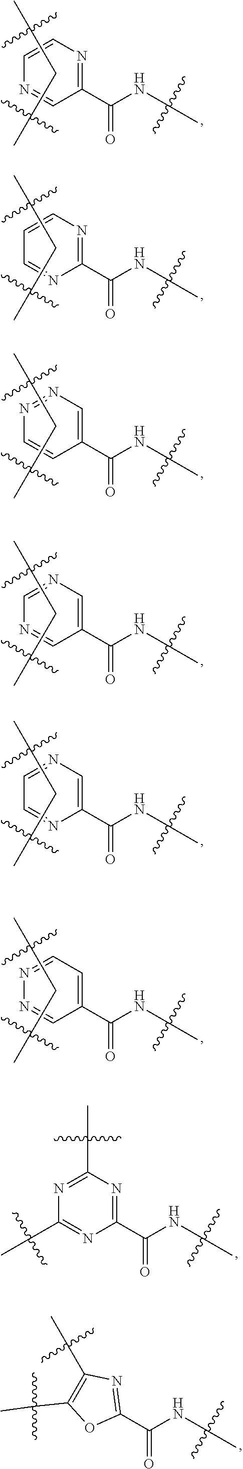 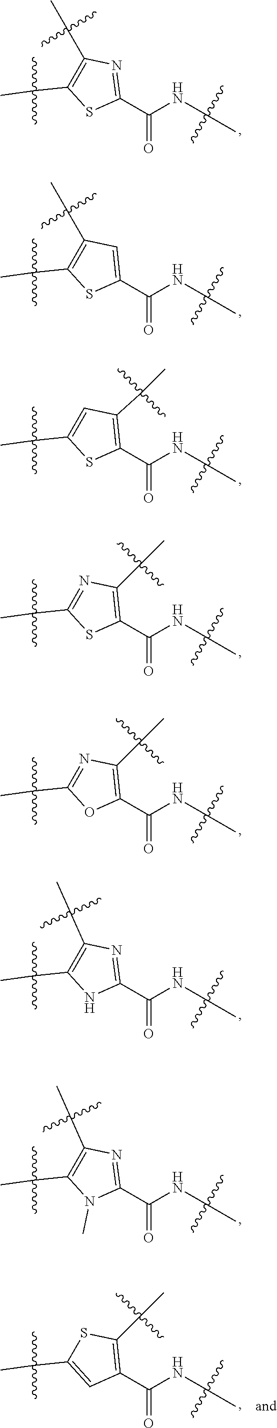 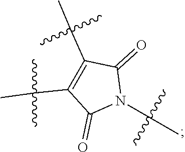 [SMILES: *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 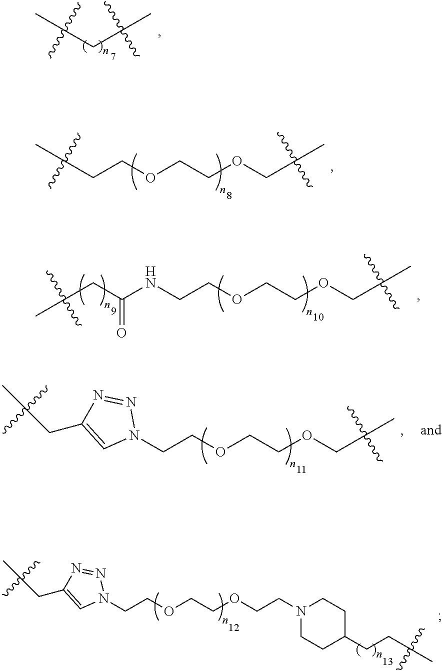
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 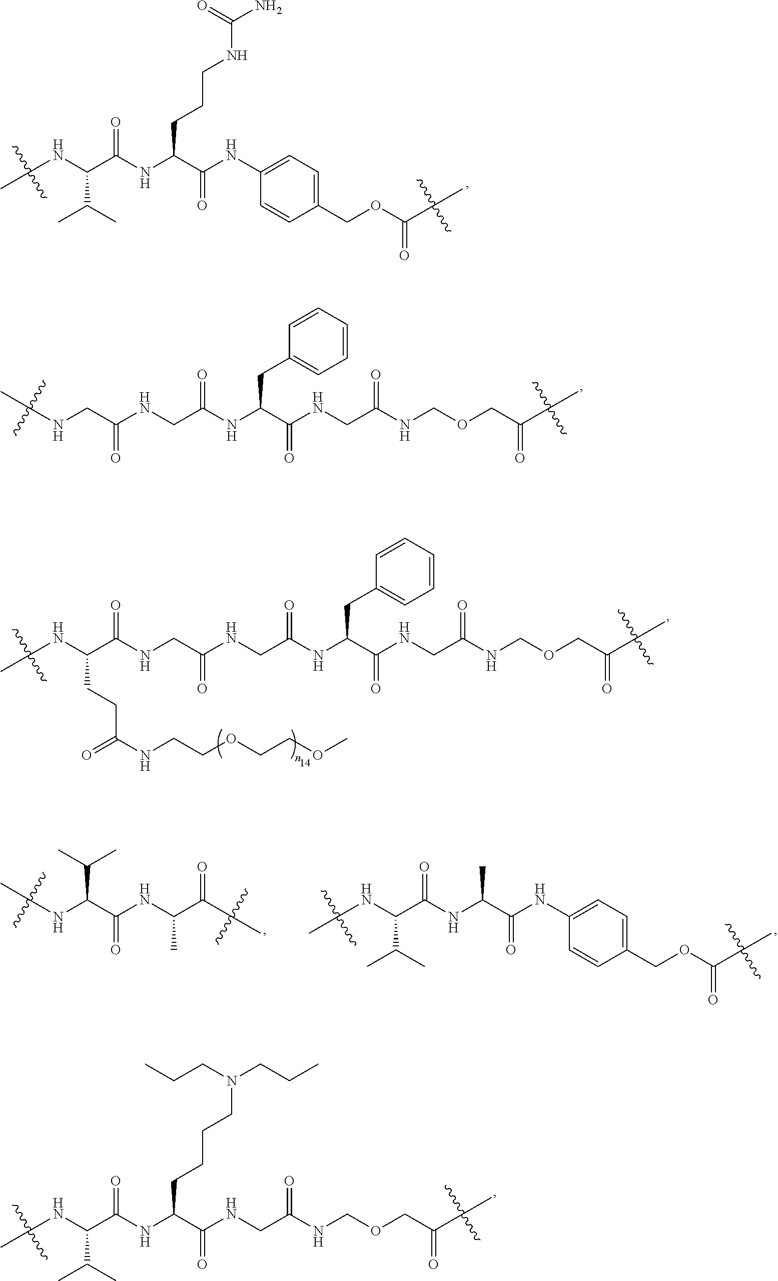 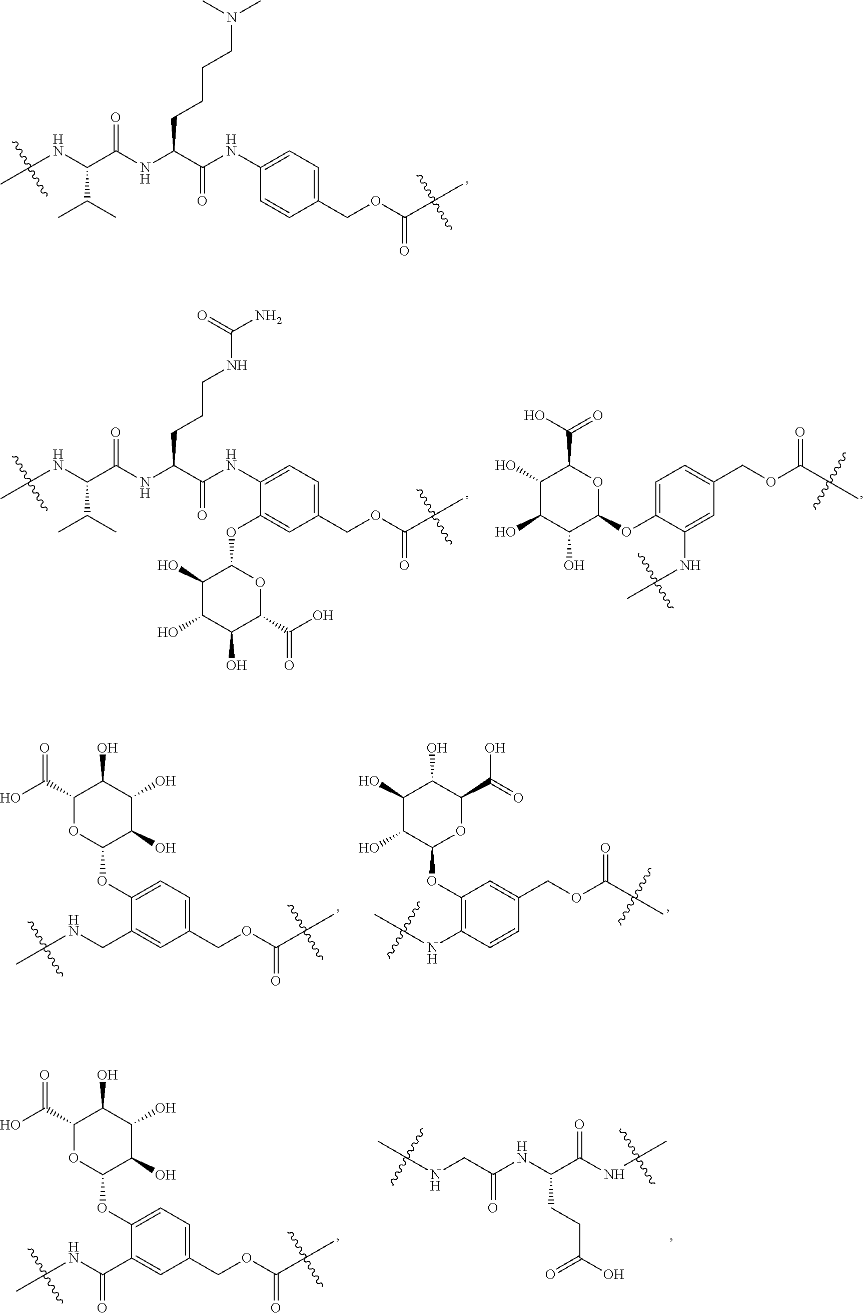 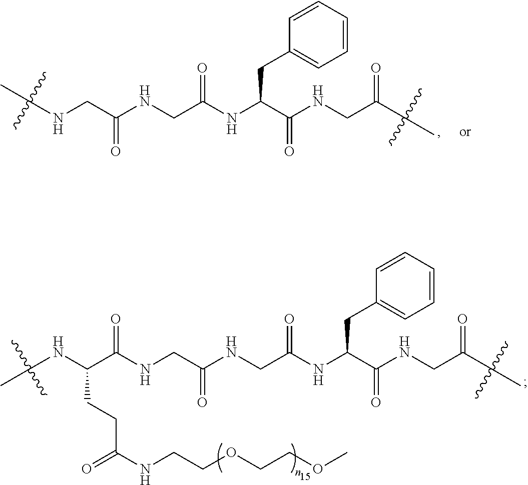
n₁₄ is an integer from 1 to 20, and n₁₅ is an integer from 1 to 20;
D is a fragment formed by the loss of one or more atoms or groups from a cytotoxic small-molecule drug or active agent;
wherein D is covalently bonded to L₃ via sulfur, nitrogen, or oxygen in D.

9. The linker-drug conjugate according to claim 8, wherein in Formula (II),
L₁ is 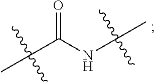 [SMILES: *NC(*)=O]
M is selected from the group consisting of 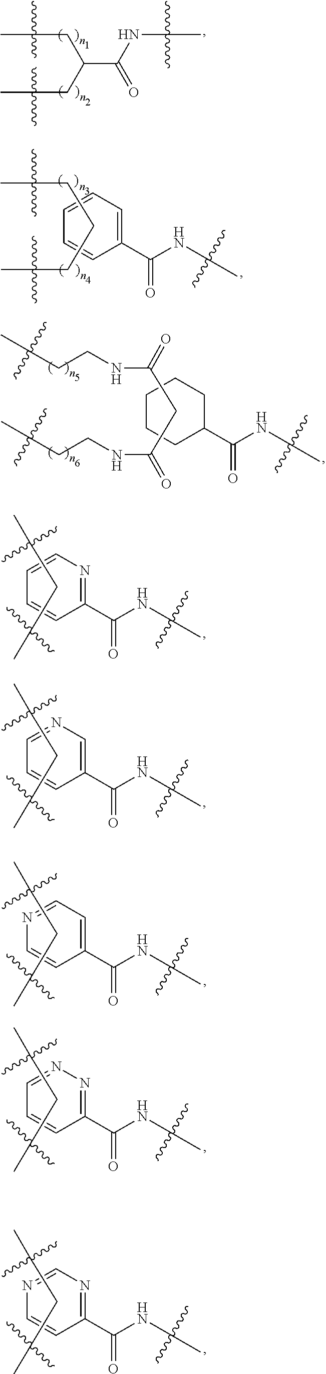 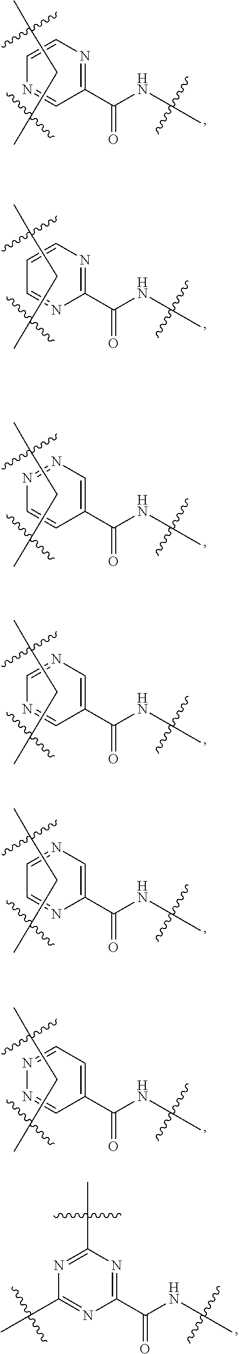 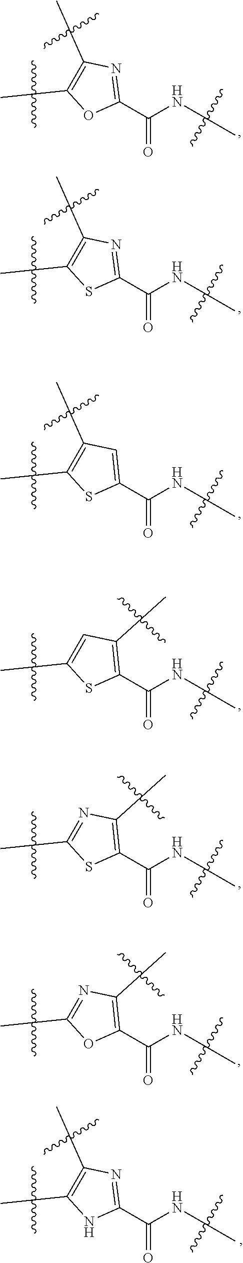 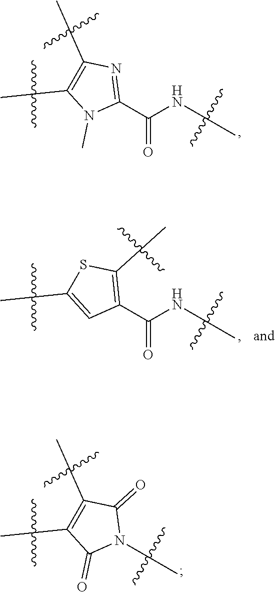 [SMILES: *NC(=O)c1nc(*)c(*)n1C ; *NC(=O)c1cc(*)sc1* ; *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 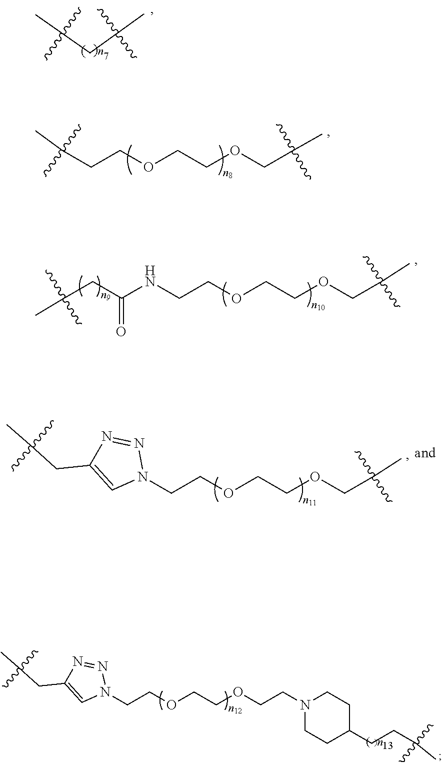
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 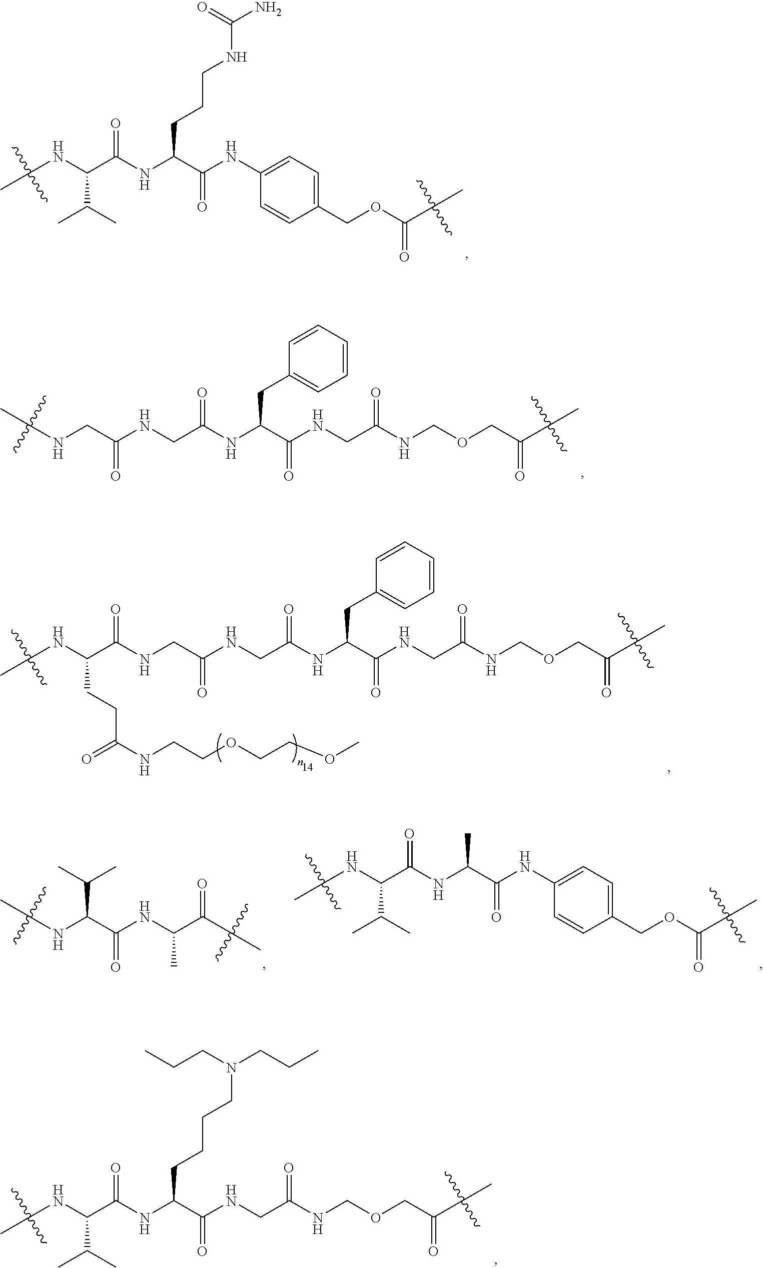 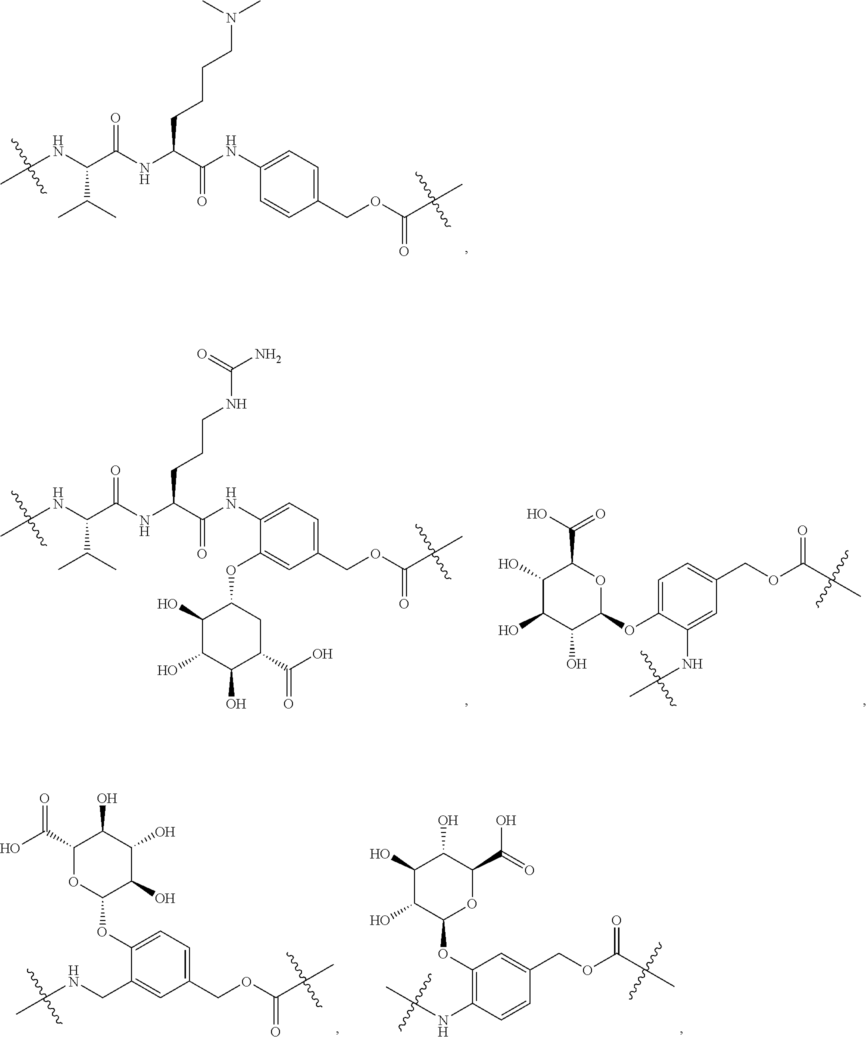 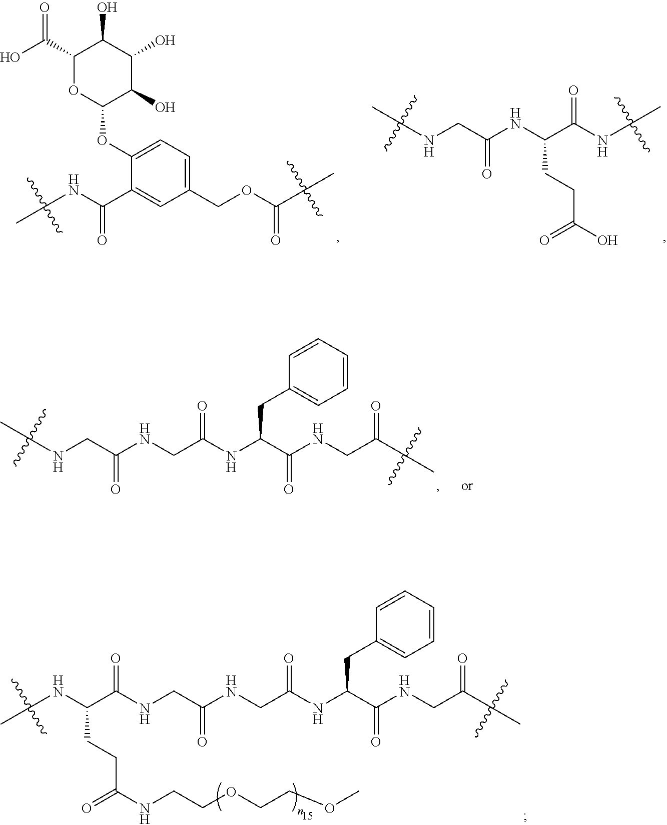
n₁₄ is an integer from 1 to 20, and nis is an integer from 1 to 20;
D is a fragment formed by the loss of one or more atoms or groups from a cytotoxic small-molecule drug or active agent;
wherein D is covalently bonded to L₃ via sulfur, nitrogen, or oxygen in D.

10. The linker-drug conjugate according to claim 8, wherein in Formula (II),
L₁ is selected from the group consisting of 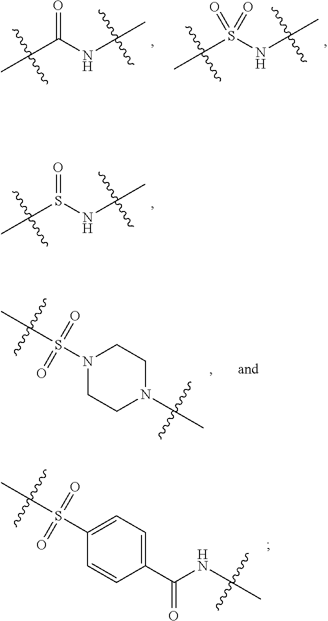 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]
M is selected from the group consisting of 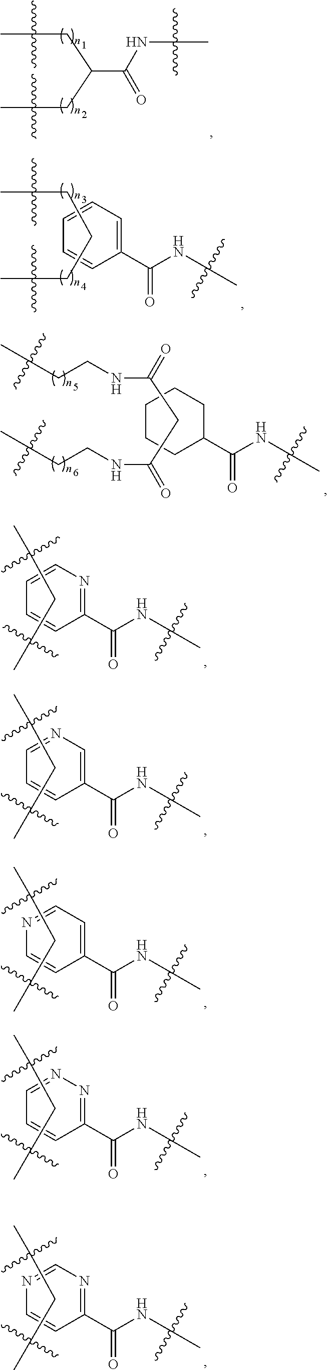 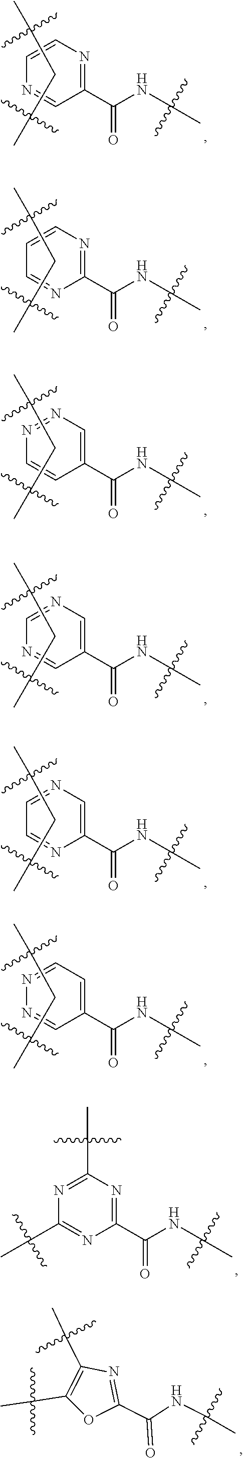 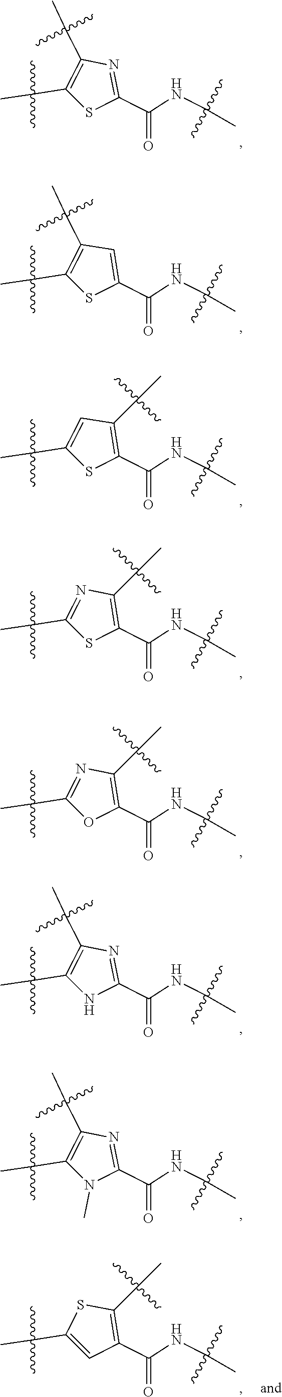 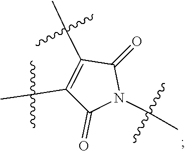 [SMILES: *C1=C(*)C(=O)N(*)C1=O]
n₁ and n₂ are different, n₃ and n₄ are the same or different, and n₅ and n₆ are the same or different; 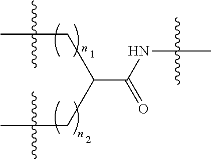
is a single chiral component or a racemate;
n₁ is an integer from 0 to 10, n₂ is an integer from 0 to 10, n₃ is an integer from 0 to 10,
n₄ is an integer from 0 to 10, n₅ is an integer from 1 to 10, and n₆ is an integer from 1 to 10;
L₂ is one or more selected from the group consisting of 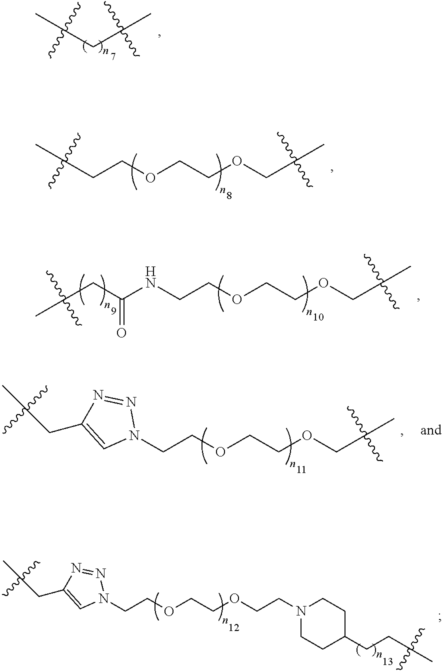
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 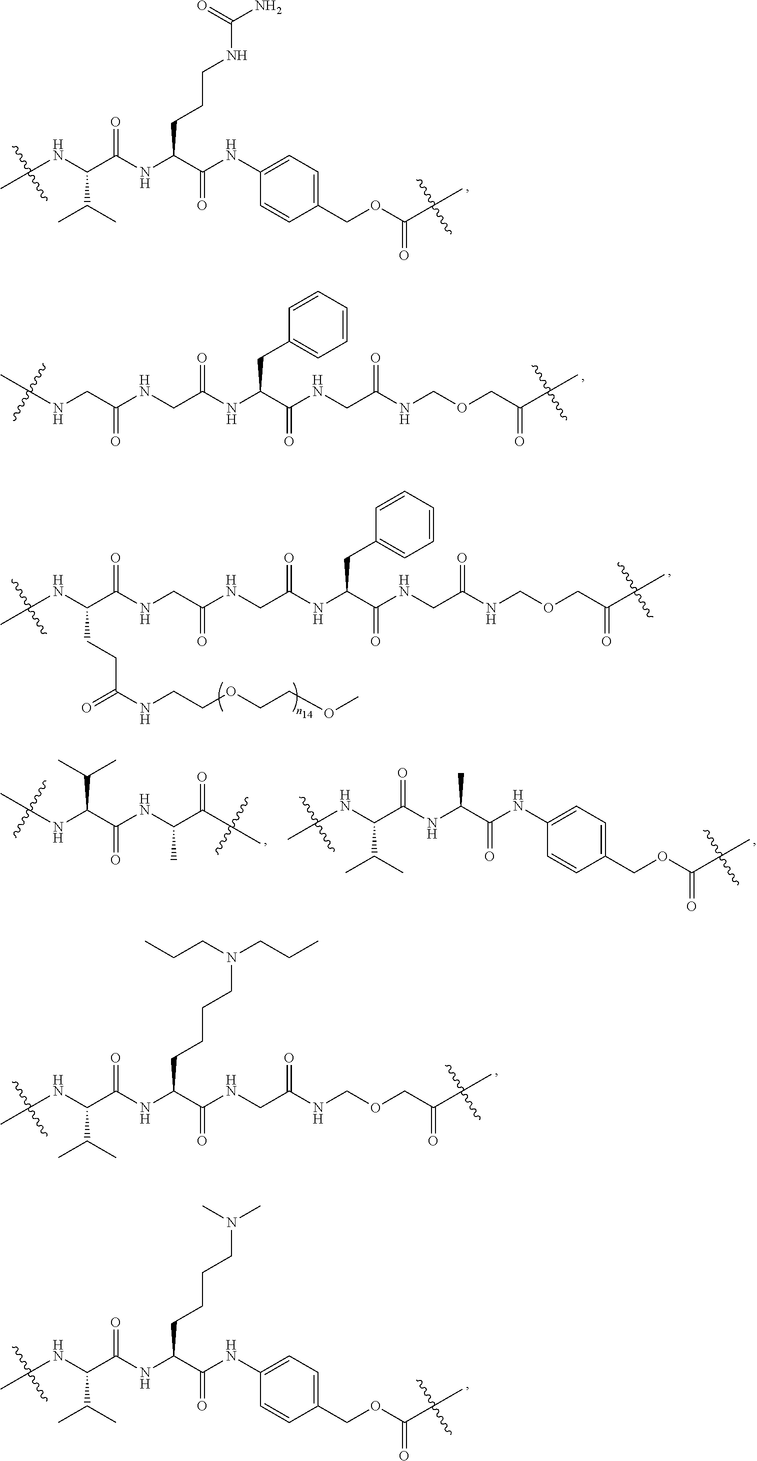 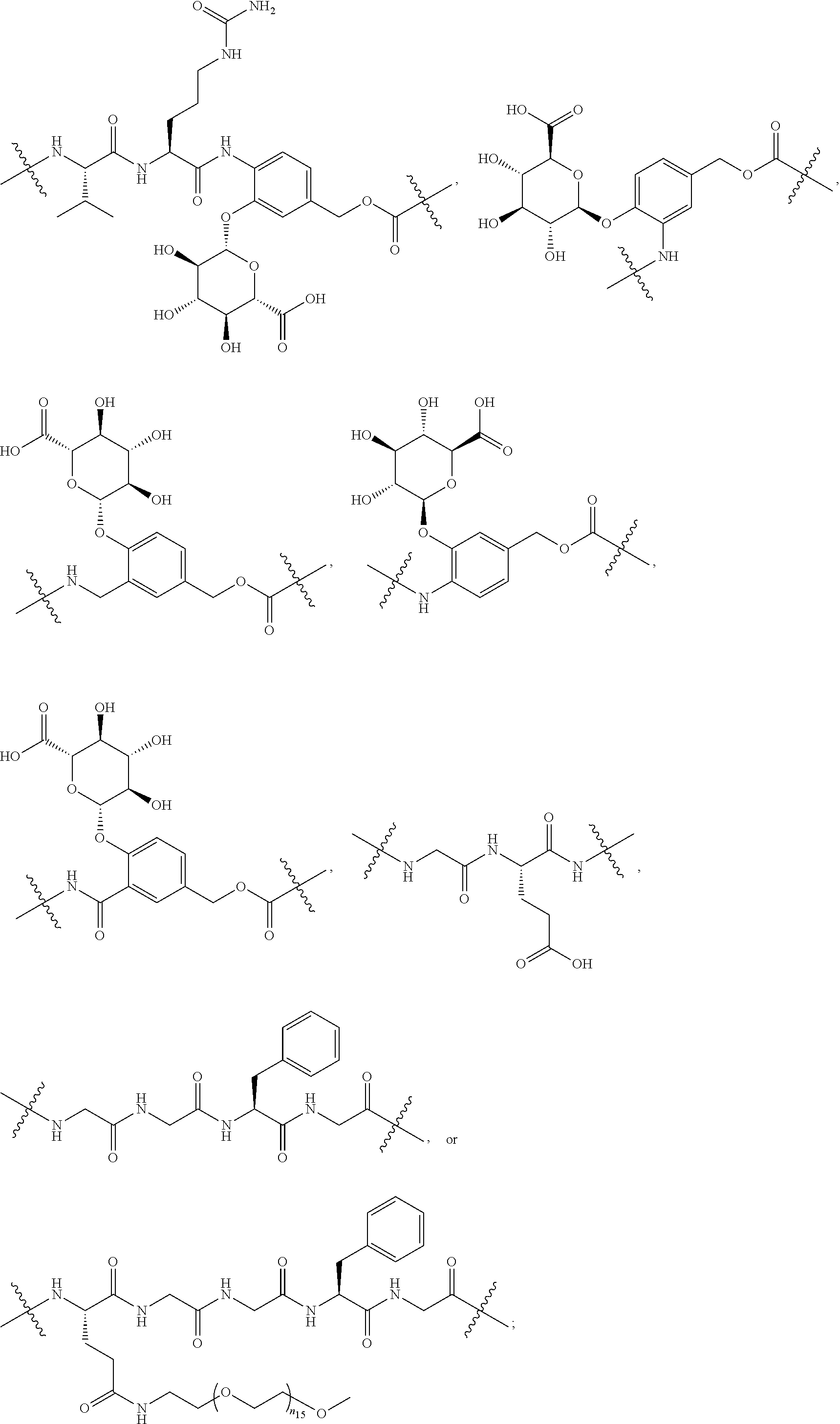
n₁₄ is an integer from 1 to 20, and nis is an integer from 1 to 20;
D is a fragment formed by the loss of one or more atoms or groups from a cytotoxic small-molecule drug or other active agent;
wherein D is covalently bonded to L₃ via sulfur, nitrogen, or oxygen in D.

11. The linker-drug conjugate according to claim 8, wherein in Formula (II),
L₁ is 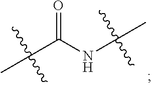 [SMILES: *NC(*)=O]
M is selected from the group consisting of 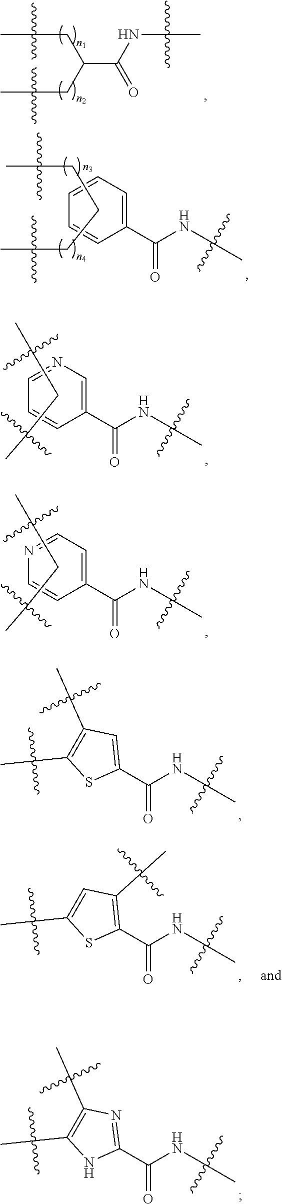
n₁ and n₂ are the same and each independently represent an integer from 0 to 8, and
n₃ and n₄ are the same and each independently represent an integer from 0 to 8;
L₂ is 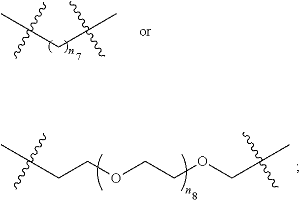
n₇ is an integer from 1 to 10, and n₈ is an integer from 1 to 10;
L₃ is or 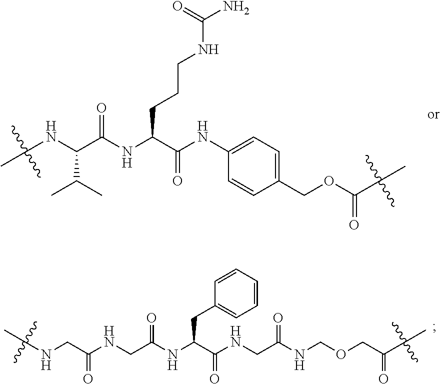 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]
D is a fragment formed by the loss of one or more atoms or groups from a cytotoxic small-molecule drug or active agent;
wherein D is covalently bonded to L₃ via sulfur, nitrogen, or oxygen in D.

12. The linker-drug conjugate according to claim 8, wherein in Formula (II),
L₁ is 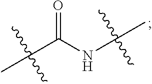 [SMILES: *NC(*)=O]
M is selected from the group consisting of 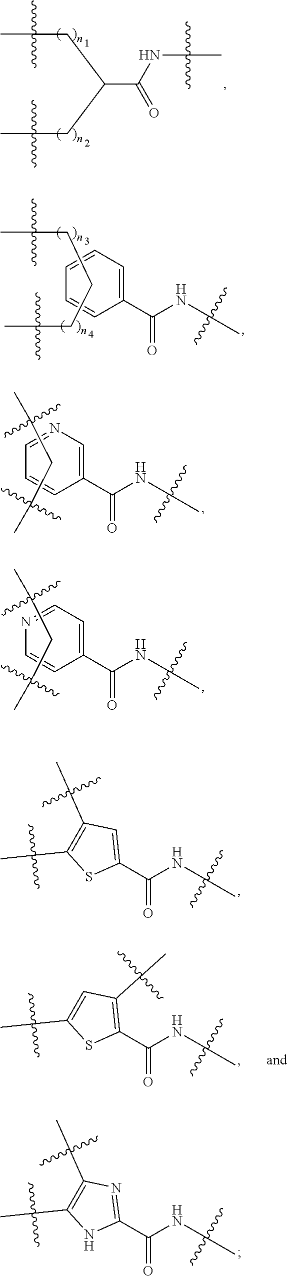
n₁ and n₂ are the same and each independently represent an integer from 0 to 8, and
n₃ and n₄ are the same and each independently represent an integer from 0 to 8;
L₂ is 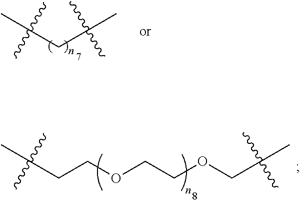
n₇ is an integer from 1 to 10, and n₈ is an integer from 1 to 10;
L₃ is 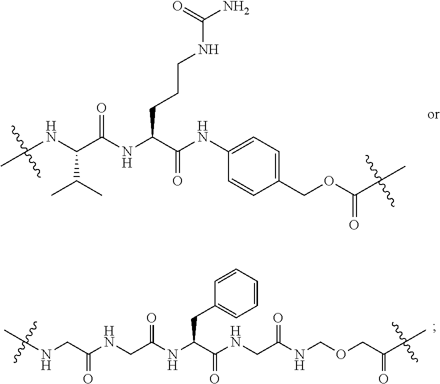 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]
the cytotoxic small-molecule drug is a drug for the treatment of tumor diseases; and
the active agent is a tubulin inhibitor, an RNA polymerase inhibitor, a topoisomerase inhibitor, an intercalator, a DNA-reactive agent, a DNA alkylating agent, or an immunosuppressant.

13. The linker-drug conjugate according to claim 8, wherein the compound of Formula (II) is one or more selected from the group consisting of the compounds in the following table:

| No. | Structure |
|---|---|
| LP1 | 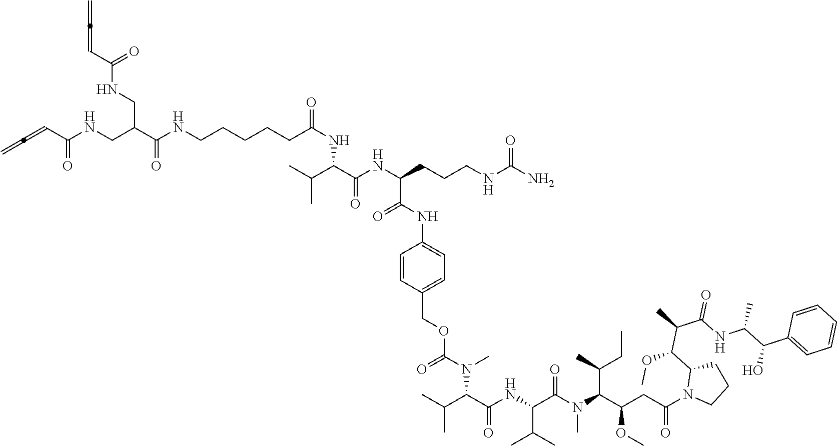 |
| LP2 | 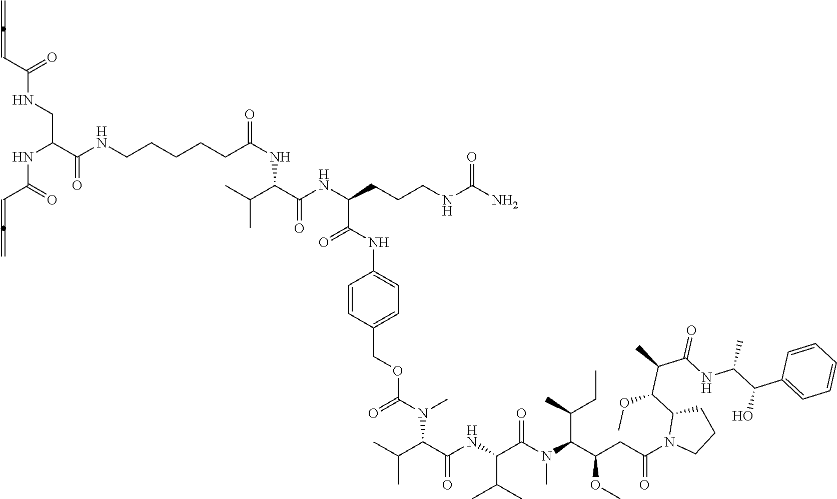 |
| LP3 | 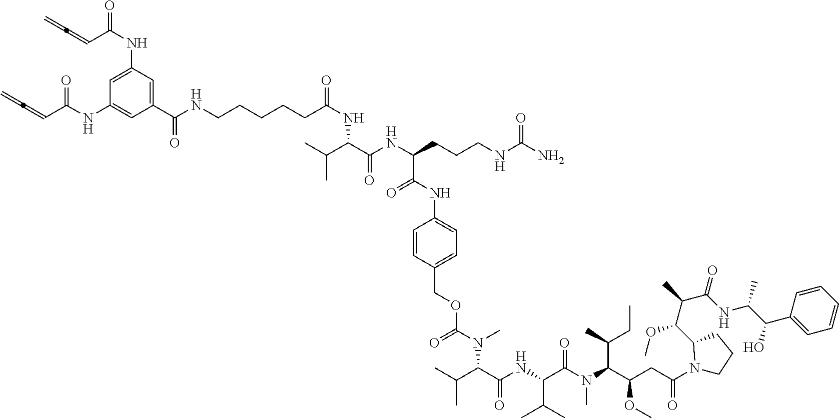 |
| LP4 | 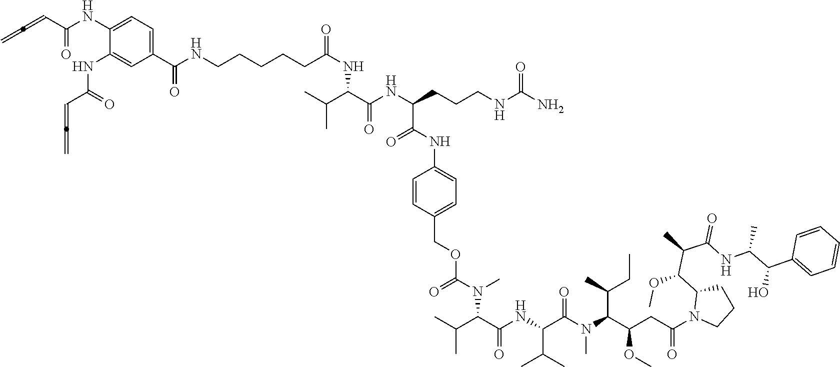 |
| LP5 | 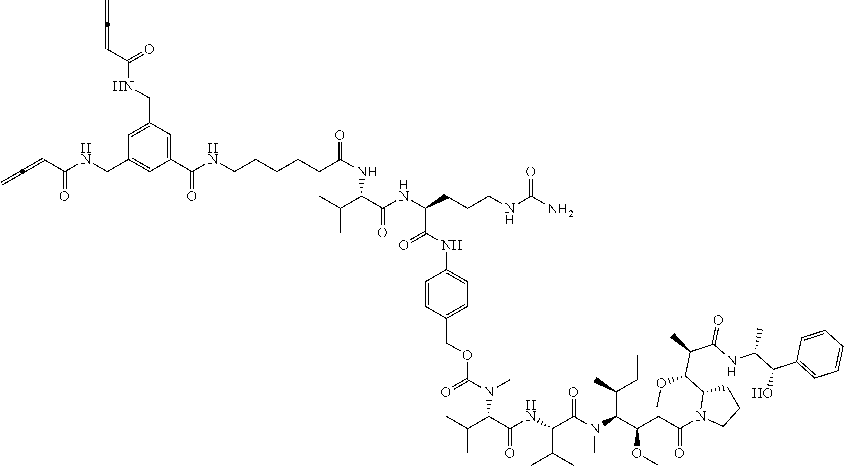 |
| LP6 | 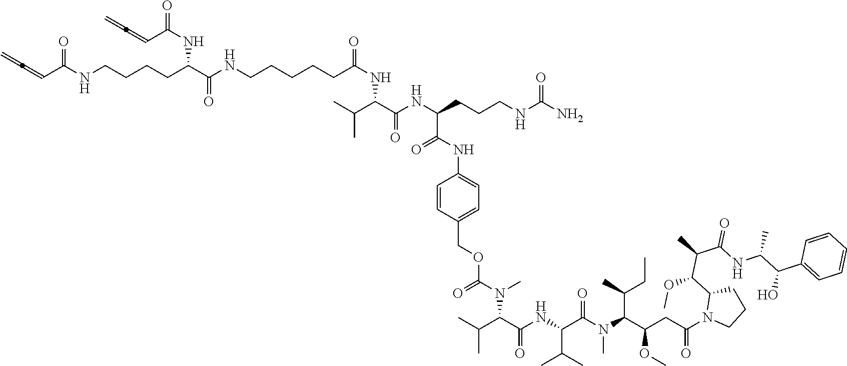 |
| LP7 | 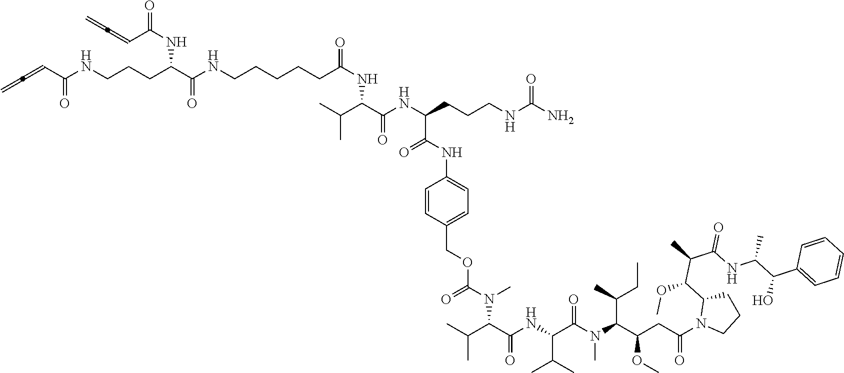 |
| LP8 | 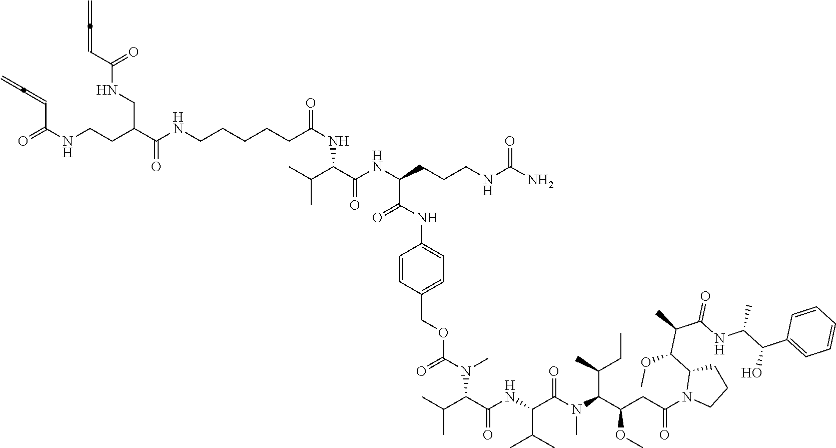 |
| LP9 | 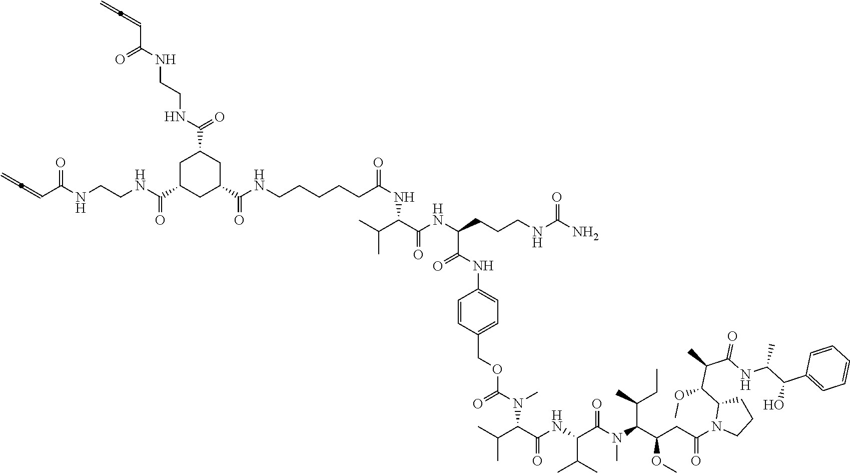 |
| LP10 | 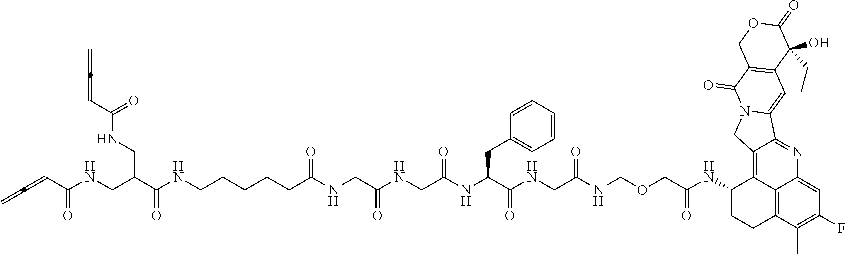 |
| LP11 | 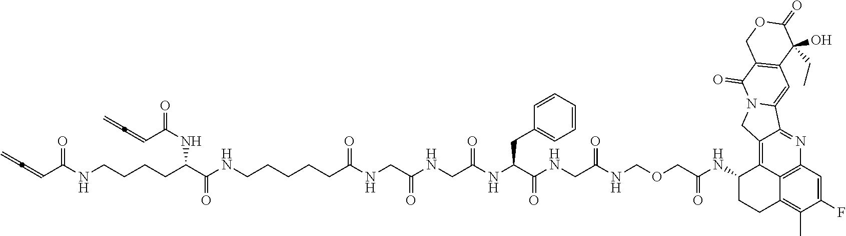 |
| LP12 | 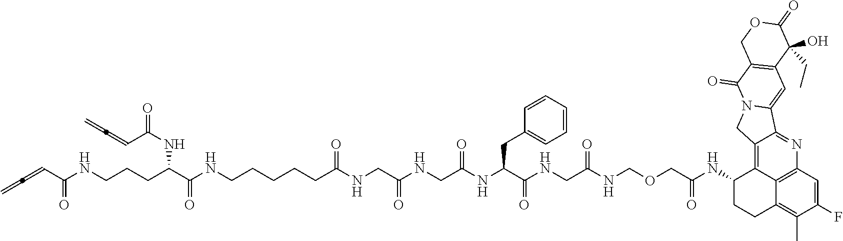 |
| LP13 | 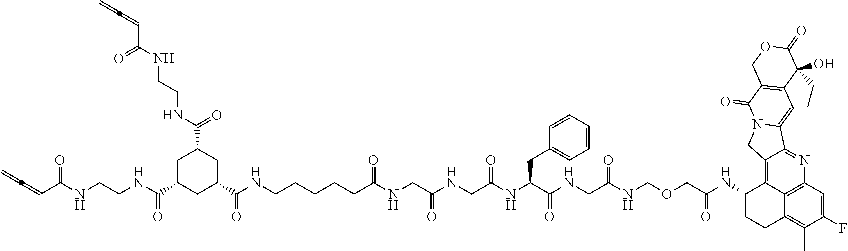 |
| LP14 | 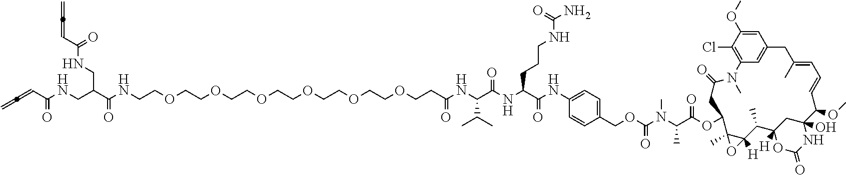 |
| LP15 | 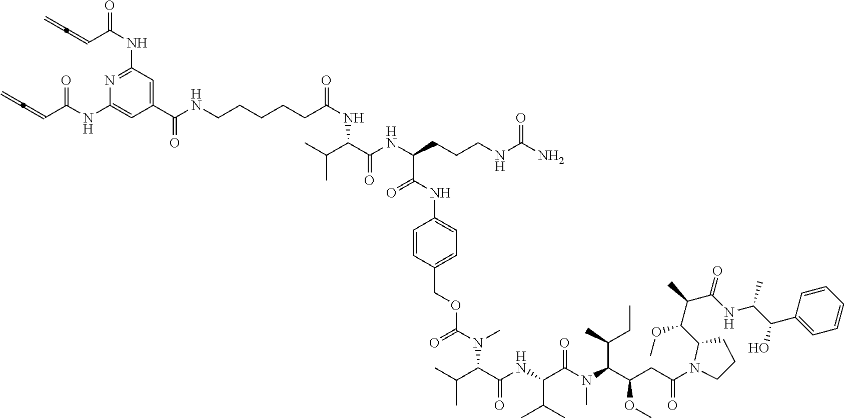 |
| LP16 | 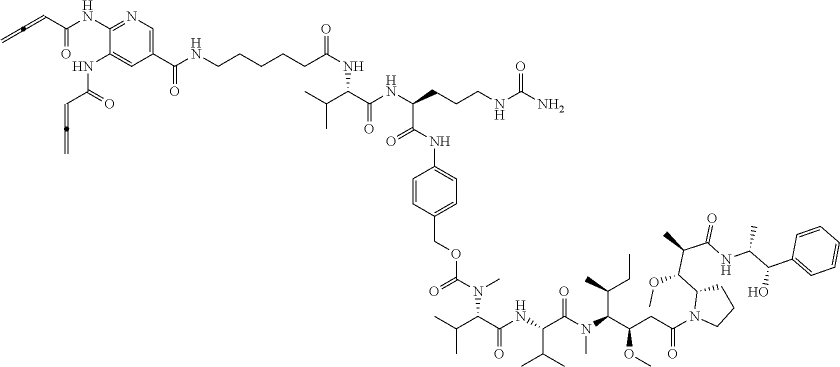 |
| LP17 | 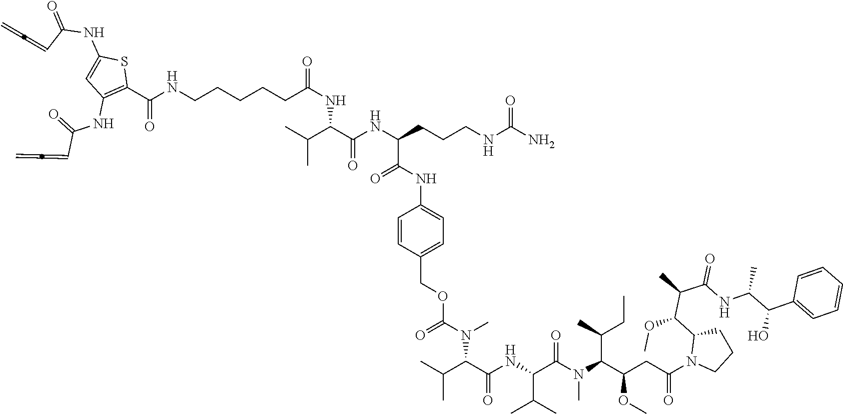 |
| LP18 | 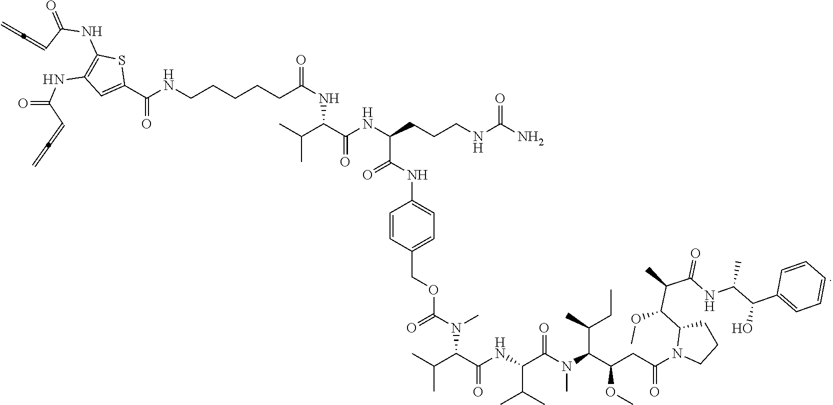 |

14. An antibody-drug conjugate, which is formed by the conjugation of the bis-allene group in the linker-drug conjugate according to claim 8 with the thiol group of the reduced Ab;
wherein the Ab is an antibody, antibody fragment, or protein.

15. An antibody-drug conjugate, comprising a structure represented by Formula (III): 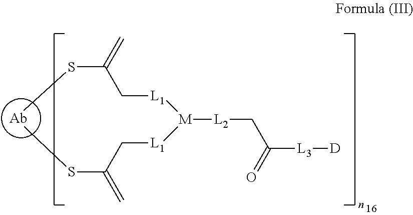
wherein in Formula (III), the carbon-carbon double bond of the bis-allene group in Formula (II) according to claim 8 is linked to a thiol group in the reduced antibody Ab via a C—S bond formed by a Michael addition reaction;
L₁ is selected from the group consisting of 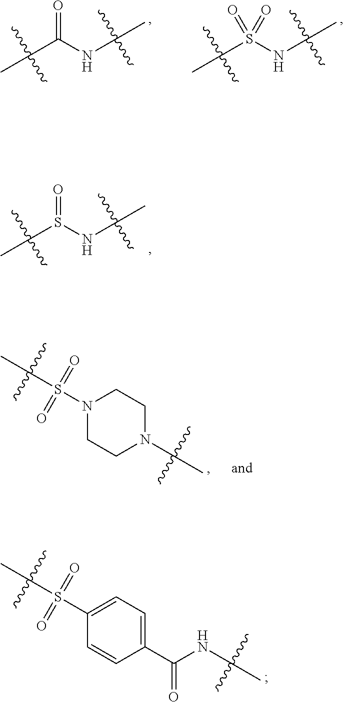
M is selected from the group consisting of 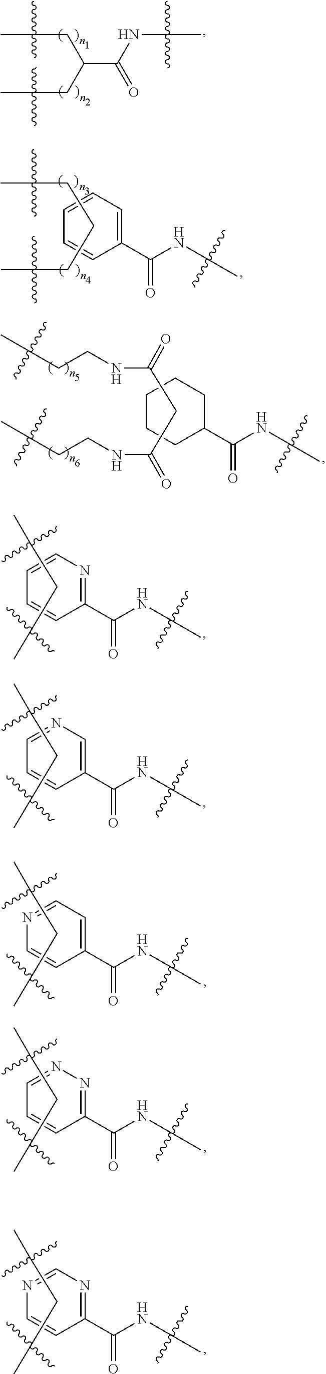 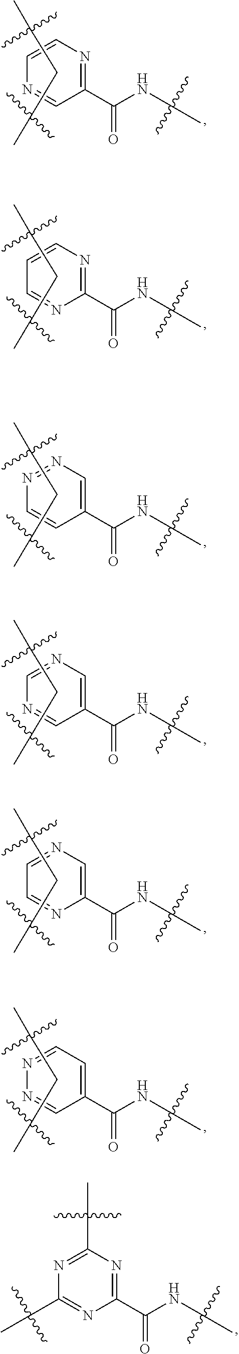 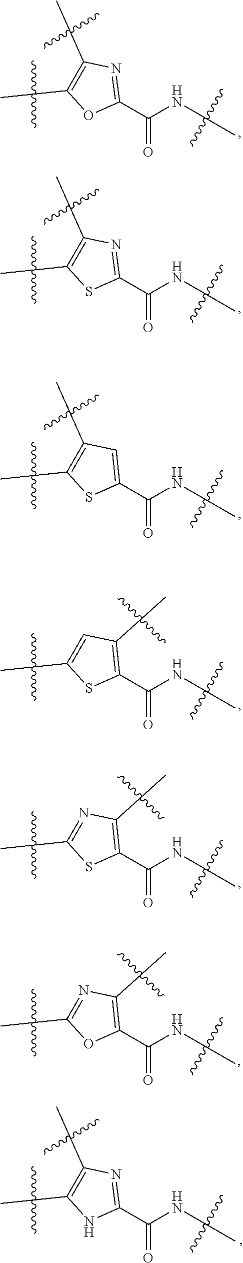 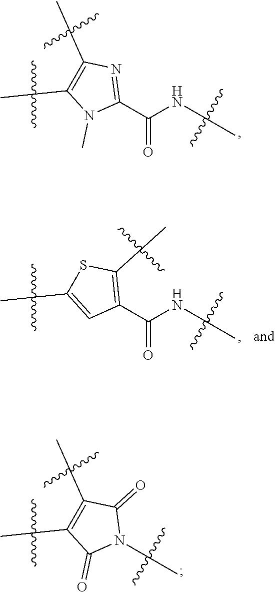 [SMILES: *NC(=O)c1nc(*)c(*)n1C ; *NC(=O)c1cc(*)sc1* ; *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 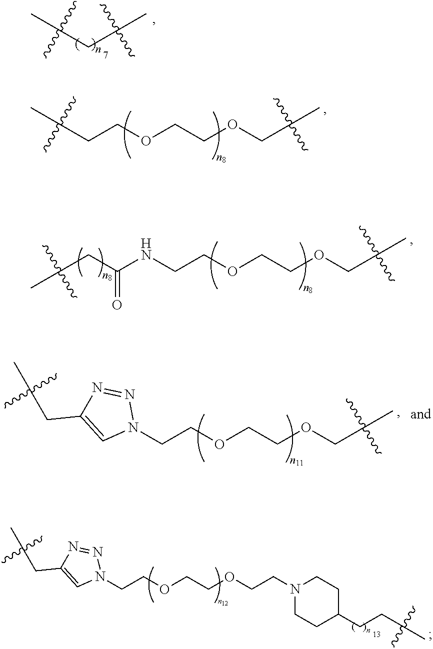
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 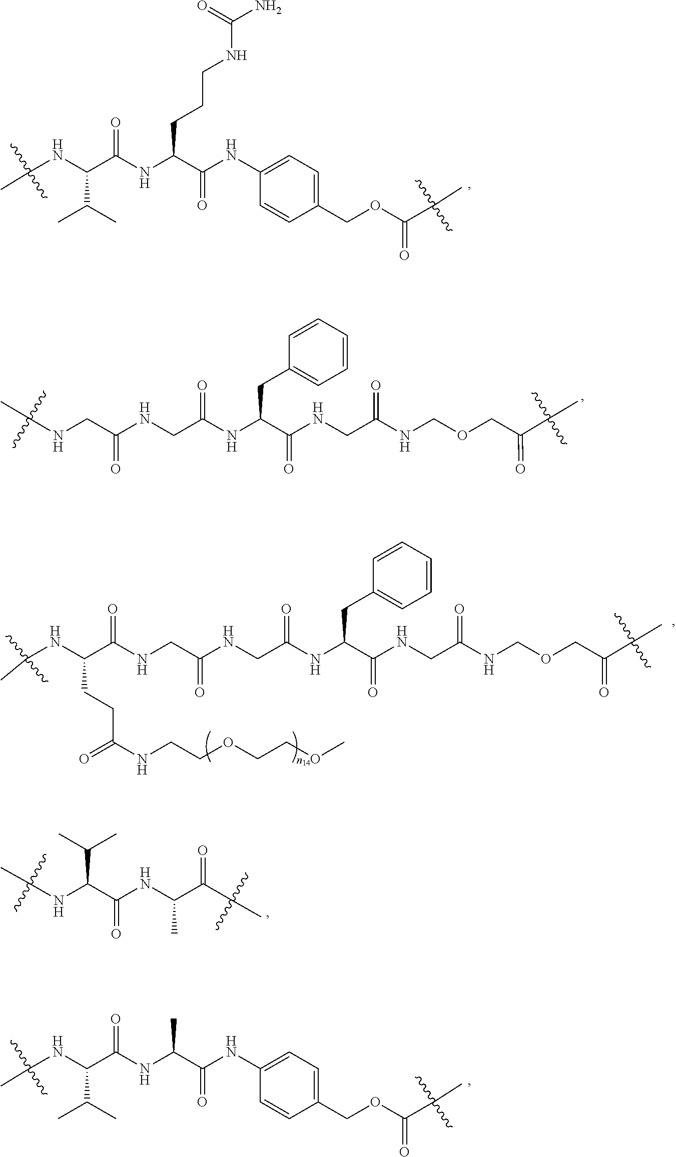 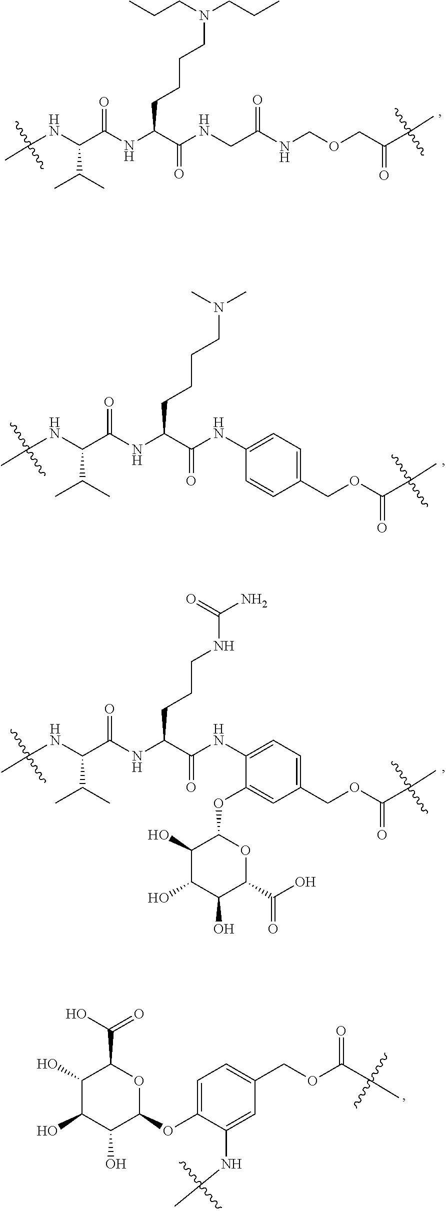 [SMILES: *N[C@H](C(=O)N[C@@H](CCCCN(CCC)CCC)C(=O)NCC(=O)NCOCC(*)=O)C(C)C ; *N[C@H](C(=O)N[C@@H](CCCCN(C)C)C(=O)Nc1ccc(COC(*)=O)cc1)C(C)C ; *N[C@H](C(=O)N[C@@H](CCCNC(N)=O)C(=O)Nc1ccc(COC(*)=O)cc1O[C@@H]1O[C@H](C(=O)O)[C@@H](O)[C@H](O)[C@H]1O)C(C)C ; *Nc1cc(COC(*)=O)ccc1O[C@@H]1O[C@H](C(=O)O)[C@@H](O)[C@H](O)[C@H]1O] 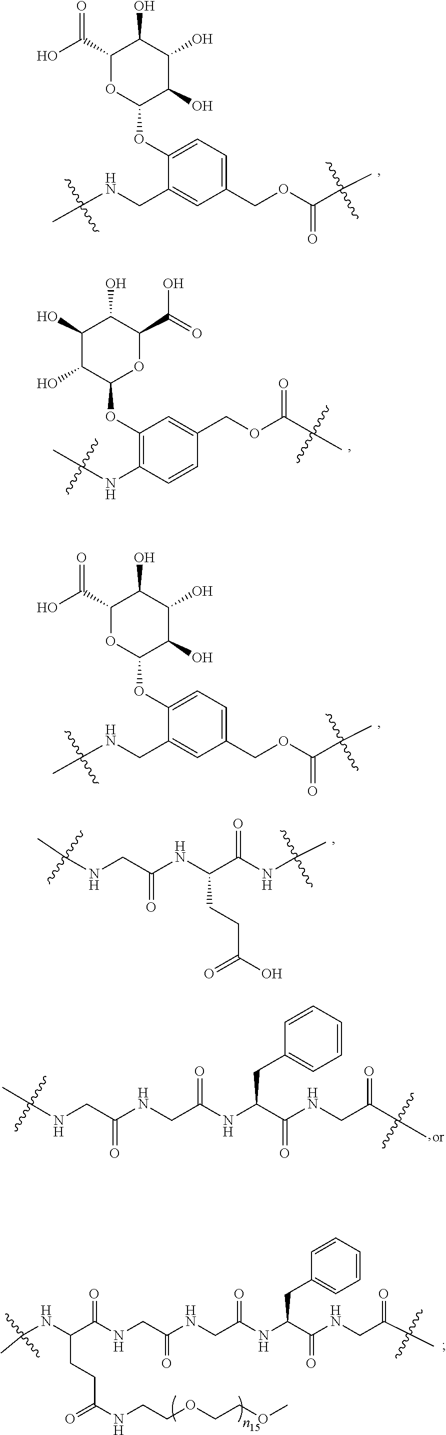
n₁₄ is an integer from 1 to 20, and nis is an integer from 1 to 20;
D is a fragment formed by the loss of one or more atoms or groups from a cytotoxic small-molecule drug or active agent, wherein D is covalently bonded to L₃ via sulfur, nitrogen, or oxygen in D;
n₁₆ is an integer from 1 to 20;
the Ab is an antibody, antibody fragment, or protein.

16. The antibody-drug conjugate according to claim 15, wherein in Formula (III),
L₁ is 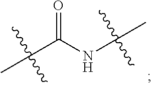 [SMILES: *NC(*)=O]
M is selected from the group consisting of 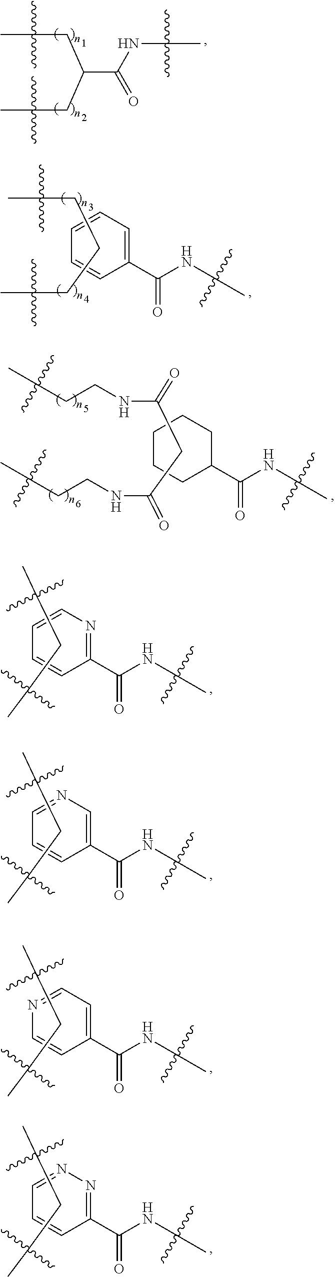 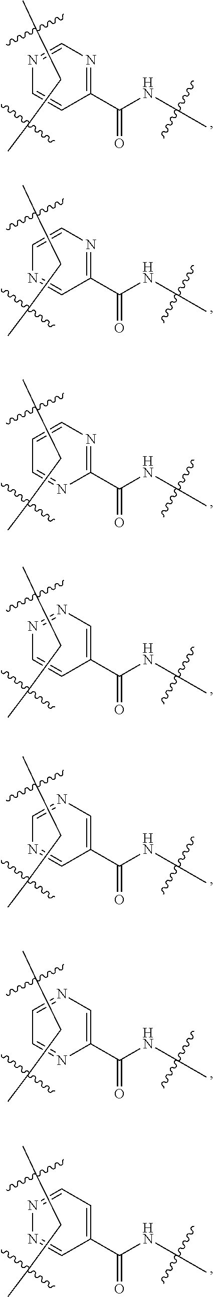 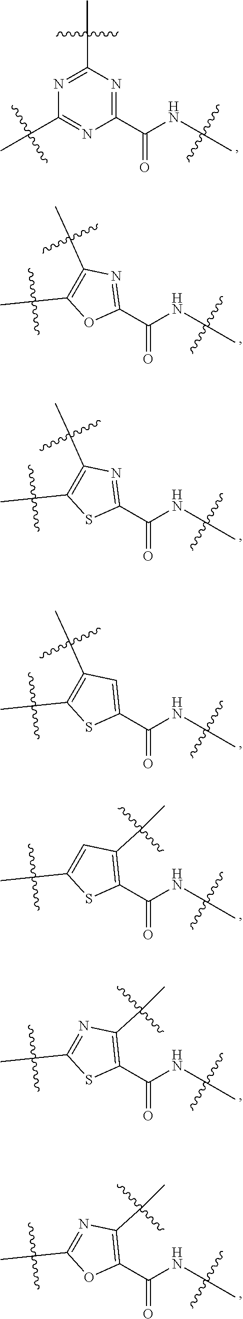 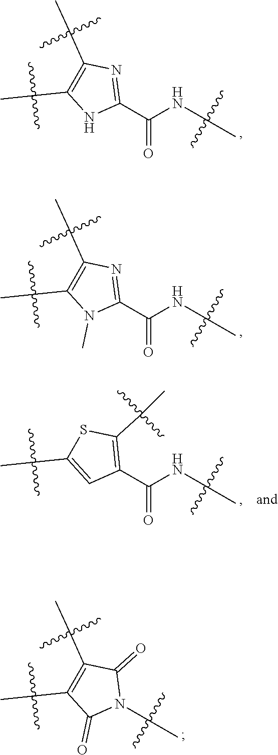 [SMILES: *NC(=O)c1nc(*)c(*)[nH]1 ; *NC(=O)c1nc(*)c(*)n1C ; *NC(=O)c1cc(*)sc1* ; *C1=C(*)C(=O)N(*)C1=O]
n₁ and n₂ are the same or different and each independently represent an integer from 0 to 10,
n₃ and n₄ are the same or different and each independently represent an integer from 0 to 10, and
n₅ and n₆ are the same or different and each independently represent an integer from 1 to 10;
L₂ is one or more selected from the group consisting of 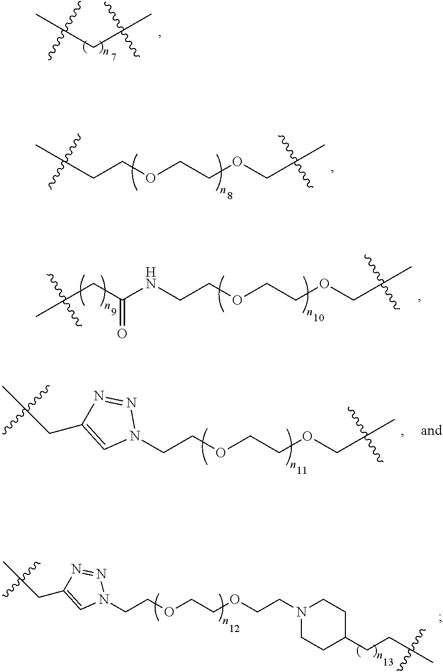
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 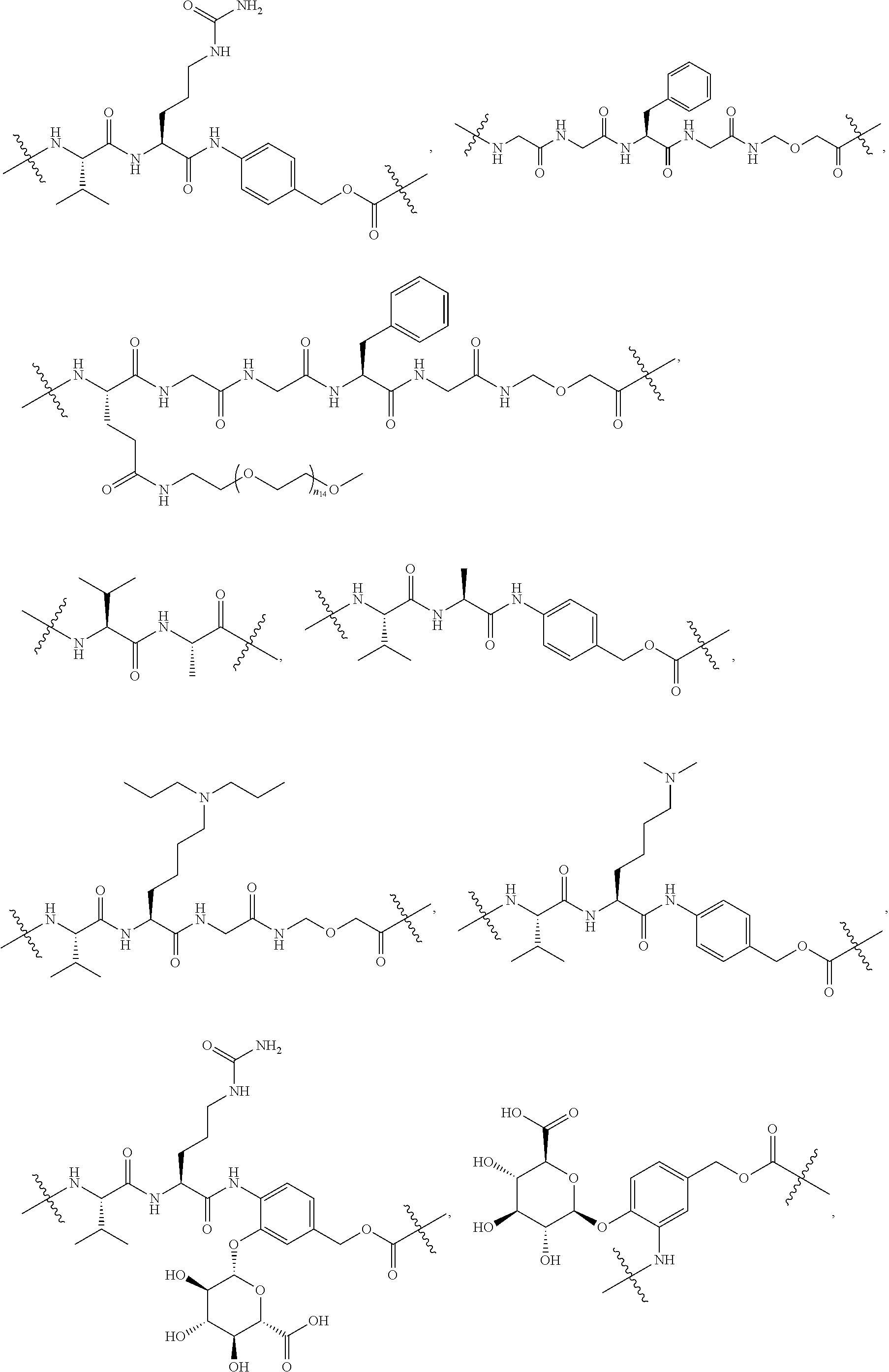 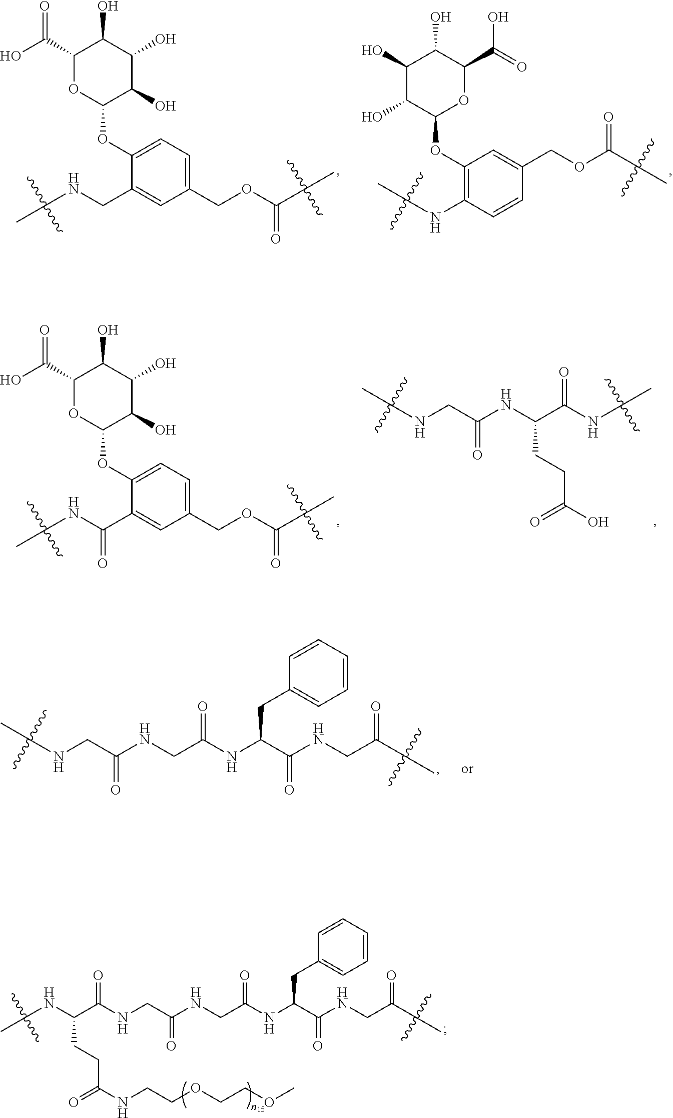
n₁₄ is an integer from 1 to 20, and nis is an integer from 1 to 20;
D is a fragment formed by the loss of one or more atoms or groups from a cytotoxic small-molecule drug or active agent, wherein D is covalently bonded to L₃ via sulfur, nitrogen, or oxygen in D;
n₁₆ is an integer from 1 to 20;
the Ab is an antibody, antibody fragment, or protein.

17. The antibody-drug conjugate according to claim 15, wherein in Formula (III),
L₁ is selected from the group consisting of 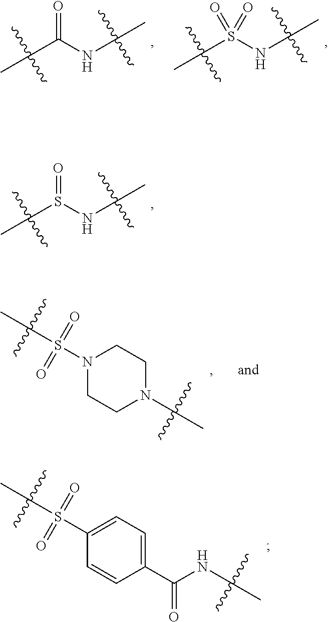 [SMILES: *NC(*)=O ; *NS(*)(=O)=O ; *NS(*)=O ; *N1CCN(S(*)(=O)=O)CC1 ; *NC(=O)c1ccc(S(*)(=O)=O)cc1]
M is selected from the group consisting of 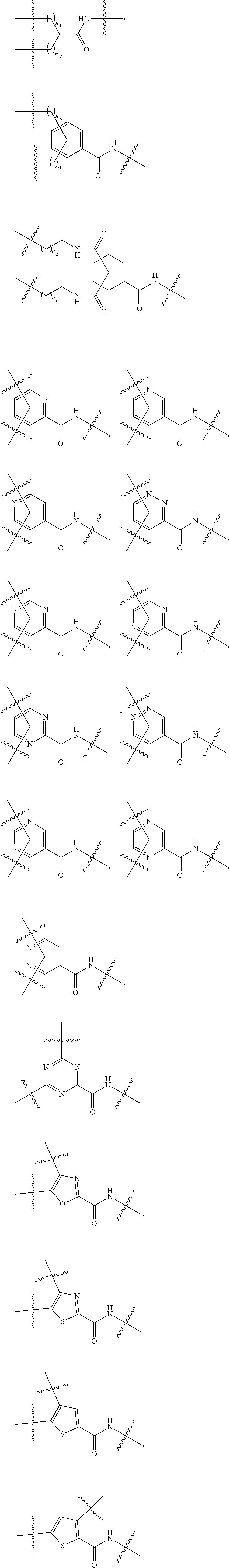 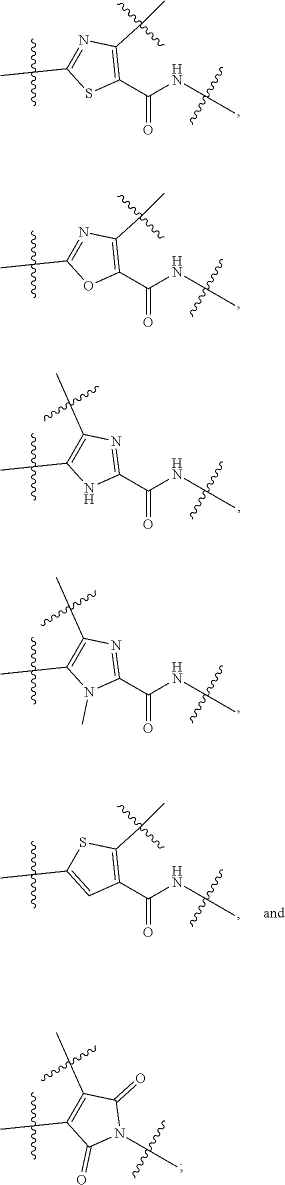 [SMILES: *NC(=O)c1sc(*)nc1* ; *NC(=O)c1oc(*)nc1* ; *NC(=O)c1nc(*)c(*)[nH]1 ; *NC(=O)c1nc(*)c(*)n1C ; *NC(=O)c1cc(*)sc1* ; *C1=C(*)C(=O)N(*)C1=O]
n₁ and n₂ are different, n₃ and n₄ are the same or different, and n₅ and n₆ are the same or different; 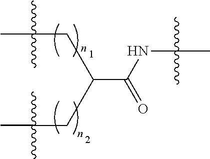
is a single chiral component or a racemate;
n₁ is an integer from 0 to 10, n₂ is an integer from 0 to 10, n₃ is an integer from 0 to 10,
n₄ is an integer from 0 to 10, n₅ is an integer from 1 to 10, and n₆ is an integer from 1 to 10;
L₂ is one or more selected from the group consisting of 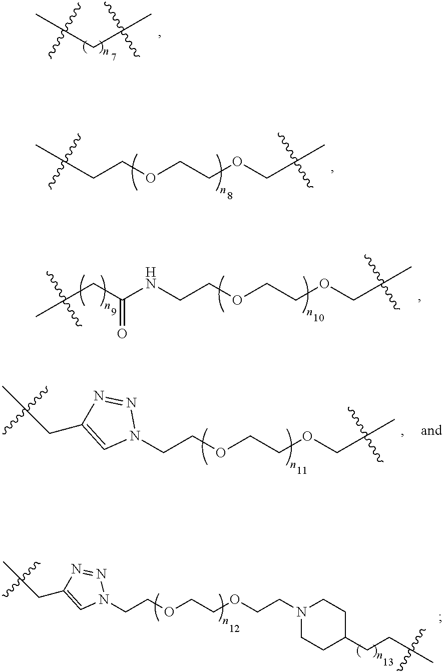
n₇ is an integer from 1 to 20, n₈ is an integer from 1 to 20, n₉ is an integer from 1 to 20,
n₁₀ is an integer from 1 to 20, n₁₁ is an integer from 1 to 20, n₁₂ is an integer from 1 to 20, and n₁₃ is an integer from 1 to 20;
L₃ is 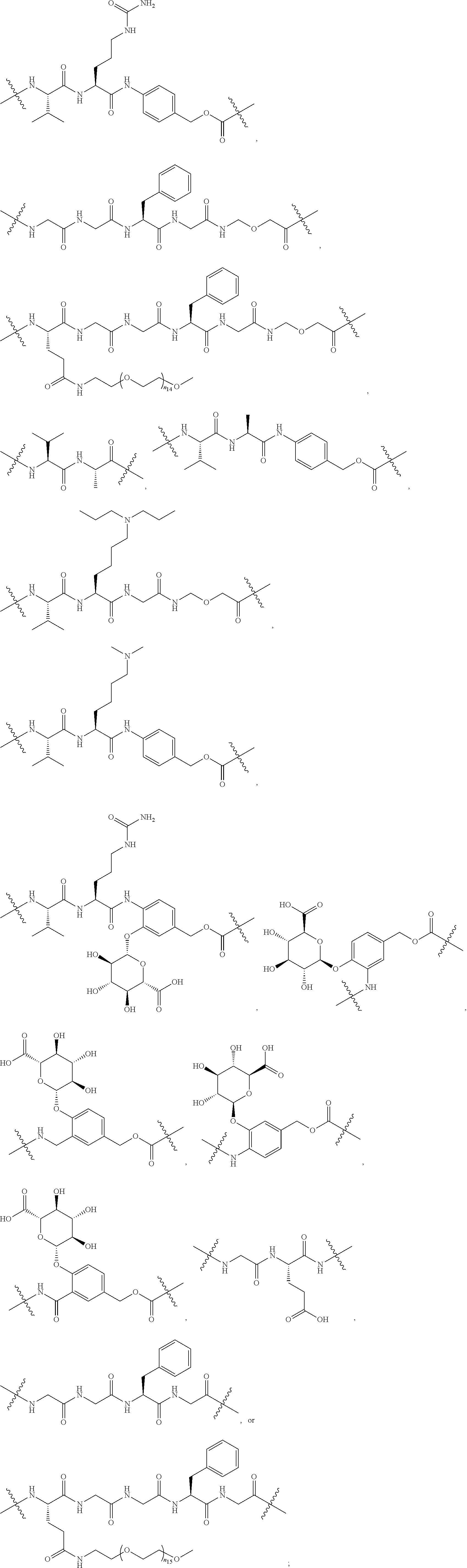
n₁₄ is an integer from 1 to 20, and n₁₅ is an integer from 1 to 20;
D is a fragment formed by the loss of one or more atoms or groups from a cytotoxic small-molecule drug or active agent, wherein D is covalently bonded to L₃ via sulfur, nitrogen, or oxygen in D;
n₁₆ is an integer from 1 to 20;
the Ab is an antibody, antibody fragment, or protein.

18. The antibody-drug conjugate according to claim 15, wherein in Formula (III),
L₁ is 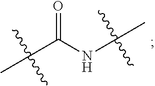 [SMILES: *NC(*)=O]
M is selected from the group consisting of 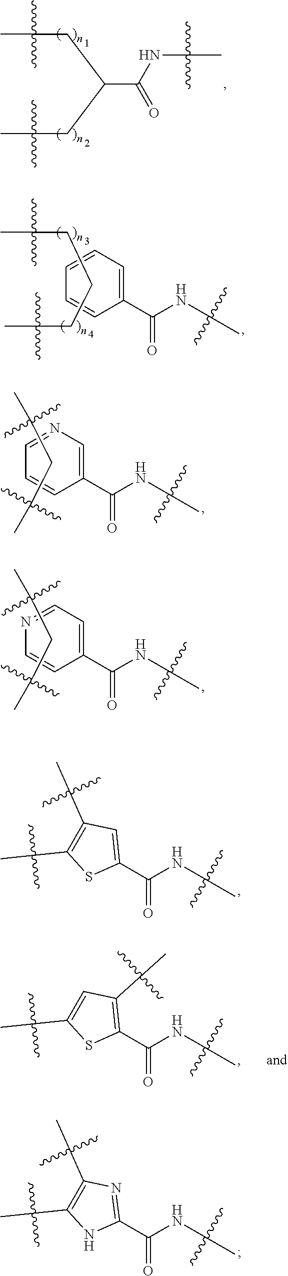
n₁ and n₂ are the same and each independently represent an integer from 0 to 8, and
n₃ and n₄ are the same and each independently represent an integer from 0 to 8;
L₂ is 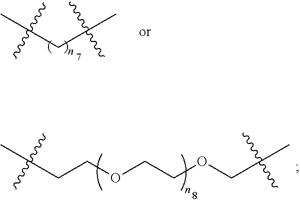
n₇ is an integer from 1 to 10, and n₈ is an integer from 1 to 10;
L₃ is 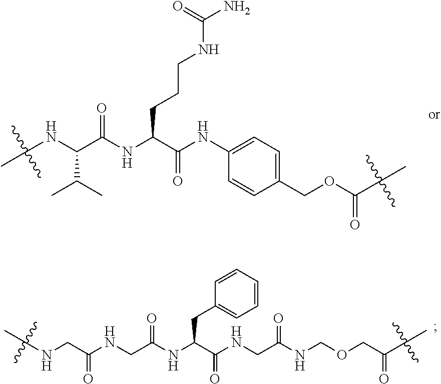 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]
the cytotoxic small-molecule drug is a drug for the treatment of tumor diseases;
the active agent is a tubulin inhibitor, an RNA polymerase inhibitor, a topoisomerase inhibitor, an intercalator, a DNA-reactive agent, a DNA alkylating agent, or an immunosuppressant;
n₁₆ is an integer from 1 to 20;
the Ab is an antibody, antibody fragment, or protein.

19. The antibody-drug conjugate according to claim 15, wherein in Formula (III),
L₁ is 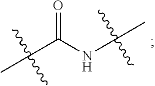 [SMILES: *NC(*)=O]
M is selected from the group consisting of 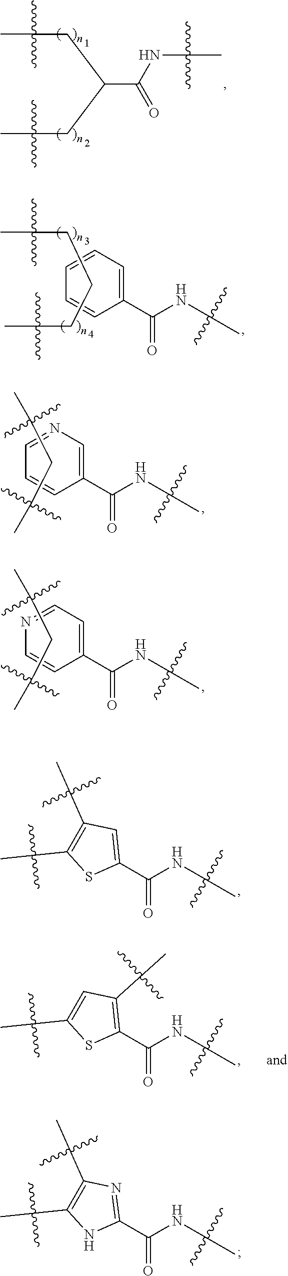
n₁ and n₂ are the same and each independently represent an integer from 0 to 8, and
n₃ and n₄ are the same and each independently represent an integer from 0 to 8;
L₂ is 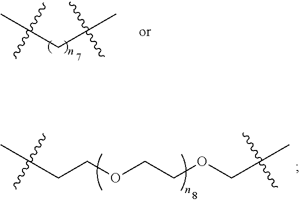
n₇ is an integer from 1 to 10, and n₈ is an integer from 1 to 10;
L₃ is 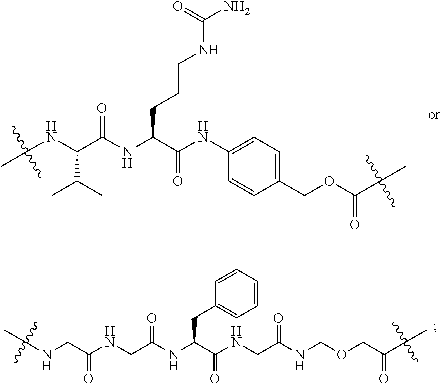 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]
the cytotoxic small-molecule drug is a drug for the treatment of tumor diseases;
the active agent is a tubulin inhibitor, an RNA polymerase inhibitor, a topoisomerase inhibitor, an intercalator, a DNA-reactive agent, a DNA alkylating agent, or an immunosuppressant;
n₁₆ is an integer from 1 to 20;
the Ab is Abciximab, Alemtuzumab, Anetumab, Atezolizumab, Avelumab, Basiliximab, Bevacizumab, Blinatomumab, Brentuximab, Catumaxomab, Cetuximab, Cirmtuzumab, Coltuximab, Daclizumab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Omalizumab, Palivizumab, Panitumumab, Patritumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, Trastuzumab, an anti-B7-H3 monoclonal antibody, an anti-CD4 antibody, an anti-CD5 antibody, an anti-CD13 antibody, an anti-CD30 antibody, or a mutant of the above antibodies at positions 226 or 229 of the heavy chain, an antigen-binding fragment thereof, or an immunologically active portion thereof.

20. The antibody-drug conjugate according to claim 15, wherein in Formula (III),
L₁ is 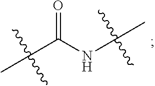 [SMILES: *NC(*)=O]
M is selected from the group consisting of 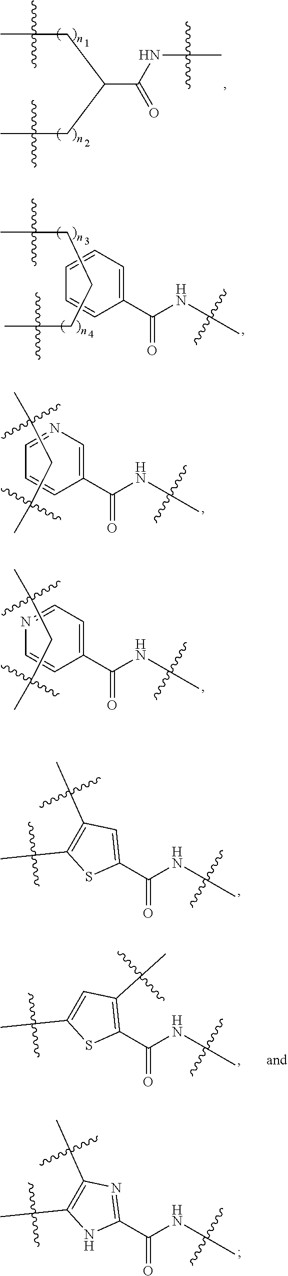
n₁ and n₂ are the same and each independently represent an integer from 0 to 8, and
n₃ and n₄ are the same and each independently represent an integer from 0 to 8;
L₂ is 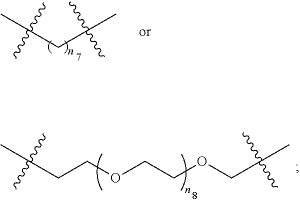
n₇ is an integer from 1 to 10, and n₈ is an integer from 1 to 10;
L₃ is 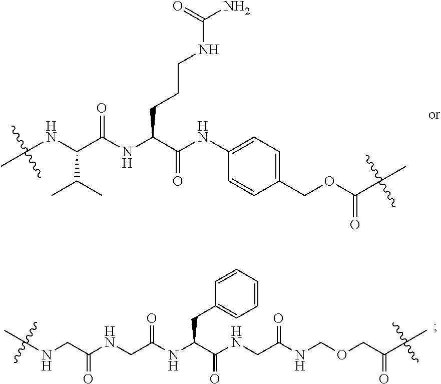 [SMILES: *N[C@H](C(=O)N[C@@H](CCCNC(N)=O)C(=O)Nc1ccc(COC(*)=O)cc1)C(C)C ; *NCC(=O)NCC(=O)N[C@@H](Cc1ccccc1)C(=O)NCC(=O)NCOCC(*)=O]
the cytotoxic small-molecule drug is a drug for the treatment of tumor diseases;
the active agent is a tubulin inhibitor, an RNA polymerase inhibitor, a topoisomerase inhibitor, an intercalator, a DNA-reactive agent, a DNA alkylating agent, or an immunosuppressant;
n₁₆ is an integer from 2 to 6;
the Ab is Patritumab, Sacituzumab, Mirvetuximab, Cetuximab, Trastuzumab, an anti-B7-H3 monoclonal antibody, Cirmtuzumab, or a mutant of the above antibodies at positions 226 or 229 of the heavy chain.

21. The antibody-drug conjugate according to claim 15, wherein the antibody-drug conjugate of Formula (III) is one or more selected from the group consisting of the compounds represented by the following structural formulas: 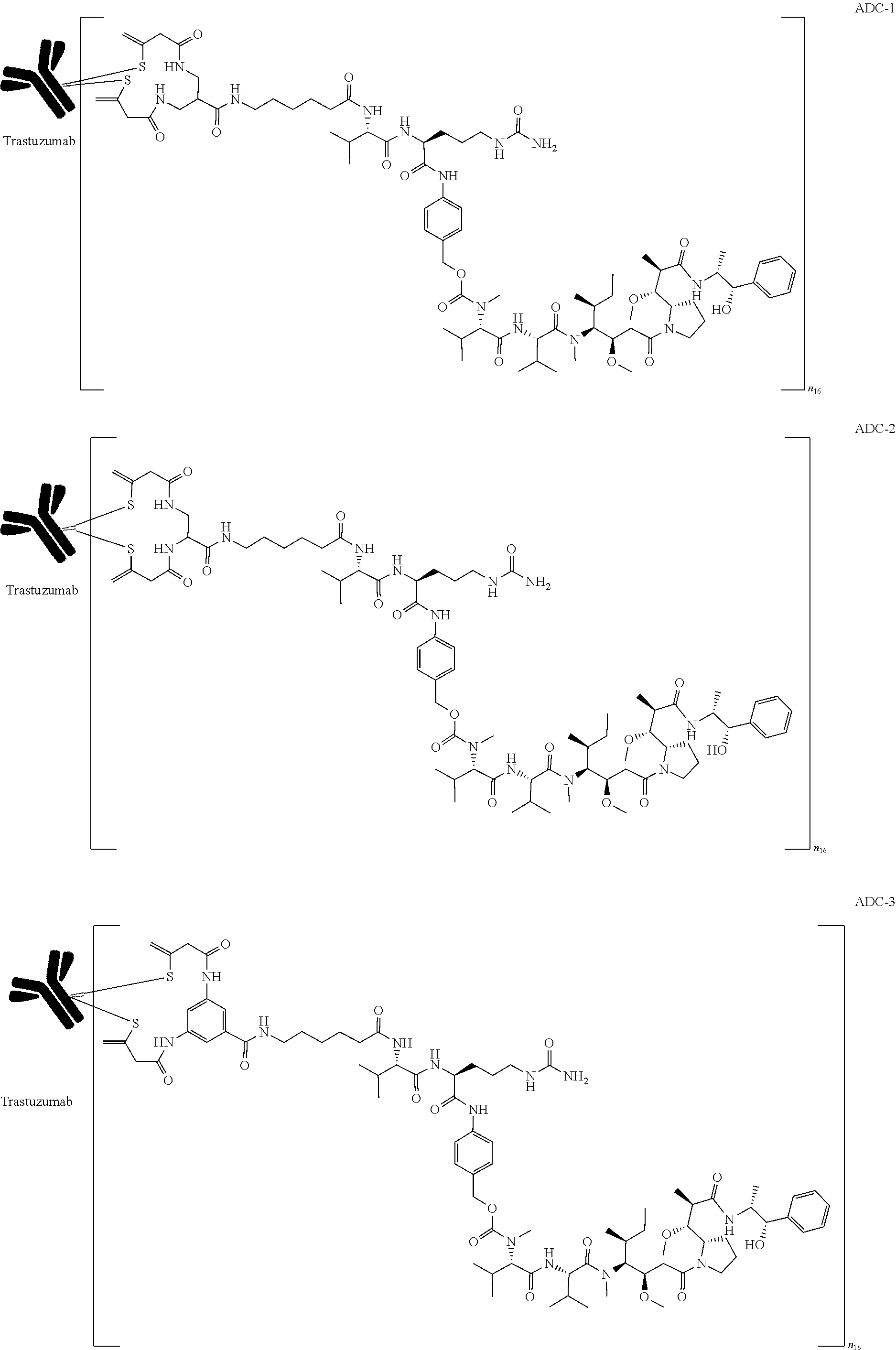 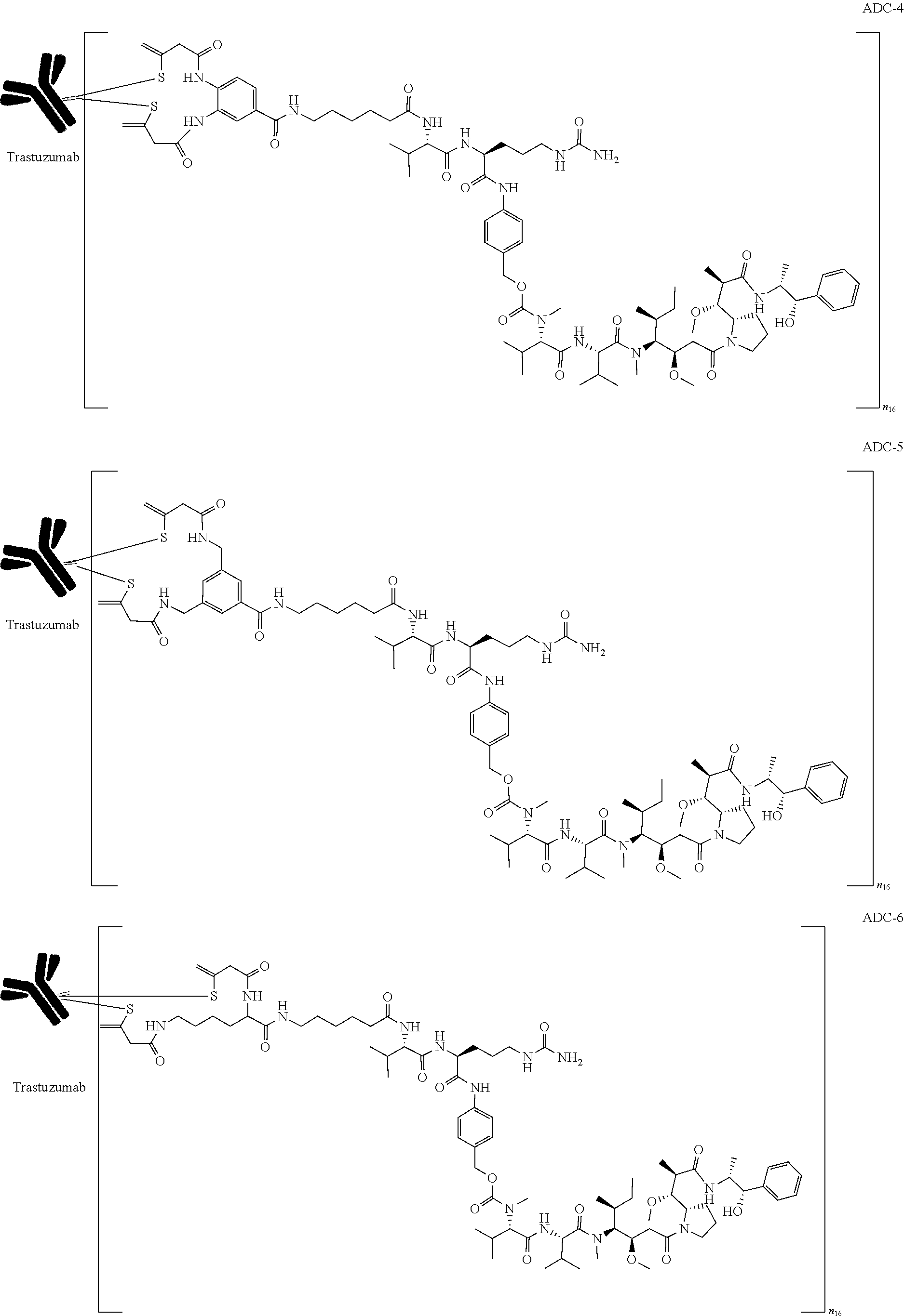 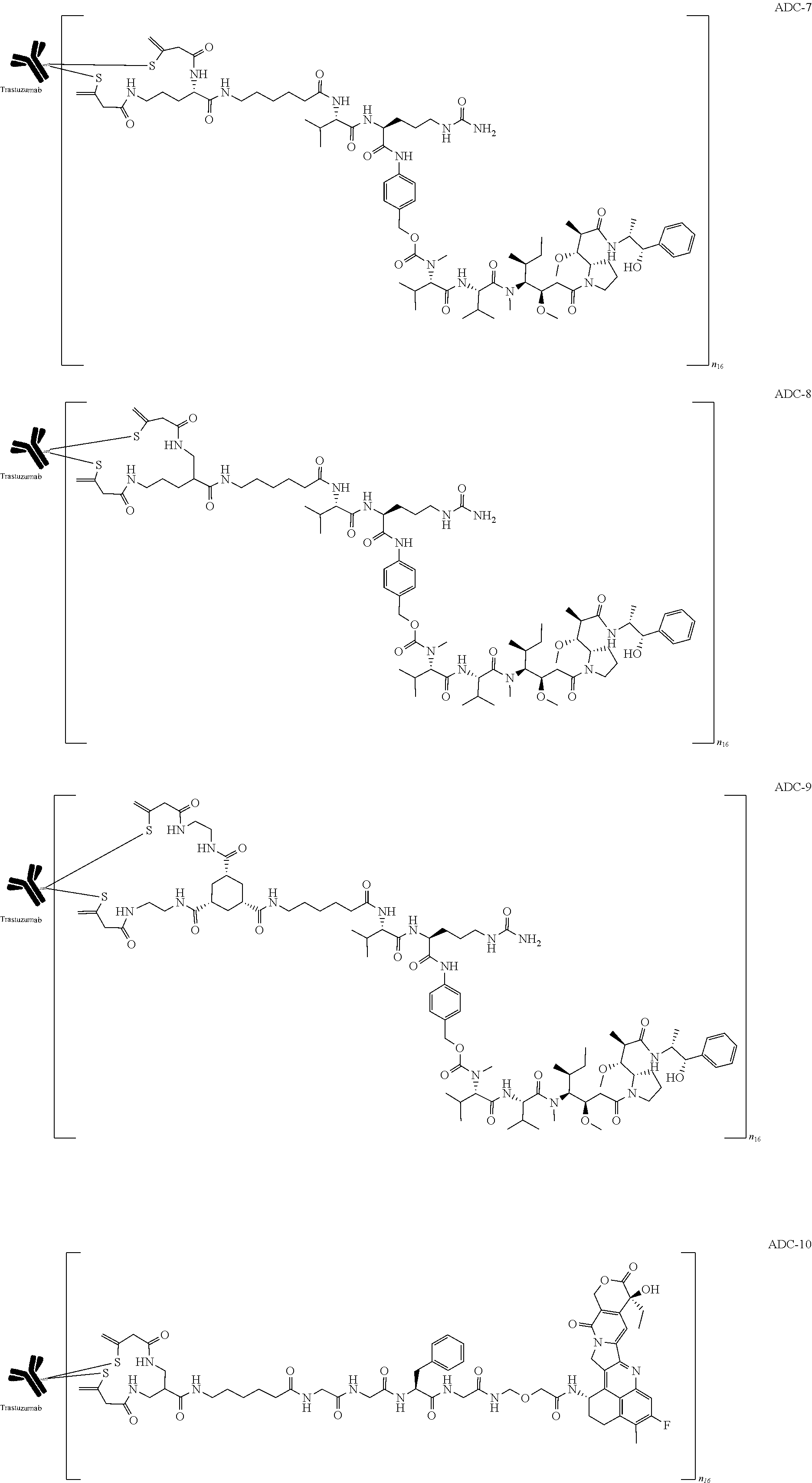 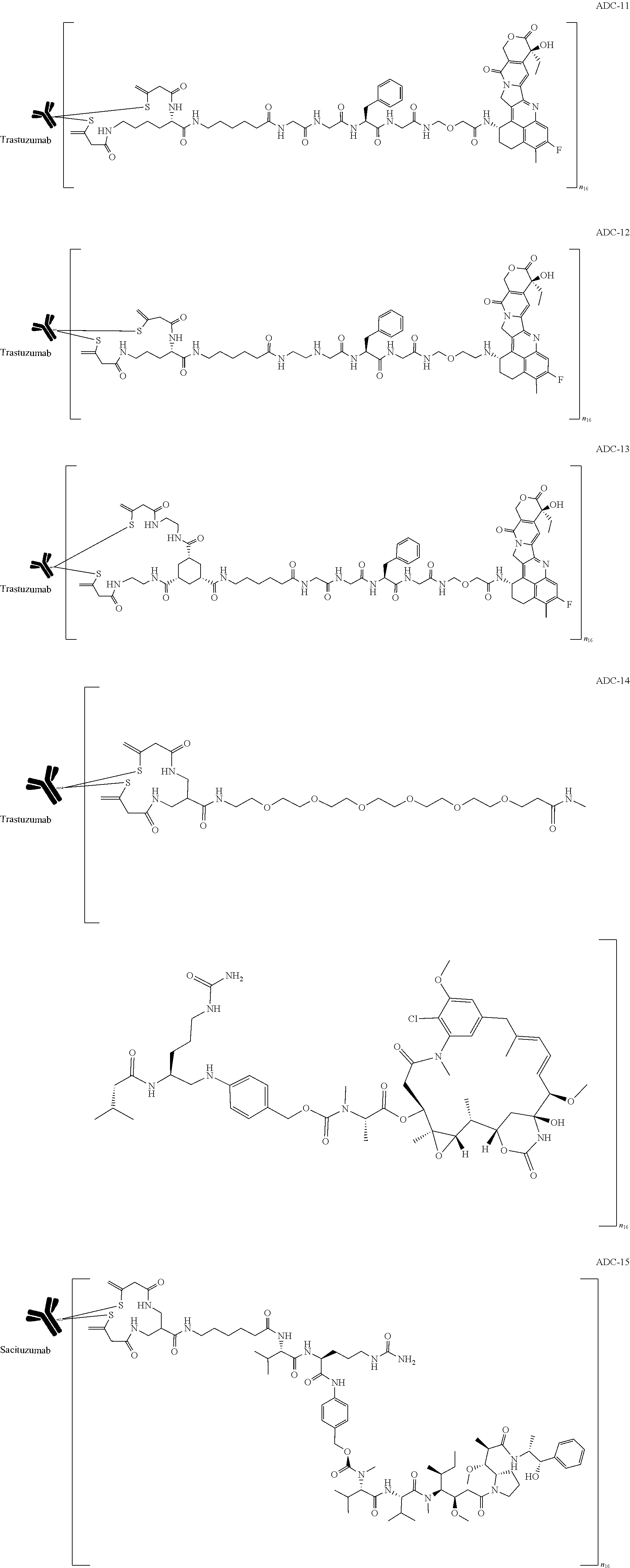 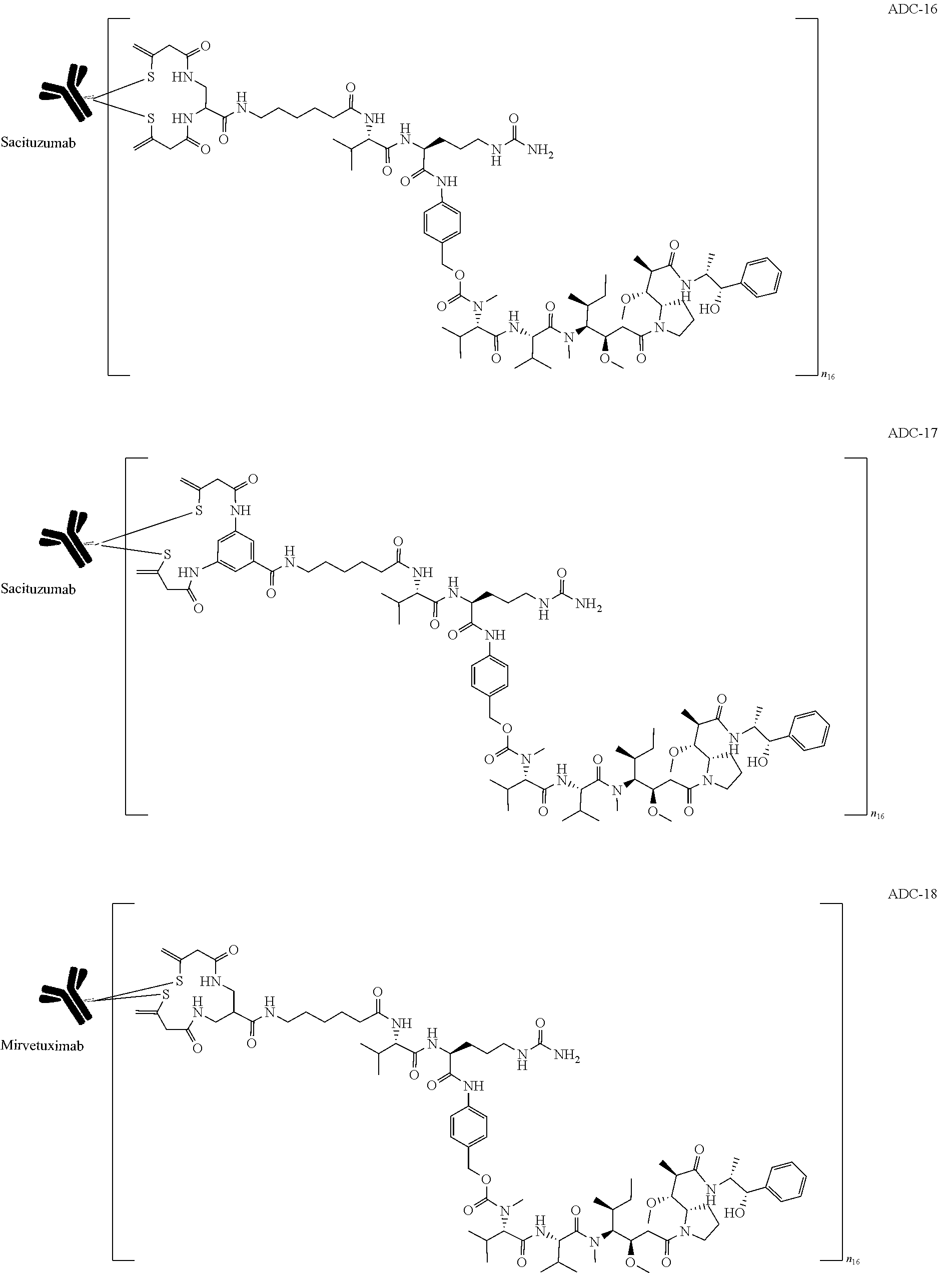 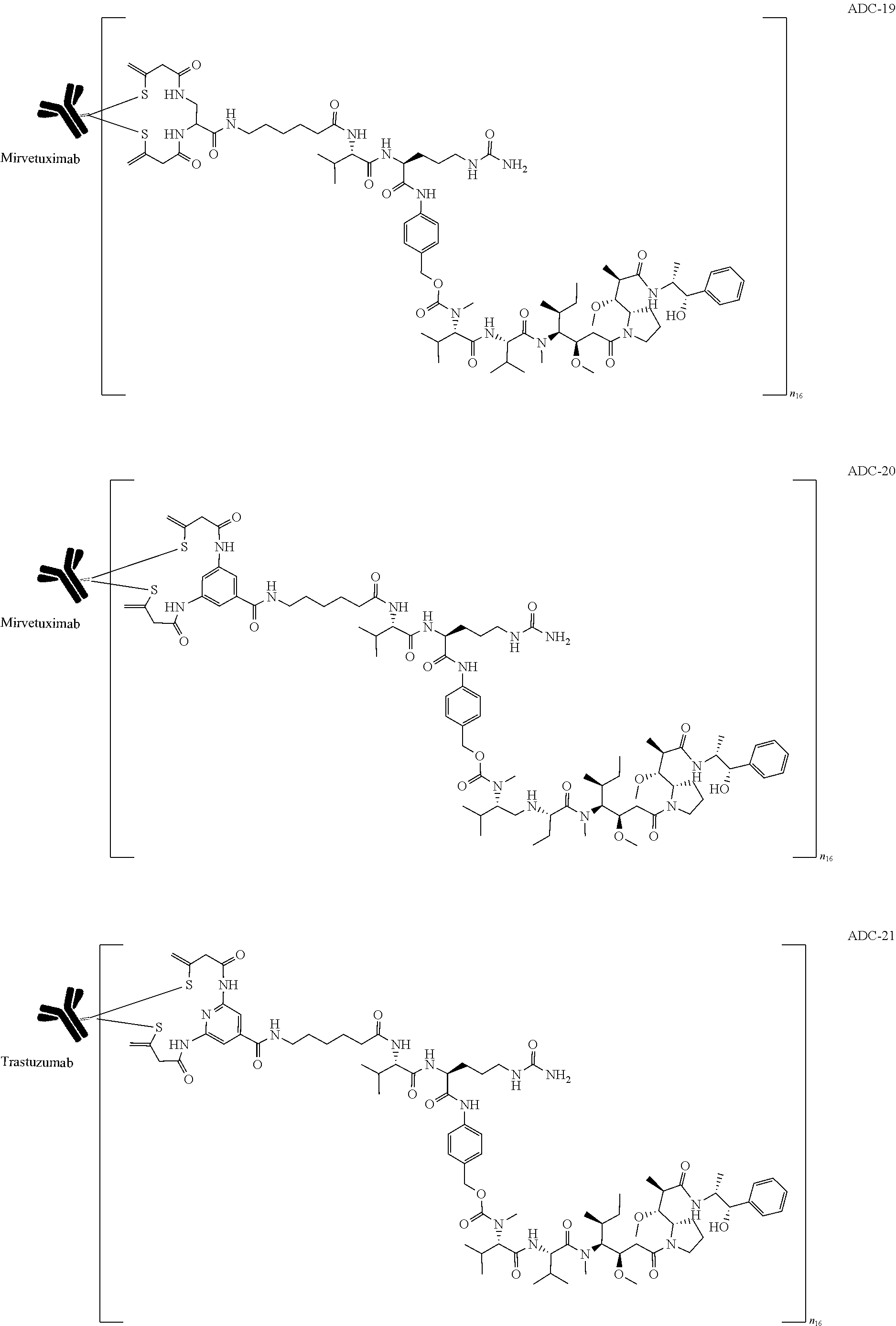 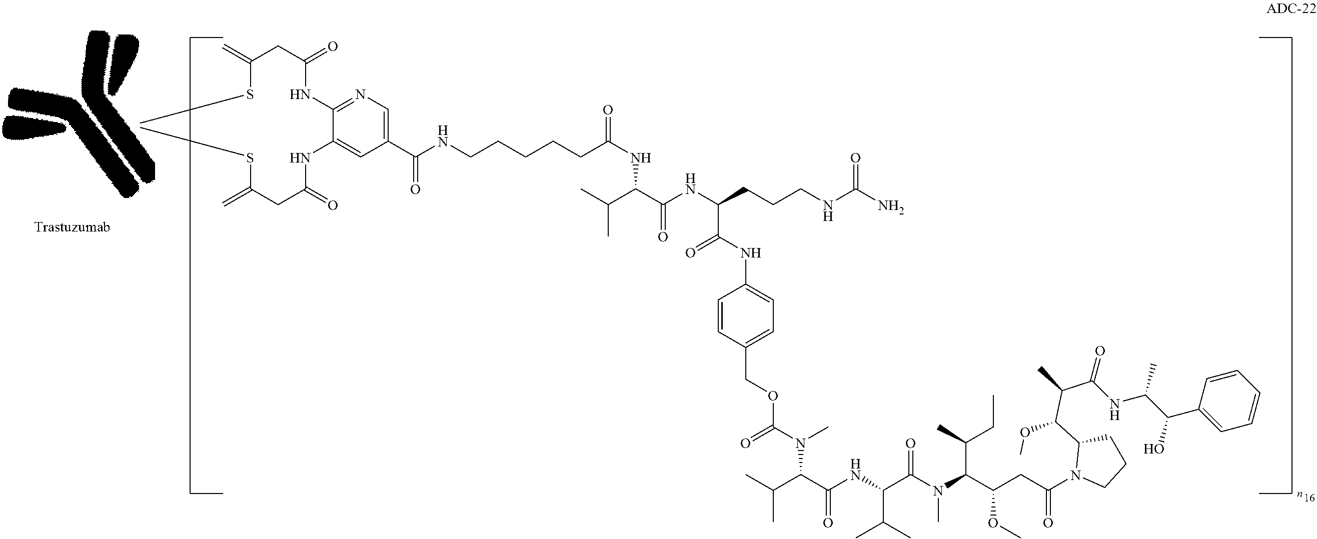 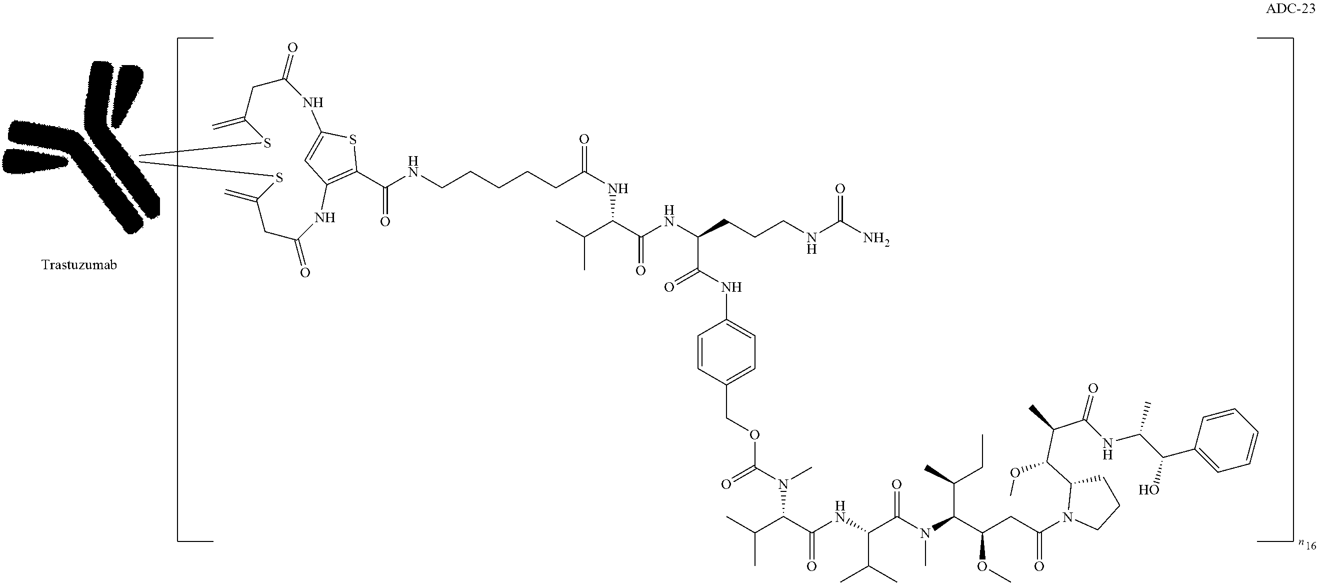 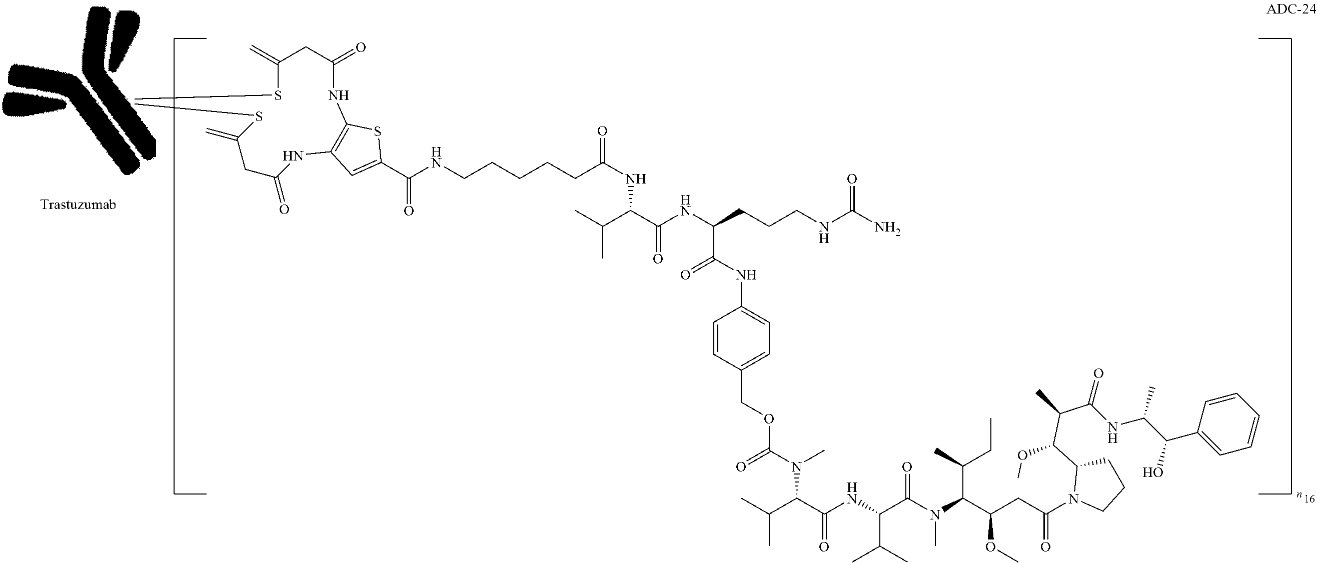 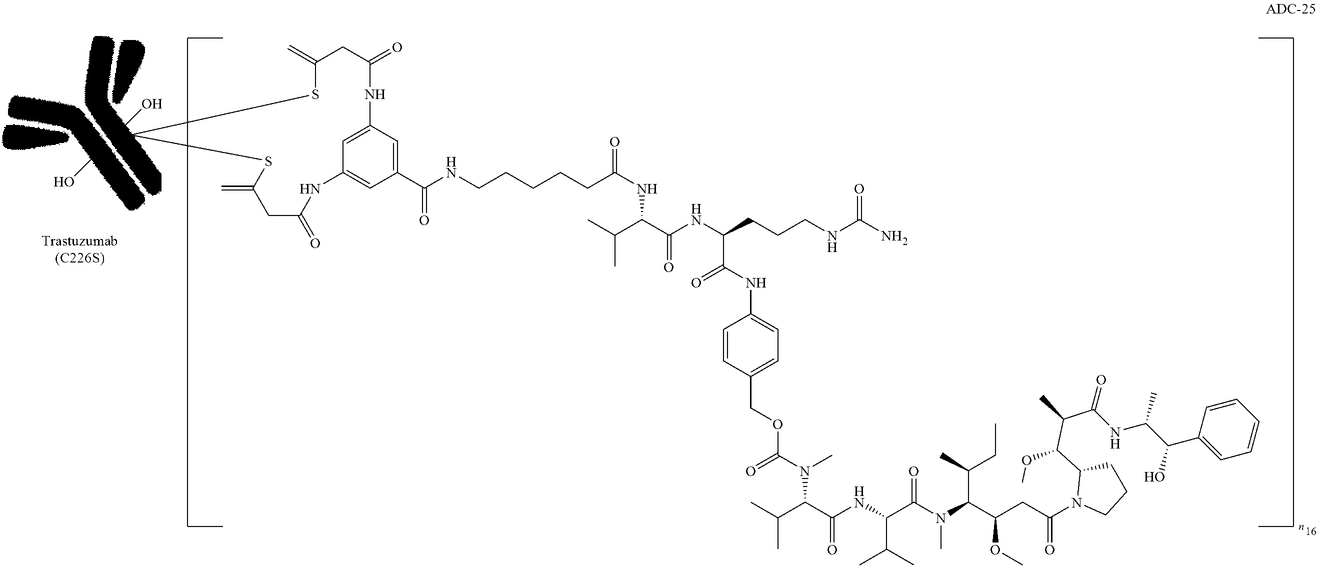 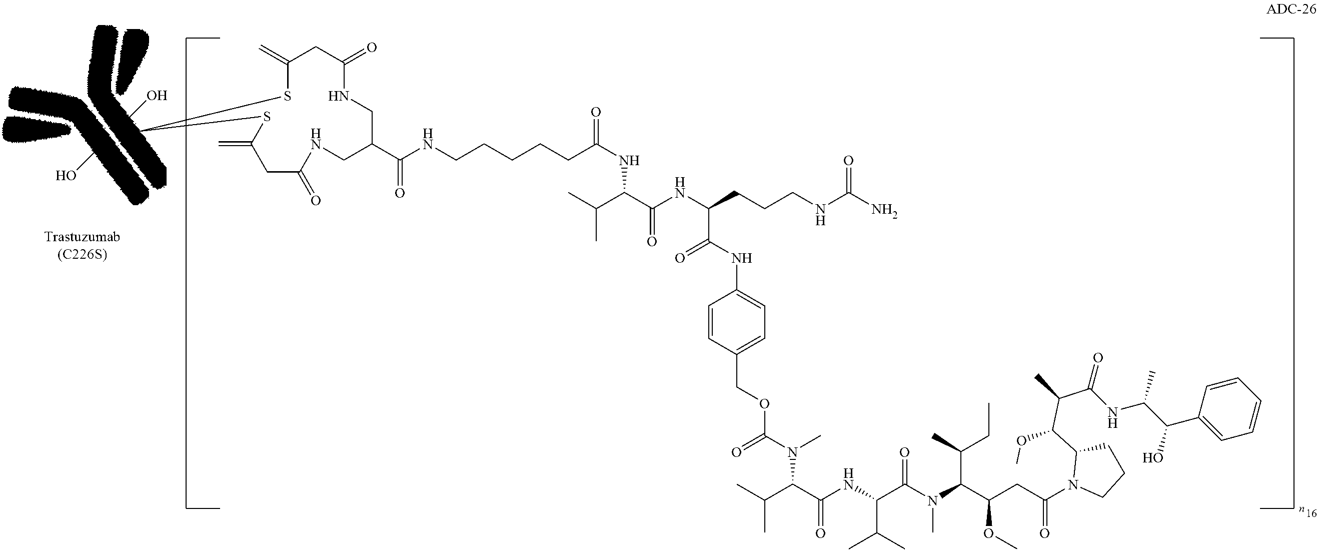 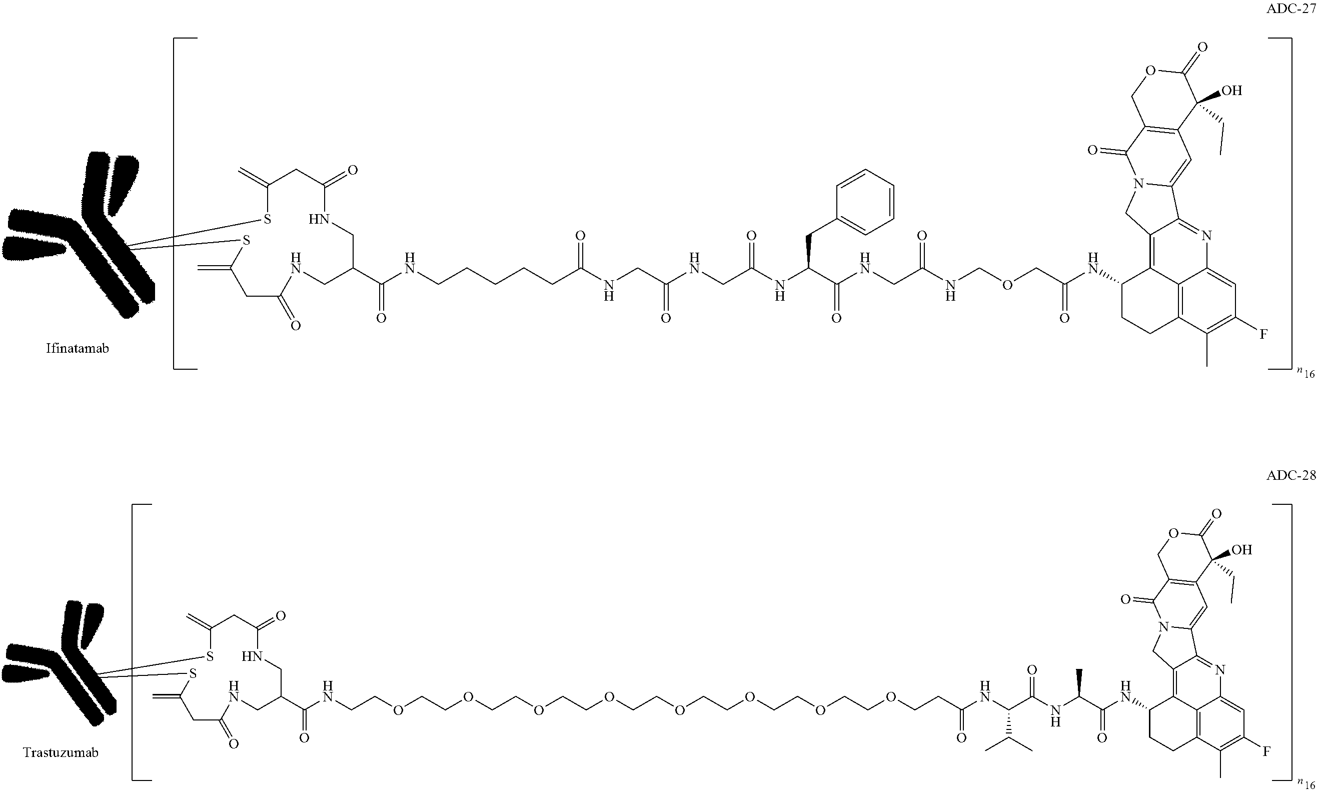 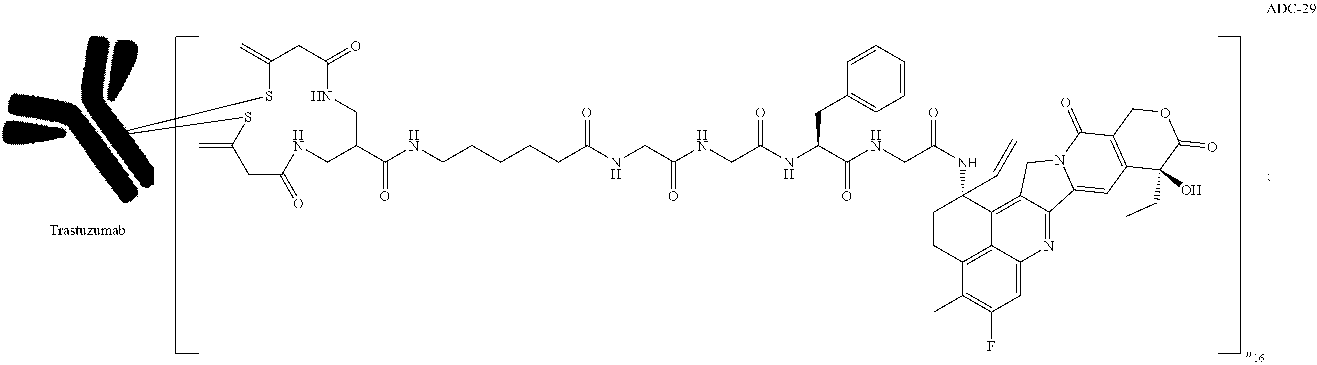
wherein n₁₆ is an integer from 2 to 6.

22. A method for the treatment of cancer comprising administering the antibody-drug conjugate according to claim 15 to a subject in need thereof.

23. The method according to claim 22, wherein the cancer is selected from the group consisting of lymphoma, blastoma, sarcoma, leukemia or lymphoid malignancy, squamous cell carcinoma, lung cancer, peritoneal cancer, liver cancer, gastric cancer, stomach cancer, pancreatic cancer, glioma, cervical tumor, ovarian cancer, oral cancer, bladder cancer, urinary tract cancer, breast cancer, HER2-positive breast cancer, colon cancer, rectal cancer, colorectal cancer, endometrial cancer or uterine carcinoma, salivary gland cancer, kidney cancer, prostate cancer, thyroid cancer, melanoma, multiple myeloma, B-cell lymphoma, brain cancer, head and neck cancer, and related metastatic tumors.
